SEQUENCE LISTING (21 sequences)

SEQ ID NO: 1 - RNA, 1653 nt, synthetic construct
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 2 - RNA, 1632 nt, synthetic construct
  source 1..1632: mol_type = other RNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 3 - protein, 532 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - RNA, 1602 nt, synthetic construct
  source 1..1602: mol_type = other RNA
atgtgccaccagcagctggtgatcagctggttcagcctggtgttcctggccagccccctggtggccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgatgagctacctgaacgccagctaatag

SEQ ID NO: 5 - DNA, 1602 bp, synthetic construct
  source 1..1602: mol_type = other DNA
atgtgccaccagcagctggtgatcagctggttcagcctggtgttcctggccagccccctggtggccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgatgagctacctgaacgccagctaatag

SEQ ID NO: 6 - RNA, 1835 nt, synthetic construct
  source 1..1835: mol_type = other RNA
aggaaattccatttggctgcagcttctggagggagccgacaggagacgtggggagacggccaccatgtgccaccagcagctggtgatcagctggttcagcctggtgttcctggccagccccctggtggccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgatgagctacctgaacgccagctaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 1835 bp, synthetic construct
  source 1..1835: mol_type = other DNA
aggaaattccatttggctgcagcttctggagggagccgacaggagacgtggggagacggccaccatgtgccaccagcagctggtgatcagctggttcagcctggtgttcctggccagccccctggtggccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgatgagctacctgaacgccagctaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 64 nt, synthetic construct
  source 1..64: mol_type = other RNA
aggaaattccatttggctgcagcttctggagggagccgacaggagacgtggggagacggccacc

SEQ ID NO: 9 - RNA, 94 nt, synthetic construct
  source 1..94: mol_type = other RNA
gctgccttctgcggggcttgccttctggccatgcccttcttctctcccttgcacctgtacctcttggtctttgaataaagcctgagtaggaagt

SEQ ID NO: 10 - DNA, 64 bp, synthetic construct
  source 1..64: mol_type = other DNA
aggaaattccatttggctgcagcttctggagggagccgacaggagacgtggggagacggccacc

SEQ ID NO: 11 - DNA, 94 bp, synthetic construct
  source 1..94: mol_type = other DNA
gctgccttctgcggggcttgccttctggccatgcccttcttctctcccttgcacctgtacctcttggtctttgaataaagcctgagtaggaagt

SEQ ID NO: 12 - RNA, 75 nt, synthetic construct
  source 1..75: mol_type = other RNA
aaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 75 bp, synthetic construct
  source 1..75: mol_type = other DNA
aaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 17 bp, synthetic construct
  source 1..17: mol_type = other DNA
taatacgactcactata

SEQ ID NO: 15 - DNA, 27 bp, synthetic construct
  source 1..27: mol_type = other DNA
ttggaccctcgtacagaagctaatacg

SEQ ID NO: 16 - DNA, 105 bp, synthetic construct
  source 1..105: mol_type = other DNA
tttttttttttttttttttttttttttttttttttttttttttttttttttttttttttttttttttttttttttacttcctactcaggctttattcaaagacca

SEQ ID NO: 17 - protein, 532 aa, synthetic construct; identical to SEQ ID NO: 3

SEQ ID NO: 18 - RNA, 1602 nt, synthetic construct
  source 1..1602: mol_type = other RNA
tgtgaccaccagcagctggtgatcagctggttcagcctggtgttcctggccagccccctggtggccatctgggagctgaagaaggacgtgtacgtggtggagctggactggtaccccgacgcccccggcgagtgagtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgtgagtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggctgattcccctgcctgcaccacagccagaacctgctgcgcgccgtgagcaactgactgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttctgtgaagccctgtgcctgagcagcatctacgaggacctgaagtgataccaggtggagttcaagacctgaaacgccaagctgctgtgagaccccaagcgccagatcttcctggaccagaactgactggccgtgatcgacgagctgtgacaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgtgaagctacctgaacgccagctaatag

SEQ ID NO: 19 - DNA, 1602 bp, synthetic construct
  source 1..1602: mol_type = other DNA
tgtgaccaccagcagctggtgatcagctggttcagcctggtgttcctggccagccccctggtggccatctgggagctgaagaaggacgtgtacgtggtggagctggactggtaccccgacgcccccggcgagtgagtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgtgagtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggctgattcccctgcctgcaccacagccagaacctgctgcgcgccgtgagcaactgactgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttctgtgaagccctgtgcctgagcagcatctacgaggacctgaagtgataccaggtggagttcaagacctgaaacgccaagctgctgtgagaccccaagcgccagatcttcctggaccagaactgactggccgtgatcgacgagctgtgacaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgtgaagctacctgaacgccagctaatag

SEQ ID NO: 20 - RNA, 1835 nt, synthetic construct
  source 1..1835: mol_type = other RNA
aggaaattccatttggctgcagcttctggagggagccgacaggagacgtggggagacggccacctgtgaccaccagcagctggtgatcagctggttcagcctggtgttcctggccagccccctggtggccatctgggagctgaagaaggacgtgtacgtggtggagctggactggtaccccgacgcccccggcgagtgagtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgtgagtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggctgattcccctgcctgcaccacagccagaacctgctgcgcgccgtgagcaactgactgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttctgtgaagccctgtgcctgagcagcatctacgaggacctgaagtgataccaggtggagttcaagacctgaaacgccaagctgctgtgagaccccaagcgccagatcttcctggaccagaactgactggccgtgatcgacgagctgtgacaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgtgaagctacctgaacgccagctaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

SEQ ID NO: 21 - DNA, 1835 bp, synthetic construct
  source 1..1835: mol_type = other DNA
aggaaattccatttggctgcagcttctggagggagccgacaggagacgtggggagacggccacctgtgaccaccagcagctggtgatcagctggttcagcctggtgttcctggccagccccctggtggccatctgggagctgaagaaggacgtgtacgtggtggagctggactggtaccccgacgcccccggcgagtgagtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgcgctgcgaggccaagaactacagcggccgcttcacctgctggtggctgaccaccatcagcaccgacctgaccttcagcgtcaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgtgagtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaacgccagcatcagcgtgcgcgcccaggaccgctactacagcagcagctggagcgagtgggccagcgtgccctgcagcggcggcggcggcggcggcagccgcaacctgcccgtggccacccccgaccccggctgattcccctgcctgcaccacagccagaacctgctgcgcgccgtgagcaactgactgcagaaggcccgccagaccctggagttctacccctgcaccagcgaggagatcgaccacgaggacatcaccaaggacaagaccagcaccgtggaggcctgcctgcccctggagctgaccaagaacgagagctgcctgaacagccgcgagaccagcttcatcaccaacggcagctgcctggccagccgcaagaccagcttctgtgaagccctgtgcctgagcagcatctacgaggacctgaagtgataccaggtggagttcaagacctgaaacgccaagctgctgtgagaccccaagcgccagatcttcctggaccagaactgactggccgtgatcgacgagctgtgacaggccctgaacttcaacagcgagaccgtgccccaaaagagcagcctggaggagcccgacttctacaagaccaagatcaagctgtgcatcctgctgcacgccttccgcatccgcgccgtgaccatcgaccgcgtgtgaagctacctgaacgccagctaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0387486 A1
(43) Pub. Date: Dec. 25, 2025

(54) LIPID COMPOSITION

(71) Applicant: STEMIRNA THERAPEUTICS CO., LTD., Shanghai (CN)

(72) Inventors: Yu HANG, Shanghai (CN); Lei HUANG, Shanghai (CN); Haifa SHEN, Shanghai (CN); Hangwen LI, Shanghai (CN)

(21) Appl. No.: 19/102,638

(22) PCT Filed: Aug. 8, 2023
(86) PCT No.: PCT/CN2023/111750

§ 371 Date: Feb. 10, 2025

(30) Foreign Application Priority Data
Aug. 9, 2022 (CN) 202210951311.0

(51) Int. Cl. A61K 47/18; A61K 9/127; C07C 229/34; C12N 15/11

(52) U.S. Cl. CPC A61K 47/18; A61K 9/127; C07C 229/34; C12N 15/11

(57) ABSTRACT

Provided is a drug delivery system suitable for tumor administration, which particularly relates to a lipid composition suitable for intratumoral administration. A therapeutic agent and/or a preventive the lipid composition comprises is RNA and suitable for intratumoral administration, and the lipid composition can be used to deliver the therapeutic agent and/or the preventive to a mammalian tumor so as to regulate and control the expression of a polypeptide, a protein or a gene.

### CROSS-REFERENCE TO RELATED APPLICATIONS

The present application claims priority to Chinese Patent Application No. 202210951311.0 filed on Aug. 9, 2022, which is incorporated herein by reference in their entirety.

### REFERENCE TO AN ELECTRONIC SEQUENCE LISTING

The contents of the electronic sequence listing (sequence listing.xml; Size: 37,868 bytes; and Date of Creation: Feb. 10, 2025) is herein incorporated by reference in its entirety.

### TECHNICAL FIELD

The present application relates to a tumor drug delivery system, and in particular, to a lipid composition suitable for intratumoral injection, a related product, and use in cancer therapy.

### BACKGROUND

A lipid-containing nanoparticle composition, a liposome and a lipoplex, as a transport vehicle, can effectively transport a bioactive substance such as a small molecule drug, a protein and a nucleic acid into a cell and/or an intracellular compartment. These lipid compositions generally include cationic lipids, structured lipids, helper lipids and/or surfactants.

At present, a plurality of lipid-based drug delivery systems have been developed, such as liposome and lipid nanoparticle (LNP) drug delivery systems. However, in practical use, especially when applied to tumor administration, it has been found that these lipid-based drug delivery systems have many problems. For example, when LNP combinations are used for intratumoral injection, they can be expressed locally in the tumor, and a large part thereof can be expressed in the liver, thus posing a risk of hepatotoxicity. Thus, despite significant advances in the research of lipid-based drug delivery systems, there remains a need for lipid delivery systems that are more efficient, stable, and have a good targeting effect.

At present, many companies mainly solve the poor tumor targeting effect of lipid drug delivery systems by design (see, for example, S. L. Hewitt et al., Intratumoral IL12 mRNA therapy promotes TH1 transformation of the tumor microenvironment, Clinical Cancer Research 26(23)(2020)6284-6298) or modification (see, for example, C. Hotz, T. R. et al., Local delivery of mRNA-encoded cytokines promotes antitumor immunity and tumor eradication across multiple preclinical tumor models, Science Translational Medicine 13(610)(2021)eabc7804) of mRNA sequences. This technical problem is solved by further optimizing the lipid drug delivery system.

### BRIEF SUMMARY OF THE INVENTION

An aspect of the present disclosure provides a lipid composition, including a therapeutic agent or a prophylactic agent and a lipid encapsulating the therapeutic agent or the prophylactic agent, wherein the lipid encapsulating the therapeutic agent or the prophylactic agent includes a cationic lipid, a phospholipid, a steroid, and a polyethylene glycol modified lipid; and the composition further includes a cationic polymer, wherein the cationic polymer and the therapeutic agent or the prophylactic agent are associated as a complex and co-encapsulated in the lipid to form a lipopolyplex; and wherein the cationic lipid includes a lipid compound of formula (I), or a pharmaceutically acceptable salt thereof, as defined herein.

In an embodiment, the therapeutic agent or the prophylactic agent is a nucleic acid, such as an RNA, in particular an mRNA.

In an embodiment, the cationic lipid is M5.

In an embodiment, the cationic lipid is SW-II-127, SW-II-135-1, or SW-II-138-1.

In an embodiment, the lipid composition includes [0012]10-70 mol % of the cationic lipid, 10-70 mol % of the phospholipid, 10-70 mol % of the steroid, and 0.05-20 mol % of the polyethylene glycol modified lipid;
preferably including 30-45 mol % of the cationic lipid, 10-20 mol % of the phospholipid, 30-48.5 mol % of the steroid, and 1-1.5 mol % of the polyethylene glycol modified lipid; and/or
the cationic lipid, DOPE, the cholesterol, and DMG-PEG; and
most preferably including 40% of the cationic lipid, 15% of DOPE, 43.5% of the cholesterol, and 1.5% of DMG-PEG.

In an embodiment, the therapeutic agent or the prophylactic agent is a polynucleotide, the polynucleotide includes a coding region, and the coding region encodes IL-12, wherein the IL-12 includes an amino acid sequence of SEQ ID NO: 3 or an amino acid sequence having at least 95% identity to the amino acid sequence of SEQ ID NO: 3; and wherein the polynucleotide is an RNA, wherein the coding region includes a nucleotide sequence of SEQ ID NO: 4 or a nucleotide sequence having at least 85% identity to the nucleotide sequence of SEQ ID NO: 4; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In some embodiments, the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

In an aspect, the present disclosure further provides a pharmaceutical composition, including the lipid composition of the present disclosure, and optionally a pharmaceutically acceptable excipient.

In another aspect, the lipid composition provided by the present disclosure or the pharmaceutical composition of the present disclosure is used for tumor administration; the tumor administration preferably includes intratumoral administration, peritumoral subcutaneous administration, or administration in an artery that supplies blood to a tumor, and most preferably intratumoral injection.

In yet another aspect, the present disclosure further provides an intratumoral injectant, including the lipid composition of the present disclosure, and optionally a pharmaceutically acceptable excipient for preparation of an injectable preparation.

In yet another aspect, the present disclosure further provides use of the lipid composition of the present disclosure, the pharmaceutical composition of the present disclosure, or the intratumoral injectant of the present disclosure in preparation of a drug for treating or preventing a cancer of a subject in need thereof.

In yet another aspect, the present disclosure further provides a method for preventing or treating a cancer of a subject in need thereof, including administrating to the subject in need thereof the lipid composition of the present disclosure, the pharmaceutical composition of the present disclosure, or the intratumoral injectant of the present disclosure. The lipid composition and the pharmaceutical composition can be applied by intratumoral administration, peritumoral subcutaneous administration, or administration in an artery that supplies blood to a tumor, and preferably intratumoral injection.

In yet another aspect, the present disclosure further provides a method for delivering a therapeutic agent or a prophylactic agent to a mammalian tumor of a subject, including administering to the subject the lipid composition or the pharmaceutical composition of the present disclosure, the administering including bringing the tumor into contact with the lipid composition or the pharmaceutical composition, thereby delivering the therapeutic agent and/or the prophylactic agent to the tumor.

In another aspect, the present disclosure further provides a method for producing a polypeptide of interest in a mammalian tumor of a subject, including bringing the tumor into contact with the lipid composition or the pharmaceutical composition of the present disclosure, wherein the therapeutic agent or the prophylactic agent is an mRNA, and wherein the mRNA encodes a polypeptide of interest, whereby the mRNA is capable of being translated in the tumor to produce the polypeptide of interest.

### BRIEF DESCRIPTION OF THE DRAWINGS

FIGS. 1A-FIG. 1D show results of luciferase expression of LNP or LPP preparations prepared by different prescriptions with a cationic lipid being M5 by intratumoral injection. FIG. 1A shows luciferase expression in mice. FIG. 1B shows results of luciferase expression in livers and tumors of mice. FIG. 1C shows ratios of luciferase expression of liver/tumor. FIG. 1D shows ratios of luciferase expression of tumor/whole body.

FIGS. 2A-2B show effects of tumor suppression of LNP or LPP preparations prepared by different prescriptions with a cationic lipid being M5 on B16F10 tumor-bearing mice. FIG. 2A shows results of tumor volumes. FIG. 2B shows results of weight changes.

FIGS. 3A-3B show effects of tumor suppression of LNP or LPP preparations prepared by different prescriptions with a cationic lipid being M5 on A20 tumor-bearing mice. FIG. 3A shows results of tumor volumes. FIG. 3B shows results of weight changes.

FIG. 4 shows expression of SW0715 in A375 tumor-bearing mice.

FIG. 5 shows expression of SW0715 in A375 cells and MDA-MB-231 cells being dose-dependent.

FIG. 6 shows an expression product of SW0715 that can effectively activate primary CD8⁺T cells in vitro.

FIG. 7 shows effects of tumor suppression of SW0715 on a humanized mouse MDA-MB-231 subcutaneous graft tumor model.

### DETAILED DESCRIPTION OF THE INVENTION

### General Definitions and Terms

All patents, patent applications, scientific publications, manufacturer's instructions and guidelines, regardless of the preceding or following text, cited herein are incorporated herein by reference in their entirety. Any content herein needs not to be construed as an admission that the present disclosure is not entitled to antedate such disclosure.

Unless otherwise indicated, scientific and technical terms used herein have the meaning commonly understood by those skilled in the art. Furthermore, the terms related to protein and nucleic acid chemistry, molecular biology, cell and tissue culture, and microbiology used herein are all widely used in the corresponding fields. Meanwhile, to better understand the present disclosure, definitions and explanations of related terms are provided below.

As used herein, the expressions “comprising”, “including”, “containing” and “having” are inclusive and mean including the listed elements, steps or components but not excluding other unlisted elements, steps or components. The expression “consisting of” does not include any element, step or component not specified. The expression “consisting substantially of” means the scope limited to the specified element, step or component, plus an optionally present element, step or component that does not significantly affect the basic and novel properties of the claimed subject matter. It needs to be understood that the expressions “consisting substantially of” and “consisting of” are included within the meaning of the expression “comprising”.

As used herein, the expression in singular form “a”, “an”, or “the” includes plural references unless the context indicates otherwise. The term “one or more” or “at least one” encompasses 1, 2, 3, 4, 5, 6, 7, 8, 9 or more.

The list of the range of values herein is solely for use as a shorthand method of referring individually to each different value falling within the range. Unless otherwise indicated herein, each individual value is incorporated into the specification as individually listed herein. Unless explicitly stated to the contrary, a numerical value or range shown herein is modified by “about”, meaning that the listed or claimed numerical value or range is ±20%, ±10%, +5%, or +3%.

Unless otherwise indicated, all methods described herein may be performed in any suitable order.

Herein, “nucleotide” includes deoxyribonucleotide, ribonucleotide, and derivatives thereof. As used herein, “ribonucleotide” is a constitutive substance of ribonucleic acid (RNA), consists of one molecular of base, one molecule of pentose, and one molecule of phosphoric acid, and refers to a nucleotide having a hydroxyl at a 2′-position of a β-D-ribofuranosyl group. However, “deoxyribonucleotide” is a constitutive substance of deoxyribonucleic acid (DNA), also consists of one molecular of base, one molecule of pentose, and one molecule of phosphoric acid, refers to a nucleotide having a hydroxyl substituted with hydrogen at a 2′-position of a 3-D-ribofuranosyl group, and is a main chemical component of a chromosome. “Nucleotide” is generally referred to by a single letter representing the base therein: “A(a)” refers to an adenine-containing deoxyadenylic acid or adenylic acid, “C(c)” refers to a cytosine-containing deoxycytidylic acid or cytidylic acid, “G(g)” refers to a guanine-containing deoxyguanylic acid or guanylic acid, “U(u)” refers to a uracil-containing uridylic acid, and “T(t)” refers to a thymine-containing deoxythymidylic acid.

As used herein, the terms “polynucleotide” and “nucleic acid” are used interchangeably to refer to a polymer of deoxyribonucleotides (deoxyribonucleic acid, DNA) or a polymer of ribonucleotides (ribonucleic acid, RNA). “Polynucleotide sequence”, “nucleic acid sequence”, and “nucleotide sequence” are used interchangeably to denote the ordering of nucleotides in a polynucleotide. Those skilled in the art need to understand that a DNA coding strand (sense strand) and an RNA encoded thereby can be regarded as having the same nucleotide sequence, and a deoxythymidylic acid in a DNA coding strand sequence corresponds to a uridylic acid in an RNA sequence encoded thereby.

As used herein, the term “% identity” in reference to a sequence refers to the percentage of nucleotides or amino acids that are the same in an optimal alignment between the sequences to be compared. The difference between two sequences may be distributed in the local region (segment) or across the entire length of the sequences to be compared. The identity between two sequences is generally determined after the optimal alignment of segments or “comparison windows”. The optimal alignment may be performed manually, or with the aid of algorithms known in the art, including but not limited to local homology algorithms described in Smith and Waterman, 1981, Ads App. Math. 2,482 and Neddleman and Wunsch, 1970, J. Mol. Biol. 48,443, and a similarity search method described in Pearson and Lipman, 1988, Proc. Natl Acad. Sci. USA 88,2444, or performed by using computer programs, such as GAP, BESTFIT, FASTA, BLAST P, BLAST N, and TFASTA in Wisconsin Genetics Software Package, Genetics Computer Group, 575 Science Drive, Madison, Wis. For example, the percentage identity of two sequences can be determined by using the BLASTN or BLASTP algorithms publicly available at the National Center for Biotechnology Information (NCBI) website.

The % identity is obtained by determining the number of identical positions corresponding to the sequences to be compared, dividing the number by the number of positions compared (e.g., the number of positions in a reference sequence), and multiplying the result by 100. In some embodiments, the degree of identity is given to at least about 50%, at least about 55%, at least about 60%, at least about 65%, at least about 70%, at least about 75%, at least about 80%, at least about 85%, at least about 90%, at least about 95%, or about 100% of the region. In some embodiments, the degree of identity is given to the entire length of the reference sequence. The alignment for determining sequence identity can be performed by using tools known in the art, preferably by using optimal sequence alignment, e.g., by using Align, and by using standard settings, preferably EMBOSS::needle, Matrix:Blosum62, Gap Open 10.0, and Gap Extend 0.5.

As used herein, “modified” refers to unnatural. For example, an RNA may be a modified RNA. That is, the RNA may include one or more unnatural nucleobases, nucleosides, nucleotides, or linker groups. A “modified” group may also be referred to herein as an “altered” group. The group may be modified or altered chemically, structurally, or functionally. For example, a modified nucleobase may include one or more unnatural substitutions.

As used herein, the term “expression” includes transcription and/or translation of a nucleotide sequence. Thus, the expression may involve the production of a transcript and/or a polypeptide. The term “transcription” relates to the process of transcribing a genetic code in a DNA sequence into an RNA (a transcript). The term “in vitro transcription” refers to the in vitro synthesis of an RNA, in particular an mRNA, in a cell-free system (e.g., in an appropriate cell extract). A vector that can be used to produce a transcript is also referred to as “transcription vector”, which includes a regulatory sequence required for transcription. The term “transcription” encompasses “in vitro transcription”.

As used herein, the term “host cell” refers to a cell which is used to receive, maintain, replicate, and express a polynucleotide or vector.

As used herein, an “aliphatic” group is a non-aromatic group in which carbon atoms are connected into a chain, and may be saturated or unsaturated.

As used herein, the term “alkyl” refers to an optionally substituted straight or branched chain saturated hydrocarbon including one or more carbon atoms. The term “C₁-C₁₂ alkyl” or “C₁₋₁₂ alkyl” refers to an optionally substituted straight or branched chain saturated hydrocarbon including 1-12 carbon atoms. As used herein, the term “alkoxyl” refers to an alkyl described herein, which is connected to the remainder of a molecule via an oxygen atom. The term “alkylene” refers to a divalent group formed by the corresponding alkyl that loses one hydrogen atom. The term “C₁-C₁₂ alkylene” or “C₁₋₁₂ alkylene” refers to an optionally substituted straight or branched chain alkylene including 1-12 carbon atoms.

As used herein, the term “alkenyl” refers to an optionally substituted straight or branched chain hydrocarbon including two or more carbon atoms and at least one double bond. The term “C₂-C₁₂ alkenyl” or “C₂₋₁₂ alkenyl” refers to an optionally substituted straight or branched chain hydrocarbon including 2-12 carbon atoms and at least one carbon-carbon double bond. The alkenyl may include one, two, three, four or more carbon-carbon double bonds.

As used herein, the term “carbocycle” refers to a monocyclic or polycyclic non-aromatic system including one or more rings composed of carbon atoms. The term “C₃₋₈ carbocycle” means a carbocycle including 3-8 carbon atoms. A carbocycle may include one or more carbon-carbon double bonds or triple bonds. Instances of carbocycle include but are not limited to cyclopropyl, cyclopentyl, cyclohexyl, etc. As used herein, when the carbocycle is saturated (i.e., includes no unsaturated bond), the carbocycle also refers to a corresponding cycloalkyl. Unless specifically indicated otherwise, the carbocycle described herein refers to unsubstituted and substituted, i.e., optionally substituted, carbocycles.

As used herein, the term “heterocycle” refers to a monocyclic or polycyclic system including one or more rings and including at least one heteroatom. The heteroatom may be, for example, a nitrogen, oxygen, phosphorus or sulfur atom. The heterocycle may include one or more double bonds or triple bonds, and may be non-aromatic. Instances of heterocycle include but are not limited to imidazolidinyl, oxazolidinyl, thiazolidinyl, pyrazolidinyl, isoxazolidinyl, isothiazolidinyl, morpholinyl, pyrrolidinyl, tetrahydrofuranyl, and piperidinyl. The heterocycle may include, for example, 3-10 atoms (non-hydrogen), i.e., 3-10 membered heterocycle (e.g., 3, 4, 5, 6, 7, 8, 9, or 10 membered), wherein one or more atoms are heteroatoms (e.g., N, O, S, or P). When the heterocycle is saturated (i.e., includes no unsaturated bond), the heterocycle also refers to a corresponding heterocyclylalkyl. Unless specifically indicated otherwise, the heterocycle described herein refers to unsubstituted and substituted heterocyclic groups, i.e., optionally substituted heterocycles.

As used herein, the term “aryl” refers to a all-carbon monocyclic or fused polycyclic aromatic ring group having a conjugated π-electron system. For example, C₆-C₁₀ alkylaryl may have 6-10 carbon atoms, e.g., 6, 7, 8, 9, 10 carbon atoms. Instances of aryl include but are not limited to phenyl, naphthyl, etc.

As used herein, the term “heteroaryl” refers to a monocyclic or fused polycyclic system including at least one ring atom selected from N, O, or S, with the remaining ring atoms being C, and having at least one aromatic ring. The heteroaryl may have 5-10 ring atoms (5-10 membered heteroaryl), including 5, 6, 7, 8, 9 or 10 membered, in particular 5 or 6 membered heteroaryl. Instances of heteroaryl include but are not limited to pyrrolyl, furanyl, thienyl, imidazolyl, oxazolyl, pyrazolyl, pyridyl, pyrimidinyl, pyrazinyl, quinolinyl, isoquinolinyl, tetrazolyl, triazolyl, triazinyl, benzofuranyl, benzothienyl, indolyl, isoindolyl, etc.

Unless specifically indicated otherwise, the groups described herein (e.g., any one of R₁—R₇, such as alkyl, alkylene, alkenyl, aryl, amino, etc.) can be optionally substituted. The optional substituent group may be selected from, but is not limited to: halogen atom (e.g., chloro, bromo, fluoro, or iodo), carboxyl (e.g., —C(O)OH), alcohol group (e.g., hydroxyl, —OH), ester group (e.g., —C(O)OR or —OC(O)R), aldehyde group (e.g., —C(O)H), carbonyl (e.g., —C(O)R, or represented by C═O), acyl halide (e.g., —C(O)X, where X is a halo selected from bromo, fluoro, chloro, and iodo), carbonate group (e.g., —OC(O)OR), alkoxyl (e.g., —OR), acetal (e.g., —C(OR)₂R″″, where each OR is the identical or different alkoxyl and R″″ is alkyl or alkenyl), phosphate radical (e.g., P(O)₄³⁻), thiol (e.g., —SH), sulfinyl (e.g., —S(O)R), sulfino (e.g., —S(O)OH), sulfo (e.g., —S(O)₂OH), thioformyl (e.g., —C(S)H), sulfate radical (e.g., S(O)₄²⁻), sulfonyl (e.g., —S(O)₂—), acylamino (e.g., —C(O)NR₂ or —N(R)C(O)R), azido (e.g., —N₃), nitro (e.g., —NO₂), cyano (e.g., —CN), isocyano (e.g., —NC), acyloxy (e.g., —OC(O)R), amino (e.g., —NR₂, NRH, or —NH₂), carbamoyl (e.g., —OC(O)NR₂, —OC(O)NRH, or —OC(O)NH₂), sulfonamido (e.g., —S(O)₂NR₂, —S(O)₂NRH, —S(O)₂NH₂, —N(R)S(O)₂R, —N(H)S(O)₂R, —N(R)S(O)₂H, —N(H)S(O)₂H), C₁-C₁₂ alkyl, C₂-C₁₂ alkenyl, C₆-C₁₀ aryl, 5-10 membered heteroaryl, or 3-10 membered heterocycle. In any of the foregoing, each R independently may be a substituent group as defined herein, such as alkyl, alkoxyl, aryl, heteroaryl, or alkenyl. In some embodiments, the substituent group itself may be further substituted e.g., with one, two, three, four, five, or six substituent groups as defined herein. For example, the alkyl may be further substituted with one, two, three, four, five, or six substituent groups as described herein.

As used herein, the term “compound” is intended to include isotope compounds of the depicted structure. “Isotopes” refer to atoms having the same number of atoms but different mass numbers due to the different numbers of neutrons in a nucleus, e.g., deuterium isotopes. For example, isotopes of hydrogen include tritium and deuterium. In addition, the compound, salt, or complex of the present disclosure may be prepared in combination with a solvent or water molecule to form a solvate and a hydrate by a conventional method.

The term “optional” or “optionally” (e.g., optionally substituted) means that an event described subsequently may or may not occur, and the description includes instances where the event or situation occurs and instances where the event or situation does not occur. For example, “optionally substituted alkyl” means that alkyl may or may not be substituted, and the description includes substituted alkyl free radicals and unsubstituted alkyl free radicals.

It needs to be understood that when a chemical group is written in a particular order, the reverse order is also encompassed unless otherwise indicated. For example, in the general formula —(R)ᵢ-(M1)ₖ—(R)ₘ— where M₁ is defined as —C(O)NH— (i.e., —(R), —C(O)—NH—(R)ₘ—), a compound where M₁ is —NHC(O)— (i.e., —(R), —NHC(O)—(R)ₘ—) is also encompassed unless otherwise indicated.

As used herein, the term “contact” refers to the establishment of a physical connection between two or more entities. For example, bringing a mammalian cell into contact with a lipid composition means that the mammalian cell and the lipid nanoparticle share a physical connection. Methods for bringing a cell into contact with an external entity in vivo and in vitro are well known in the biological field. For example, bringing a lipid composition into contact with a mammalian cell in a mammalian body can be performed via different routes of administration (such as intratumoral), and may involve different amounts of lipid compositions. In addition, the lipid composition may make contact with more than one mammalian cell.

As used herein, the term “delivery” refers to providing an entity to a target. For example, delivering a therapeutic agent or a prophylactic agent to a subject may involve administering a composition including the therapeutic agent or the prophylactic agent to the subject.

As used herein, the term “subject” describes that an organism using the composition of the present disclosure can be provided thereto. Subjects that are expected to receive these compositions include but are not limited to humans, other primates, and other mammals such as cattle, swine, horses, sheep, cats, dogs, mice, or rats. Preferably, the subjects may be mammals, particularly humans.

As used herein, “lipid component” refers to a component of a composition including one or more lipids. For example, the lipid component may include one or more cationic lipids, pegylated lipids, structural lipids, or helper lipids.

The phrase “pharmaceutically acceptable” is used herein to refer to compounds, salts, materials, combinations, and/or dosage forms that are within a reasonable medical judgment range, suitable for use in contact with human and animal tissues without excessive toxicity, irritation, allergic reactions, or other issues or complications, and conform to a reasonable benefit/risk ratio.

As used herein, “pharmaceutically acceptable salt” refers to a derivative of a disclosed compound in which a parent compound is altered by converting an existing acid or base moiety into its salt form (e.g., by reacting a free basic group with a suitable organic acid). Instances of pharmaceutically acceptable salts include but are not limited to inorganic or organic acid salts of basic residues such as amine; and basic metals or organic salts of acidic residues such as carboxylic acid. Representative acid addition salts include but are not limited to acetate, adipate, alginate, ascorbate, aspartate, benzene sulfonate, benzoate, bisulfate, borate, butyrate, camphorate, camphorsulfonate, citrate, cyclopentylpropionate, digluconate, dodecyl sulfate, ethanesulphonate, fumarate, gluceptate, glycerophosphate, hemisulphate, enanthate, caproate, hydrobromide, hydrochloride, hydriodate, 2-hydroxy-ethanesulphonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectate, persulfate, 3-phenylpropionate, phosphate, picrate, pivalate, propionate, stearate, succinate, sulfate, tartrate, thiocyanate, cresylsulfonate, undecanoate, valerate, etc. Representative basic metal or alkaline earth metal salts include but are not limited to sodium, lithium, potassium, calcium, magnesium salts, etc.; and non-toxic ammonium, quaternary ammonium, and amine cations, including but not limited to ammonium, tetramethylammonium, tetraethylammonium, methylamine, dimethylamine, trimethylamine, triethylamine, ethylamine, etc. Pharmaceutically acceptable salts of the present disclosure include, for example, conventional non-toxic salts of a parent compound formed from non-toxic inorganic or organic acids. The pharmaceutically acceptable salt of the present disclosure may be synthesized by a parent compound including a basic or acidic moiety via a conventional chemical method. Generally speaking, these salts may be prepared by reacting the free acid or base form of these compounds with a stoichiometric amount of an appropriate base or acid in water or an organic solvent, or in a mixture of both; non-aqueous media such as ether, ethyl acetate, ethanol, isopropanol, or acetonitrile are generally preferred.

As used herein, the term “treating” refers to partially or completely alleviating, mitigating, ameliorating, or relieving one or more symptoms or features of a specific infection, disease, disorder, or condition, delaying its outbreak, inhibiting its progression, reducing its severity, or reducing its occurrence. “Preventing” refers to preventing potential diseases or preventing the deterioration of symptoms or the development of diseases.

The term “prophylactically or therapeutically effective amount” refers to the amount of a reagent (e.g., nucleic acid, drug, composition, therapeutic agent, diagnostic agent, prophylactic agent, etc.) that is sufficient to prevent or inhibit the occurrence of a disease or symptom and/or slow down, alleviate, and delay the development or severity of the disease or symptom. The prophylactically or therapeutically effective amount is influenced by factors including but not limited to: the development speed and severity of diseases or symptoms, the age, gender, weight, and physiological condition of a subject, the duration of treatment, and the specific route of administration. The prophylactically or therapeutically effective amount may be administered in one or more doses. The prophylactically or therapeutically effective amount may be achieved through continuous or discontinuous administration.

### Lipid Composition

Provided herein is a lipid composition. The lipid composition is a lipid delivery vector. A lipid can encapsulate a therapeutic agent or a prophylactic agent (e.g., a nucleotide) to form a nanoparticle so as to be delivered into an organism.

As used herein, the term “lipid” refers to an organic compound that includes a hydrophobic moiety and optionally also a hydrophilic moiety. The lipid is generally insoluble in water but soluble in many organic solvents. Generally, an amphiphilic lipid including a hydrophobic moiety and a hydrophilic moiety can be organized into a lipid bilayer structure in an aqueous environment, e.g., being present in the form of vesicle. Lipids can include but are not limited to: fatty acids, glycerides, phospholipids, sphingolipids, glycolipids, and steroid and cholesterol esters.

As used herein, “lipid nanoparticle” or “LNP” refers to a lipid vesicle with a uniform lipid core, which is a particle formed by lipids, in which the lipid components intermolecularly interact with each other to form a nanostructured entity. The therapeutic agent or the prophylactic agent (such as a nucleic acid, e.g., an mRNA) is encapsulated in the lipid.

Particularly preferably, the lipid composition may be, for example, a lipopolyplex (LPP) as described herein. The method for preparing such compositions is as described herein. LPP is a particle with a core-shell structure, in which a therapeutic agent or a prophylactic agent (such as a nucleic acid, e.g., an mRNA) is included in a polymer complex, and the polymer complex itself is encapsulated in a biocompatible lipid bilayer shell to form the lipid nanoparticle of the present disclosure. In some embodiments, the lipid composition of the present disclosure is a lipopolyplex (LPP). In some embodiments, the composition of the present disclosure is a lipopolyplex (LPP) including an RNA.

In some embodiments, the lipid encapsulating a therapeutic agent or a prophylactic agent (such as a nucleic acid, e.g., an mRNA) is selected from one or more of: a cationic lipid, a phospholipid, a steroid, and/or a polyethylene glycol modified lipid. In a preferred embodiment, the cationic lipid is an ionizable cationic lipid.

In an embodiment, the lipid composition includes the cationic lipid, wherein the cationic lipid includes DOTMA, DOTAP, DDAB, DOSPA, DODAC, DODAP, DC-Chol, DMRIE, DMOBA, DLinDMA, DLenDMA, CLinDMA, DMORIE, DLDMA, DMDMA, DOGS, N4-cholesteryl-spermine, DLin-KC2-DMA, DLin-MC3-DMA, a compound of formula (I), (II), (III) or (IV) described herein, or a combination thereof. In a preferred embodiment, the cationic lipid includes M5. In a preferred embodiment, the cationic lipid includes SW-II-127, SW-II-135-1, or SW-II-138-1. In a preferred embodiment, the cationic lipid includes M5, SW-II-127, SW-II-135-1, or SW-II-138-1.

In an embodiment, the lipid composition includes the phospholipid and/or the steroid. In an embodiment, the lipid composition includes the phospholipid as described herein, wherein the phospholipid includes 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleylphosphatidylethanolamine (POPE), distearoyl-phosphatidyl-ethanolamine (DSPE), dipalmitoylphosphatidylethanolamine (DPPE), dimyristoylphosphoethanolamine (DMPE), 1-stearoyl-2-oleyl-stearylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), or a combination thereof. In an embodiment, the lipid composition includes the steroid as described herein, wherein the steroid includes cholesterol, coprosterol, sitosterol, ergosterol, campesterol, stigmasterol, brassicasterol, tomatidine, ursolic acid, α-tocopherol, and derivatives thereof. In an embodiment, the lipid composition includes the phospholipid and the steroid as described herein. In an embodiment, the lipid composition includes DOPE. In an embodiment, the lipid composition includes DSPC. In an embodiment, the lipid composition includes the cholesterol. In an embodiment, the lipid composition includes DOPE and the cholesterol. In an embodiment, the lipid composition includes DSPC and the cholesterol.

In an embodiment, the lipid composition includes the cationic lipid M5, SW-II-127, SW-II-135-1, or SW-II-138-1, the phospholipid DOPE, and the cholesterol. In an embodiment, the lipid composition includes the cationic lipid M5, SW-II-127, SW-II-135-1, or SW-II-138-1, the phospholipid DSPC, and the cholesterol.

In some embodiments, the lipid encapsulating a polynucleotide further includes a polyethylene glycol modified lipid. In an embodiment, the polyethylene glycol modified lipid includes DMG-PEG (e.g., DMG-PEG 2000), DOG-PEG, and DSPE-PEG, or a combination thereof. In an embodiment, the polyethylene glycol modified lipid is DSPE-PEG. In an embodiment, the polyethylene glycol modified lipid is DMG-PEG (e.g., DMG-PEG 2000).

In an embodiment, the lipid composition includes the cationic lipid, DOPE, the cholesterol, and DSPE-PEG.

In an embodiment, the lipid composition includes the cationic lipid, DSPC, the cholesterol, and DSPE-PEG.

In an embodiment, the lipid composition includes the cationic lipid, DSPC, the cholesterol, and DMG-PEG.

In a preferred embodiment, the lipid composition includes the cationic lipid, DOPE, the cholesterol, and DMG-PEG.

In an embodiment, the lipid composition includes the cationic lipid M5, SW-II-127, SW-II-135-1, or SW-II-138-1, DOPE, the cholesterol, and DMG-PEG.

In some embodiments, the lipid composition of the present disclosure further includes a cationic polymer, and the cationic polymer and the therapeutic agent or the prophylactic agent (such as a nucleic acid, e.g., an mRNA) are associated as a complex and co-encapsulated in the lipid.

In an embodiment, the cationic polymer comprises poly-L-lysine, protamine, polyethyleneimine (PEI), or a combination thereof. In an embodiment, the cationic polymer is the protamine. In an embodiment, the cationic polymer is PEI.

In an embodiment, the amount of the lipid in the lipid composition is calculated in mole percent (mol %), and the mole percent is determined based on the total mole of the lipid in the composition. Unless otherwise indicated, the sum of the amount (mol %) of each lipid in the composition is 100 mol %, i.e., the sum of the amount (mol %) of the cationic lipid, the phospholipid, the steroid, and the polyethylene glycol modified lipid is 100 mol %.

In an embodiment, the amount of the cationic lipid in the lipid composition is about 10-about 70 mol %. In some embodiments, the amount of the cationic lipid in the lipid composition is about 20-about 60 mol %, about 30-about 50 mol %, about 30-about 45 mol %, about 35-about 50 mol %, about 35-about 45 mol %, about 38-about 45 mol %, about 40-about 45 mol %, about 40-about 50 mol %, or about 45-about 50 mol %. For example, the amount of the cationic lipid may be about 30, 32.5, 35, 37.5, 40, 42.5, 45, 46.1, 47.5, 50, 52.5, 55, 57.5, or 60 mol %.

In an embodiment, the amount of the phospholipid in the lipid composition is about 10-about 70 mol %. In an embodiment, the amount of the phospholipid in the lipid composition is about 20-about 60 mol %, about 30-about 50 mol %, about 10-about 30 mol %, about 10-about 20 mol %, or about 10-about 15 mol %. For example, the amount of the phospholipid may be about 5, 10, 15, 20, 23, 25, 30, 35, or 40 mol %.

In an embodiment, the amount of the cholesterol in the lipid composition is about 10-about 70 mol %. In an embodiment, the amount of the cholesterol in the lipid composition is about 20-about 60 mol %, about 24-44 mol %, about 30-about 50 mol %, about 30-about 48.5 mol %, about 35-about 40 mol %, about 35-about 45 mol %, about 40-about 45 mol %, or about 45-about 50 mol %. For example, the amount of the cholesterol may be about 10, 15, 17.5, 18.75, 20, 22.5, 25, 27.5, 28.75, 29, 30, 32.5, 33.75, 34, 35, 38.5, 38.75, 40, 42.5, 43.5, 43.75, 44, 45, 46.25, 47.5, 48.5, 48.75, 49, 50, 52.5, 53.75, 55, 60, 62.5, 63.75, 65, or 70 mol %.

In an embodiment, the amount of the polyethylene glycol modified lipid in the lipid composition is about 0.05-about 20 mol %. In an embodiment, the amount of the polyethylene glycol modified lipid in the lipid composition is about 0.5-about 15 mol %, about 1-about 10 mol %, about 5-about 15 mol %, about 1-about 5 mol %, about 1-about 1.5 mol %, about 1.5-about 3 mol %, or about 2-5 mol %. For example, the amount of the polyethylene glycol modified lipid may be about 0.05, 0.9, 1, 1.25, 1.5, 2, 2.5, 3, 3.5, 4, 4.5, 5, 5.5, 6, 6.5, 7, 7.5, 8, 8.5, 9, 9.5, 10, 15, or 20 mol %.

In an embodiment, the lipid composition includes 10-70 mol % of the cationic lipid, 10-70 mol % of the phospholipid, 10-70 mol % of the steroid, and 0.05-20 mol % of the polyethylene glycol modified lipid. In a preferred embodiment, the lipid composition includes 30-45 mol % of the cationic lipid, 10-20 mol % of the phospholipid, 30-48.5 mol % of the steroid, and 1-1.5 mol % of the polyethylene glycol modified lipid.

In an embodiment, LPP includes the therapeutic agent or the prophylactic agent (such as a nucleic acid, e.g., an mRNA) of the present disclosure, which associates with the cationic polymer as a complex; and a lipid encapsulating the complex, wherein the lipid encapsulating the complex includes a cationic lipid, a phospholipid, a steroid, and a polyethylene glycol modified lipid. In an embodiment, the phospholipid is selected from 1,2-dioleoyl-sn-glycero-3-phosphoethanolamine (DOPE), 1,2-distearoyl-sn-glycero-3-phosphocholine (DSPC), or a combination thereof. In an embodiment, the steroid is a cholesterol. In an embodiment, the cationic polymer is protamine. In an embodiment, the polyethylene glycol modified lipid is selected from 1,2-dimyristoyl-rac-glycero-3-methoxypolyethylene glycol (DMG-PEG), 1,2-distearoyl-sn-glycero-3-phosphoethanolamine-poly(ethylene glycol) (DSPE-PEG), or a combination thereof. In an embodiment, the cationic lipid is selected from M5, SW-II-127, SW-II-135-1, or SW-II-138-1.

In an embodiment, the lipid encapsulating the complex includes 40 mol % of M5, SW-II-127, SW-II-135-1, or SW-II-138-1, 15 mol % of DOPE, 43.5 mol % of the cholesterol, and 1.5 mol % of DMG-PEG.

In an embodiment, the therapeutic agent or the prophylactic agent is a polynucleotide, the polynucleotide includes a coding region, and the coding region encodes IL-12, wherein the IL-12 includes an amino acid sequence of SEQ ID NO: 3 or an amino acid sequence having at least 95% identity to the amino acid sequence of SEQ ID NO: 3; and wherein the polynucleotide is an RNA, wherein the coding region includes a nucleotide sequence of SEQ ID NO: 4 or a nucleotide sequence having at least 85% identity to the nucleotide sequence of SEQ ID NO: 4; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In an embodiment, the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

In an embodiment, the lipid encapsulating the complex includes 40 mol % of M5, SW-II-127, SW-II-135-1, or SW-II-138-1, 15 mol % of DOPE, 43.5 mol % of the cholesterol, and 1.5 mol % of DMG-PEG; and the therapeutic agent or the prophylactic agent is a polynucleotide, the polynucleotide includes a coding region, and the coding region encodes IL-12, wherein the IL-12 includes an amino acid sequence of SEQ ID NO: 3 or an amino acid sequence having at least 95% identity to the amino acid sequence of SEQ ID NO: 3; and wherein the polynucleotide is an RNA, wherein the coding region includes a nucleotide sequence of SEQ ID NO: 4 or a nucleotide sequence having at least 85% identity to the nucleotide sequence of SEQ ID NO: 4; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In an embodiment, the lipid encapsulating the complex includes 40 mol % of M5, 15 mol % of DOPE, 43.5 mol % of the cholesterol, and 1.5 mol % of DMG-PEG; and the therapeutic agent or the prophylactic agent is a polynucleotide, the polynucleotide includes a coding region, and the coding region encodes IL-12, wherein the IL-12 includes an amino acid sequence of SEQ ID NO: 3 or an amino acid sequence having at least 95% identity to the amino acid sequence of SEQ ID NO: 3; and wherein the polynucleotide is an RNA, wherein the coding region includes a nucleotide sequence of SEQ ID NO: 4 or a nucleotide sequence having at least 85% identity to the nucleotide sequence of SEQ ID NO: 4; or wherein the polynucleotide is a DNA, wherein the coding region includes a nucleotide sequence of SEQ ID NO: 5 or a nucleotide sequence having at least 85% identity to the nucleotide sequence of SEQ ID NO: 5.

In an embodiment, the lipid encapsulating the complex includes 40 mol % of M5, SW-II-127, SW-II-135-1, or SW-II-138-1, 15 mol % of DOPE, 43.5 mol % of the cholesterol, and 1.5 mol % of DMG-PEG; and the therapeutic agent or the prophylactic agent is a polynucleotide, and the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

In an embodiment, the lipid encapsulating the complex includes 40 mol % of M5, 15 mol % of DOPE, 43.5 mol % of the cholesterol, and 1.5 mol % of DMG-PEG; and the therapeutic agent or the prophylactic agent is a polynucleotide, and the polynucleotide is an RNA, including a nucleotide sequence of SEQ ID NO: 6 or a nucleotide sequence having at least 85% identity to the nucleotide sequence of SEQ ID NO: 6; or the polynucleotide is a DNA, including a nucleotide sequence of SEQ ID NO: 7 or a nucleotide sequence having at least 85% identity to the nucleotide sequence of SEQ ID NO: 7.

### Cationic Lipid

A cationic lipid is a lipid that can carry a net positive charge at a given pH. The lipid with the net positive charge can associate with a nucleic acid via an electrostatic interaction.

Instances of cationic lipids include but are not limited to 1,2-di-O-octadecenyl-3-trimethylammonium-propane (DOTMA), 1,2-dioleoyl-3-trimethylammonium-propane (DOTAP), didecyldimethylammonium bromide (DDAB), 2,3-dioleoyloxy-N-[2(spermine carboxamide)ethyl]-N,N-dimethyl-l-propanamium trifluoroacetate (DOSPA), dioctadecyldimethyl ammonium chloride (DODAC), 1,2-dioleoyl-3-dimethylammonium-propane (DODAP), 3-(N—(N′,N′-dimethylaminoethane)-carbamoyl)cholesterol (DC-Chol), 2,3-di(tetradecoxy)propyl-(2-hydroxyethyl)-dimethylazanium (DMRIE), N,N-dimethyl-3,4-dioleyloxybenzylamine (DMOBA), 1,2-dilinoleyloxy-N,N-dimethylaminopropane (DLinDMA), 1,2-dilinolenyloxy-N,N-dimethylaminopropane (DLenDMA), 3-dimethylamino-2-(cholest-5-en-3-beta-oxybutan-4-oxy)-1-(cis,cis-9,12-oc-tadecadienoxy)propane (CLinDMA), N-(2-aminoethyl)-N,N-dimethyl-2,3-bis(tetradecyloxy)propan-1-aminium bromide (DMORIE), N,N-dimethyl-2,3-bis(dodecyloxy)propan-1-amine (DLDMA), N,N-dimethyl-2,3-bis(tetradecyloxy)propan-1-amine (DMDMA), dioctadecylamidoglycyl spermine (DOGS), N4-cholesteryl-spermine, 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), a compound of formula (I), (II), (III) or (IV) as described herein, or a combination thereof.

In some embodiments, the cationic lipid is preferably an ionizable cationic lipid. The ionizable cationic lipid has a net positive charge, e.g., at acidic pH, and is neutral at higher pH (e.g., physiological pH). Instances of ionizable cationic lipids include but are not limited to: dioctadecylamidoglycyl spermine (DOGS), N4-cholesteryl-spermine, 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), a compound of formula (I), (II), (III) or (IV) as described herein, or a combination thereof.

In a preferred embodiment, the cationic lipid includes a compound of formula (I), or a pharmaceutically acceptable salt thereof:

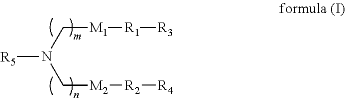

where
R₁ and R₂ are each independently selected from C₁-C₁₂ alkyl and C₂-C₁₂ alkenyl;
R₃ and R₄ are each independently selected from C₁-C₁₂ alkyl, C₂-C₁₂ alkenyl, C₆-C₁₀ aryl, and 5-10 membered heteroaryl,
R₃ and R₄ are each independently optionally substituted with t R₆, t being an integer selected from 1-5; and R₆ is independently selected from C₁-C₁₂ alkyl and C₂-C₁₂ alkenyl;
M₁ and M₂ are each independently selected from —OC(O)—, —C(O)O—, —SC(S)—, and —C(S)S—;
R₅ is selected from —C₁₋₁₂ alkylene-Q, Q is selected from —OR₇ and —SR₇, and R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl; and
m and n are each independently an integer selected from 1-12.

In an embodiment, the cationic lipid includes a lipid compound having a structure shown below, or a pharmaceutically acceptable salt thereof.

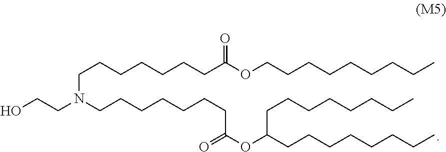 [SMILES: CCCCCCCCCOC(=O)CCCCCCCN(CCO)CCCCCCCC(=O)OC(CCCCCCCC)CCCCCCCC]

In an embodiment, at least one of R₃ and R₄ is C₆-C₁₀ aryl or 5-10 membered heteroaryl.

In an embodiment, R₂ is selected from C₁-C₁₂ alkyl. In another embodiment, R₂ is selected from C₁-C₆ alkyl.

In an embodiment, one of R₃ and R₄ is C₆-C₁₀ aryl or 5-10 membered heteroaryl, and the other is C₁-C₁₂ alkyl or C₂-C₁₂ alkenyl.

In a specific embodiment, R₃ and R₄ are each independently selected from C₁-C₁₂ alkyl and phenyl, with a proviso that at least one of R₃ and R₄ is phenyl. In another embodiment, one of R₃ and R₄ is phenyl, and the other is C₁-C₁₂ alkyl.

In yet another embodiment, R₃ and R₄ are each independently substituted with t R₆, t being an integer selected from 1-5, e.g., 1, 2, 3, 4, or 5. Preferably, t is an integer from 1 to 3, e.g., 1, 2, or 3, in particular 1 or 2.

In an embodiment, R₆ is independently selected from C₁-C₁₂ alkyl, e.g., C₁-C₁₀ alkyl.

In an embodiment, t is 1, and R₆ is substituted at a meta-position or a para-position on a benzene ring relative to R₁ or R₂.

In another embodiment, t is 2, and R₆ is substituted at a meta-position and a para-position on a benzene ring relative to R₁ or R₂.

In an embodiment, R₄ is substituted at a 1-position or a last position of R₂. The 1-position refers to a position of a C atom in R₂ directly connected to M₂. The last position refers to a position of a C atom in R₂ farthest away from M₂. In a specific embodiment, R₄ is selected from C₁-C₁₂ alkyl, and R₃ is phenyl.

In an embodiment, R₃ is substituted at a 1-position or a last position of R₁. The 1-position refers to a position of a C atom in R₁ directly connected to M₁. The last position refers to a position of a C atom in R₁ farthest away from M₁. In a specific embodiment, R₃ is selected from C₁-C₁₂ alkyl, and R₄ is phenyl.

In an embodiment, M₁ and M₂ are each independently selected from —OC(O)— and —C(O)O—.

In an embodiment, R₅ is selected from —C₁₋₅ alkylene-Q, e.g., C₁, C₂, C₃, C₄, or C₅ alkylene-Q. In an exemplary embodiment, R₅ is selected from —C₁₋₃ alkylene-Q, e.g., C₁, C₂, or C₃ alkylene-Q.

In another embodiment, Q is selected from —OH and —SH, in particular —OH.

In some embodiments, m and n are each independently an integer selected from 2-9, e.g., 2, 3, 4, 5, 6, 7, 8, or 9. Preferably, m and n are each independently an integer selected from 2-7, e.g., 2, 3, 4, 5, 6, or 7, and more preferably, m and n are each independently an integer selected from 5-7, e.g., 5, 6, or 7.

In certain embodiments, the compound of formula (I) includes a compound shown in formula (II):

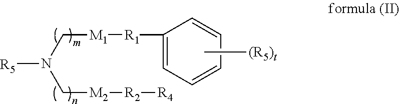

or a pharmaceutically acceptable salt thereof, wherein each group is as defined herein.

In an embodiment,
R₁ is selected from C₁-C₆ alkyl;
R₂ is selected from C₁-C₁₀ alkyl;
R₄ is selected from C₁-C₁₀ alkyl;
M₁ and M₂ are each independently selected from —OC(O)— and —C(O)O—;
R₅ is selected from —C₁₋₅ alkylene-Q, Q is selected from —OR₇ and —SR₇, and R₇ is independently selected from H, C₁-C₁₂ alkyl, and C₂-C₁₂ alkenyl;
R₆ is independently selected from C₁-C₁₂ alkyl and C₂-C₁₂ alkenyl, in particular C₁-C₁₂ alkyl;
m and n are each independently an integer selected from 2-9, e.g., 2, 3, 4, 5, 6, 7, 8, or 9; and
t is an integer selected from 1-3.

In an embodiment, R₅ is selected from —C₁₋₃ alkylene-Q, and Q is selected from —OH and —SH, in particular —OH.

In an embodiment, m and n are each independently an integer selected from 2-7, e.g., 2, 3, 4, 5, 6, or 7.

In some embodiments, t is 1 or 2.

In an embodiment, R₄ is substituted at a 1-position or a last position of R₂. The 1-position refers to a position of a C atom in R₂ directly connected to M₂. The last position refers to a position of a C atom in R₂ farthest away from M₂.

In an embodiment, t is 1, R₆ is substituted at a meta-position or a para-position on a benzene ring relative to R₁.

In another embodiment, t is 2, and R₆ is substituted at a meta-position and a para-position on a benzene ring relative to R₁.

In certain embodiments, the compound of formula (I) includes a compound shown in formula (III):

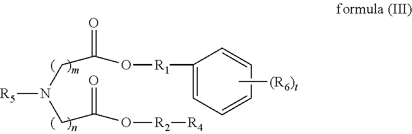

or a pharmaceutically acceptable salt thereof, wherein each group is as defined herein.

In an embodiment,
R₁ is selected from C₁-C₆ alkyl;
R₂ is selected from C₁-C₁₀ alkyl;
R₄ is selected from C₁-C₁₀ alkyl;
R₅ is selected from —C₁₋₃ alkylene-Q, and Q is selected from —OH and —SH, in particular —OH;
t is 1 or 2;
R₆ is selected from C₁-C₁₂ alkyl and C₂-C₁₂ alkenyl, in particular C₁-C₁₂ alkyl; and
m and n are each independently an integer selected from 2-7, e.g., 2, 3, 4, 5, 6, or 7.

In an embodiment, R₄ is substituted at a 1-position or a last position of R₂. The 1-position refers to a position of a C atom in R₂ directly connected to

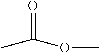

moiety. The last position refers to a position of a C atom in R₂ farthest away from

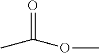

moiety.

In an embodiment, t is 1, R₆ is substituted at a meta-position or a para-position on a benzene ring relative to R₁.

In another embodiment, t is 2, and R₆ is substituted at a meta-position and a para-position on a benzene ring relative to R₁.

In certain embodiments, the compound of formula (I) includes a compound shown in formula (IV):

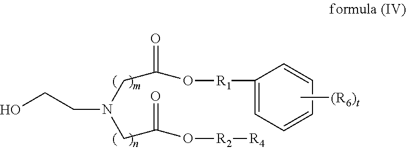

or a pharmaceutically acceptable salt thereof, wherein each group is as defined herein.

In an embodiment,
R₁ is selected from C₁-C₆ alkyl;
R₂ is selected from C₁-C₁₀ alkyl;
R₄ is selected from C₁-C₁₀ alkyl;
t is 1 or 2;
R₆ is independently selected from C₁-C₁₂ alkyl and C₂-C₁₂ alkenyl, in particular C₁-C₁₂ alkyl;
m and n are each independently an integer selected from 2-7, e.g., 2, 3, 4, 5, 6, or 7.

In an embodiment, R₄ is substituted at a 1-position or a last position of R₂. The 1-position refers to a position of a C atom in R₂ directly connected to

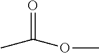

moiety. The last position refers to a position of a C atom in R₂ farthest away from

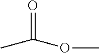

moiety.

In an embodiment, t is 1, R₆ is substituted at a meta-position or a para-position on a benzene ring relative to R₁.

In another embodiment, t is 2, and R₆ is substituted at a meta-position and a para-position on a benzene ring relative to R₁.

In a particular embodiment, the substituent groups (e.g., R₁—R₇) in the lipid compounds of the present disclosure include no alkenyl.

In a preferred embodiment, the cationic lipid includes a lipid compound having a structure shown below, or a pharmaceutically acceptable salt thereof

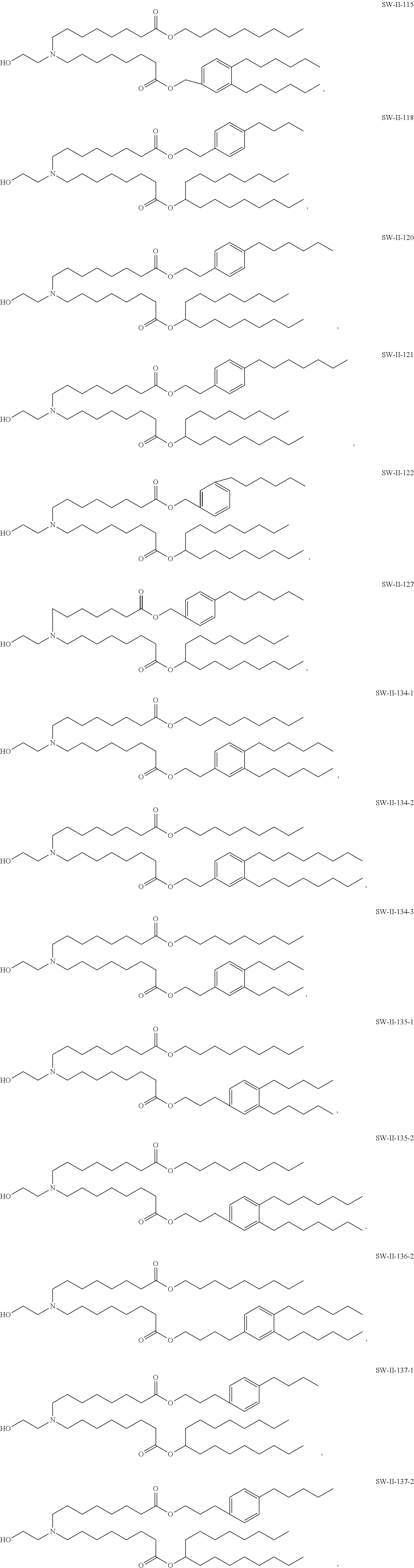

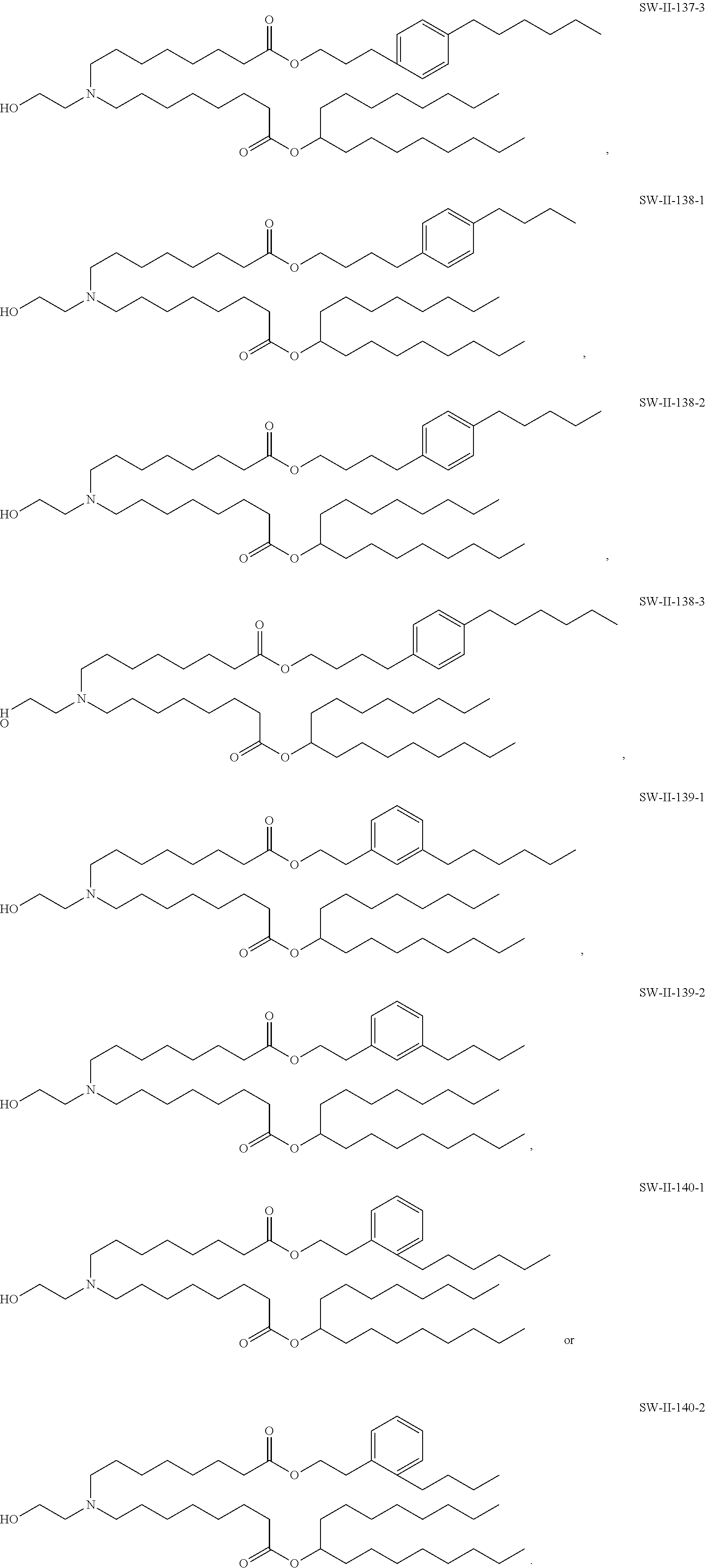

In a preferred embodiment, the cationic lipid includes the following lipid compounds: SW-II-127, SW-II-135-1, or SW-II-138-1.

### Phospholipid

The lipid composition of the present disclosure includes a phospholipid, which can assist the cell permeation of the lipid composition.

Instances of phospholipids include but are not limited to: 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleylphosphatidylethanolamine (POPE), distearoyl-phosphatidyl-ethanolamine (DSPE), dipalmitoylphosphatidylethanolamine (DPPE), dimyristoylphosphoethanolamine (DMPE), 1-stearoyl-2-oleyl-stearylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), or a combination thereof.

### Steroid

The lipid composition of the present disclosure includes a steroid, which can serve as a structural component of the lipid composition.

Instances of steroids include but are not limited to, for example, cholesterol, coprosterol, sitosterol, ergosterol, campesterol, stigmasterol, brassicasterol, tomatidine, ursolic acid, α-tocopherol, and derivatives thereof.

### Polyethylene Glycol Modified Lipid

As used herein, the term “polyethylene glycol modified lipid” or “PEG modified lipid” or “PEG lipid” refers to a molecule including a polyethylene glycol moiety and a lipid moiety, and is a lipid modified with polyethylene glycol. The PEG lipid may be selected from the non-limiting groups consisting of PEG-modified phosphatidylethanolamine, PEG-modified phosphatidic acid, PEG-modified ceramide (PEG-CER), PEG-modified dialkylamine, PEG-modified diacylglycerol (PEG-DEG), PEG-modified dialkylglycerol, or a combination thereof. For example, instances of polyethylene glycol modified lipids include but are not limited to: 1,2-dimyristoyl-rac-glycero-3-methoxypolyethylene glycol (DMG-PEG), 1,2-dioleoyl-rac-glycerol, methoxypolyethylene glycol (DOGPEG), and 1,2-distearoyl-sn-glycero-3-phosphoethanolamine-poly(ethylene glycol) (DSPE-PEG).

In an embodiment, the polyethylene glycol modified lipid is DMG-PEG, e.g., DMG-PEG 2000. In an embodiment, DMG-PEG 2000 has the following structure:

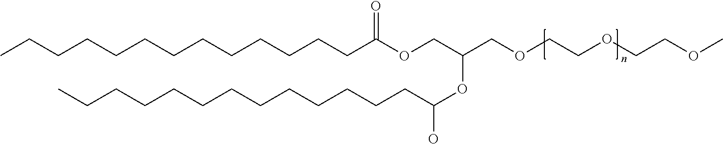

where an average value of n is 44.

### Cationic Polymer

As used herein, the term “cationic polymer” relates to any ionic polymer capable of carrying a net positive charge at a specified pH to bind electrostatically to a nucleic acid. Instances of cationic polymers include but are not limited to: poly-L-lysine, protamine, polyethyleneimine (PEI), or a combination thereof. PEI may be linear or branched PEI.

The term “protamine” refers to an arginine-rich, low molecular weight, basic protein that is present in sperm cells of various animals (particularly fish) and binds to a DNA instead of histone. In a preferred embodiment, the cationic polymer is the protamine (e.g., protamine sulfate).

### Pharmaceutical Composition

The present disclosure further provides a pharmaceutical composition, including the lipid composition of the present disclosure, and a pharmaceutically acceptable carrier.

Pharmaceutically acceptable carriers may include but are not limited to: diluents, binders and adhesives, lubricants, disintegrants, preservatives, vehicles, dispersing agents, glidants, sweeteners, coatings, excipients, preservatives, antioxidants (e.g., ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, propyl gallate, α-tocopherol, citric acid, ethylenediaminetetraacetic acid (EDTA), sorbitol, tartaric acid, phosphoric acid, etc.), solubilizing agents, gelling agents, softeners, solvents (e.g., water, alcohol, acetic acid, and syrup), buffering agents (e.g., phosphate buffer, histidine buffer, and acetate buffer), surfactants (e.g., non-ionic surfactants such as polysorbate 80, polysorbate 20, poloxamer, or polyethylene glycol), antibacterial agents, antifungal agents, isotonic agents (e.g., trehalose, sucrose, mannitol, sorbitol, lactose, and glucose), absorption delaying agents, chelating agents, and emulsifying agents. For the pharmaceutical composition, an appropriate carrier may be selected from a buffer (e.g., citrate buffer, acetate buffer, phosphate buffer, histidine buffer, and histidine salt buffer), an isotonic agent (e.g., trehalose, sucrose, mannitol, sorbitol, lactose, and glucose), a non-ionic surfactant (e.g., polysorbate 80, polysorbate 20, and poloxamer), or a combination thereof.

The pharmaceutical composition provided herein may be in various dosage forms, including but not limited to solid, semi-solid, liquid, powder, or lyophilized forms. For the pharmaceutical composition, the preferred dosage forms may generally be, for example, injection solution and lyophilized powder. The pharmaceutical composition may be prepared in various forms suitable for various routes and methods of administration. For example, the pharmaceutical composition may be prepared as liquid dosage forms (e.g., emulsions, microemulsions, nanoemulsions, solutions, suspensions, syrups, and elixirs), injectable forms, solid dosage forms (e.g., capsules, tablets, pills, powders, and granules), surface and/or transdermal administration dosage forms (e.g., ointments, pastes, creams, lotions, gels, powders, solutions, sprays, inhalants, and patches), suspensions, powders, and other forms.

The pharmaceutical composition of the present disclosure may be a pharmaceutical composition for injection, which may correspondingly include a pharmaceutically acceptable excipient for injection, and preferably, an excipient for intratumoral injection.

Therefore, the present disclosure further provides an intratumoral injectant, including the lipid composition of the present disclosure, and a pharmaceutically acceptable excipient for injection. For example, a sterile injectable aqueous or oily suspension may be included. A sterile injectable preparation may be a sterile injectable solution, suspension, and/or emulsion in a non-toxic diluent and/or solvent.

The above excipient for injection may include one or more of water, a saccharide solution, an electrolyte solution, an amino acid solution, or fat emulsion. For example, the excipients for injection may include one or more of 5% and 10% glucose injection, 0.9% sodium chloride injection, sterilized injection water, 5-10% fructose solution, 5% sodium bicarbonate solution, seal oil, or lactose hydrate.

### Therapeutic Agent/Prophylactic Agent

A lipid composition may include one or more therapeutic agents or prophylactic agents. The present disclosure provides methods for delivering a therapeutic agent or a prophylactic agent to a mammalian tumor, producing a polypeptide of interest in the mammalian tumor, and treating a cancer in a mammal in need thereof, including administering to the mammal a lipid composition including the therapeutic agent or the prophylactic agent.

The therapeutic agent or the prophylactic agent includes a biologically active substance and is alternatively referred to as “active agent”. The therapeutic agent or the prophylactic agent may be a substance that causes a desired change in a tumor after delivery to the tumor. In some embodiments, the therapeutic agent or the prophylactic agent is a small molecule drug that can be used to treat a particular tumor. Instances of drugs capable of being used for a composition include but are not limited to antineoplastic agents (e.g., vincristine, doxorubicin, mitoxantrone, camptothecin, cisplatin, bleomycin, cyclophosphamide, methotrexate, and streptozotocin), and antitumor agents (e.g., actinomycin D, vincristine, vinblastine, alkylating agents, platinum compounds, antimetabolites, and nucleoside analogues, e.g., methotrexate and purine and pyrimidine analogues).

In some embodiments, the therapeutic agent or the prophylactic agent is a cytotoxin, a radioactive ion, a chemotherapeutic agent, a vaccine, a compound that elicits an immune response, or another therapeutic agent or prophylactic agent. Cytotoxins or cytotoxic agents include any agent that is detrimental to cells. Instances include but are not limited to taxol, cytochalasin B, gramicidin D, ethidium bromide, emetine, mitomycin, etoposide, teniposide, vincristine, vinblastine, colchicine, doxorubicin, daunorubicin, dihydroxy anthracin dione, mitoxantrone, mithramycin, actinomycin D, 1-dehydrotestosterone, glucocorticoid, procaine, tetracaine, lidocaine, propranolol, puromycin, maytansinoid such as maytansinol, rachelmycin (CC-1065), and analogues or homologues thereof. Radioactive ions include but are not limited to iodine (e.g., iodine 125 or iodine 131), strontium 89, phosphorus, palladium, cesium, iridium, phosphate radical, cobalt, yttrium 90, samarium 153, and praseodymium. Vaccines may include compounds and preparations that direct the immune response against cancer cells, and may include an mRNA encoding a tumor cell derived antigen, epitope, and/or neoepitope. Other therapeutic agents or prophylactic agents include but are not limited to antimetabolites (e.g., methotrexate, 6-mercaptopurine, 6-thioguanine, cytarabine and 5-fluorouracil dacarbazine), alkylating agents (e.g., mechlorethamine, thiotepa, chlorambucil, rachelmycin (CC-1065), melphalan, carmustine, (BSNU), lomustine (CCNU), cyclophosphamide, busulfan, dibromannitol, streptozotocin, mitomycin C, and cis-diamminedichloroplatinum (II)(DDP), cis-platinum), anthracycline (e.g., daunorubicin (formerly referred to as daunomycin), and doxorubicin), antibiotics (e.g., dactinomycin (formerly referred to as actinomycin), bleomycin, mithramycin, and anthramycin (AMC)), and antimitotic agents (e.g., vincristine, vinblastine, taxol, and maytansinoid).

### Polynucleotide

In some embodiments, the therapeutic agent or the prophylactic agent is a polynucleotide or a nucleic acid (e.g., a ribonucleic acid or a deoxyribonucleic acid).

In some embodiments, the therapeutic agent or the prophylactic agent of the present disclosure is an RNA. As used herein, the definition of “RNA” encompasses single-stranded, double-stranded, linear, and circular RNAs. The RNA of the present disclosure may be chemically synthesized, recombinantly produced, and in vitro transcribed RNAs. In an embodiment, the RNA of the present disclosure is used for expression of a polypeptide in a host cell.

In an embodiment, the therapeutic agent or the prophylactic agent of the present disclosure is a single-stranded RNA. In an embodiment, the RNA of the present disclosure is an in vitro transcribed RNA (IVT-RNA). IVT-RNA may be obtained by in vitro transcription via an RNA polymerase using a DNA template.

In some embodiments, the therapeutic agent or the prophylactic agent of the present disclosure is a messenger RNA (mRNA). Generally speaking, the mRNA may include a 5′-UTR sequence, a coding sequence for polypeptides, a 3′-UTR sequence, and an optionally present poly(A) sequence. mRNA may be produced, for example, via in vitro transcription or chemical synthesis. In an embodiment, the mRNA of the present disclosure includes (1) 5′-UTR, (2) a coding sequence, (3) 3′-UTR, and (4) an optionally present poly(A) sequence. In an embodiment, the RNA of the present disclosure is a nucleoside modified mRNA. In an embodiment, the mRNA of the present disclosure includes an optionally present 5′ cap.

As used herein, the term “untranslated region (UTR)” generally refers to a region (non-coding region) in an RNA (e.g., an mRNA) that is not translated into an amino acid sequence, or a corresponding region in a DNA. Generally, UTR located at a 5′ end (upstream) of an open reading frame (initiation codon) may be referred to as 5′ untranslated region 5′-UTR; and UTR located at a 3′ end (downstream) of an open reading frame (termination codon) may be referred to as 3′-UTR. In the presence of a 5′ cap, the 5′-UTR is located downstream of the 5′ cap, for example, directly adjacent to the 5′ cap. In a particular embodiment, an optimized “Kozak sequence” may be included in the 5′-UTR, for example, near the initiation codon, to improve translation efficiency. In the presence of a poly(A) sequence, the 3′-UTR is located upstream of the poly(A) sequence, for example, directly adjacent to the poly(A) sequence.

As used herein, the term “poly(A) sequence” or “poly(A) tail” refers to a nucleotide sequence including consecutive or inconsecutive adenosines. The poly(A) sequence is generally located at a 3′ end of RNA, for example, 3′ end (downstream) of 3′-UTR. In some embodiments, the poly(A) sequence does not include any nucleotides other than adenosine at its 3′ end. The poly(A) sequence may be produced by transcription by a DNA-dependent RNA polymerase based on a coding sequence of a DNA template during the preparation of IVT-RNA, or connected to a free 3′ end of IVT-RNA, e.g., 3′ end of 3′-UTR, via a DNA-independent RNA polymerase (poly(A) polymerase).

As used herein, the term “5′ cap” generally involves an N7-methylguanosine structure (also referred to as “m7G cap”, and “m7Gppp-”) connected to a 5′ end of mRNA via a 5′ to 5′ triphosphate bond. The 5′ cap may be co-transcribed and added to an RNA during in vitro transcription (e.g., using an anti-reverse cap analog “ARCA”), or may be connected to an RNA after transcription using a capping enzyme.

In some embodiments, the therapeutic agent or the prophylactic agent of the present disclosure is a DNA. Such DNA may be, for example, a DNA template for in vitro transcription of the RNA of the present disclosure, or a DNA vaccine for expression of a polypeptide antigen in a host cell. The DNA may be double-stranded, single-stranded, linear, and circular DNA.

The DNA template may be provided in an appropriate transcription vector. Generally speaking, the DNA template may be a double-stranded complex including a nucleotide sequence (coding strand) that is identical to the coding sequence described herein, and a nucleotide sequence (template strand) that is complementary to the coding sequence described herein. As is known to those skilled in the art, the DNA template may include a promoter, 5′-UTR, a coding sequence, 3′-UTR, and an optionally present poly(A) sequence. The promoter may be a promoter available to an appropriate RNA polymerase (in particular a DNA-dependent RNA polymerase) known to those skilled in the art, including but not limited to promoters of SP6, T3 and T7RNA polymerases. The 5′-UTR, coding sequence, 3′-UTR, and poly(A) sequence in the DNA template are corresponding or complementary to those included in the RNA described herein. The polynucleotide as a DNA vaccine may be provided in a plasmid vector (e.g., a circular plasmid vector).

In some embodiments, the therapeutic agent or the prophylactic agent of the present disclosure is an mRNA encoding a cytokine. The cytokines include but are not limited to IL-2, IL-10, IL-12, IL-15, IL-21, IFN-α, IFN-3, or IFN-γ.

In a preferred embodiment, the therapeutic agent or the prophylactic agent of the present disclosure is IL-12 mRNA. For exemplary nucleic acid sequences thereof, reference may be made to SEQ ID NO: 2, SEQ ID NO: 4, or SEQ ID NO: 6.

### Interleukin-12 (IL-12)

In some embodiments, the therapeutic agent or the prophylactic agent herein is a polynucleotide encoding interleukin-12 (IL-12). In some embodiments, the therapeutic agent or the prophylactic agent herein is an RNA encoding IL-12. In some embodiments, the therapeutic agent or the prophylactic agent herein is a DNA encoding IL-12. IL-12 is a proinflammatory cytokine that plays an important role in innate immunity and adaptive immunity, can promote Th1 cell differentiation and enhance the cytotoxic effects of cytotoxic T cells (CTL cells) and natural killer cells (NK cells), and plays an important role in cellular immunity. IL-12 mainly functions as a 70 kDa heterodimeric protein (p70) consisting of p35 and p40 subunits. The IL-12p40 subunit, also referred to as IL12B, as used herein, has a protein sequence as shown in NCBI accession No. NP_002178.2. The IL-12p35 subunit, also referred to as IL12A, as used herein, has a protein sequence as shown in NCBI accession No. NP_000873.2. Unless otherwise indicated, interleukin 12 (IL-12) or IL-12 (P70) herein is a heterodimeric protein consisting of p35 and p40 subunits. In some embodiments, IL-12 or IL-12 (p70) is a fusion protein including a p40 subunit, a peptide linker, and a p35 subunit in sequence from a N-terminus to a C-terminus. In a preferred embodiment, IL-12 or IL-12 (p70) includes an amino acid sequence of SEQ ID NO: 3.

In some embodiments, the polypeptide encoded by the polynucleotide of the present disclosure includes an amino acid sequence of SEQ ID NO: 3 or has at least 90%, 91%, 92%, 93%, 94%, 95%, 96%, 97%, 98%, or 99% identity to the amino acid sequence of SEQ ID NO: 3. The polypeptide encoded by the polynucleotide of the present disclosure can promote Th1 cell differentiation and enhance the cytotoxic effects of CTL cells and NK cells.

In some embodiments, the polynucleotide includes a coding region that encodes IL-12.

As used herein, “coding sequence” refers to being capable of serving as a template in a polynucleotide for the synthesis of a nucleotide sequence with a defined nucleotide sequence (e.g., a tRNA and an mRNA) or a defined amino acid sequence in a biological process. The coding sequence may be a DNA sequence or an RNA sequence. If the mRNA corresponding to the DNA sequence (including the same coding strand as the mRNA sequence and a template strand of a strand complementary thereto) is translated into a polypeptide in a biological process, the DNA sequence or the mRNA sequence may be considered to encode the polypeptide.

As used herein, “codon” refers to three consecutive nucleotide sequences (also referred to as triplet code) in a polynucleotide that encode a particular amino acid. Synonymous codons (codons encoding the same amino acid) are used at different frequencies in different species, referred to as “codon bias”. It is generally believed that for a given species, coding sequences using their biased codons may have higher translation efficiency and accuracy in an expression system of the species. Thus, “codon optimization” may be carried out on the polynucleotide, i.e., the codons in the polynucleotide are altered to reflect codons preferred by a host cell, and preferably, the amino acid sequences encoded thereby are not altered. Those skilled in the art will understand that due to the degeneracy of codons, the polynucleotide of the present disclosure may include such a coding sequence that differs from the coding sequence described herein (e.g., having about 70%, 75%, 80%, 85%, 90%, 95%, 96%, 97%, 98%, or 99% identity to the coding sequence described herein) but encodes the same amino acid sequence. In a particular embodiment, the RNA of the present disclosure includes codons optimized for cells of a host (e.g., a subject, in particular a human), such that the polypeptide of the present disclosure is optimally expressed in the host (e.g., a subject, in particular a human).

In an embodiment, the polynucleotide of the present disclosure includes the coding sequence of the polypeptide as described herein. In an embodiment, the polynucleotide of the present disclosure includes a nucleotide sequence complementary to the coding sequence of the polypeptide as described herein. In an embodiment, the coding sequence includes an initiation codon at a 5′ end thereof and a termination codon at a 3′ end thereof. In an embodiment, the coding sequence includes the open reading frame (ORF) described herein.

In an embodiment, the coding sequence of the present disclosure encodes a polypeptide, the polypeptide including:

(1) an amino acid sequence of SEQ ID NO: 3; or (2) an amino acid sequence having at least 90%, 91%, 92%, 93%, 94%, 95%, 96%, 97%, 98%, or 99% identity to the amino acid sequence of SEQ ID NO: 3.

In an embodiment, the coding sequence of the polypeptide described herein includes a nucleotide sequence, the nucleotide sequence including: (1) a nucleotide sequence of SEQ ID NO: 4; (2) a nucleotide sequence having at least 70%, 75%, 80%, 85%, 90%, 95%, 96%, 97%, 98%, or 99% identity to the nucleotide sequence of SEQ ID NO: 4; (3) a nucleotide sequence of SEQ ID NO: 5; or (4) a nucleotide sequence having at least 70%, 75%, 80%, 85%, 90%, 95%, 96%, 97%, 98%, or 99% identity to the nucleotide sequence of SEQ ID NO: 5.

In some embodiments, the polynucleotide of the present disclosure is an RNA. In some embodiments, the RNA of the present disclosure further includes a structural element, including but not limited to a 5′ cap, 5′-UTR, 3′-UTR, and a poly(A) sequence, that helps to improve the stability and/or translation efficiency of the RNA.

In some embodiments, the RNA of the present disclosure includes the 5′-UTR. In a preferred embodiment, the 5′-UTR includes a nucleotide sequence of SEQ ID NO: 8. In a preferred embodiment, the 3′-UTR includes a nucleotide sequence of SEQ ID NO: 9. In some embodiments, the RNA of the present disclosure includes the 5′-UTR and the 3′-UTR. In a specific embodiment, the 5′-UTR includes a nucleotide sequence of SEQ ID NO: 8, and the 3′-UTR includes a nucleotide sequence of SEQ ID NO: 9.

In some embodiments, the RNA of the present disclosure includes the poly(A) sequence. In an embodiment, the poly(A) sequence includes consecutive adenosines. In an embodiment, the poly(A) sequence may include at least 20, 30, 40, 50, 60, 70, 75, 80, 85, 95, or 100 and up to 120, 150, 180, 200, or 300 adenosines. In an embodiment, the poly(A) sequence includes at least 50 nucleotides. In an embodiment, the poly(A) sequence includes at least 80 nucleotides. In an embodiment, the poly(A) sequence includes at least 100 nucleotides. In some embodiments, the poly(A) sequence includes about 70, 80, 90, 100, 120, or 150 nucleotides. In an embodiment, a consecutive adenosine sequence in the poly(A) sequence is interrupted by a sequence including a U, C, or G nucleotide. In an embodiment, the poly(A) sequence includes a nucleotide sequence of SEQ ID NO: 12.

In an embodiment, the RNA of the present disclosure includes a nucleotide sequence of SEQ ID NO: 4. In an embodiment, the RNA of the present disclosure includes a nucleotide sequence of SEQ ID NO: 6.

In an embodiment, the RNA of the present disclosure (a) includes a nucleotide sequence having at least 70%, 75%, 80%, 85%, 90%, 95%, 96%, 97%, 98%, or 99% identity to the nucleotide sequence of SEQ ID NO: 4 or 6; and (b) encodes an amino acid sequence having at least 90%, 91%, 92%, 93%, 94%, 95%, 96%, 97%, 98%, or 99% identity to the amino acid sequence of SEQ ID NO:3.

In some embodiments, the polynucleotide of the present disclosure is a DNA. In some embodiments, the DNA of the present disclosure includes the coding sequence of the polypeptide as described herein. In some embodiments, the DNA of the present disclosure includes, from a 5′ end to a 3′ end, (1) a T7 promoter, (2) 5′-UTR, (3) a coding sequence, (4) 3′-UTR, and (5) an optionally present poly(A) sequence as described herein.

In some embodiments, the T7 promoter includes a nucleotide sequence of SEQ ID NO: 14.

In some embodiments, the DNA of the present disclosure includes the 5′-UTR. In a preferred embodiment, the 5′-UTR includes a nucleotide sequence of SEQ ID NO: 10. In a preferred embodiment, the 3′-UTR includes a nucleotide sequence of SEQ ID NO: 11. In some embodiments, the RNA of the present disclosure includes the 5′-UTR and the 3′-UTR. In a specific embodiment, the 5′-UTR includes a nucleotide sequence of SEQ ID NO: 10, and the 3′-UTR includes a nucleotide sequence of SEQ ID NO: 11.

In some embodiments, the DNA of the present disclosure includes the poly(A) sequence. In an embodiment, the poly(A) sequence includes consecutive deoxyadenosines. In an embodiment, the poly(A) sequence may include at least 20, 30, 40, 50, 60, 70, 75, 80, 85, 95, or 100 and up to 120, 150, 180, 200, or 300 deoxyadenosine. In an embodiment, a consecutive adenosine sequence in the poly(A) sequence is interrupted by a sequence including a T, C, or G nucleotide. In an embodiment, the poly(A) sequence includes a nucleotide sequence of SEQ ID NO: 13.

In an embodiment, the DNA of the present disclosure includes a nucleotide sequence of SEQ ID NO: 5. In an embodiment, the DNA of the present disclosure includes a nucleotide sequence of SEQ ID NO: 7.

In an embodiment, the DNA of the present disclosure (a) includes a nucleotide sequence having at least 70%, 75%, 80%, 85%, 90%, 95%, 96%, 97%, 98%, or 99% identity to the nucleotide sequence of SEQ ID NO: 5 or 7; and (b) encodes an amino acid sequence having at least 90%, 91%, 92%, 93%, 94%, 95%, 96%, 97%, 98%, or 99% identity to the amino acid sequence of SEQ ID NO:3.

### Modified Nucleotide

In some embodiment, the mRNA herein includes a modified nucleotide, wherein the modified nucleotide is selected from one or more of the following nucleotides: 2-aminoadenosine, 2-thiothymidine, inosine, pyrrolopyrimidine, 3-methyladenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, N-1-methyl-pseudouridine, 2-thiouridine, and 2-thiocytidine; methylated bases; insertion bases; 2′-fluororibose, ribose, 2′-deoxyribose, arabinose, and hexose; thiophosphoryl and 5′-N-phosphoramidite bond; Moreover, the modified nucleotides described in PCT/CN2020/074825 and PCT/CN2020/106696 are modified.

In an embodiment, the RNA (e.g., an mRNA) of the present disclosure is modified by including one or more modified nucleobases. In an embodiment, the modified nucleobase includes a modified cytosine, a modified uracil, or a combination thereof. In an embodiment, the modified uracil is independently selected from pseudouracil, 1-methyl-pseudouracil, 5-methyl-uracil, or a combination thereof. In an embodiment, the modified cytosine is independently selected from 5-methylcytosine, 5-hydroxymethylcytosine, or a combination thereof. In an embodiment, the proportion of modified nucleobases in the RNA of the present disclosure is 10%-100%, that is, the RNA of the present disclosure may be modified by replacing 10%-100% of the nucleobases therein with modified nucleobases.

In some embodiments, the RNA (e.g., an mRNA) of the present disclosure is modified by replacing one or more uracils with the modified uracil. In an embodiment, the modified uracil includes 1-methyl-pseudouracil, pseudouracil, 5-methyl-uracil, or a combination thereof. In an embodiment, the modified uracil includes pseudouracil. In an embodiment, the modified uracil includes 5-methyl-uracil. In an embodiment, the modified uracil includes 1-methyl-pseudouracil.

In an embodiment, the RNA is modified by replacing at least one uracil with the modified uracil. In an embodiment, the RNA is modified by replacing all uracils with the modified uracil. In an embodiment, the proportion of modified uracils in the RNA is 10%-100%, e.g., 10%, 20%, 30%, 40%, 50%, 60%, 70%, 80%, 90%, or 100%. In an embodiment, the proportion of modified uracils in the RNA is 20%-100%. In an embodiment, 20%-100% of uracils in the RNA are replaced with 1-methyl-pseudouracil. In a preferred embodiment, 100% of uracils in the RNA are replaced with 1-methyl-pseudouracil.

1-Methyl-pseudouridine has the following structure:

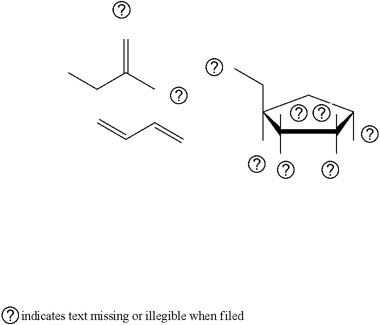

In a specific embodiment, the mRNA of the present disclosure includes a nucleotide sequence of SEQ ID NO: 6, and 100% of uracils therein are replaced with 1-methyl-pseudouracil.

### Use of Lipid Composition and Pharmaceutical Composition

The lipid compositions, pharmaceutical compositions, and intratumoral injectants of the present disclosure may be used for treating cancers. To be precise, these lipid compositions, pharmaceutical compositions, and intratumoral injectants may be used for treating cancers that feature loss or abnormal protein or polypeptide activity. For example, a lipid composition and a pharmaceutical composition including an mRNA with code loss or abnormal polypeptide may be administered or delivered to a tumor. The polypeptide can be produced in the subsequent translation of the mRNA, thereby reducing or eliminating problems due to the absence or aberrant activity of the polypeptide. The therapeutic agent or the prophylactic agent included in the lipid composition can also alter the rate of transcription of a given mRNA, thereby affecting gene expression.

As used herein, “cancers” where the lipid compositions, pharmaceutical compositions, or intratumoral injectants can be administrated include but are not limited to solid tumors or hematologic malignancy. The solid tumor herein includes, for example, squamous cell carcinoma, adenocarcinoma, basal cell carcinoma, renal cell carcinoma, ductal breast carcinoma, soft tissue sarcoma, osteosarcoma, melanoma, small cell lung cancer, non-small cell lung cancer, lung adenocarcinoma, peritoneal carcinoma, hepatocellular carcinoma, gastrointestinal carcinoma, gastric carcinoma, pancreatic carcinoma, neuroendocrine carcinoma, glioblastoma, cervical cancer, ovarian cancer, hepatic carcinoma, bladder cancer, brain cancer, hepatoma, breast carcinoma, colon carcinoma, colorectal carcinoma, endometrial carcinoma or uterine carcinoma, esophageal carcinoma, salivary gland carcinoma, renal carcinoma, hepatic carcinoma, prostate carcinoma, vulvar cancer, thyroid cancer, head and neck cancer, etc., or any combination thereof. The hematologic malignancy includes, for example, leukemia, lymphoma, myeloma, acute myeloid leukemia, chronic myeloid leukemia, acute lymphocytic leukemia, chronic lymphocytic leukemia, hairy cell leukemia, Hodgkin's lymphoma, non-Hodgkin's lymphoma, or multiple myeloma. The cancer may also be a metastatic cancer. “Metastatic” refers to the spread of cancer cells from their original site to other parts of the body.

The method provided by the present disclosure relates to administrating the lipid composition including one or more therapeutic agents or prophylactic agents, and a pharmaceutical composition and an intratumoral injectant including these compositions. For the features and embodiments of the present disclosure, the terms, therapeutic agent and prophylactic agent, are used interchangeably herein. The lipid compositions, the pharmaceutical compositions, and the intratumoral injectants may be administered to a subject using any reasonable amount of administration, and the reasonable amount can effectively achieve the prevention, treatment, and diagnosis of cancers, or be for any other purpose. The specific amount administered to a given subject may vary depending on the species, age and general condition of the subject; the purpose of administration; the specific composition, etc.

The lipid composition provided by the present disclosure or the pharmaceutical composition of the present disclosure is used for tumor administration; the tumor administration preferably includes intratumoral administration, peritumoral subcutaneous administration, or administration in an artery that supplies blood to a tumor, and most preferably intratumoral injection.

In some embodiments, the lipid composition and the pharmaceutical composition including a therapeutic agent or a prophylactic agent of the present disclosure may be administrated to a subject by intratumoral injection.

### Beneficial Effects

The lipid composition, the pharmaceutical composition, or the intratumoral injectant provided by the present disclosure can exhibit excellent effects, for example, but not limited to: (1) improving the expression efficiency of an included mRNA in a tumor; (2) reducing the expression of mRNA in the liver, thereby reducing and decrease hepatotoxicity; (3) reducing the expression of mRNA outside a tumor, thereby reducing systemic toxicity; and (4) improving the effect of tumor treatment.

Further, The lipid composition, the pharmaceutical composition, or the intratumoral injectant including an IL-12 nucleic acid provided by the present disclosure can exhibit excellent effects, for example, but not limited to: (1) high expression in vivo and long half-life of IL-12 expressed; (2) in vitro expression showing dose-dependent; (3) inducing cellular immune response, thereby effectively activating CD8⁺T cells; and (4) a good tumor suppression effect, thereby significantly reducing a tumor volume.

### Example

The present disclosure is further described by reference to the examples below. It needs to be understood that these examples are only exemplary and do not constitute a limitation to the present disclosure. The following materials and instruments are all commercially available or prepared according to methods well known in the art. The following experiments are performed according to the manufacturer's instructions or according to the methods and steps well known in the art.

### Experimental Materials

The cationic lipid, the compound shown in formula (I), was synthesized by Stemirna Therapeutics or might be prepared by reference to CN110520409A; phospholipid (DOPE) was purchased from CordenPharma; cholesterol was purchased from Sigma-Aldrich; mPEG2000-DMG (i.e., DMG-PEG 2000) was purchased from Avanti Polar Lipids, Inc.; PBS was purchased from Invitrogen; protamine sulfate was purchased from Beijing Scrianen Pharmaceutical Co., Ltd.

### Example 1 Synthesis of the Compound According to Formula (I)

### General Consideration

Unless otherwise indicated, all solvents and reagents used were commercially available and used as received. ¹H NMR spectra were recorded in CDCl₃ using Bruker Ultrashield 300 MHz instrument at 300 K. Chemical shifts were reported in parts per million (ppm) for ¹H relative to TMS (0.00). Silica gel column chromatography was performed on ISCO CombiFlash Rf+Lumen instrument using ISCO RediSep Rf Gold flash column (particle size: 20-40 microns).

The procedures described below may be used for synthesis of compounds SW-II-115 to SW-II-140-2.

The following abbreviations are used herein:
THF: Tetrahydrofuran
MeCN: Methyl cyanide
LAH: Lithium aluminium hydride
DCM: Dichloromethane
DMAP: 4-Dimethylaminopyridine
LDA: Lithium diisopropylamide
rt: Room temperature
DME: 1,2-Dimethoxyethane
n-BuLi: n-Butyllithium
CPME: Cyclopentyl methyl ether
EDCI N-(3-Dimethylaminopropyl)-N′-ethylcarbodiimide
DIEA: N,N-iisopropylethylamine
PE: Petroleum ether
EA: Ethyl acetate

### A. Compound SW-II-115

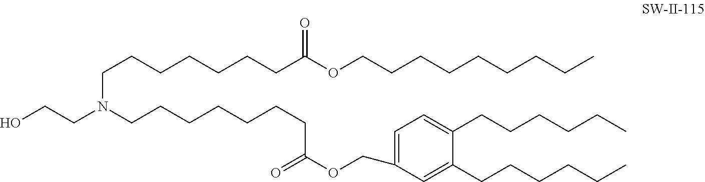 [SMILES: CCCCCCCCCOC(=O)CCCCCCCN(CCO)CCCCCCCC(=O)OCc1ccc(CCCCCC)c(CCCCCC)c1]

### 1. Synthesis of Intermediate 3

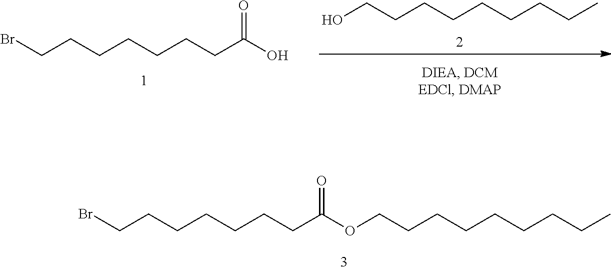

EDCI (17.3 g, 90 mmol, 2 eq.) and DMAP (2.2 g, 18 mmol, 0.4 eq.) were added into a DCM solution (100 mL) containing compound 1 (10 g, 45 mmol, 1 eq.) and compound 2 (7.8 g, 54 mmol, 1.2 eq.), and then DIEA (23.2 g, 180 mmol, 4 eq.) was added. The reaction mixture was stirred at room temperature for 16 hours under N₂ protection. TLC (petroleum ether:ethyl acetate=30:1) showed that compound 1 was consumed and the desired product was formed. The reaction mixture was diluted with DCM (20 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-20:1) to obtain colorless oily compound 3 (4.365 g, 28%).

### 2. Synthesis of Intermediate 5

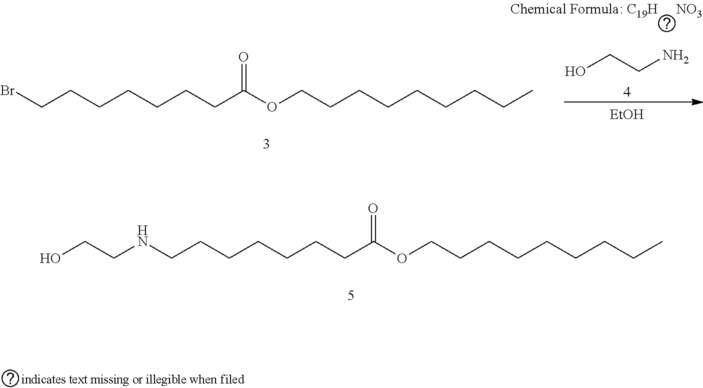

An EtOH solution of compound 3 (500 mg, 1.437 mmol, 1 eq.) and compound 4 (2.63 g, 43.103 mmol, 30 eq.) was stirred at 60° C. for 16 hours under N₂ protection. TLC (DCM:MeOH=10:1) showed that compound 3 was consumed. TLC (DCM/MeOH=10/1) showed that a new major spot was observed. The reaction mixture was concentrated under reduced pressure. The residue was diluted with EtOAc (50 mL) and washed with H₂O (3×50 mL). The organic layer was dried with anhydrous Na₂SO₄, filtered, and concentrated under reduced pressure. The residue was purified by silica gel column chromatography, and eluted with DCM/MeOH (1:0-10:1, v/v) to obtain yellow oily compound 5 (264 mg, 56%).

### 3. Synthesis of Intermediate 8

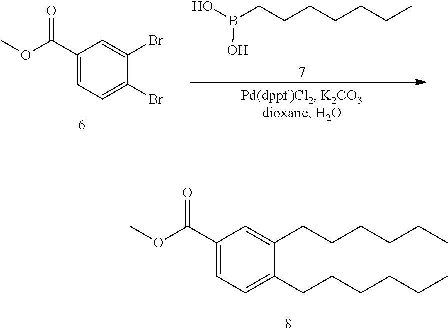

Pd(dppf)Cl₂ (112 mg, 0.171 mmol, 0.1 eq.) and potassium carbonate (709 mg, 5.136 mmol, 3 eq.) were added into a dioxane/water (5 mL/0.5 mL) mixed solvent of compound 6 (500 mg, 1.712 mmol, 1 eq.) and compound 7 (1.113 g, 8.562 mmol, 5 eq.). The mixture was stirred overnight at 100° C. under N₂. TLC (PE:EA=15:1) showed that the reaction was complete and a new major spot was observed. The mixture was extracted with EA and washed with water. The organic layer was dried with anhydrous Na₂SO₄, filtered, and concentrated under vacuum. The residue was purified by silica gel column chromatography, and eluted with PE:EA (1:0-10:1) to obtain colorless oily compound 8 (455 mg, 88%).

### 4. Synthesis of Intermediate 9

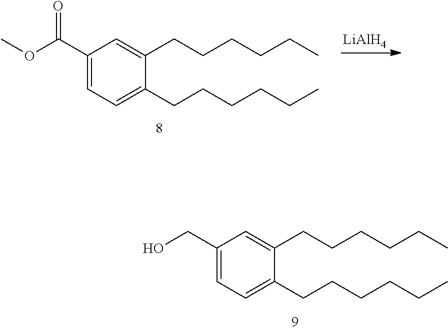 [SMILES: CCCCCCc1ccc(C(=O)OC)cc1CCCCCC ; "LiAlH4" ; CCCCCCc1ccc(CO)cc1CCCCCC]

At 0° C. and under N₂ protection, LiAlH₄ (1.5 mL, 1.497 mmol, 1 M, in THF, 1 eq.) was added into a THF (5 mL) solution of compound 8 (455 mg, 1.497 mmol, 1 eq.). The mixture was stirred at room temperature for 2 hours under N₂. TLC (PE:EtOAc=5:1) showed that the reaction was complete and a new major spot was observed. The mixture was quenched with water (1.5 mL) and treated with 2N HCl to regulate the pH between 6 and 7, extracted with EA and washed with brine. The organic layer was dried with anhydrous Na₂SO₄, filtered, and concentrated under vacuum to obtain crude compound 9 (419 mg, >100%), which was colorless and oily and did not need to be further purified.

### 5. Synthesis of Intermediate 10

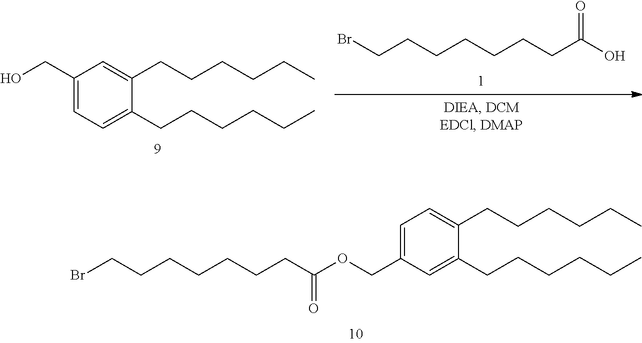 [SMILES: O=C(O)CCCCCCCBr ; CCCCCCc1ccc(CO)cc1CCCCCC ; CCCCCCc1ccc(COC(=O)CCCCCCCBr)cc1CCCCCC]

EDCI (583 mg, 3.036 mmol, 2 eq.) and DMAP (74 mg, 0.607 mmol, 0.4 eq.) were added into a DCM (4 mL) solution containing compound 1 (339 mg, 1.518 mmol, 1 eq.) and compound 9 (419 mg, 1.518 mmol, 1 eq.), and then DIEA (783 mg, 6.072 mmol, 4 eq.) was added. The reaction mixture was stirred at room temperature for 16 hours under N₂ protection. TLC (petroleum ether:ethyl acetate=10:1) showed that the desired product was formed. The reaction mixture was extracted with EA and washed with water. The organic layer was dried with anhydrous Na₂SO₄, filtered, and concentrated under vacuum. The residue was purified by silica gel column chromatography, and eluted with petroleum ether:ethyl acetate (1:0-10:1) to obtain compound 10 (443 mg, 60.7%), which was colorless oil.

### 6. Synthesis of Final Product SW-II-115

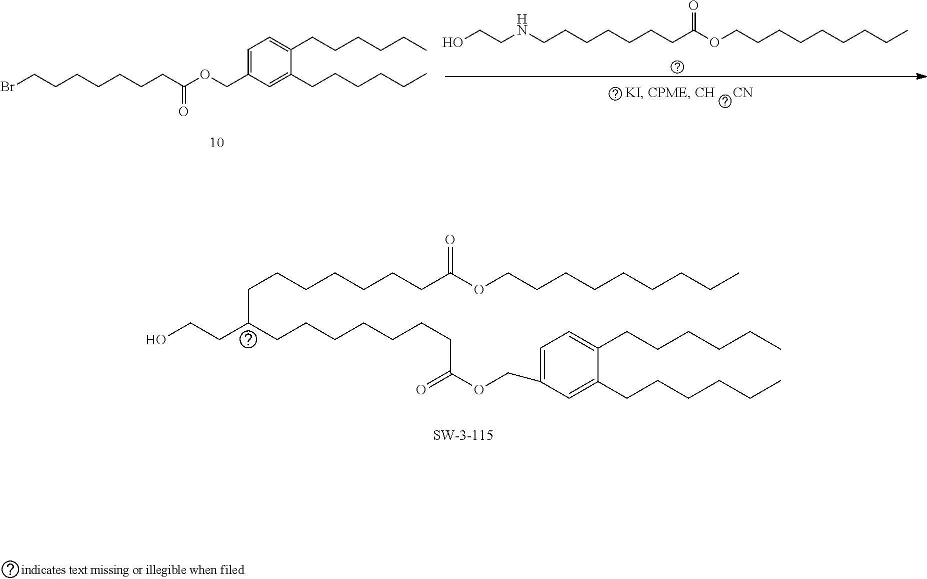

K₂CO₃ (530 mg, 3.84 mmol, 6 eq.) and KI (212 mg, 1.28 mmol, 2 eq.) were added into a mixed solvent CPME/CH₃CN (3 mL/3 mL) containing compound 10 (307 mg, 0.64 mmol, 1 eq.) and compound 5 (210 mg, 0.64 mmol, 1 eq.). After the addition was completed, the mixture was stirred overnight at 90° C. under N₂. TLC (DCM:MeOH=10:1) showed that the reaction was complete and a new major spot was observed. The mixture was extracted with EA and washed with water. The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily compound SW-II-115 (266 mg, 57%).

LCMS: Rt: 1.293 min; MS m/z (ELSD): 730.5[M+H]⁺;

HPLC: 99.472% purity, ELSD; RT=4.895 min.

¹H NMR (400 MHz, CDCl₃) δ 7.21-6.99 (m, 3H), 5.05 (s, 2H), 4.05 (t, J=6.8 Hz, 2H), 3.58 (t, J=5.3 Hz, 2H), 2.69-2.46 (m, 10H), 2.31 (dt, J=20.0, 7.5 Hz, 4H), 1.69-1.18 (m, 51H), 0.89 (dt, J=12.4, 6.3 Hz, 9H).

¹³C NMR (101 MHz, CDCl₃) δ 173.90 (s), 173.68 (s), 140.80 (d, J=13.0 Hz), 133.31 (s), 129.25 (d, J=16.2 Hz), 128.30 (s), 125.75 (s), 77.30 (d, J=11.5 Hz), 77.04 (s), 76.72 (s), 66.22 (s), 64.43 (s), 58.12 (s), 55.72 (s), 53.90 (s), 34.32 (d, J=1.9 Hz), 32.69 (s), 32.48 (s), 31.81 (d, J=11.2 Hz), 31.25 (s), 29.59-28.91 (m), 28.66 (s), 27.17 (s), 26.64 (s), 25.94 (s), 24.91 (d, J=5.1 Hz), 22.65 (d, J=3.3 Hz), 14.10 (s).

### B. Compound SW-II-118

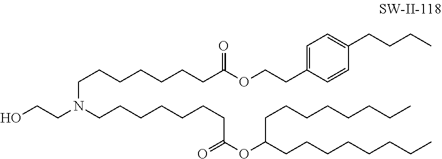 [SMILES: CCCCCCCCC(CCCCCCCC)OC(=O)CCCCCCCN(CCO)CCCCCCCC(=O)OCCc1ccc(CCCC)cc1]

### 1. Synthesis of Intermediate 3

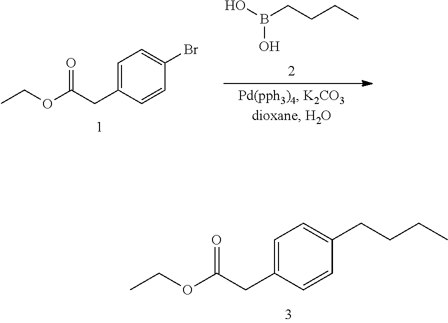

A toluene (10 ml) and H₂O (1 ml) solution of compound 1 (1.22 g, 5.0 mmol, 1.0 eq.), compound 2 (765 mg, 7.5 mmol, 1.5 eq.), Pd(PPh₃)₄(tetrakis(triphenylphosphine)palladium, 289 mg, 0.25 mmol, 0.05 eq.) and K₂CO₃ (1.38 g, 10.0 mmol, 2.0 eq.) was stirred at 110° C. for 1 hour under N₂ protection. TLC (petroleum ether:ethyl acetate=19:1) showed that compound 1 was consumed and a new spot was observed. The reaction mixture was diluted with DCM (50 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-10:1) to obtain colorless oily compound 3 (0.5 g, 45%).

¹H NMR (400 MHz, CDCl₃) δ 7.16 (dd, J=23.5, 8.1 Hz, 4H), 4.14 (q, J=7.1 Hz, 2H), 3.57 (s, 2H), 2.64-2.48 (m, 2H), 1.66-1.51 (m, 2H), 1.35 (dd, J=15.0, 7.4 Hz, 2H), 1.25 (t, J=7.1 Hz, 3H), 0.92 (t, J=7.3 Hz, 3H).

### 2. Synthesis of Intermediate 4

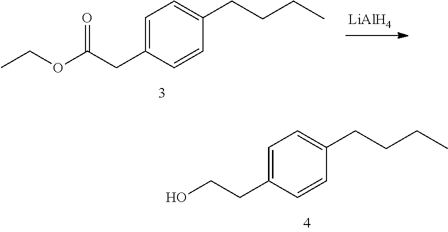

LiAlH₄ (193 mg, 5.09 mmol, 4.0 eq.) was added into a THF (10 mL) solution containing compound 3 (280 mg, 1.27 mmol, 1.0 eq.) at −78° C., and then the reaction mixture reacted at 10° C. for 3 hours. TLC showed that the reaction was very good. The reaction mixture was concentrated, diluted with Na₂SO₄ (20 mL), and extracted with EA (30 mL×2). The organic phase was dried with anhydrous Na₂SO₄, filtered, and concentrated under reduced pressure to obtain yellow oily compound 4 (3.12 g, crude product).

### 3. Synthesis of Intermediate 6

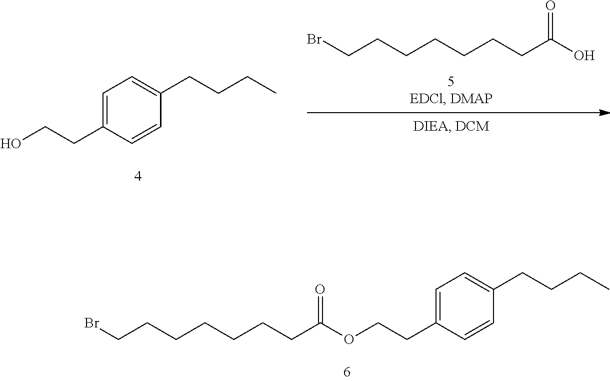 [SMILES: O=C(O)CCCCCCCBr ; CCCCc1ccc(CCO)cc1 ; CCCCc1ccc(CCOC(=O)CCCCCCCBr)cc1]

A DCM (5 mL) solution containing compound 4 (215 mg, 1.2 mmol, 1.0 eq.), compound 5 (404 mg, 1.8 mmol, 1.5 eq.), EDCI (1.15 g, 6.0 mmol, 5.0 eq.), DMAP (732 mg, 1.8 eq.), DIEA (1.29 g, 12.0 mmol, 10.0 eq.) and DIEA (1.29 g, 12.0 mmol, 10.0 eq.) was stirred at 10° C. for 16 hours under N₂ protection. TLC (DCM:MeOH=10:1) showed that the reaction was complete and a new major spot was observed. The mixture was concentrated under reduced pressure. The residue was purified by silica gel column chromatography, and eluted with PE:EA (1:0-10:1, v/v) to obtain colorless oily compound 6 (145 mg, 31%).

¹H NMR (400 MHz, CDCl₃) δ7.12 (s, 4H), 4.27 (t, J=7.1 Hz, 2H), 3.52 (t, J=6.7 Hz, 1H), 3.40 (t, J=6.8 Hz, 1H), 2.90 (t, J=7.1 Hz, 2H), 2.65-2.50 (m, 2H), 2.28 (t, J=7.5 Hz, 2H), 1.93-1.70 (m, 2H), 1.64-1.56 (m, 4H), 1.44-1.27 (m, 8H), 0.92 (t, J=7.3 Hz, 3H).

### 4. Synthesis of Final Product SW-II-118

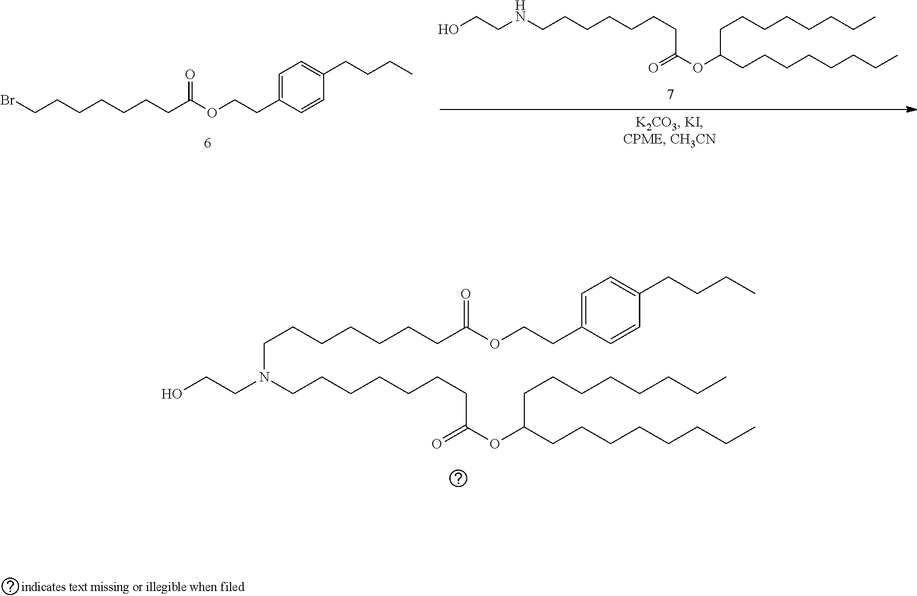 [SMILES: CCCCc1ccc(CCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccc(CCCC)cc1]

A CPME (1 mL) and CH₃CN (1 mL) mixed solvent containing a mixture of compound 6 (140 mg, 0.37 mmol, 1.0 eq.), compound 7 (243 mg, 0.55 mmol, 1.5 eq.), K₂CO₃ (153 mg, 1.11 mmol, 3.0 eq.) and KI (123 mg, 0.74 mmol, 2.0 eq.) was stirred at 90° C. for 16 hours under N₂. The reaction mixture was concentrated under reduced pressure. The residue was diluted with EtOAc (50 mL) and washed with NaHCO₃ (30 mL). The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily SW-11-118 (105 mg, 61%).

LCMS: Rt: 1.946 min; MS m/z (ELSD): 744.4[M+H]⁺;

HPLC: 99.64% purity, ELSD; RT=5.875 min.

¹H NMR (400 MHz, CDCl₃) δ7.11 (s, 4H), 4.91-4.79 (m, 1H), 4.26 (t, J=7.2 Hz, 2H), 3.80-3.68 (m, 2H), 2.90 (t, J=7.1 Hz, 4H), 2.81-2.67 (m, 4H), 2.62-2.52 (m, 2H), 2.28 (td, J=7.5, 2.6 Hz, 4H), 1.64-1.51 (m, 11H), 1.38-1.17 (m, 42H), 0.93-0.82 (m, 9H).

¹³C NMR (101 MHz, CDCl₃) δ 173.61 (d, J=11.7 Hz), 141.11 (s), 134.90 (s), 128.74 (s), 128.51 (s), 77.40 (s), 77.08 (s), 76.77 (s), 74.17 (s), 64.90 (s), 57.4 8 (s), 56.24 (s), 53.98 (s), 35.25 (s), 34.66 (d, J=14.4 Hz), 34.16 (d, J=5.1 Hz), 33.67 (s), 31.86 (s), 29.52 (d, J=2.4 Hz), 29.24 (s), 29.21-28.74 (m), 26.90 (d, J=4.9 Hz), 25.42-24.92 (m), 24.92-24.88 (m), 24.74 (s), 22.67 (s), 22.37 (s), 14.04 (d, J=15.7 Hz).

### C. Compound SW-II-120

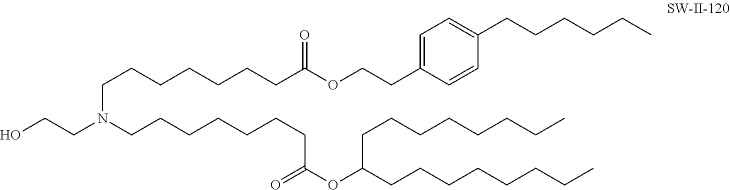 [SMILES: CCCCCCCCC(CCCCCCCC)OC(=O)CCCCCCCN(CCO)CCCCCCCC(=O)OCCc1ccc(CCCCCC)cc1]

### 1. Synthesis of Intermediate 3

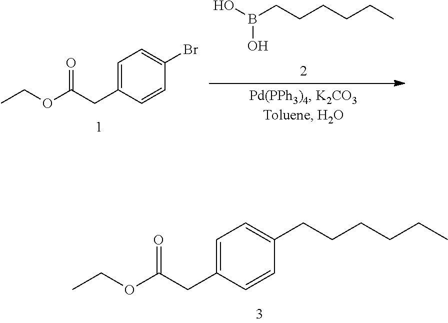

A toluene (10 ml) and H₂O (1 ml) mixed solution containing compound 1 (1.22 g, 5.0 mmol, 1.0 eq.), compound 2 (1.30 mg, 10.0 mmol, 2.0 eq.), Pd(PPh₃)₄ (289 mg, 0.25 mmol, 0.05 eq.) and K₂CO₃ (1.38 g, 10.0 mmol, 2.0 eq.) was stirred at 110° C. for 1 hour under N₂ protection. TLC (petroleum ether:ethyl acetate=19:1) showed that compound 1 was consumed and a new spot was observed. The reaction mixture was diluted with DCM (50 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-10:1) to obtain colorless oily compound 3 (0.78 g, 62%).

¹H NMR (400 MHz, CDCl₃) δ7.19 (d, J=8.1 Hz, 2H), 7.13 (d, J=8.1 Hz, 2H), 4.14 (q, J=7.1 Hz, 2H), 3.57 (s, 2H), 2.62-2.51 (m, 2H), 1.58 (d, J=11.1 Hz, 2H), 1.35-1.21 (m, 9H), 0.88 (t, J=6.7 Hz, 3H).

### 2. Synthesis of Intermediate 4

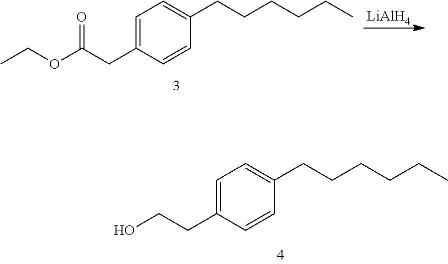

LiAlH₄ (477 mg, 12.56 mmol, 4.0 eq.) was added into a THF (10 mL) solution containing compound 3 (780 mg, 3.14 mmol, 1.0 eq.) at −78° C., and then the reaction mixture was stirred at 10° C. for 3 hours. Thin layer chromatography showed that the reaction proceeded well. The reaction mixture was concentrated, diluted with Na₂SO₄ (20 mL), and extracted with EA (30 mL*2). The organic phase was dried with anhydrous Na₂SO₄, filtered, and concentrated under reduced pressure to obtain colorless oily compound 4 (640 mg, crude product).

### 3. Synthesis of Intermediate 6

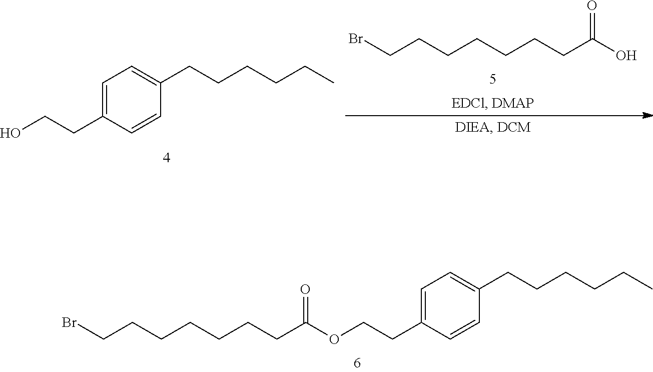 [SMILES: O=C(O)CCCCCCCBr ; CCCCCCc1ccc(CCO)cc1 ; CCCCCCc1ccc(CCOC(=O)CCCCCCCBr)cc1]

A DCM (10 mL) solution containing compound 4 (640 mg, 3.10 mmol, 1.0 eq.), compound 5 (1.06 g, 4.70 mmol, 1.5 eq.), EDCI (2.98 g, 15.5 mmol, 5.0 eq.), DMAP (1.85 g, 15.0 eq.) and DIEA (4.0 g, 31.0 mmol, 10.0 eq.) was stirred at 10° C. for 16 hours under N₂ protection. TLC (DCM:MeOH=10:1) showed that the reaction was complete and a new major spot was observed. The mixture was concentrated under reduced pressure. The residue was purified by silica gel column chromatography, and eluted with PE:EA (1:0-10:1, v/v) to obtain colorless oily compound 6 (465 mg, 36%).

### 4. Synthesis of Final Product SW-II-120

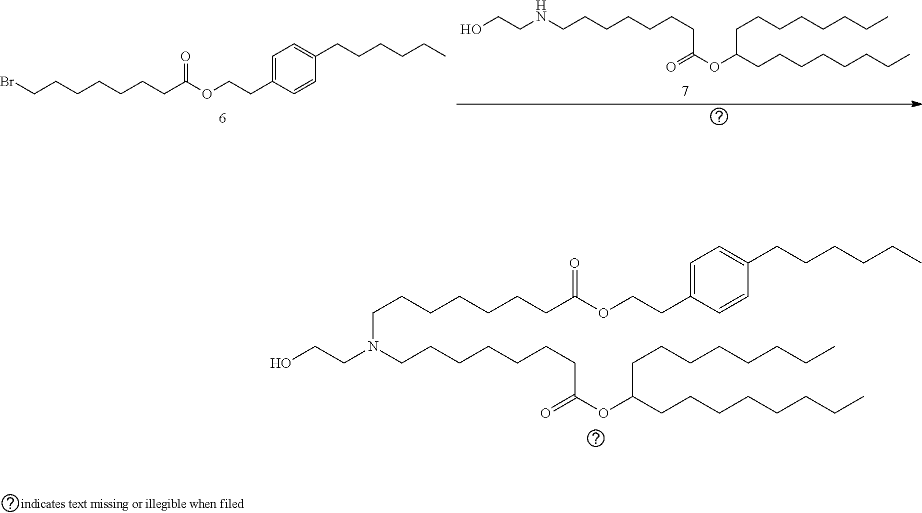 [SMILES: CCCCCCc1ccc(CCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccc(CCCCCC)cc1]

A CPME (1 mL) and CH₃CN (1 mL) mixed solvent containing a mixture of compound 6 (100 mg, 0.25 mmol, 1.0 eq.), compound 7 (161 mg, 0.36 mmol, 1.5 eq.), K₂CO₃ (104 mg, 0.75 mmol, 3.0 eq.) and KI (83 mg, 0.50 mmol, 2.0 eq.) was stirred at 90° C. for 16 hours under N₂. The reaction mixture was concentrated under reduced pressure. The residue was diluted with EtOAc (50 mL) and washed with NaHCO₃ (30 mL). The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily SW-II-120 (100 mg, 52%).

LCMS: Rt: 2.500 min; MS m/z (ELSD): 772.4[M+H]⁺;

HPLC: 99.70% purity, ELSD; RT=8.675 min.

¹H NMR (400 MHz, CDCl₃) δ7.07 (d, J=8.9 Hz, 4H), 4.89-4.73 (m, 1H), 4.23 (t, J=7.2 Hz, 2H), 3.83-3.65 (m, 2H), 2.87 (t, J=7.2 Hz, 4H), 2.82-2.67 (m, 4H), 2.61-2.45 (m, 2H), 2.25 (td, J=7.5, 2.5 Hz, 4H), 1.65-1.44 (m, 15H), 1.27 (dd, J=13.2, 11.3 Hz, 42H), 0.85 (t, J=6.8 Hz, 9H).

¹³C NMR (101 MHz, CDCl₃) δ 173.57 (d, J=11.5 Hz), 141.13 (s), 134.88 (s), 128.73 (s), 128.48 (s), 77.45 (s), 77.13 (s), 76.81 (s), 74.14 (s), 64.89 (s), 57.3 4 (s), 56.17 (s), 53.92 (s), 35.57 (s), 34.64 (d, J=16.1 Hz), 34.14 (d, J=3.3 Hz), 31.79 (d, J=13.4 Hz), 31.49 (s), 29.50 (d, J=2.2 Hz), 29.23 (s), 29.10-28.71 (m), 26.85 (d, J=5.0 Hz), 25.49-25.38 (m), 25.13 (d, J=35.4 Hz), 24.72 (s), 22.63 (d, J=5.8 Hz), 14.11 (s).

### D. Compound SW-II-121

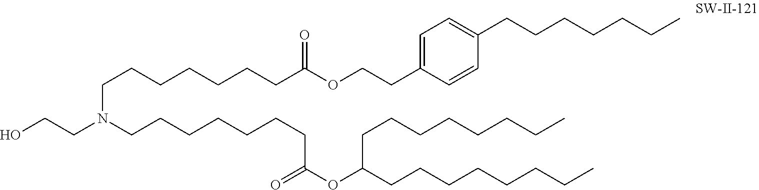 [SMILES: CCCCCCCCC(CCCCCCCC)OC(=O)CCCCCCCN(CCO)CCCCCCCC(=O)OCCc1ccc(CCCCCCC)cc1]

### 1. Synthesis of Intermediate 3

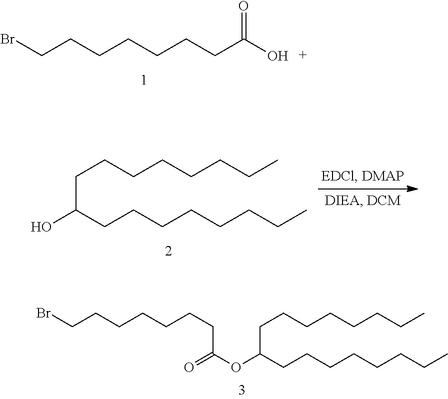

EDCI (1.495 g, 7.8 mmol, 2.0 eq.), DMAP (0.19 g, 1.56 mmol, 0.4 eq.) and DIEA (2.57 mL, 15.6 mmol, 4.0 eq.) were added into a DCM (20 mL) solution containing compound 1 (1.3 g, 5.86 mmol, 1.5 eq.) and compound 2 (1 g, 3.9 mmol, 1.0 eq.). The reaction mixture was stirred at room temperature for 16 hours under N₂. TLC (petroleum ether:ethyl acetate=19:1) showed that compound 2 was consumed and the desired product was formed. The reaction mixture was diluted with DCM (20 mL), washed with H₂O (40 mL), dried with anhydrous Na₂SO₄, filtered, and concentrated under reduced pressure. The residue was purified by silica gel column chromatography, and eluted with petroleum ether:ethyl acetate (1:0-10:1) to obtain yellow oily compound 3 (1.2 g, 66.9%).

¹H NMR (400 MHz, CDCl₃) δ4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Intermediate 5

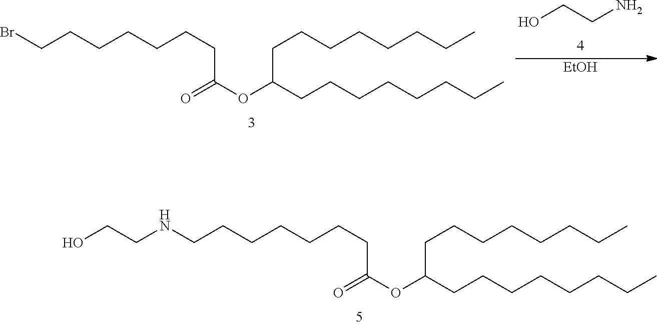

An EtOH (5 mL) solution containing compound 3 (5.2 g, 11.30 mmol, 1.0 eq.) and compound 4 (20.6 g, 339 mmol, 30 eq.) was stirred at 60° C. for 16 hours under N₂ protection. TLC (petroleum ether:ethyl acetate=19:1) showed that compound 3 was consumed. Moreover, TLC (DCM/MeOH=10/1) showed that a new major spot was observed. The reaction mixture was concentrated under reduced pressure. The residue was diluted with EtOAc (50 mL) and washed with H₂O (3×50 mL). The organic layer was dried with anhydrous Na₂SO₄, filtered, and concentrated under vacuum. The residue was purified by silica gel column chromatography, and eluted with DCM:MeOH (1:0-10:1, v/v) to obtain yellow oily compound 5 (3 g, 60%).

### 3. Synthesis of Intermediate 8

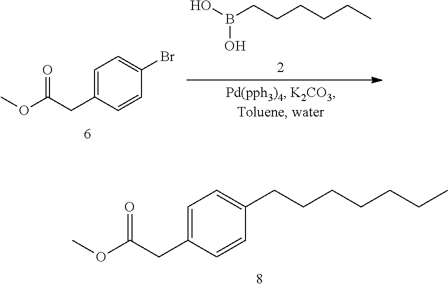

Pd(pph₃)₄ (238 mg, 0.206 mmol, 0.05 eq.) and K₂CO₃ (1.7 g, 12.35 mmol, 3 eq.) were added into a toluene/water (10 mL/1 mL) mixed solution containing compound 6 (1 g, 4.115 mmol, 1 eq.) and compound 7 (889 mg, 6.173 mmol, 1.5 eq.). The mixture was stirred at 110° C. for 2 hours under N₂. TLC (PE:EA=10:1) showed that the reaction was complete and a new major spot was observed. The mixture was extracted with EA and washed with water. The organic layer was dried with anhydrous Na₂SO₄, filtered, and concentrated under vacuum. The residue was purified by silica gel column chromatography, and eluted with PE:EA (1:0-10:1) to obtain colorless oily compound 8 (714 mg, 66%).

### 4. Synthesis of Intermediate 9

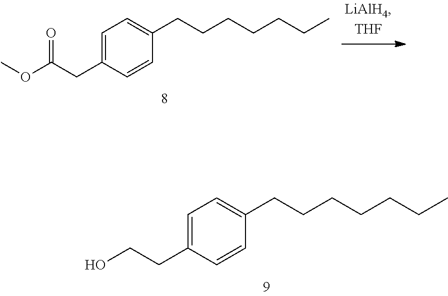 [SMILES: CCCCCCCc1ccc(CC(=O)OC)cc1 ; CCCCCCCc1ccc(CCO)cc1]

LiAH₄ (2.7 mL, 2.725 mmol, 1 M, in THF, 1 eq.) was added into a mixture of compound 8 (714 mg, 2.725 mmol, 1 eq.) in a THF (7 mL) solution at 0° C. under N₂ protection. The mixture was stirred at room temperature for 2 hours. TLC (PE:EtOAc=10:1) showed that the reaction was complete and a new major spot was observed. The mixture was quenched with water (2.7 mL) and treated with 2N HCl to regulate the pH between 6 and 7, extracted with EA and washed with brine. The organic layer was dried with anhydrous Na₂SO₄, filtered, and concentrated under vacuum. The residue was purified by silica gel column chromatography, and eluted with PE:EA (1:0-10:1) to obtain colorless oily compound 9 (103 mg, 63%).

### 5. Synthesis of Intermediate 11

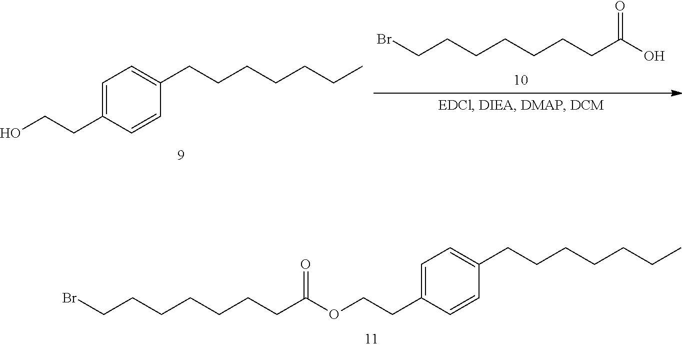

EDCI (524 mg, 2.728 mmol, 2 eq.), DMAP (67 mg, 0.546 mmol, 0.4 eq.), and DIEA (704 mg, 5.456 mmol, 4 eq.) were added into DCM (3 mL) containing compound 9 (300 mg, 1.364 mmol, 1 eq.) and compound 10 (363 mg, 1.64 mmol, 1.2 eq.). The reaction mixture was stirred at room temperature for 16 hours under N₂. TLC (petroleum ether:ethyl acetate=10:1) showed that the desired product was formed. The reaction mixture was extracted with EA and washed with water. The organic layer was dried with anhydrous Na₂SO₄, filtered, and concentrated under vacuum. The residue was purified by silica gel column chromatography, and eluted with petroleum ether:ethyl acetate (1:0-10:1) to obtain colorless oily compound 11 (169 mg, 29%).

### 6. Synthesis of Final Product SW-II-121

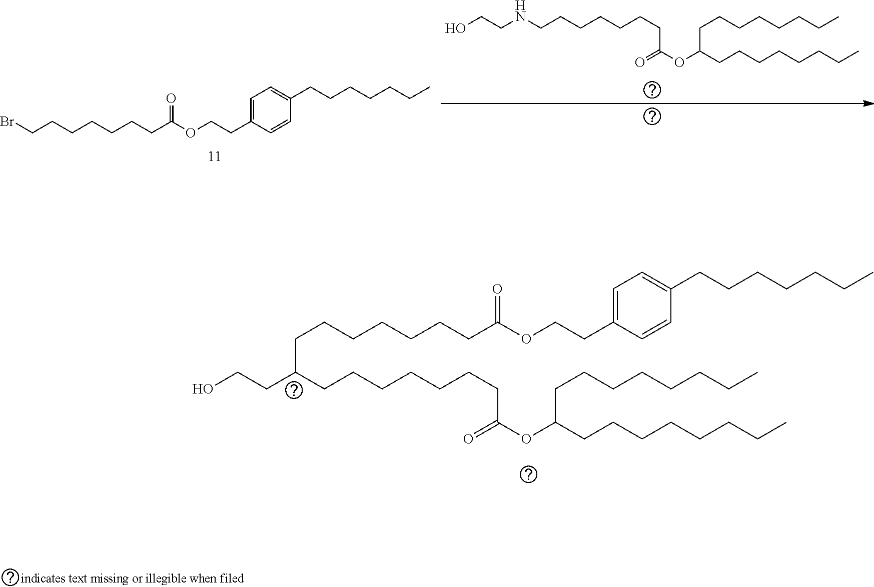 [SMILES: CCCCCCCCC(CCCCCCCC)OC(=O)CCCCCCCNCCO ; CCCCCCCc1ccc(CCOC(=O)CCCCCCCBr)cc1 ; CCCCCCCCC(CCCCCCCC)OC(=O)CCCCCCCC(CCO)CCCCCCCC(=O)OCCc1ccc(CCCCCCC)cc1]

K₂CO₃ (330 mg, 2.394 mmol, 6 eq.) and KI (132 mg, 0.798 mmol, 2 eq.) were added into a CPME/CH₃CN (2 mL/2 mL) mixed solvent containing compound 11 (169 mg, 0.399 mmol, 1 eq.) and compound 5 (176 mg, 0.399 mmol, 1 eq.). After the addition was completed, the mixture was stirred overnight at 90° C. under N₂. TLC (DCM:MeOH=10:1) showed that the reaction was complete and a new major spot was observed. The mixture was extracted with EA and washed with water. The organic layer was dried with anhydrous Na₂SO₄, filtered, and concentrated under reduced pressure. The residue was purified by silica gel column chromatography, and eluted with DCM:MeOH (1:0-10:1, v/v) to obtain yellow oily compound SW-II-121 (145 mg, 46%).

LCMS: Rt: 1.493 min; MS m/z (ELSD): 786.5[M+H]⁺;

HPLC: 99.869% purity, ELSD; RT=10.655 min.

¹H NMR (400 MHz, CDCl₃) δ7.11 (s, 4H), 4.92-4.80 (m, 1H), 4.26 (t, J=7.2 Hz, 2H), 3.80 (s, 2H), 2.87 (dd, J=26.6, 19.4 Hz, 7H), 2.62-2.51 (m, 2H), 2.28 (td, J=7.2, 3.6 Hz, 4H), 1.75-1.45 (m, 14H), 1.42-1.09 (m, 45H), 0.88 (t, J=6.8 Hz, 9H).

¹³C NMR (101 MHz, CDCl₃) δ 173.61 (d, J=12.3 Hz), 141.20 (s), 134.90 (s), 128.75 (s), 128.51 (s), 77.35 (s), 77.03 (s), 76.72 (s), 74.21 (s), 64.93 (s), 54.15 (s), 35.59 (s), 34.66 (d, J=16.6 Hz), 34.16 (d, J=3.0 Hz), 31.85 (d, J=4.4 Hz), 31.55 (s), 29.64-29.15 (m), 29.15-28.78 (m), 26.85 (d, J=4.5 Hz), 25.33 (s), 24.95 (s), 24.72 (s), 22.68 (s), 14.12 (s).

### E. Compound SW-II-122

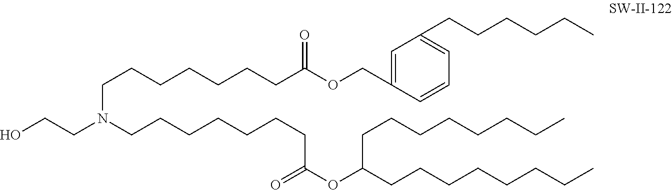 [SMILES: CCCCCCCCC(CCCCCCCC)OC(=O)CCCCCCCN(CCO)CCCCCCCC(=O)OCc1cccc(CCCCCC)c1]

### 1. Synthesis of Compound 3

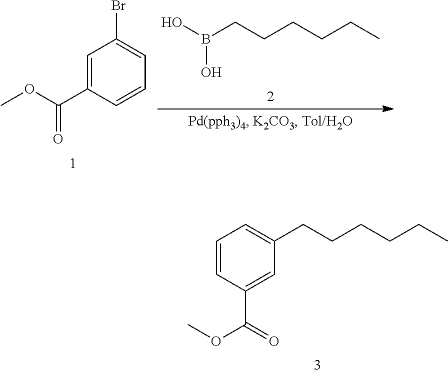

Compound 1 (1 g, 4.65 mmol, 1 eq.) and compound 2 (726 mg, 5.58 mmol, 1.2 eq.) were dissolved in toluene/water (10/1, 20 mL), and then K₂CO₃ (1.92 g, 13.9 mmol, 3 eq.) and Pd(pph₃)₄ (269 mg, 0.23 mmol, 0.05 eq.) were added into the mixture. The reaction mixture was placed in N₂, heated to 110° C., and stirred for 2 hours. TLC (petroleum ether/ethyl acetate=19/1) showed that compound 1 was consumed and a new spot was observed. The reaction mixture was quenched with H₂O (80 mL) and extracted with ethyl acetate (60 mL×3). The organic layer was dried with anhydrous sodium sulfate, filtered, and concentrated under reduced pressure. The residue was purified by silica gel column chromatography, and eluted with petroleum ether/ethyl acetate (1/0-10/1) to obtain yellow oily compound 3 (800 mg, 78%).

### 2. Synthesis of Compound 4

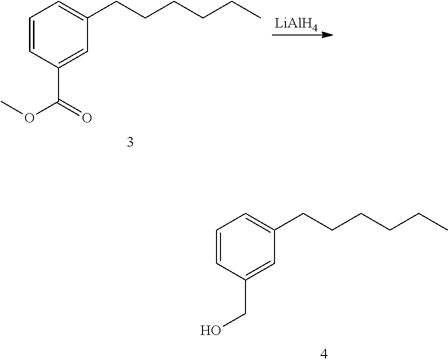

LiAlH₄ (3.2 mL, 3.18 mmol, 1 eq.) was added into compound 3 (700 mg, 3.18 mmol, 1.0 eq.) dissolved in THF (14 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with water (3.2 mL) and 1 M HCl (3.2 mL) respectively. Water (6 mL) was then added into the mixture. The mixture was extracted with ethyl acetate (60 mL×3). The organic layer was washed with brine (30 mL×2), dried with anhydrous sodium sulfate, filtered, and concentrated under reduced pressure. The residue was purified by silica gel column chromatography, and eluted with ethyl acetate/petroleum ether=1/10 to obtain yellow oily compound 4 (600 mg, 98%).

### 3. Synthesis of Compound 6

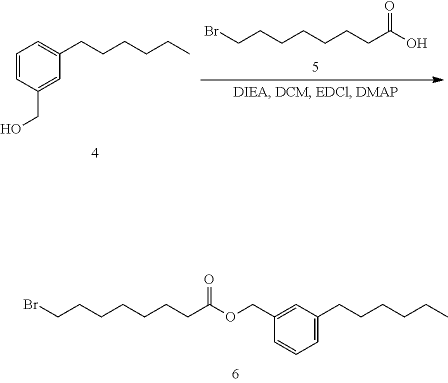 [SMILES: CCCCCCc1cccc(CO)c1 ; O=C(O)CCCCCCCBr ; CCCCCCc1cccc(COC(=O)CCCCCCCBr)c1]

Compound 4 (680 mg, 3.5 mmol, 1.0 eq.) and compound 5 (1.13 g, 5.1 mmol, 1.5 eq.) were dissolved in DCM (10 mL). EDCI (1.20 g, 6.25 mmol, 2.0 eq.), DMAP (166 mg, 1.36 mmol, 0.4 eq.), and DIEA (1.78 g, 13.8 mmol, 4.0 eq.) were added into the mixture. After the addition was completed, the reaction mixture was stirred overnight at room temperature under nitrogen protection. TLC (DCM/MeOH=30/1) showed that a starting material was consumed and a new spot was formed. The mixture was quenched with water (70 mL) and extracted with DCM (80 mL×3). The combined organic layer was washed with brine (2×20 mL), dried with anhydrous sodium sulfate, filtered, and concentrated under reduced pressure. The residue was purified by silica gel column chromatography, and eluted with a solution with ethyl acetate/petroleum ether=3/97 to obtain yellow oily compound 6 (680 mg, 48.5%).

### 4. Synthesis of SW-II-122

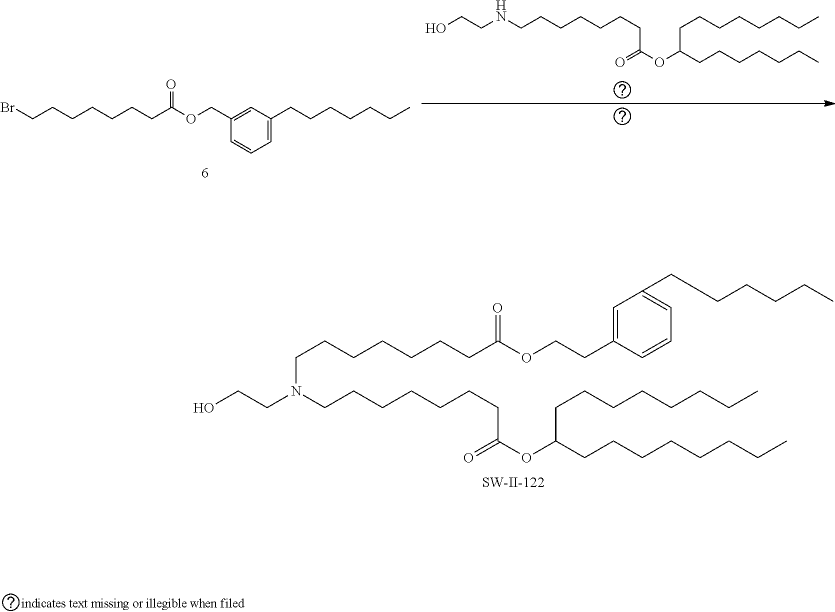 [SMILES: CCCCCCCCC(CCCCCCC)OC(=O)CCCCCCCNCCO ; CCCCCCCc1cccc(COC(=O)CCCCCCCBr)c1 ; CCCCCCCCC(CCCCCCCC)OC(=O)CCCCCCCN(CCO)CCCCCCCC(=O)OCCc1cccc(CCCCCC)c1]

Compound 6 (108 mg, 0.27 mmol, 1.2 eq.) and compound 7 (100 mg, 0.23 mmol, 1 eq.) were dissolved in a CPME (2 mL) and CH₃CN (2 mL) mixed solvent. Potassium carbonate (157 mg, 1.14 mmol, 5.0 eq.) and potassium iodide (75 mg, 0.45 mmol, 2.0 eq.) were added into the mixture. After the addition was completed, the reaction mixture was stirred at 90° C. for 16 hours under nitrogen protection. TLC (DCM/MeOH=10/1) showed that the reaction was complete. The reaction mixture was concentrated under reduced pressure. The residue was purified by silica gel column chromatography, and eluted with DCM/MeOH (1/0-10:1, v/v) to obtain SW-II-122 (68 mg, 40%), which was colorless and oily.

LCMS: Rt: 1.487 min; MS m/z (ELSD): 758.5[M+H]⁺;

HPLC: 97.3% purity, ELSD; RT=7.622 min.

¹H NMR (400 MHz, CDCl₃) δ7.32 (d, J=26.4 Hz, 1H), 7.17 (dd, J=27.2, 21.1 Hz, 3H), 5.09 (s, 2H), 4.91-4.79 (m, 1H), 3.85 (s, 2H), 2.98 (s, 2H), 2.87 (s, 4H), 2.65-2.54 (m, 2H), 2.35 (t, J=7.6 Hz, 2H), 2.28 (t, J=7.6 Hz, 2H), 1.74-1.57 (m, 9H), 1.50 (d, J=5.6 Hz, 4H), 1.37-1.15 (m, 43H), 0.94-0.80 (m, 9H).

¹³C NMR (101 MHz, CDCl₃) δ 173.55 (d, J=2.4 Hz), 143.35 (s), 135.92 (s), 128.67-128.19 (m), 125.47 (s), 77.36 (s), 77.04 (s), 76.73 (s), 74.22 (s), 66.27 (s), 57.15 (s), 56.74 (s), 54.14 (s), 35.88 (s), 34.55 (s), 34.15 (d, J=3.6 Hz), 31.79 (d, J=15.2 Hz), 31.43 (s), 29.52 (d, J=2.8 Hz), 29.25 (s), 28.92 (dd, J=14.2, 5.8 Hz), 26.77 (d, J=4.8 Hz), 25.33 (s), 24.92 (s), 24.71 (s), 24.48 (s), 22.64 (d, J=6.8 Hz), 14.12 (s).

### F. Compound SW-II-127

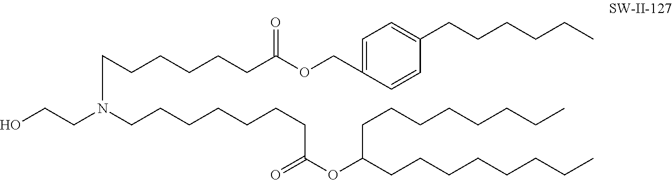 [SMILES: CCCCCCCCC(CCCCCCCC)OC(=O)CCCCCCCN(CCO)CCCCCCC(=O)OCc1ccc(CCCCCC)cc1]

1. Synthesis of compound 3

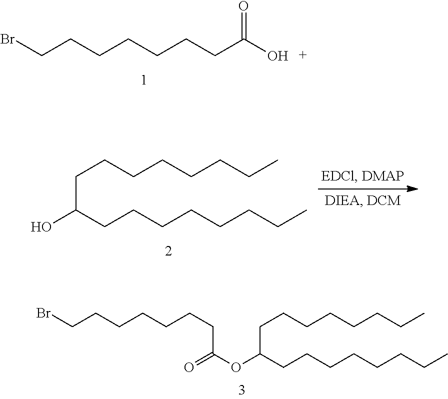

Compound 1 (1.3 g, 5.86 mmol, 1.5 eq.) and compound 2 (1 g, 3.9 mmol, 1.0 eq.) were dissolved in DCM (20 mL). EDCI (1.495 g, 7.8 mmol, 2.0 eq.) and DMAP (0.19 g, 1.56 mmol, 0.4 eq.) were added into the mixture. Then, DIEA (2.57 mL, 15.6 mmol, 4.0 eq.) was added. The reaction mixture was stirred at room temperature for 16 hours under nitrogen protection. TLC (petroleum ether/ethyl acetate=19/1) showed that compound 2 was consumed and the desired product was formed. The reaction mixture was diluted with DCM (20 mL), washed with H₂O (40 mL), dried with anhydrous Na₂SO₄, filtered, and concentrated under reduced pressure. The residue was purified by silica gel column chromatography, and eluted with petroleum ether/ethyl acetate (1/0-10/1) to obtain yellow oily compound 3 (1.2 g, 66.9%).

¹H NMR (400 MHz, CDCl₃) δ4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Compound 5

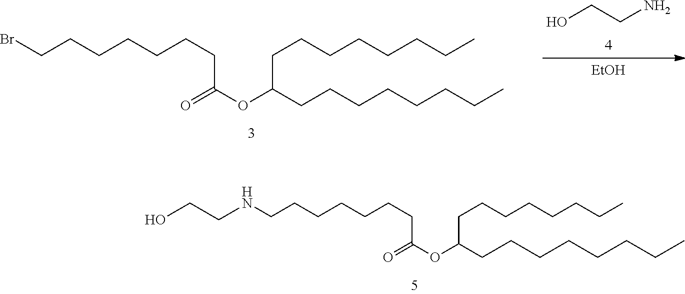

Compound 3 (5.2 g, 11.30 mmol, 1.0 eq.) and compound 4 (20.6 g, 339 mmol, 30 eq.) were added into EtOH (5 mL). Then, the mixture was stirred at 60° C. for 16 hours under nitrogen protection. TLC (petroleum ether/ethyl acetate=19/1) showed that compound 3 was consumed. Moreover, TLC (DCM/MeOH=10/1) showed that a new major spot was observed. The reaction mixture was concentrated under reduced pressure. The residue was diluted with EtOAc (50 mL) and washed with H₂O (3×50 mL). The organic layer was dried with anhydrous sodium sulfate, filtered, and concentrated under reduced pressure. The residue was purified by silica gel column chromatography, and eluted with DCM/MeOH (1/0-10:1, v/v) to obtain yellow oily compound 5 (3 g, 60%).

¹HNMR (400 MHz, CDCl₃) δ4.95-4.75 (m, 1H), 3.74-3.58 (m, 2H), 2.87-2.74 (m, 2H), 2.69-2.56 (m, 2H), 2.36 (s, 2H), 2.28 (t, J=7.5 Hz, 2H), 1.65-1.42 (m, 8H), 1.38-1.17 (m, 30H), 0.88 (t, J=6.8 Hz, 6H).

### 3. Synthesis of Compound 8

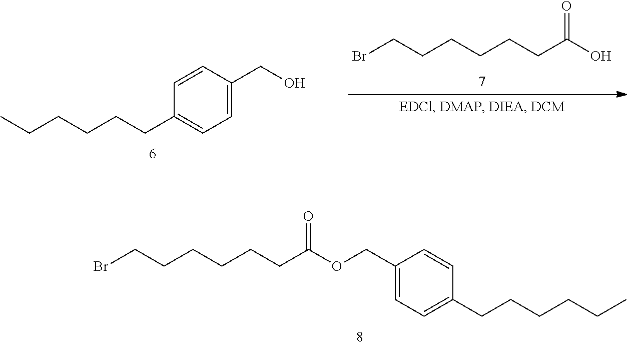 [SMILES: O=C(O)CCCCCCBr ; CCCCCCc1ccc(CO)cc1 ; CCCCCCc1ccc(COC(=O)CCCCCCBr)cc1]

Compound 7 (522 mg, 2.5 mmol, 1.2 eq.) and compound 6 (400 mg, 2.083 mmol, 1 eq.) were dissolved in DCM (4 mL). EDCI (800 mg, 4.166 mmol, 2 eq.), DMAP (102 mg, 0.833 mmol, 0.4 eq.), and DIEA (1.075 mg, 8.332 mmol, 4 eq.) were added into the mixture. After the addition was completed, the reaction mixture was stirred overnight at room temperature under nitrogen protection. TLC (PE:EA=10:1) showed that a starting material was consumed and a new spot was formed. The reaction mixture was concentrated under reduced pressure. The residue was purified by silica gel column chromatography, and eluted with PE/EA (1/0-10/1) to obtain colorless oily compound 8 (454 mg, 57%).

### 4. Synthesis of SW-II-127

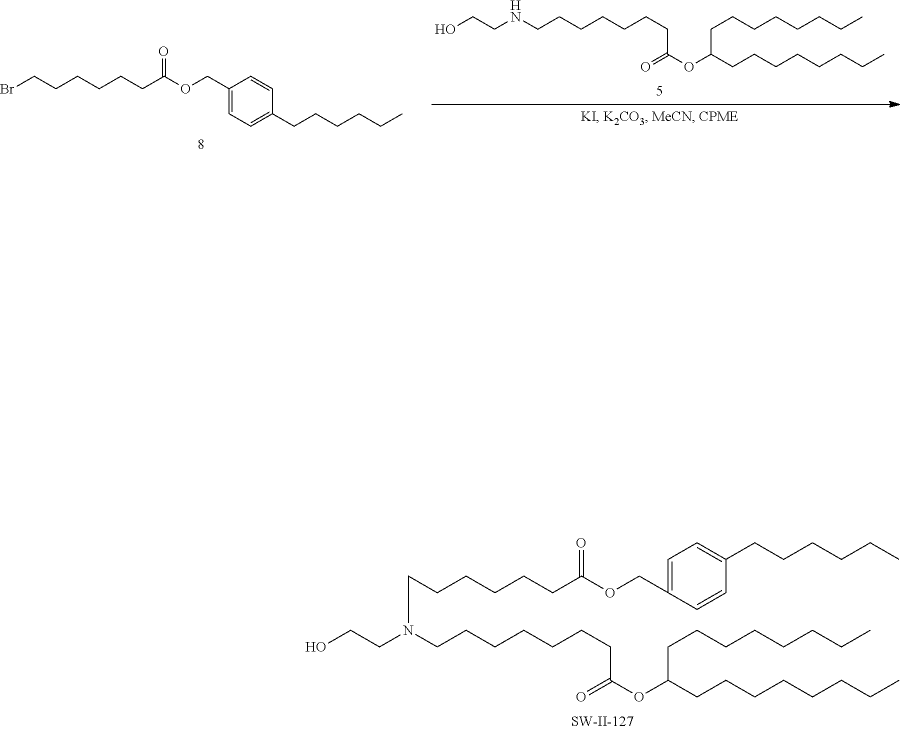 [SMILES: CCCCCCc1ccc(COC(=O)CCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCC(=O)OCc1ccc(CCCCCC)cc1]

Compound 8 (100 mg, 0.262 mmol, 1 eq.) and compound 5 (139 mg, 0.314 mmol, 1.2 eq.) were dissolved in CPME/CH₃CN (1 mL/1 mL). Potassium carbonate (217 mg, 1.572 mmol, 6 eq.) and potassium iodide (87 mg, 0.524 mmol, 2 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10:1, v/v) to obtain yellow oily compound SW-II-127 (42.49 mg, 22%).

LCMS: Rt: 1.323 min; MS m/z (ELSD): 744.5[M+H]⁺;

HPLC: 99.742% purity, ELSD; RT=7.339 min.

¹H NMR (400 MHz, CDCl₃) δ7.25 (s, 2H), 7.17 (d, J=8.0 Hz, 2H), 5.07 (s, 2H), 4.91-4.82 (m, 1H), 3.83 (s, 2H), 2.90 (d, J=44.8 Hz, 5H), 2.64-2.55 (m, 2H), 2.35 (t, J=7.4 Hz, 2H), 2.28 (t, J=7.5 Hz, 2H), 1.76-1.46 (m, 14H), 1.42-1.19 (m, 41H), 0.88 (t, J=6.8 Hz, 9H).

¹³C NMR (101 MHz, CDCl₃) δ 173.50 (d, J=8.5 Hz), 133.17 (s), 128.61 (s), 128.34 (s), 77.29 (d, J=11.4 Hz), 77.03 (s), 76.71 (s), 74.23 (s), 66.19 (s), 54.20 (s), 35.71 (s), 34.56 (s), 34.10 (d, J=8.8 Hz), 31.80 (d, J=15.4 Hz), 31.43 (s), 29.53 (d, J=2.5 Hz), 29.25 (s), 28.95 (d, J=10.5 Hz), 28.63 (s), 26.71 (d, J=18.2 Hz), 25.33 (s), 24.93 (s), 24.62 (s), 22.65 (d, J=6.6 Hz), 14.13 (s).

### G. Compound SW-II-134-1

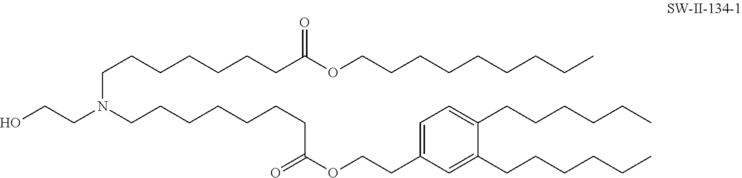 [SMILES: CCCCCCCCCOC(=O)CCCCCCCN(CCO)CCCCCCCC(=O)OCCc1ccc(CCCCCC)c(CCCCCC)c1]

### 1. Synthesis of Compound 3

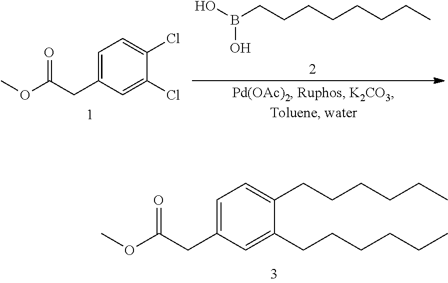 [SMILES: COC(=O)Cc1ccc(Cl)c(Cl)c1 ; CCCCCCCCB(O)O ; CCCCCCc1ccc(CC(=O)OC)cc1CCCCCC]

Palladium acetate (51 mg, 0.228 mmol, 0.1 eq.), Ruphos (213 mg, 0.457 mmol, 0.2 eq.), and potassium carbonate (945 mg, 6.849 mmol, 3 eq.) were added into a mixture of compound 1 (500 mg, 2.283 mmol, 1 eq.) and compound 2 (890 mg, 6.849 mmol, 3 eq.) in toluene/water (5 mL/1 mL). The mixture was stirred overnight at 110° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (723 mg, 99.6%).

### 2. Synthesis of Compound 4

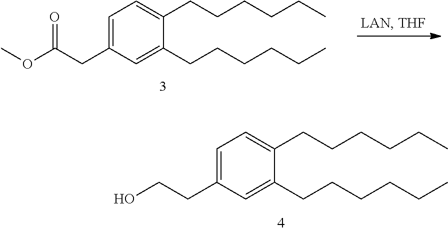 [SMILES: CCCCCCc1ccc(CC(=O)OC)cc1CCCCCC ; CCCCCCc1ccc(CCO)cc1CCCCCC]

At 0° C. and under nitrogen environment, lithium aluminium hydride (2.3 mL, 2.27 mmol, 1 M, in THF, 1 eq.) was added into a mixture of compound 3 (723 mg, 2.27 mmol, 1 eq.) in THF (8 mL). The mixture was stirred at room temperature for 3 hours. TLC (PE/EA=5/1) showed that the reaction was complete and a new major spot was observed. The mixture was quenched with water (2.3 mL) and treated with 2N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (381 mg, >58%), which did not need to be further purified.

### 3. Synthesis of Compound 6

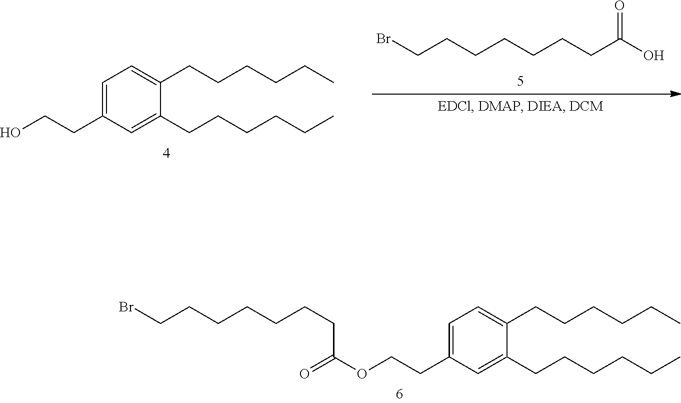 [SMILES: O=C(O)CCCCCCCBr ; CCCCCCc1ccc(CCO)cc1CCCCCC ; CCCCCCc1ccc(CCOC(=O)CCCCCCCBr)cc1CCCCCC]

EDCI (499 mg, 2.6 mmol, 2 eq.) and DMAP (63 mg, 0.52 mmol, 0.4 eq.) were added into a mixture of compound 4 (381 mg, 1.3 mmol, 1 eq.) and compound 5 (352 mg, 1.6 mmol, 1.2 eq.) in DCM (4 mL), and then DIEA (671 mg, 5.2 mmol, 4 eq.) was added. The reaction mixture was stirred at room temperature for 16 hours under nitrogen. TLC (petroleum ether/ethyl acetate=20/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (272 mg, 44%).

### 4. Synthesis of SW-II-134-1

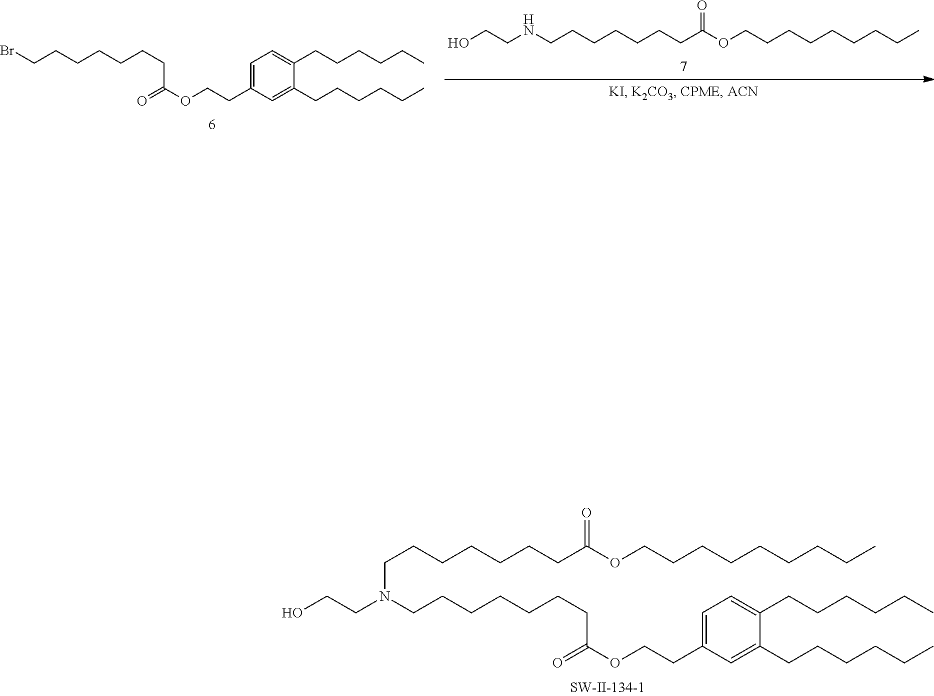 [SMILES: CCCCCCCCCOC(=O)CCCCCCCNCCO ; CCCCCCc1ccc(CCOC(=O)CCCCCCCBr)cc1CCCCCC ; CCCCCCCCCOC(=O)CCCCCCCN(CCO)CCCCCCCC(=O)OCCc1ccc(CCCCCC)c(CCCCCC)c1]

Potassium carbonate (251 mg, 1.818 mmol, 6 eq.) and potassium iodide (101 mg, 0.61 mmol, 2 eq.) were added into a mixture of compound 6 (150 mg, 0.303 mmol, 1 eq.) and compound 7 (110 mg, 0.333 mmol, 1.1 eq.) in CPME/CH₃CN (2 mL/2 mL). After the addition, the mixture was stirred overnight at 90° C. under nitrogen. TLC (DCM/MeOH=15/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10:1, v/v) to obtain yellow oily compound SW-II-134-1 (168 mg, 75%).

LCMS: Rt: 1.276 min; MS m/z (ELSD): 744.4[M+H]⁺;

HPLC: 98.481% purity, ELSD; RT=10.724 min.

¹H NMR (400 MHz, CDCl₃) δ7.06 (d, J=7.6 Hz, 1H), 7.01-6.93 (m, 2H), 4.25 (t, J=7.3 Hz, 2H), 4.05 (t, J=6.8 Hz, 2H), 3.85-3.72 (m, 2H), 2.98-2.69 (m, 8H), 2.62-2.48 (m, 4H), 2.29 (t, J=7.5 Hz, 4H), 1.72-1.48 (m, 14H), 1.45-1.17 (m, 36H), 0.89 (dt, J=11.9, 6.0 Hz, 9H).

¹³CNMR (101 MHz, CDCl₃) δ 173.78 (d, J=16.7 Hz), 140.72 (s), 138.81 (s), 134.91 (s), 129.70 (s), 129.22 (s), 126.19 (s), 77.30 (d, J=11.4 Hz), 77.03 (s), 76.72 (s), 65.02 (s), 64.49 (s), 57.42 (s), 56.36 (s), 54.08 (s), 34.76 (s), 34.22 (d, J=4.2 Hz), 32.74 (s), 32.36 (s), 31.81 (d, J=9.1 Hz), 31.35 (d, J=5.3 Hz), 29.49 (d, J=2.8 Hz), 29.24 (d, J=2.2 Hz), 28.92 (s), 28.66 (s), 26.86 (s), 25.93 (s), 25.04 (s), 24.78 (d, J=6.6 Hz), 22.65 (d, J=2.6 Hz), 14.10 (s).

### H. Compound SW-II-134-2

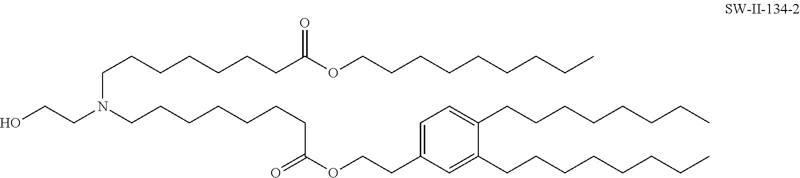 [SMILES: CCCCCCCCCOC(=O)CCCCCCCN(CCO)CCCCCCCC(=O)OCCc1ccc(CCCCCCCC)c(CCCCCCCC)c1]

### 1. Synthesis of Compound 3

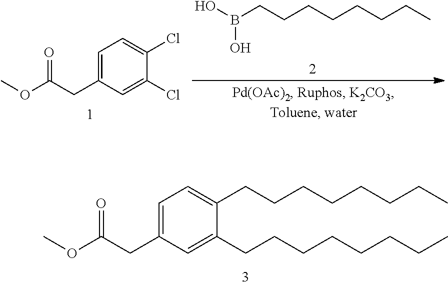 [SMILES: COC(=O)Cc1ccc(Cl)c(Cl)c1 ; CCCCCCCCB(O)O ; CCCCCCCCc1ccc(CC(=O)OC)cc1CCCCCCCC]

Palladium acetate (51 mg, 0.228 mmol, 0.1 eq.), Ruphos (213 mg, 0.457 mmol, 0.2 eq.), and potassium carbonate (945 mg, 6.849 mmol, 3 eq.) were added into a mixture of compound 1 (500 mg, 2.283 mmol, 1 eq.) and compound 2 (1.08 g, 6.849 mmol, 3 eq.) in toluene/water (5 mL/1 mL). The mixture was stirred overnight at 110° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (854 mg, 100%).

### 2. Synthesis of Compound

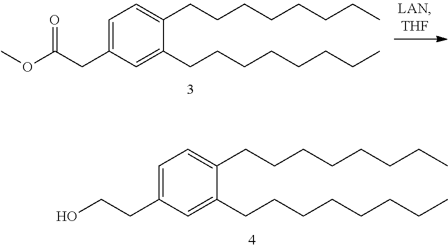 [SMILES: CCCCCCCCc1ccc(CC(=O)OC)cc1CCCCCCCC ; CCCCCCCCc1ccc(CCO)cc1CCCCCCCC]

At 0° C. and under nitrogen environment, lithium aluminium hydride (2.3 mL, 2.28 mmol, 1 M, in THF, 1 eq.) was added into a mixture of compound 3 (854 mg, 2.28 mmol, 1 eq.) in THF (9 mL). The mixture was stirred at room temperature for 3 hours. TLC (PE/EA=5/1) showed that the reaction was complete and a new major spot was observed. The mixture was quenched with water (2.3 mL) and treated with 2N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (724 mg, 92%), which did not need to be further purified.

### 3. Synthesis of Compound 6

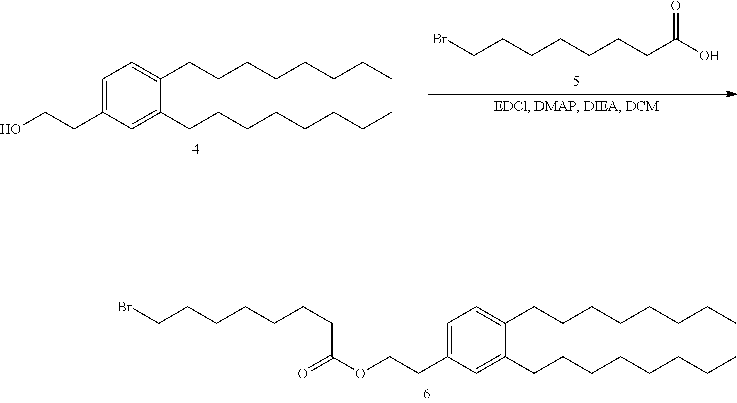 [SMILES: O=C(O)CCCCCCCBr ; CCCCCCCCc1ccc(CCO)cc1CCCCCCCC ; CCCCCCCCc1ccc(CCOC(=O)CCCCCCCBr)cc1CCCCCCCC]

EDCI (803 mg, 4.18 mmol, 2 eq.) and DMAP (102 mg, 0.84 mmol, 0.4 eq.) were added into a mixture of compound 4 (724 mg, 2.09 mmol, 1 eq.) and compound 5 (560 mg, 2.51 mmol, 1.2 eq.) in DCM (8 mL), and then DIEA (1.078 g, 8.36 mmol, 4 eq.) was added. The reaction mixture was stirred at room temperature for 16 hours under nitrogen. TLC (petroleum ether/ethyl acetate=20/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (473 mg, 41%).

### 4. Synthesis of SW-II-134-2

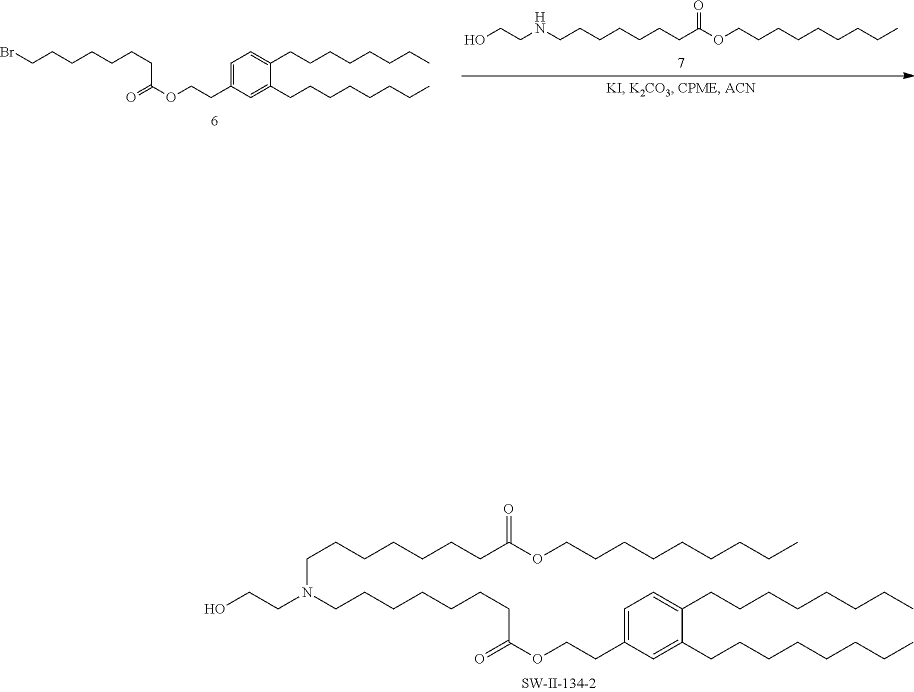 [SMILES: CCCCCCCCCOC(=O)CCCCCCCNCCO ; CCCCCCCCc1ccc(CCOC(=O)CCCCCCCBr)cc1CCCCCCCC ; CCCCCCCCCOC(=O)CCCCCCCN(CCO)CCCCCCCC(=O)OCCc1ccc(CCCCCCCC)c(CCCCCCCC)c1]

Potassium carbonate (225 mg, 1.63 mmol, 6 eq.) and potassium iodide (90 mg, 0.54 mmol, 2 eq.) were added into a mixture of compound 6 (150 mg, 0.27 mmol, 1 eq.) and compound 7 (108 mg, 0.33 mmol, 1.1 eq.) in CPME/CH₃CN (2 mL/2 mL). After the addition, the mixture was stirred overnight at 90° C. under nitrogen. TLC (DCM/MeOH=15/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10:1, v/v) to obtain yellow oily compound SW-II-134-2 (71.77 mg, 33%).

LCMS: Rt: 1.527 min; MS m/z (ELSD): 800.4[M+H]⁺;

HPLC: 97.311% purity, ELSD; RT=9.025 min.

¹H NMR (400 MHz, CDCl₃) δ7.06 (d, J=7.6 Hz, 1H), 6.96 (d, J=9.6 Hz, 2H), 4.25 (t, J=7.3 Hz, 2H), 4.05 (t, J=6.8 Hz, 2H), 3.80-3.66 (m, 2H), 2.86 (dd, J=12.8, 5.6 Hz, 4H), 2.78-2.67 (m, 4H), 2.60-2.52 (m, 4H), 2.29 (t, J=7.5 Hz, 4H), 1.57 (dt, J=15.8, 7.3 Hz, 14H), 1.30 (d, J=20.3 Hz, 45H), 0.88 (t, J=6.7 Hz, 9H).

¹³C NMR (101 MHz, CDCl₃) δ 173.82 (d, J=16.9 Hz), 140.73 (s), 138.82 (s), 134.91 (s), 129.71 (s), 129.23 (s), 126.19 (s), 77.36 (s), 77.14 (d, J=20.4 Hz), 76.72 (s), 65.03 (s), 64.49 (s), 57.57 (s), 56.13 (s), 54.02 (s), 34.76 (s), 34.25 (d, J=4.2 Hz), 32.76 (s), 32.37 (s), 31.89 (d, J=5.3 Hz), 31.40 (d, J=6.0 Hz), 29.84 (d, J=3.7 Hz), 29.63-29.14 (m), 28.97 (s), 28.65 (s), 26.93 (s), 25.66 (d, J=54.4 Hz), 24.80 (d, J=6.6 Hz), 22.68 (d, J=1.8 Hz), 14.12 (s).

### I. Compound SW-II-134-3

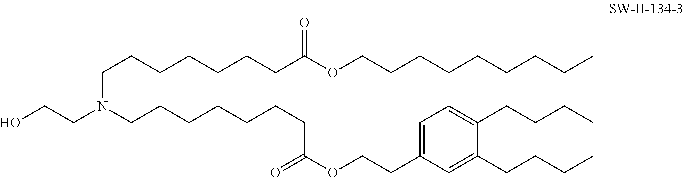 [SMILES: CCCCCCCCCOC(=O)CCCCCCCN(CCO)CCCCCCCC(=O)OCCc1ccc(CCCC)c(CCCC)c1]

### 1. Synthesis of Compound 3

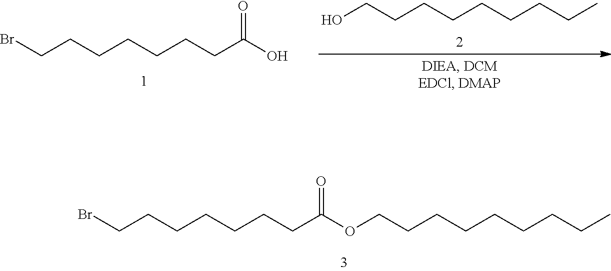

EDCI (17.3 g, 90 mmol, 2 eq.) and DMAP (2.2 g, 18 mmol, 0.4 eq.) were added into a mixture of compound 1 (10 g, 45 mmol, 1 eq.) and compound 2 (7.8 g, 54 mmol, 1.2 eq.) in DCM (100 mL), and then DIEA (23.2 g, 180 mmol, 4 eq.) was added. The reaction mixture was stirred at room temperature for 16 hours under nitrogen. TLC (petroleum ether/ethyl acetate=30/1) showed that compound 1 was consumed and the desired product was formed. The reaction mixture was extracted with ethyl acetate (20 mL), washed with water (40 mL×3), dried with anhydrous sodium sulfate, filtered, and concentrated under reduced pressure. The residue was purified by silica gel column chromatography, and eluted with petroleum ether/ethyl acetate (1/0-20/1) to obtain colorless oily compound 3 (4.365 g, 28%).

### 2. Synthesis of Compound 5

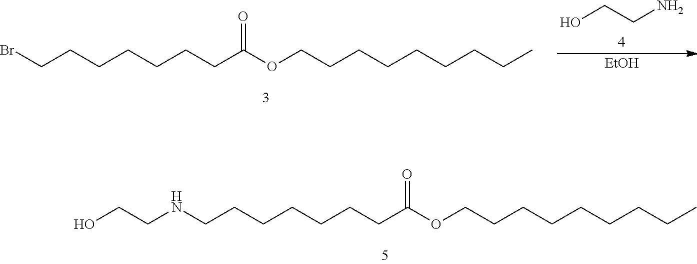

A mixture of compound 3 (5 g, 14.38 mmol, 1 eq.) and compound 4 (8.8 g, 143.7 mmol, 10 eq.) in ethanol (2 mL) was stirred at 55° C. for 16 hours under nitrogen. TLC (DCM/MeOH=10/1) showed that a new major spot was observed. The reaction mixture was extracted with ethyl acetate (50 mL) and washed with water (3×50 mL). The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound 5 (1.008 g, 21%).

### 3. Synthesis of Compound 8

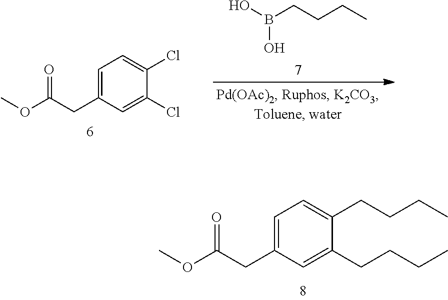

Palladium acetate (51 mg, 0.228 mmol, 0.1 eq.), Ruphos (213 mg, 0.457 mmol, 0.2 eq.), and potassium carbonate (945 mg, 6.849 mmol, 3 eq.) were added into a mixture of compound 6 (500 mg, 2.283 mmol, 1 eq.) and compound 7 (699 mg, 6.849 mmol, 3 eq.) in toluene/water (5 mL/1 mL). The mixture was stirred overnight at 110° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 8 (507 mg, 85%).

### 4. Synthesis of Compound 9

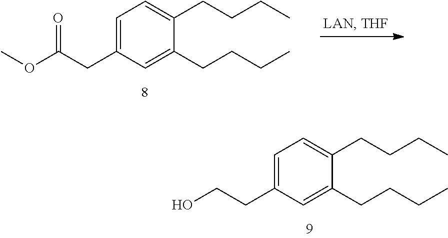 [SMILES: CCCCc1ccc(CC(=O)OC)cc1CCCC ; CCCCc1ccc(CCO)cc1CCCC]

At 0° C. and under nitrogen environment, lithium aluminium hydride (2 mL, 1.935 mmol, 1 M, in THF, 1 eq.) was added into a mixture of compound 8 (507 mg, 1.935 mmol, 1 eq.) in THF (5 mL). The mixture was stirred at room temperature for 3 hours. TLC (PE/EA=5/1) showed that the reaction was complete and a new major spot was observed. The mixture was quenched with water (2 mL) and treated with 2N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 9 (492 mg, >100%), which did not need to be further purified.

### 5. Synthesis of Compound 10

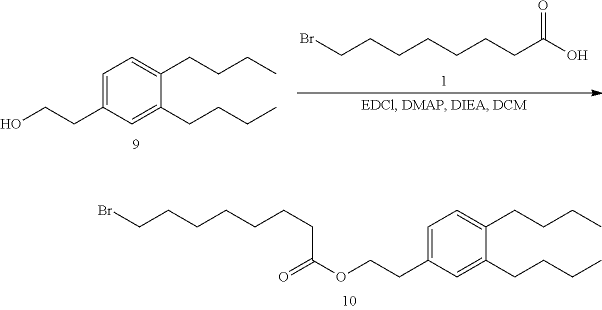 [SMILES: O=C(O)CCCCCCCBr ; CCCCc1ccc(CCO)cc1CCCC ; CCCCc1ccc(CCOC(=O)CCCCCCCBr)cc1CCCC]

EDCI (808 mg, 4.206 mmol, 2 eq.) and DMAP (103 mg, 0.84 mmol, 0.4 eq.) were added into a mixture of compound 9 (492 mg, 2.103 mmol, 1 eq.) and compound 1 (563 mg, 2.523 mmol, 1.2 eq.) in DCM (5 mL), and then DIEA (1.085 g, 8.412 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-10/1) to obtain colorless oily compound 10 (329 mg, 36%).

### 6. Synthesis of SW-II-134-3

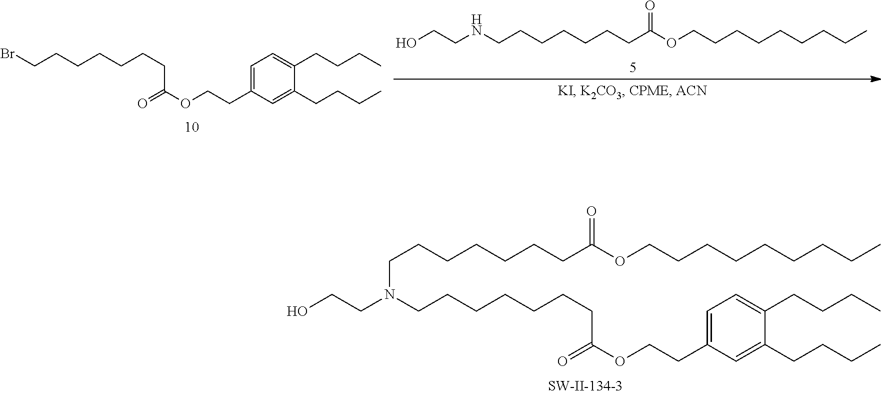

Potassium carbonate (282 mg, 2.04 mmol, 6 eq.) and potassium iodide (113 mg, 0.68 mmol, 2 eq.) were added into a mixture of compound 10 (150 mg, 0.34 mmol, 1 eq.) and compound 5 (134 mg, 0.41 mmol, 1.2 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-134-3 (63.59 mg, 25%).

LCMS: Rt: 1.247 min; MS m/z (ELSD): 688.3[M+H]⁺;

HPLC: 95.945% purity, ELSD; RT=6.186 min.

¹H NMR (400 MHz, CDCl₃) δ7.07 (d, J=7.6 Hz, 1H), 6.97 (dd, J=9.9, 2.2 Hz, 2H), 4.26 (t, J=7.2 Hz, 2H), 4.05 (t, J=6.8 Hz, 2H), 2.88 (dd, J=14.8, 7.6 Hz, 4H), 2.78-2.74 (m, 2H), 2.67-2.54 (m, 8H), 2.29 (t, J=7.5 Hz, 4H), 1.68-1.47 (m, 15H), 1.37-1.22 (m, 27H), 0.98-0.86 (m, 9H).

¹³C NMR (101 MHz, CDCl₃) δ 173.86 (d, J=17.1 Hz), 140.66 (s), 138.76 (s), 134.93 (s), 129.74 (s), 129.24 (s), 126.19 (s), 77.36 (s), 77.04 (s), 76.72 (s), 65.01 (s), 64.48 (s), 57.73 (s), 55.73 (s), 53.93 (s), 34.76 (s), 34.28 (d, J=3.9 Hz), 33.54 (d, J=4.5 Hz), 32.41 (s), 31.95 (d, J=16.5 Hz), 29.49 (s), 29.15 (dd, J=21.1, 2.4 Hz), 28.66 (s), 27.04 (s), 25.95 (d, J=3.3 Hz), 24.85 (d, J=6.6 Hz), 22.98-22.58 (m), 14.08 (d, J=7.5 Hz).

### J. SW-II-135-1

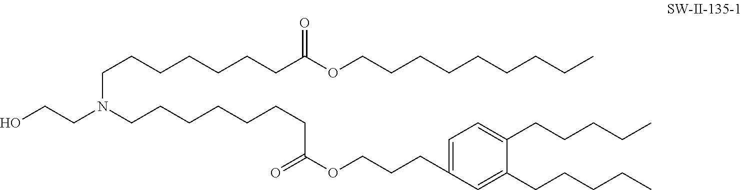 [SMILES: CCCCCCCCCOC(=O)CCCCCCCN(CCO)CCCCCCCC(=O)OCCCc1ccc(CCCCC)c(CCCCC)c1]

### 1. Synthesis of Compound 3

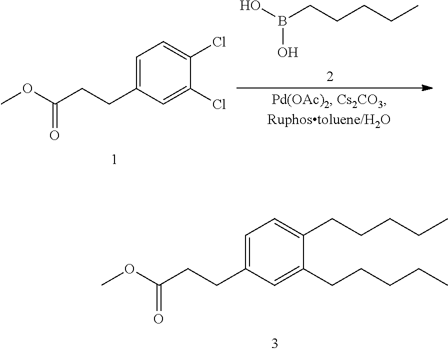

Compound 1 (500 mg, 2.16 mmol, 1.0 eq.) and compound 2 (750 mg, 6.46 mmol, 3.0 eq.) were dissolved in toluene/H₂O (5 mL/1 mL). Ruphos (201 mg, 0.43 mmol, 0.2 eq.), Pd(OAc)₂ (48.5 mg, 0.22 mmol, 0.1 eq.), and Cs₂CO₃ (2.10 g, 6.46 mmol, 3.0 eq.) were added into the mixture. The reaction mixture was subjected to heating reflux at 110° C. for 16 hours under nitrogen protection. TLC (petroleum ether/ethyl acetate=10/1) showed that the reaction was complete and the desired product was formed. The reaction mixture was washed with H₂O (40 mL) and extracted three times with EA (50 mL). The obtained organic phase was washed twice with brine (20 mL), dried with anhydrous Na₂SO₄, filtered, and concentrated under reduced pressure. The residue was purified by silica gel column chromatography, and eluted with petroleum ether/ethyl acetate (1/0-30/1) to obtain yellow oily compound 3 (540 mg, 82.44%).

### 2. Synthesis of Compound 4

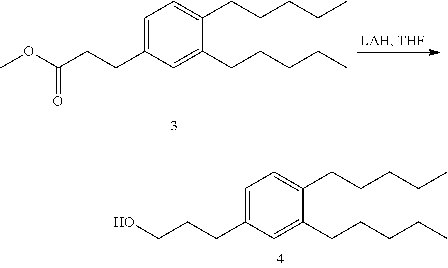 [SMILES: CCCCCc1ccc(CCC(=O)OC)cc1CCCCC ; CCCCCc1ccc(CCCO)cc1CCCCC]

LiAlH₄ (3.55 mL, 3.55 mmol, 1 M in THF, 2 eq.) was added into compound 3 (540 mg, 1.78 mmol, 1.0 eq.) dissolved in THF (5 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with water (10 mL), then regulated with 1 M hydrochloric acid to pH=6-7, and extracted three times with ethyl acetate (50 mL). The organic layer was washed with brine, dried with anhydrous sodium sulfate, filtered, and concentrated under reduced pressure. The residue was purified by silica gel column chromatography, and eluted with PE/EA (1/0-10/1) to obtain colorless oily compound 4 (442 mg, 90.2%).

### 3. Synthesis of Compound 6

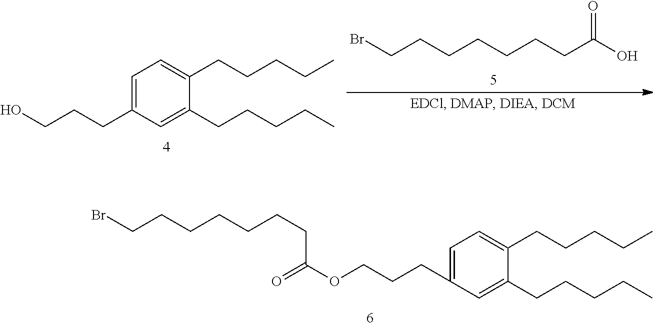

Compound 4 (442 mg, 1.60 mmol, 1.0 eq.) and compound 5 (428.5 mg, 1.92 mmol, 1.2 eq.) were dissolved in DCM (5 mL). EDCI (612 mg, 3.2 mmol, 2.0 eq.) and DMAP (78.2 mg, 0.64 mmol, 0.4 eq.) were added into the mixture, and then DIEA (826 mg, 6.4 mmol, 4.0 eq.) was added. The reaction mixture was stirred at room temperature for 16 hours under nitrogen protection. TLC (petroleum ether/ethyl acetate=10/1) showed that compound 4 was consumed and the desired product was formed. The reaction mixture was washed with H₂O (40 mL) and extracted three times with EA (50 mL). The obtained organic phase was washed twice with brine (20 mL), dried with anhydrous Na₂SO₄, filtered, and concentrated under reduced pressure. The residue was purified by silica gel column chromatography, and eluted with petroleum ether/ethyl acetate (1/0-10/1) to obtain yellow oily compound 3 (342 mg, 44.5%).

### 4. Synthesis of SW-II-135-1

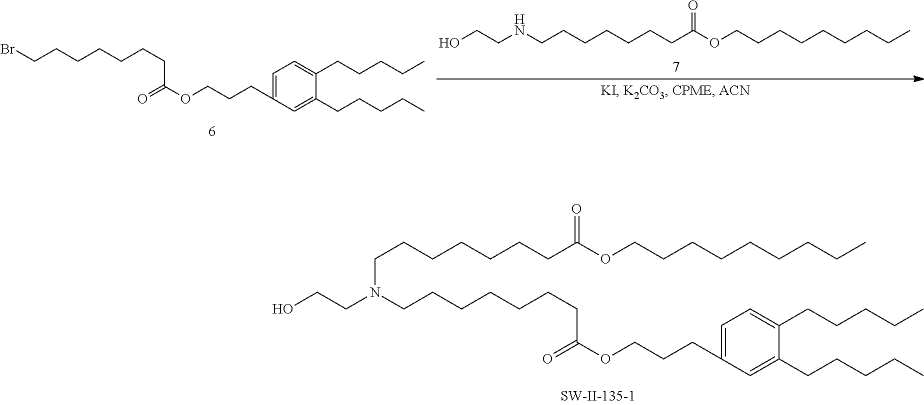

Compound 6 (175 mg, 0.365 mmol, 1.2 eq.) and compound 7 (100 mg, 0.304 mmol, 1.0 eq.) were dissolved in CPME/CH₃CN (1 mL/1 mL). Potassium carbonate (210 mg, 1.52 mmol, 5.0 eq.) and potassium iodide (101 mg, 0.61 mmol, 2.0 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-135-1 (83.89 mg, 55.6%).

LCMS: Rt: 1.356 min; MS m/z (ELSD): 730.5[M+H]⁺;

HPLC: 100% purity at ELSD; RT=12.614 min.

¹HNMR (400 MHz, CDCl₃) δ6.97 (d, J=7.6 Hz, 1H), 6.91-6.74 (m, 2H), 4.76 (s, 1H), 3.99 (dt, J=13.6, 6.4 Hz, 4H), 3.72-3.58 (m, 2H), 2.85-2.73 (m, 2H), 2.72-2.61 (m, 4H), 2.59-2.41 (m, 6H), 2.22 (dd, J=13.2, 7.2 Hz, 4H), 1.93-1.79 (m, 2H), 1.62-1.41 (m, 14H), 1.23 (d, J=24.4 Hz, 32H), 0.82 (ddd, J=13.6, 8.0, 5.6 Hz, 9H).

¹³C NMR (101 MHz, CDCl₃) δ 172.81 (d, J=6.4 Hz), 139.55 (s), 137.38 (s), 137.14 (s), 128.16 (d, J=2.4 Hz), 124.69 (s), 76.51 (s), 76.19 (s), 75.88 (s), 63.43 (s), 62.78 (s), 56.53 (s), 54.90 (s), 52.84 (s), 33.23 (d, J=2.4 Hz), 31.73 (s), 31.28 (s), 30.91 (dd, J=20.0, 6.4 Hz), 30.10 (d, J=3.2 Hz), 29.29 (s), 28.36 (d, J=22.8 Hz), 28.23 (s), 27.97 (s), 27.64 (s), 25.92 (s), 24.92 (s), 24.34 (s), 23.84 (s), 21.62 (d, J=7.6 Hz), 13.08 (d, J=4.7 Hz).

### K. Compound SW-II-135-2

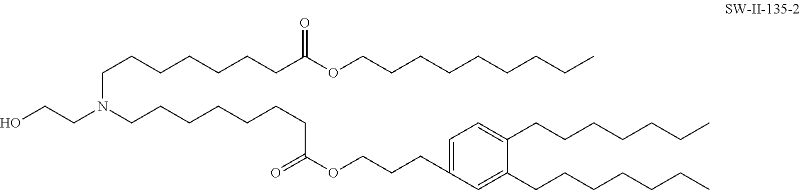 [SMILES: CCCCCCCCCOC(=O)CCCCCCCN(CCO)CCCCCCCC(=O)OCCCc1ccc(CCCCCCC)c(CCCCCCC)c1]

### 1. Synthesis of Compound 3

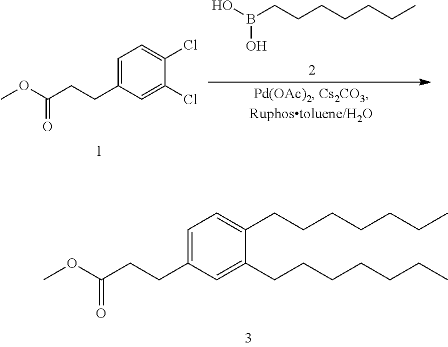

Compound 1 (500 mg, 2.16 mmol, 1.0 eq.) and compound 2 (931 mg, 6.46 mmol, 3.0 eq.) were dissolved in toluene/H₂O (5 mL/1 mL). Ruphos (201 mg, 0.43 mmol, 0.2 eq.), Pd(OAc)₂ (48.5 mg, 0.22 mmol, 0.1 eq.), and Cs₂CO₃ (2.10 g, 6.46 mmol, 3.0 eq.) were added into the mixture. The reaction mixture was subjected to heating reflux at 110° C. for 16 hours under nitrogen protection. TLC (petroleum ether/ethyl acetate=10/1) showed that the reaction was complete and the desired product was formed. The reaction mixture was washed with H₂O (40 mL) and extracted three times with EA (50 mL). The obtained organic phase was washed twice with brine (20 mL), dried with anhydrous Na₂SO₄, filtered, and concentrated under reduced pressure. The residue was purified by silica gel column chromatography, and eluted with petroleum ether/ethyl acetate (1/0-30/1) to obtain yellow oily compound 3 (651 mg, 84%).

### 2. Synthesis of Compound 4

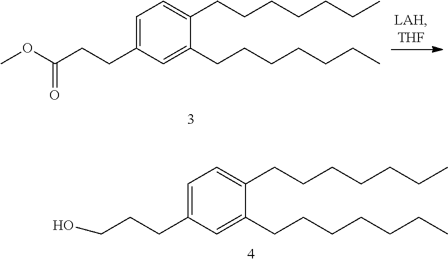 [SMILES: CCCCCCCc1ccc(CCC(=O)OC)cc1CCCCCCC ; CCCCCCCc1ccc(CCCO)cc1CCCCCCC]

LiAlH₄ (3.62 mL, 3.62 mmol, 1 M in THF, 2 eq.) was added into compound 3 (651 mg, 1.81 mmol, 1.0 eq.) dissolved in THF (7 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with water (10 mL), then regulated with 1 M hydrochloric acid to pH=6-7, and extracted three times with ethyl acetate (50 mL). The organic layer was washed with brine, dried with anhydrous sodium sulfate, filtered, and concentrated under reduced pressure. The residue was purified by silica gel column chromatography, and eluted with PE/EA (1/0-10/1) to obtain colorless oily compound 4 (571 mg, 95.2%).

3. Synthesis of compound 6

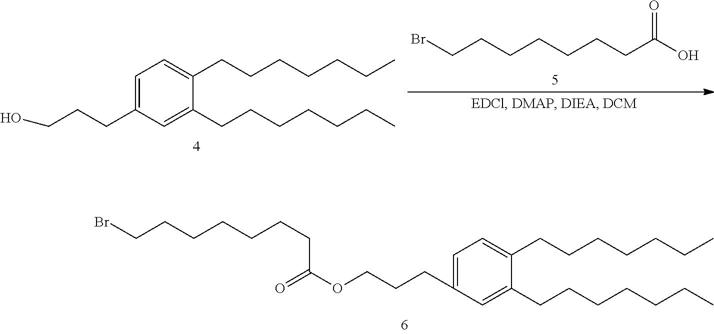

Compound 4 (571 mg, 1.72 mmol, 1.0 eq.) and compound 5 (459 mg, 2.06 mmol, 1.2 eq.) were dissolved in DCM (6 mL). EDCI (657 mg, 3.44 mmol, 2.0 eq.) and DMAP (84 mg, 0.68 mmol, 0.4 eq.) were added into the mixture, and then DIEA (887.5 mg, 6.88 mmol, 4.0 eq.) was added. The reaction mixture was stirred at room temperature for 16 hours under nitrogen protection. TLC (petroleum ether/ethyl acetate=10/1) showed that compound 4 was consumed and the desired product was formed. The reaction mixture was washed with H₂O (50 mL) and extracted three times with EA (60 mL). The obtained organic phase was washed twice with brine (25 mL), dried with anhydrous Na₂SO₄, filtered, and concentrated under reduced pressure. The residue was purified by silica gel column chromatography, and eluted with petroleum ether/ethyl acetate (1/0-10/1) to obtain yellow oily compound 3 (245 mg, 26.5%).

### 4. Synthesis of SW-II-135-2

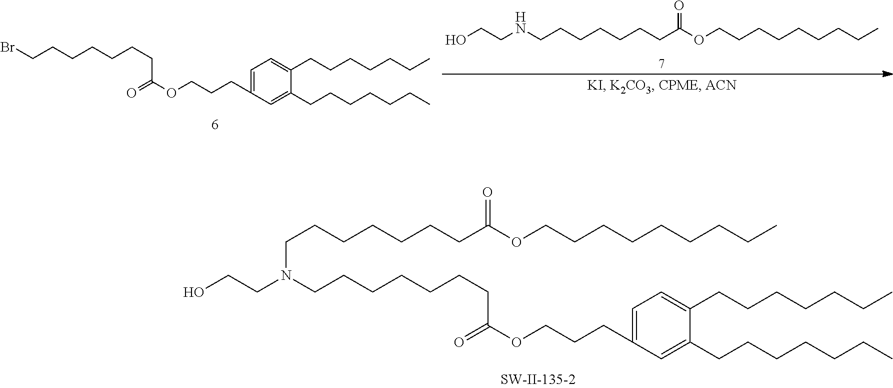

Compound 6 (245 mg, 0.456 mmol, 1.5 eq.) and compound 7 (100 mg, 0.3 mmol, 1.0 eq.) were dissolved in CPME/CH₃CN (1 mL/1 mL). Potassium carbonate (210 mg, 1.52 mmol, 5.0 eq.) and potassium iodide (101 mg, 0.61 mmol, 2.0 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-135-2 (31.41 mg, 21.9%).

LCMS: Rt: 1.608 min; MS m/z (ELSD): 786.4[M+H]⁺;

HPLC: 95.16% purity, ELSD; RT=7.919 min.

¹H NMR (400 MHz, CDCl₃) δ6.98 (d, J=7.6 Hz, 1H), 6.87 (d, J=2.4 Hz, 2H), 4.28-4.13 (m, 1H), 4.04-3.95 (m, 4H), 3.94-3.84 (m, 2H), 3.14-2.89 (m, 6H), 2.59-2.43 (m, 6H), 2.23 (dd, J=13.8, 7.2 Hz, 4H), 1.88-1.82 (m, 2H), 1.70 (s, 4H), 1.57-1.46 (m, 10H), 1.33-1.16 (m, 40H), 0.90-0.72 (m, 9H).

¹³C NMR (100 MHz, CDCl₃) δ 172.82 (d, J=6.8 Hz), 139.61 (s), 137.29 (d, J=16.4 Hz), 128.15 (s), 124.67 (s), 76.41 (s), 76.09 (s), 75.77 (s), 63.50 (s), 62.87 (s), 55.49 (s), 54.92 (s), 52.98 (s), 33.16 (d, J=2.4 Hz), 31.77 (s), 31.33 (s), 30.80 (d, J=6.5 Hz), 30.42 (d, J=3.6 Hz), 29.29 (s), 28.99-28.66 (m), 28.47 (s), 28.23 (d, J=2.8 Hz), 28.06-27.45 (m), 25.58 (s), 24.91 (s), 23.71 (s), 22.79 (s), 21.66 (s), 13.10 (s).

### L. Compound SW-II-136-2

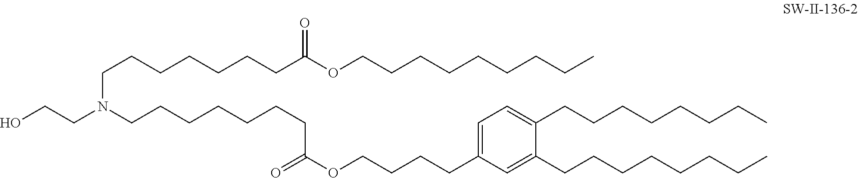 [SMILES: CCCCCCCCCOC(=O)CCCCCCCN(CCO)CCCCCCCC(=O)OCCCCc1ccc(CCCCCCCC)c(CCCCCCCC)c1]

### 1. Synthesis of Compound 3

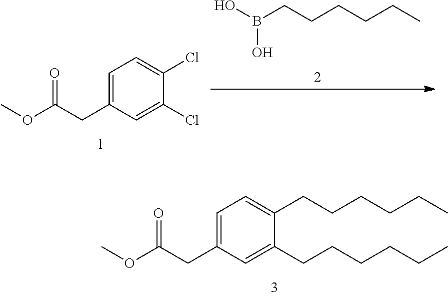

Compound 1 (3 g, 13.70 mmol, 1.0 eq.) and compound 2 (5.34 g, 41.09 mmol, 3.0 eq.) were dissolved in toluene/H₂O (30 mL/3 mL). Ruphos (1.28 g, 2.74 mmol, 0.2 eq.), Pd(OAc)₂ (308.3 mg, 1.37 mmol, 0.1 eq.), and K₂CO₃ (5.67 g, 41.10 mmol, 3.0 eq.) were added into the mixture. The reaction mixture was subjected to heating reflux at 110° C. for 16 hours under nitrogen protection. TLC (PE/EA=10/1) showed that the reaction was complete and the desired product was formed. The reaction mixture was washed with H₂O (90 mL) and extracted three times with EA (110 mL). The obtained organic phase was washed twice with brine (40 mL), dried with anhydrous Na₂SO₄, filtered, and concentrated under reduced pressure. The residue was purified by silica gel column chromatography, and eluted with PE/EA (1/0-30/1) to obtain yellow oily compound 3 (1.98 g, 45.5%).

### 2. Synthesis of Compound 4

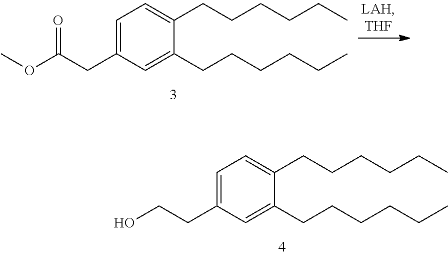 [SMILES: CCCCCCc1ccc(CC(=O)OC)cc1CCCCCC ; CCCCCCc1ccc(CCO)cc1CCCCCC]

LiAlH₄ (1 M, 12.45 mL, 2.0 eq.) was added into compound 3 (1.98 g, 6.23 mL, 1.0 eq.) dissolved in THF (20 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with H₂O (70 mL), then regulated with 1 M hydrochloric acid to pH=6-7, and extracted three times with EA (80 mL). The organic layer was washed with brine, dried with anhydrous Na₂SO₄, filtered, and concentrated under reduced pressure. The residue was purified by silica gel column chromatography, and eluted with PE/EA (1/0-10/1) to obtain colorless oily compound 4 (1.28 g, 71.1%).

### 3. Synthesis of Compound 7

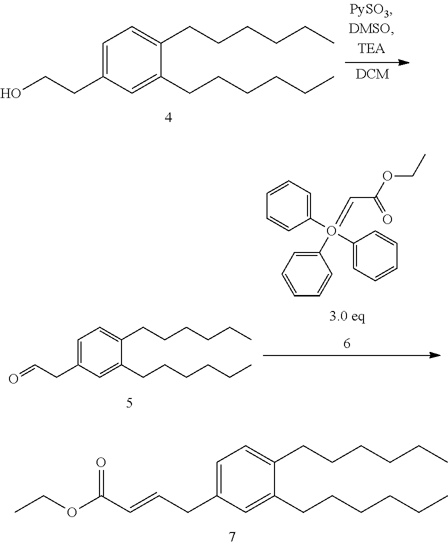

DMSO (3.63 g, 51.72 mmol, 15 eq.), TEA (1.25 g, 12.4 mmol, 4.0 eq.), and PySO₃ (1.27 g, 7.97 mmol, 2.57 eq.) were added into compound 4 (900 g, 3.1 mmol, 1.0 eq.) dissolved in DCM (9 mL) at 0° C. under nitrogen protection. The mixture was stirred at 0° C. for 30 minutes, then heated to room temperature and stirred for 90 minutes under nitrogen protection. Then, compound 6 (4.74 g, 13.62 mmol, 3.0 eq.) was added into the mixture. The reaction mixture reacted at 25° C. for 2 hours under nitrogen protection. TLC (PE/EA=10/1) showed that the reaction was complete and the desired product was formed. The reaction mixture was washed with H₂O (60 mL) and extracted three times with EA (70 mL). The obtained organic phase was washed twice with brine (40 mL), dried with anhydrous Na₂SO₄, filtered, and concentrated under reduced pressure. The residue was purified by silica gel column chromatography, and eluted with PE/EA (1/0-10/1) to obtain yellow oily compound 7 (345 mg, 27.9%).

### 4. Synthesis of Compound 8

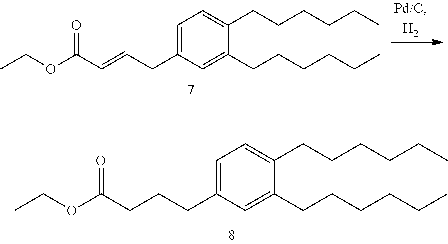

Compound 7 (340 mg, 0.95 mmol, 1.0 eq.) and Pd/C (100 mg) were added into MeOH (4 ml). The reaction mixture was stirred at room temperature for 16 hours under hydrogen protection. TLC (PE/EA=10/1) showed that the raw materials were consumed completely and the desired product was produced. The reaction mixture was filtered with diatomite, washed with MeOH (40 mL×2), dried with anhydrous Na₂SO₄, and concentrated under reduced pressure to obtain light-yellow oily compound 8 (298 mg, 88.2%).

### 5. Synthesis of Compound 9

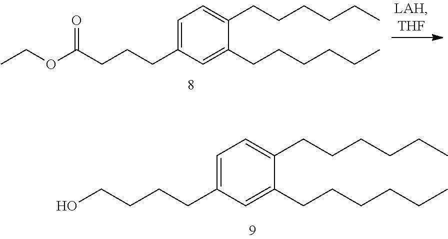 [SMILES: CCCCCCc1ccc(CCCC(=O)OCC)cc1CCCCCC ; CCCCCCc1ccc(CCCCO)cc1CCCCCC]

LiAlH₄ (1 M, 1.66 mL, 2.0 eq.) was added into compound 8 (298 mg, 0.83 mmol, 1.0 eq.) dissolved in THF (3 mL) at 0° C. under nitrogen protection. The reaction mixture was heated to room temperature, and stirred for 2 hours under nitrogen protection. TLC (PE/EtOAc=10/1) showed that the reaction was complete and a new major spot was observed. The mixture was quenched with H₂O (20 mL), then regulated with 1 M hydrochloric acid to pH=6-7, and extracted three times with EA (30 mL). The organic layer was washed with brine, dried with anhydrous Na₂SO₄, filtered, and concentrated under reduced pressure. The residue was purified by silica gel column chromatography, and eluted with PE/EA (1/0-10/1) to obtain colorless oily compound 9 (254 mg, 98.3%).

### 6. Synthesis of Compound 11

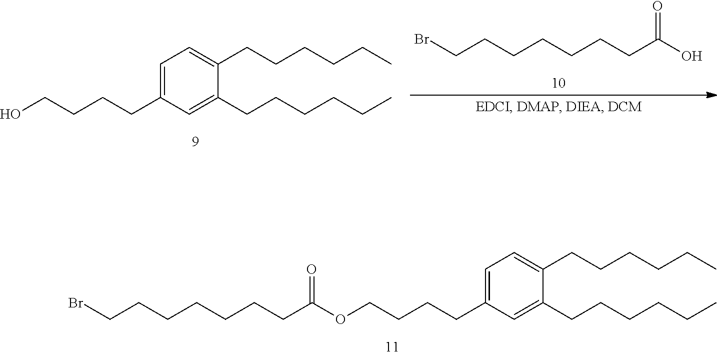

Compound 9 (254 mg, 0.80 mmol, 1.0 eq.) and compound 10 (214 mg, 0.96 mmol, 1.2 eq.) were dissolved in DCM (3 mL). EDCI (305.6 mg, 1.6 mmol, 2.0 eq.) and DMAP (39 mg, 0.32 mmol, 0.4 eq.) were added into the mixture, and then DIEA (412.8 mg, 3.2 mmol, 4.0 eq.) was added. The reaction mixture was stirred at room temperature for 16 hours under nitrogen protection. TLC (PE/EA=10/1) showed that compound 9 was consumed and the desired product was formed. The reaction mixture was regulated with 1 M hydrochloric acid to pH=4-6, and extracted three times with EA (30 mL). The obtained organic phase was washed twice with brine (15 mL), dried with anhydrous Na₂SO₄, filtered, and concentrated under reduced pressure. The residue was purified by silica gel column chromatography, and eluted with PE/EA (1/0-7/1) to obtain yellow oily compound 11 (210 mg, 50.5%).

### 7. Synthesis of SW-II-136-2

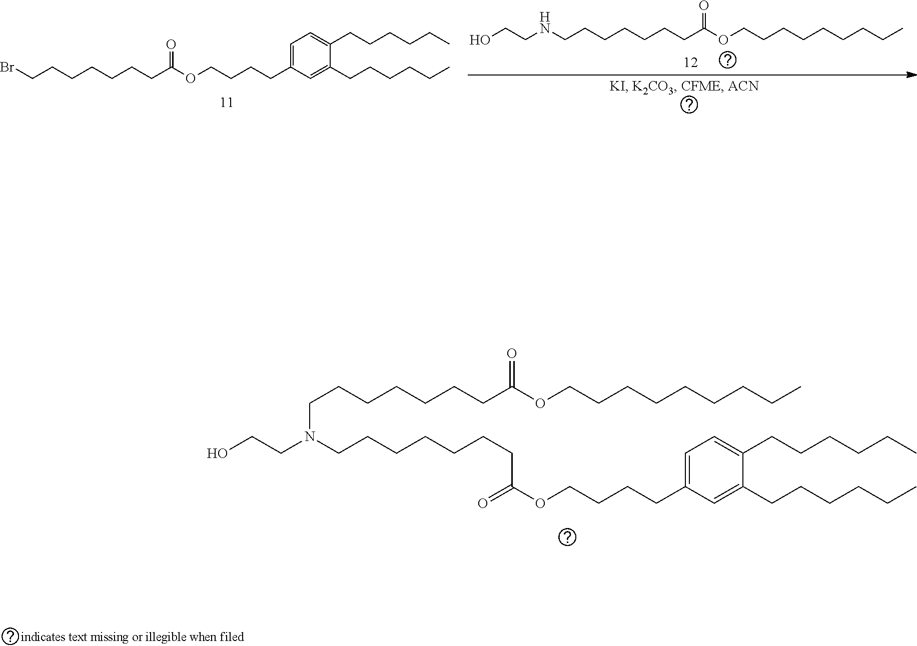

Compound 11 (200 mg, 0.38 mmol, 1.2 eq.) and compound 12 (105 mg, 0.32 mmol, 1.0 eq.) were dissolved in CPME/CH₃CN (1.5 mL/1.5 mL). K₂CO₃ (220.2 mg, 1.60 mmol, 5.0 eq.) and KI (106 mg, 0.64 mmol, 2.0 eq.) were added into the mixture. After the addition was completed, the reaction mixture was stirred overnight at 90° C. under nitrogen protection. TLC (DCM/MeOH=10/1) showed that the reaction was complete and the desired product was formed. The mixture was extracted with EA and washed with water. The organic layer was dried with anhydrous Na₂SO₄, filtered, and concentrated under reduced pressure. The residue was purified by silica gel column chromatography, and eluted with DCM/MeOH (1/0-10/1, v/v) to obtain yellow oily compound SW-II-136-2 (208 mg, 90.4%).

LCMS: Rt: 2.146 min; MS m/z (ELSD): 773.3[M+H]⁺;

HPLC: 99.49% purity, ELSD; RT=8.055 min.

¹H NMR (400 MHz, CDCl₃) δ7.04 (d, J=7.6 Hz, 1H), 6.92 (d, J=9.6 Hz, 2H), 4.45 (s, 1H), 4.06 (dd, J=12.0, 5.2 Hz, 4H), 3.64 (t, J=5.2 Hz, 2H), 2.72 (t, J=5.2 Hz, 2H), 2.65-2.50 (m, 10H), 2.29 (t, J=7.6 Hz, 4H), 1.69-1.48 (m, 18H), 1.41-1.24 (m, 36H), 0.95-0.78 (m, 9H).

¹³C NMR (101 MHz, CDCl₃) δ 173.86 (d, J=2.8 Hz), 140.48 (s), 139.24 (s), 138.01 (s), 129.13 (d, J=14.8 Hz), 125.67 (s), 77.37 (s), 77.05 (s), 76.73 (s), 64.45 (s), 64.23 (s), 57.88 (s), 55.91 (s), 53.94 (s), 35.07 (s), 34.29 (d, J=3.2 Hz), 32.79 (s), 32.35 (s), 31.82 (d, J=8.4 Hz), 31.38 (s), 29.50 (d, J=2.4 Hz), 29.16 (dd, J=18.0, 2.0 Hz), 28.66 (s), 28.35 (s), 27.78 (s), 27.08 (s), 26.02 (d, J=17.2 Hz), 24.89 (d, J=1.6 Hz), 22.65 (s), 14.10 (s).

### M. Compound SW-II-137-1

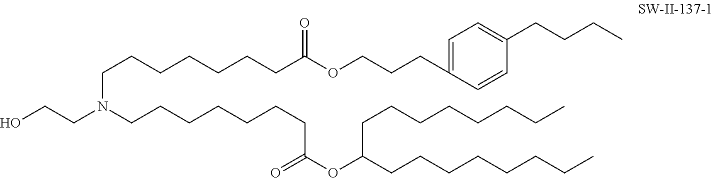 [SMILES: CCCCCCCCC(CCCCCCCC)OC(=O)CCCCCCCN(CCO)CCCCCCCC(=O)OCCCc1ccc(CCCC)cc1]

### 1. Synthesis of Compound 3

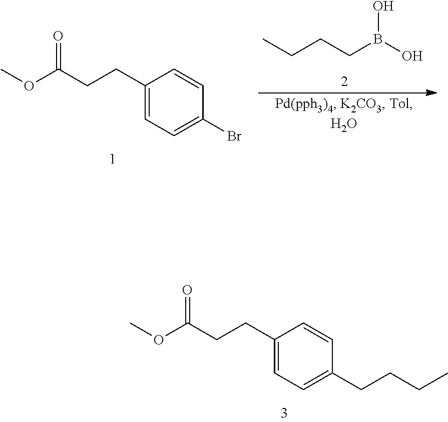

Compound 1 (500 mg, 1.95 mmol, 1.0 eq.) was dissolved in toluene (5.0 mL). Then, compound 2 (239 mg, 2.34 mmol, 1.2 eq.), Pd(PPh₃)₄ (225 mg, 0.19 mmol, 0.1 eq.), water (1 mL), and K₂CO₃ (808 g, 5.85 mmol, 3.0 eq.) were added. The reaction was carried out at 110° C. for 3 hours under nitrogen protection. TLC (PE/EA=5/1) showed that the raw materials had reacted completely and the desired product was formed. H₂O (70 mL) was added into the reaction. Extraction was carried out with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-5:1, v/v) to obtain colorless oily compound (320 mg, 70%).

### 2. Synthesis of Compound 4

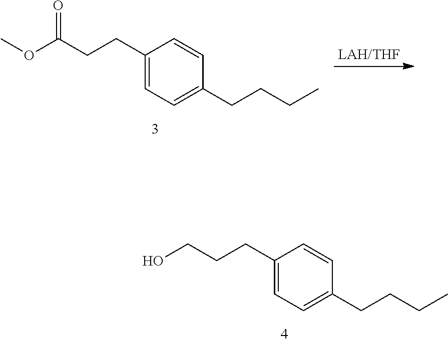 [SMILES: CCCCc1ccc(CCC(=O)OC)cc1 ; CCCCc1ccc(CCCO)cc1]

Compound 3 (300 mg, 1.28 mmol, 1.0 eq.) was dissolved in THF (4.0 mL). LAH (97 mg, 2.56 mmol, 2.0 eq.) was added at 0° C. under nitrogen protection. Then, the reaction was carried out at room temperature for 2 hours. TLC (PE/EA=10/1) showed that the raw materials reacted completely and the desired product was produced. A HCl (1 M, 4 mL) solution and H₂O (10 mL) were added for quenching. Extraction was carried out with EA (50 mL×3). The organic phase was washed with saturated salt solution (2×30 mL), dried with anhydrous Na₂SO₄, filtered, and spin-dried under reduced pressure. The residue was purified by silica gel column, and eluted with PE/EA (1/0-10/1, v/v) to obtain yellow oily compound 4 (224 mg, 84.8%).

### 3. Synthesis of Compound 6

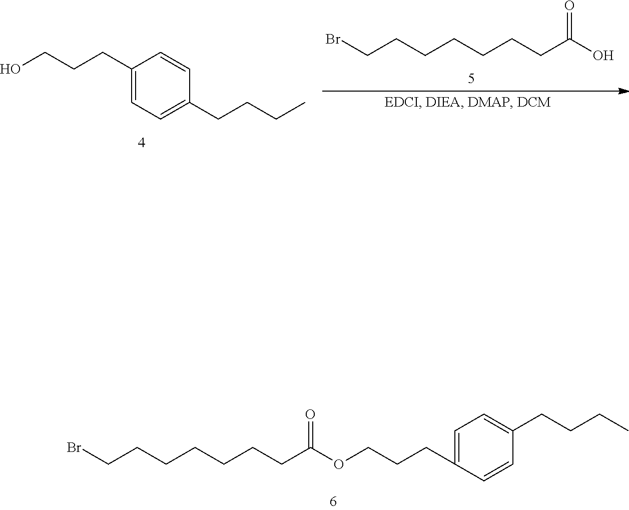 [SMILES: CCCCc1ccc(CCCO)cc1 ; O=C(O)CCCCCCCBr ; CCCCc1ccc(CCCOC(=O)CCCCCCCBr)cc1]

Compound 4 (90 mg, 0.47 mmol, 1.0 eq.) was dissolved in DCM (3.0 mL). Compound 5 (127 mg, 0.56 mmol, 1.2 eq.), EDCI (180 mg, 0.94 mmol, 2.0 eq.), DIEA (242 mg, 1.88 mmol, 4.0 eq.), and DMAP (23 mg, 0.18 mmol, 0.4 eq.) were added. Then, the reaction was carried out overnight at room temperature under nitrogen protection. TLC (PE/EA=20/1) showed that the raw materials had reacted completely and the desired product was formed. The reaction mixture was quenched with a HCl (1 M) solution, regulated to PH=4-6, and extracted with EA (40 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-20/1, v/v) to obtain colorless oily compound 6 (90 mg, 48.6%).

### 4. Synthesis of SW-II-137-1

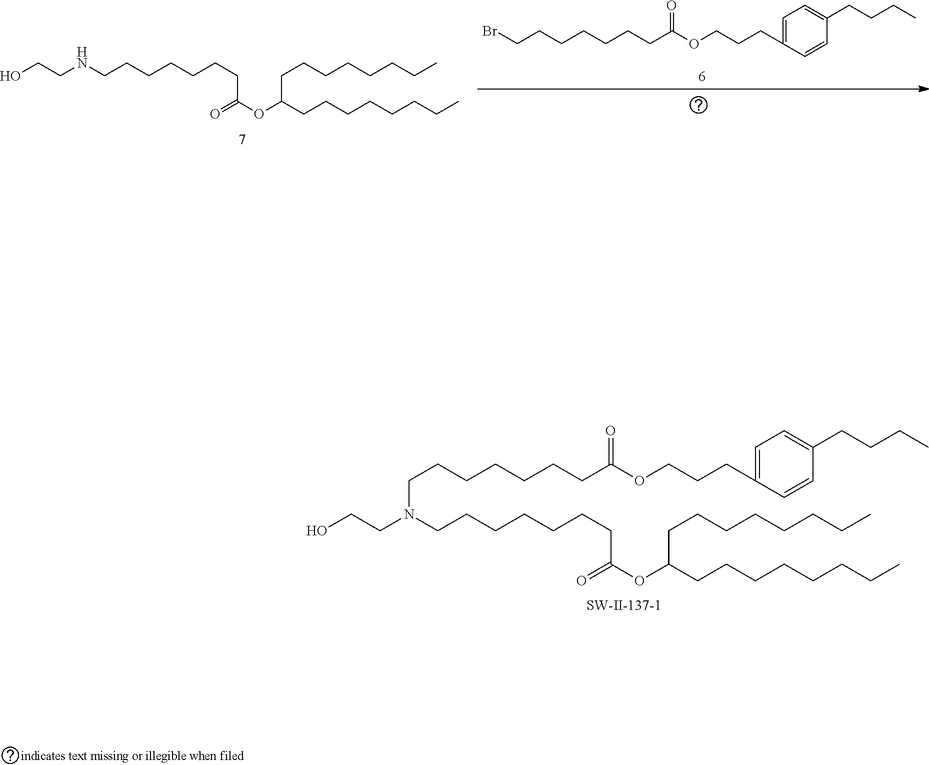 [SMILES: CCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCC)cc1]

Compound 6 (90 mg, 0.25 mmol, 1.0 eq.) was dissolved in MeCN (2 mL). Compound 7 (110 mg, 0.25 mmol, 1.0 eq.), KI (76 mg, 0.50 mmol, 2.0 eq.), CPME (2 mL), and K₂CO₃ (157 mg, 1.25 mmol, 5.0 eq.) were added. The reaction was carried out overnight at 90° C. under nitrogen protection. TLC (DCM/MeOH=10/1) showed that the raw materials reacted completely and the desired product was formed. Quenching was carried out with water (50 mL). Extraction was carried out with EA (40 mL×3). The organic phases were combined, washed with saturated salt solution (2×30 mL), dried with anhydrous Na₂SO₄, filtered, and spin-dried under reduced pressure. The residue was purified by silica gel column, and eluted with DCM/MeOH (1/0-10/1, v/v) to obtain a yellow oily compound (98 mg, 52.12%, SW-II-137-1).

LCMS: Rt: 1.596 min; MS m/z (ELSD): 758.4[M+H]⁺;

HPLC: 98.02% purity, ELSD; RT=5.993 min.

¹H NMR (400 MHz, CDCl₃) δ7.02 (d, J=8.8 Hz, 4H), 4.92-4.71 (m, 1H), 4.01 (t, J=6.4 Hz, 2H), 3.78 (s, 1H), 3.55 (t, J=5.2 Hz, 2H), 2.76-2.40 (m, 10H), 2.21 (dd, J=15.6, 7.7 Hz, 4H), 1.95-1.80 (m, 2H), 1.49 (ddd, J=24.4, 15.8, 6.2 Hz, 15H), 1.34-1.13 (m, 37H), 0.82 (dt, J=13.6, 7.2 Hz, 9H).

¹³C NMR (101 MHz, CDCl₃) δ 173.79 (s), 173.57 (s), 140.49 (s), 138.30 (s), 128.43 (s), 128.22 (s), 77.43 (s), 77.11 (s), 76.79 (s), 74.11 (s), 63.66 (s), 57.96 (s), 55.75 (s), 53.90 (s), 35.22 (s), 34.63 (s), 34.20 (d, J=11.6 Hz), 33.70 (s), 31.80 (d, J=11.2 Hz), 30.30 (s), 29.51 (d, J=2.8 Hz), 29.13 (dd, J=9.6, 6.8 Hz), 27.12 (d, J=2.8 Hz), 26.29 (s), 25.31 (s), 24.97 (d, J=15.6 Hz), 22.66 (s), 22.37 (s), 14.02 (d, J=15.2 Hz)

### N. Compound SW-II-137-2

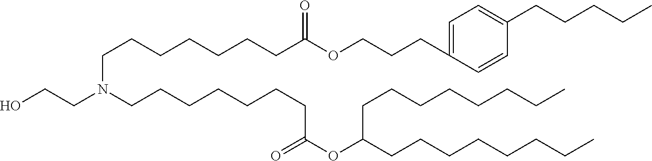 [SMILES: CCCCCCCCC(CCCCCCCC)OC(=O)CCCCCCCN(CCO)CCCCCCCC(=O)OCCCc1ccc(CCCCC)cc1]

### 1. Synthesis of Compound 3

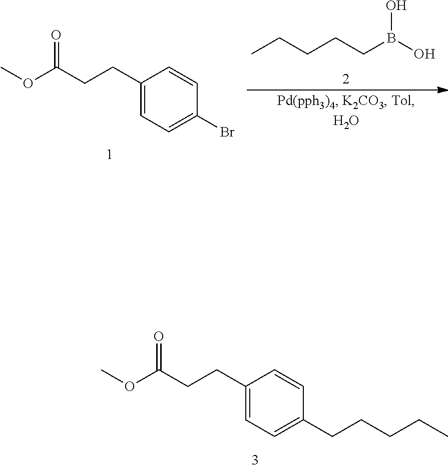

Compound 1 (500 mg, 2.06 mmol, 1.0 eq.), compound 2 (286 mg, 2.47 mmol, 1.2 eq.), Pd(PPh₃)₄ (119 mg, 0.1 mmol, 0.1 eq.), and K₂CO₃ (851 mg, 6.21 mmol, 3.0 eq.) were dissolved in toluene (5.0 mL). Water (0.5 mL) was added. Then, the reaction was carried out at 110° C. for 3 hours under nitrogen protection. TLC (PE/EA=5/1) showed that the raw materials reacted completely and the desired compound was formed. The reaction mixture was quenched with H₂O (70 mL), and extracted with EA (80 mL×3). The organic phase was washed with saturated salt solution (2×30 mL), dried with anhydrous Na₂SO₄, filtered, and spin-dried under reduced pressure. The residue was purified by silica gel column, and eluted with (PE/EA=5/1, v/v) to obtain colorless oily compound 3 (420 mg, 87.5%).

### 2. Synthesis of Compound 4

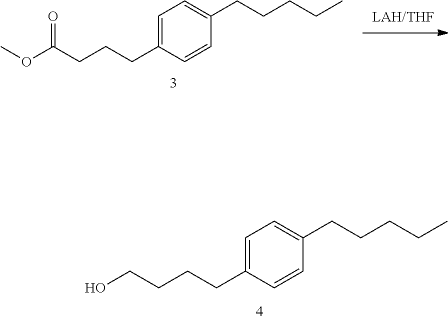 [SMILES: CCCCCc1ccc(CCCC(=O)OC)cc1 ; CCCCCc1ccc(CCCCO)cc1]

Compound 3 (420 mg, 1.78 mmol, 1.0 eq.) was dissolved in THF (3.0 mL). LAH (1 M, 7 mL, 2.0 eq.) was added dropwise at 0° C. under nitrogen protection. Then, the reaction was carried out at room temperature for 2 hours. TLC (PE/EA=5/1) showed that the raw materials reacted completely and the desired product was formed. Quenching was carried out with a HCl (1 M, 4 mL) solution and H₂O (10 mL). Extraction was carried out with EA (50 mL×3). The organic phase was washed with saturated salt solution (2×30 mL), dried with anhydrous Na₂SO₄, filtered, and spin-dried under reduced pressure. The residue was purified by silica gel column, and eluted with (PE/EA=5/1, v/v) to obtain colorless oily compound 4 (320 mg, 94%).

### 3. Synthesis of Compound 6

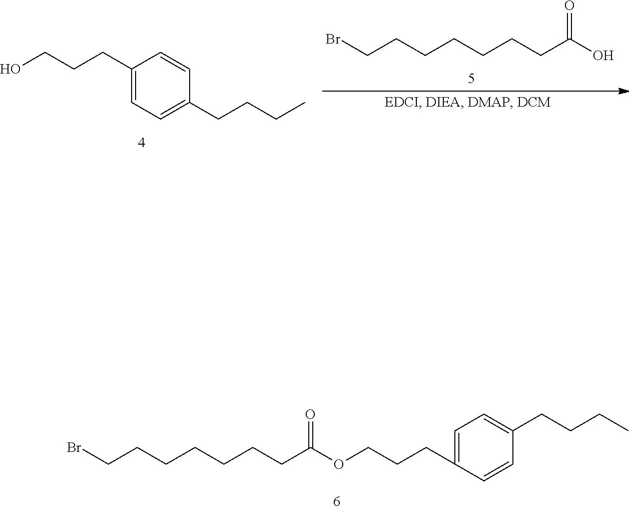 [SMILES: CCCCc1ccc(CCCO)cc1 ; O=C(O)CCCCCCCBr ; CCCCc1ccc(CCCOC(=O)CCCCCCCBr)cc1]

Compound 4 (320 mg, 1.55 mmol, 1.0 eq.) was dissolved in DCM (4.0 mL). Compound 5 (416 mg, 1.86 mmol, 1.2 eq.), EDCI (594 mg, 3.11 mmol, 2.0 eq.), DIEA (802 mg, 6.21 mmol, 4.0 eq.), and DMAP (76 mg, 0.62 mmol, 0.4 eq.) were added. Then, the reaction was carried out overnight at room temperature under nitrogen protection. TLC (PE/EA=20/1) showed that the raw materials reacted completely and the desired product was formed. The reaction mixture was quenched with a HCl (1 M) solution, regulated to PH=4-6, and extracted with DCM (60 mL×3). The organic phase was washed with saturated salt solution (2×35 mL), dried with anhydrous Na₂SO₄, filtered, and spin-dried under reduced pressure. The residue was purified by silica gel column, and eluted with (PE/EA=5/1, v/v) to obtain colorless oily compound 6 (300 mg, 47.17%).

### 4. Synthesis of SW-II-137-2

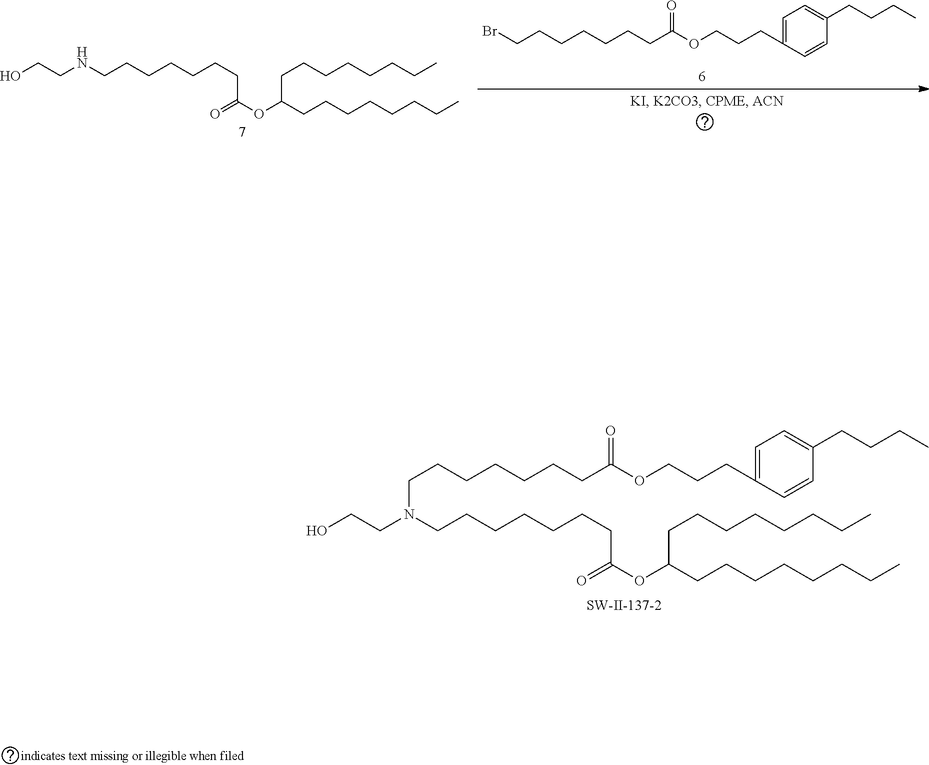 [SMILES: CCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCC)cc1]

Compound 6 (167 mg, 0.41 mmol, 1.2 eq.), compound 7 (150 mg, 0.34 mmol, 1.0 eq.), KI (113 mg, 0.68 mmol, 2.0 eq.), and CPME (2 mL) were dissolved in MeCN (2 mL). K₂CO₃ (235 mg, 1.70 mmol, 5.0 eq.) was added. The reaction was carried out overnight at 90° C. under nitrogen protection. TLC (DCM/MeOH=10/1) showed that the raw materials reacted completely and the desired product was produced. The reaction mixture was quenched with water (50 mL). Extraction was carried out with EA (60 ml×3). The organic phase was dried with anhydrous Na₂SO₄, filtered, and spin-dried under reduced pressure. The residue was purified by silica gel column, and eluted with DCM/MeOH (1/0-10/1, v/v) to obtain a light-yellow oily compound (105 mg, 40.3%, SW-II-137-2).

LCMS: Rt: 1.660 min; MS m/z (ELSD): 772.4[M+H]⁺;

HPLC: 98.38% purity, ELSD; RT=8.743 min.

¹H NMR (400 MHz, CDCl₃) δ7.10 (d, J=8.8 Hz, 4H), 5.04-4.74 (m, 1H), 4.08 (t, J=6.4 Hz, 2H), 3.58 (t, J=5.2 Hz, 2H), 2.65 (dd, J=9.6, 5.6 Hz, 4H), 2.60-2.44 (m, 6H), 2.29 (dd, J=16.4, 7.6 Hz, 4H), 2.01-1.88 (m, 2H), 1.59 (dt, J=9.2, 7.2 Hz, 6H), 1.54-1.42 (m, 8H), 1.39-1.11 (m, 41H), 0.88 (dt, J=11.8, 6.0 Hz, 9H).

¹³C NMR (101 MHz, CDCl₃) δ 173.86 (s), 173.63 (s), 140.59 (s), 138.34 (s), 128.45 (s), 128.24 (s), 77.36 (s), 77.04 (s), 76.72 (s), 74.14 (s), 63.69 (s), 58.11 (s), 55.71 (s), 53.90 (s), 35.53 (s), 34.68 (s), 34.23 (d, J=14.8 Hz), 31.82 (d, J=11.6 Hz), 31.56 (s), 31.26 (s), 30.32 (s), 29.53 (d, J=2.8 Hz), 29.19 (dd, J=8.0, 4.4 Hz), 27.20 (d, J=2.4 Hz), 26.64 (s), 25.33 (s), 25.02 (d, J=15.6 Hz), 22.62 (d, J=11.6 Hz), 14.08 (d, J=8.0 Hz).

### O. Compound SW-II-137-3

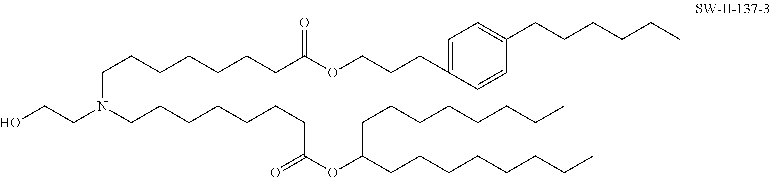 [SMILES: CCCCCCCCC(CCCCCCCC)OC(=O)CCCCCCCN(CCO)CCCCCCCC(=O)OCCCc1ccc(CCCCCC)cc1]

### 1. Synthesis of Compound 3

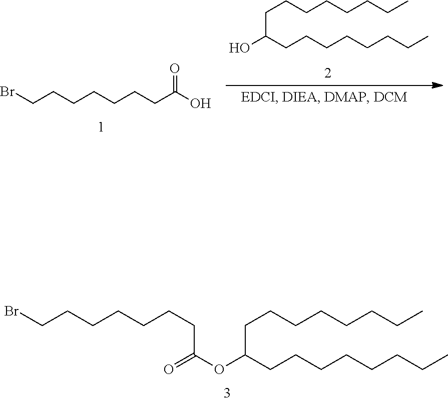 [SMILES: CCCCCCCCC(O)CCCCCCCC ; O=C(O)CCCCCCCBr ; CCCCCCCCC(CCCCCCCC)OC(=O)CCCCCCCBr]

EDCI (16.9 g, 88 mmol, 2 eq.) and DMAP (2.1 g, 18 mmol, 0.4 eq.) were added into a mixture of compound 1 (11.8 g, 53 mmol, 1.2 eq.) and compound 2 (11.2 g, 44 mmol, 1 eq.) in DCM (110 mL), and then DIEA (22.7 g, 176 mmol, 4 eq.) was added. The reaction mixture was stirred at room temperature for 16 hours under nitrogen. TLC (petroleum ether/ethyl acetate=30/1) showed that compound 1 was consumed and the desired product was formed. The reaction mixture was extracted with ethyl acetate (200 mL), washed with water (200 mL×3), dried with anhydrous sodium sulfate, filtered, and concentrated under reduced pressure. The residue was purified by silica gel column chromatography, and eluted with petroleum ether/ethyl acetate (1/0-20/1) to obtain colorless oily compound 3 (7.391 g, 37%).

### 2. Synthesis of Compound 5

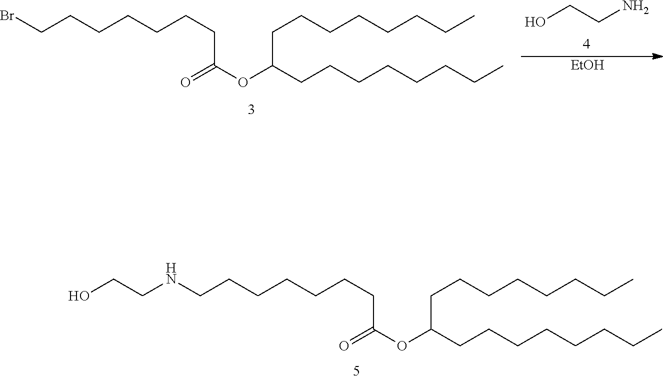

A mixture of compound 3 (7.391 mg, 16.07 mmol, 1 eq.) and compound 4 (29.4 g, 482.02 mmol, 30 eq.) in ethanol (2 mL) was stirred at 55° C. for 16 hours under nitrogen. TLC (DCM/MeOH=10/1) showed that a new major spot was observed. The reaction mixture was extracted with ethyl acetate (100 mL) and washed with water (3×100 mL). The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound 5 (3.695 g, 52%).

### 3. Synthesis of Compound 8

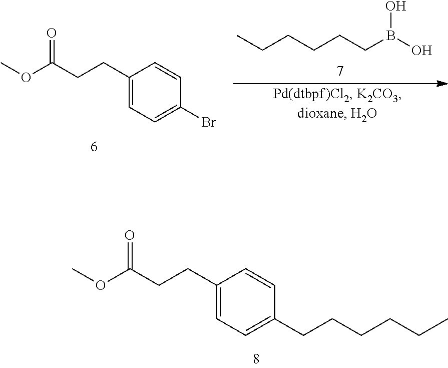

Pd(dtbpf)Cl₂ (269 mg, 0.41 mmol, 0.1 eq.) and potassium carbonate (1.7 g, 12.36 mmol, 3 eq.) were added into a mixture of compound 6 (1 g, 4.12 mmol, 1 eq.) and compound 7 (803 g, 6.17 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 8 (568 mg, 56%).

### 4. Synthesis of Compound 9

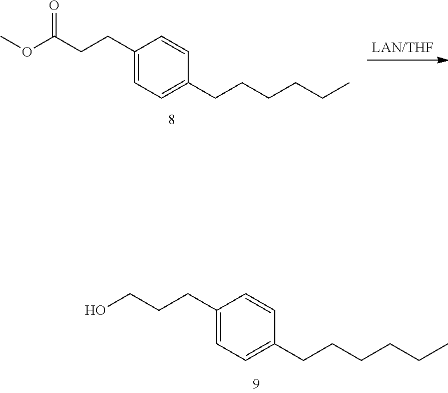 [SMILES: CCCCCCc1ccc(CCC(=O)OC)cc1 ; CCCCCCc1ccc(CCCO)cc1]

At 0° C. and under nitrogen environment, lithium aluminium hydride (2.3 mL, 2.29 mmol, 1 M, in THF, 1 eq.) was added into a mixture of compound 8 (568 mg, 2.29 mmol, 1 eq.) in THF (6 mL). The mixture was stirred at room temperature for 3 hours. TLC (PE/EA=5/1) showed that the reaction was complete and a new major spot was observed. The mixture was quenched with water (2.3 mL) and treated with 2N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 9 (541 mg, >100%), which did not need to be further purified.

### 5. Synthesis of Compound 10

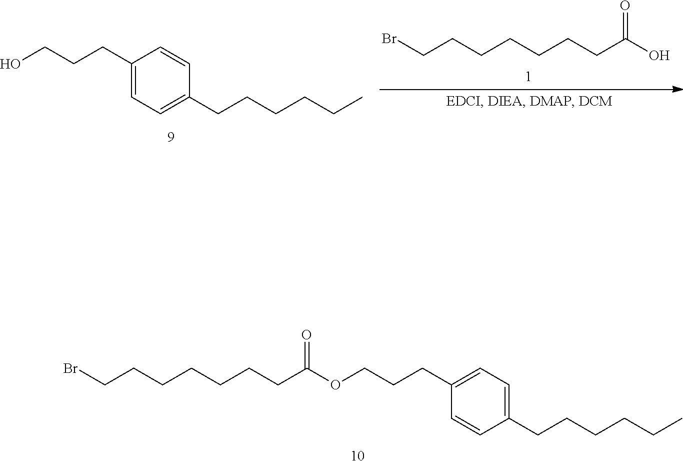 [SMILES: CCCCCCc1ccc(CCCO)cc1 ; O=C(O)CCCCCCCBr ; CCCCCCc1ccc(CCCOC(=O)CCCCCCCBr)cc1]

EDCI (768 mg, 4 mmol, 2 eq.) and DMAP (98 mg, 0.8 mmol, 0.4 eq.) were added into a mixture of compound 9 (441 mg, 2 mmol, 1 eq.) and compound 1 (536 mg, 2.4 mmol, 1.2 eq.) in DCM (5 mL), and then DIEA (1.032 g, 8 mmol, 4 eq.) was added. The reaction mixture was stirred at room temperature for 16 hours under nitrogen. TLC (petroleum ether/ethyl acetate=10/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-10/1) to obtain colorless oily compound 10 (372 mg, 44%).

### 6. Synthesis of SW-II-137-3

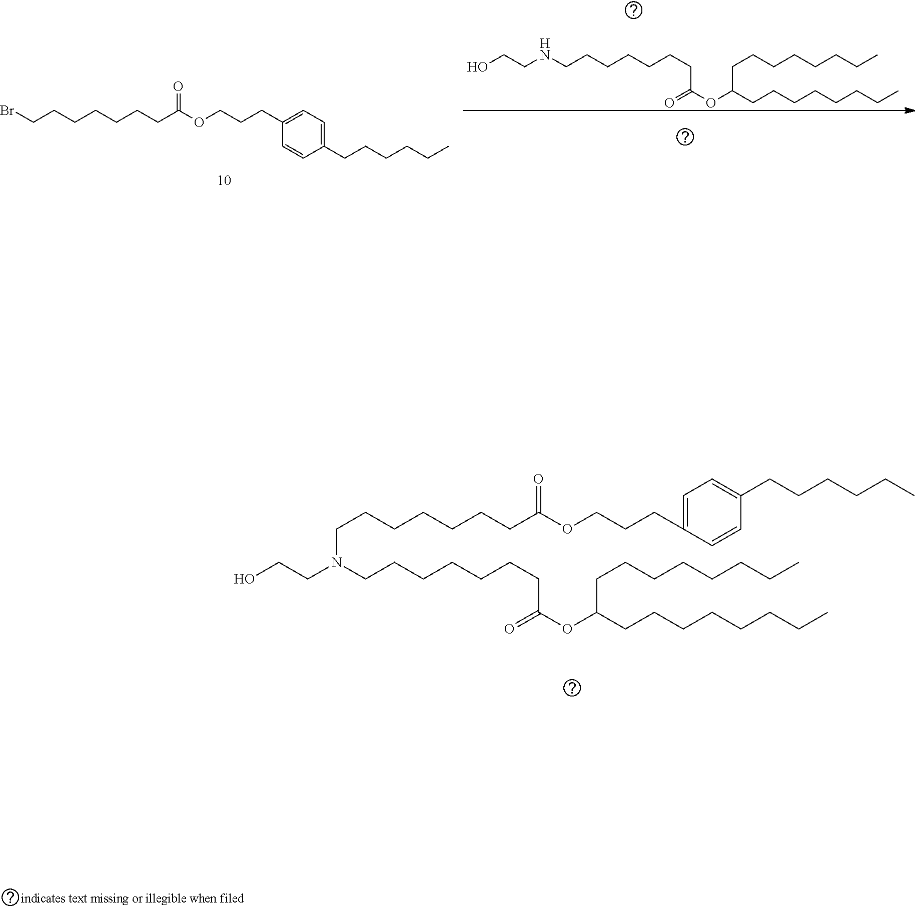 [SMILES: CCCCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCCCC)cc1]

Potassium carbonate (244 mg, 1.765 mmol, 6 eq.) and potassium iodide (117 mg, 0.706 mmol, 2 eq.) were added into a mixture of compound 10 (150 mg, 0.353 mmol, 1 eq.) and compound 5 (156 mg, 0.353 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-137-3 (56.17 mg, 20%).

LCMS: Rt: 1.550 min; MS m/z (ELSD): 786.4[M+H]⁺;

HPLC: 98.597% purity, ELSD; RT=13.153 min.

¹H NMR (400 MHz, CDCl₃) δ7.09 (s, 4H), 4.92-4.78 (m, 1H), 4.08 (t, J=6.6 Hz, 2H), 3.62 (t, J=5.2 Hz, 2H), 2.78-2.50 (m, 10H), 2.35-2.22 (m, 4H), 2.00-1.88 (m, 2H), 1.57 (ddd, J=28.9, 13.5, 4.5 Hz, 14H), 1.38-1.20 (m, 42H), 0.88 (t, J=6.8 Hz, 9H).

¹³C NMR (101 MHz, CDCl₃) δ 173.83 (s), 173.60 (s), 140.60 (s), 138.33 (s), 128.44 (s), 128.24 (s), 77.36 (s), 77.04 (s), 76.72 (s), 74.15 (s), 63.69 (s), 57.95 (s), 55.83 (s), 53.95 (s), 35.56 (s), 34.65 (s), 34.21 (d, J=13.0 Hz), 31.81 (d, J=12.3 Hz), 31.54 (s), 30.32 (s), 29.52 (d, J=3.1 Hz), 29.34-28.94 (m), 27.13 (d, J=2.5 Hz), 26.31 (s), 25.33 (s), 24.99 (d, J=15.7 Hz), 22.64 (d, J=5.7 Hz), 14.11 (s).

### P. Compound SW-II-138-1

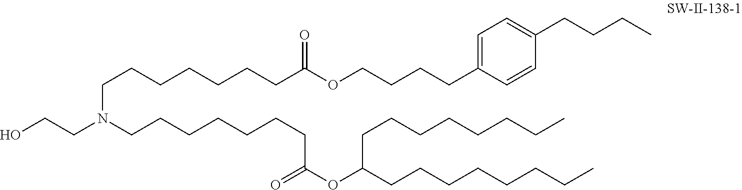 [SMILES: CCCCCCCCC(CCCCCCCC)OC(=O)CCCCCCCN(CCO)CCCCCCCC(=O)OCCCCc1ccc(CCCC)cc1]

### 1. Synthesis of Compound 2

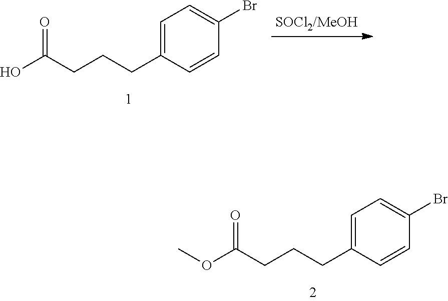

Compound 1 (4 g, 16.46 mmol, 1.0 eq.) was dissolved in MeOH (40 mL). Cooling was carried out to 0° C. SOCl₂ (3.9 g, 32.92 mmol, 2.0 eq.) was added dropwise. Then the reaction was carried out at room temperature for 1 hour. TLC (PE/EA=5/1) showed that the raw materials were consumed completely and the desired product was formed. The system was directly spin-dried under reduced pressure. A NaHCO₃ (70 mL) solution was added into the residue. Extraction was carried out with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain yellow oily compound 2 (4.1 mg, 95%).

### 2. Synthesis of Compound 4

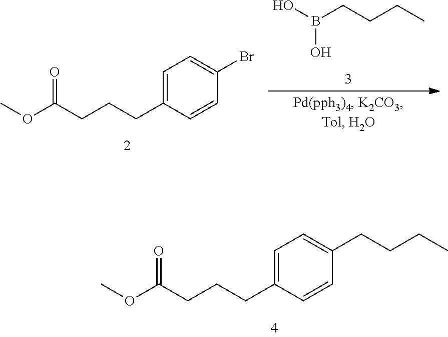

Compound 2 (500 mg, 1.95 mmol, 1.0 eq.), compound 3 (239 mg, 2.34 mmol, 1.2 eq.), Pd(PPh₃)₄ (225 mg, 0.19 mmol, 0.1 eq.), and K₂CO₃ (808 g, 5.85 mmol, 3.0 eq.) were dissolved in toluene (5.0 mL). Water (1 mL) was added. Then the reaction was carried out at 110° C. for 3 hours under nitrogen protection. TLC (PE/EA=5/1) showed that the raw materials were consumed completely and the desired product was formed. H₂O (70 mL) was added into the reaction for quenching. Extraction was carried out with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain yellow oily compound 4 (320 mg, 70%).

### 3. Synthesis of Compound 5

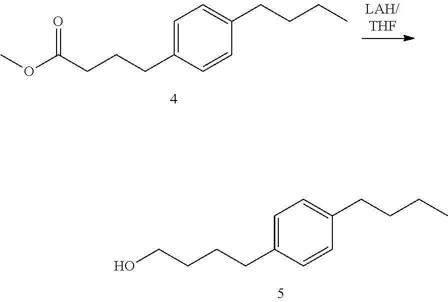 [SMILES: CCCCc1ccc(CCCC(=O)OC)cc1 ; CCCCc1ccc(CCCCO)cc1]

Compound 4 (300 mg, 1.28 mmol, 1.0 eq.) was dissolved in THF (4.0 mL). LAH (97 mg, 2.56 mmol, 2.0 eq.) was added at 0° C. Then the reaction was carried out at room temperature for 2 hours under nitrogen protection. TLC (PE/EA=5/1) showed that the raw materials were consumed completely and the desired product was formed. A HCl (1 M, 4 mL) solution and H₂O (10 mL) were added for quenching reaction. Extraction was carried out with EA (50 mL×3). The organic phase was washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain yellow oily compound 5 (224 mg, 84.8%).

### 4. Synthesis of Compound 7

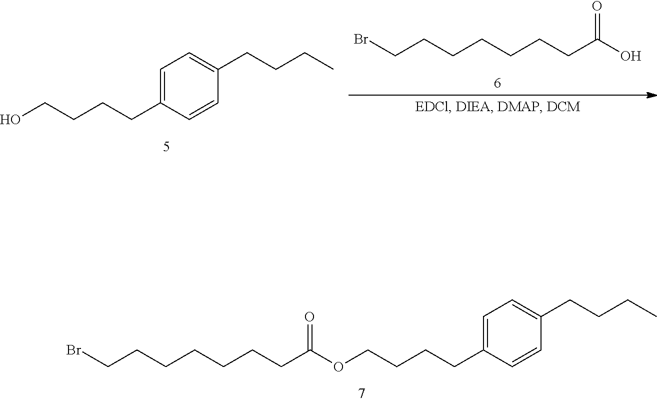

Compound 7 (224 mg, 1.09 mmol, 1.0 eq.) was dissolved in DCM (3.0 mL). Compound 6 (290 mg, 1.30 mmol, 1.2 eq.), EDCI (415 mg, 2.17 mmol, 2.0 eq.), DIEA (561 mg, 4.35 mmol, 4.0 eq.), and DMAP (53 mg, 0.43 mmol, 0.4 eq.) were added. Then, the reaction was carried out overnight at room temperature under nitrogen protection. TLC (PE/EA=30/1) showed that the raw materials were consumed completely and the desired product was formed. The reaction mixture was quenched with a HCl (1 M) solution, regulated to PH=4-6, and extracted with DCM (80 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 7 (208 mg, 46.7%).

### 5. Synthesis of SW-II-138-1

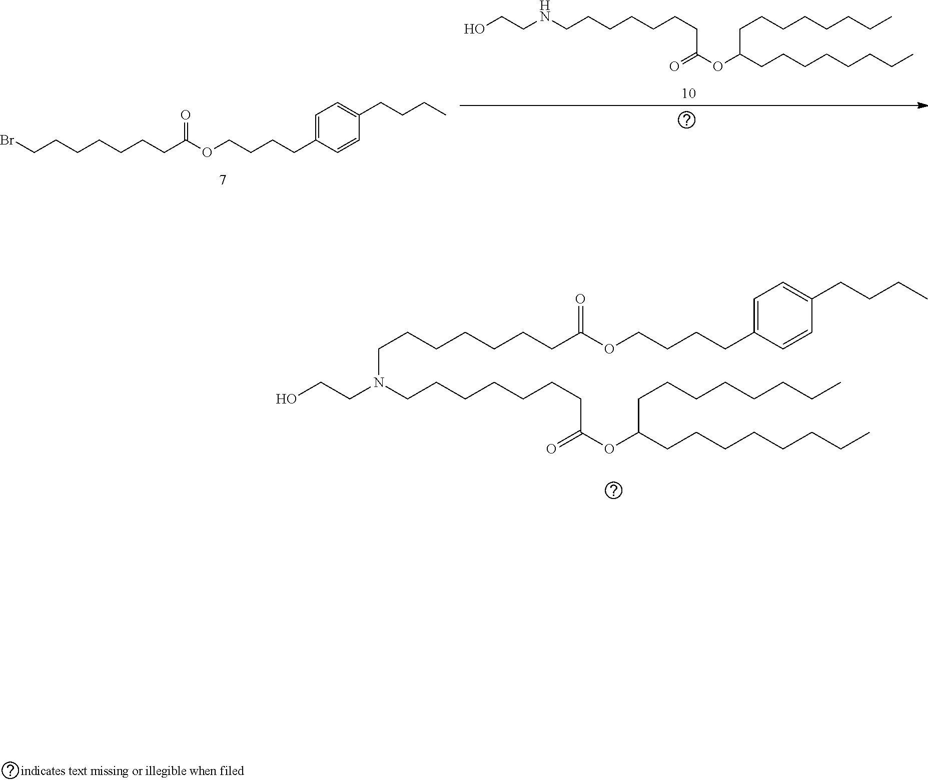 [SMILES: CCCCCCCCC(CCCCCCCC)OC(=O)CCCCCCCNCCO ; CCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCC)cc1]

Compound 10 (110 mg, 0.25 mmol, 1 eq.), compound 7 (153 mg, 0.37 mmol, 1.5 eq.), KI (83 mg, 0.50 mmol, 2.0 eq.), and CPME (2 mL) were dissolved in MeCN (2 mL). K₂CO₃ (172 mg, 1.25 mmol, 5.0 eq.) was added. The reaction was carried out overnight at 90° C. under nitrogen protection. TLC (DCM/MeOH=10/1) showed that the raw materials were consumed completely and the desired product was formed. The reaction mixture was directly spin-dried under reduced pressure. The residue was purified by silica gel column, and eluted with DCM/MeOH (1/0-10/1, v/v) to obtain a light-yellow oily compound (65 mg, 32%, SW-II-138-1).

LCMS: Rt: 1.684 min; MS m/z (ELSD): 772.4[M+H]⁺;

HPLC: 96.56% purity, ELSD; RT=6.346 min.

¹H NMR (400 MHz, CDCl₃) δ7.09 (s, 4H), 4.86 (s, 1H), 4.09 (d, J=6.0 Hz, 2H), 3.97 (s, 2H), 3.07 (d, J=38.8 Hz, 6H), 2.69-2.51 (m, 4H), 2.28 (td, J=7.3, 3.6 Hz, 4H), 1.79 (s, 4H), 1.70-1.46 (m, 16H), 1.42-1.17 (m, 37H), 0.90 (dt, J=13.6, 7.2 Hz, 9H).

¹³C NMR (101 MHz, CDCl₃) δ 173.80 (s), 173.53 (s), 140.32 (s), 139.13 (s), 128.28 (d, J=13.6 Hz), 77.43 (s), 77.11 (s), 76.80 (s), 74.21 (s), 64.22 (s), 56.85 (s), 55.98 (s), 53.93 (s), 35.22 (s), 35.01 (s), 34.54 (s), 34.14 (d, J=5.6 Hz), 33.71 (s), 31.85 (s), 29.50 (d, J=2.8 Hz), 29.22 (s), 29.12-28.60 (m), 28.26 (s), 27.78 (s), 26.70 (d, J=4.4 Hz), 25.31 (s), 24.82 (d, J=17.6 Hz), 24.28 (s), 22.65 (s), 22.37 (s), 14.03 (d, J=15.2 Hz).

### Q. SW-II-138-2

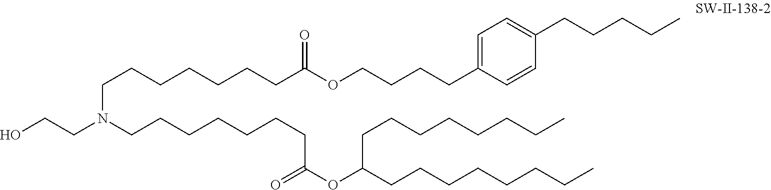 [SMILES: CCCCCCCCC(CCCCCCCC)OC(=O)CCCCCCCN(CCO)CCCCCCCC(=O)OCCCCc1ccc(CCCCC)cc1]

### 1. Synthesis of Compound 3

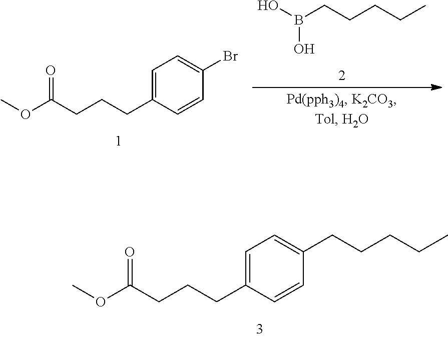

Compound 1 (500 mg, 1.95 mmol, 1.0 eq.), compound 2 (271 mg, 2.34 mmol, 1.2 eq.), Pd(PPh₃)₄ (225 mg, 0.20 mmol, 0.1 eq.), and K₂CO₃ (809 g, 5.86 mmol, 3.0 eq.) were dissolved in toluene (5.0 mL). Water (1 mL) was added. Then the reaction was carried out at 110° C. for 3 hours under nitrogen protection. TLC (PE/EA=5/1) showed that the raw materials were consumed completely and the desired product was formed. The reaction mixture was quenched with water (70 mL), and extracted with EA (80 mL×3). The organic phases were combined, washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 3 (320 mg, 70%).

### 2. Synthesis of Compound 4

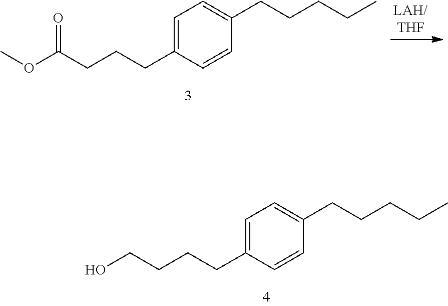 [SMILES: CCCCCc1ccc(CCCC(=O)OC)cc1 ; CCCCCc1ccc(CCCCO)cc1]

Compound 3 (320 mg, 1.29 mmol, 1.0 eq.) was dissolved in THF (3.0 mL). LAH (67 mg, 1.77 mmol, 2.0 eq.) was added at 0° C. Then, the reaction was carried out at room temperature for 2 hours under nitrogen protection. TLC (PE/EA=5/1) showed that the raw materials were consumed completely and the desired product was formed. The reaction mixture was quenched with a HCl (1 M, 2 mL) solution and H₂O (10 mL), and extracted with EA (50 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 4 (180 mg, 64%).

### 3. Synthesis of Compound 6

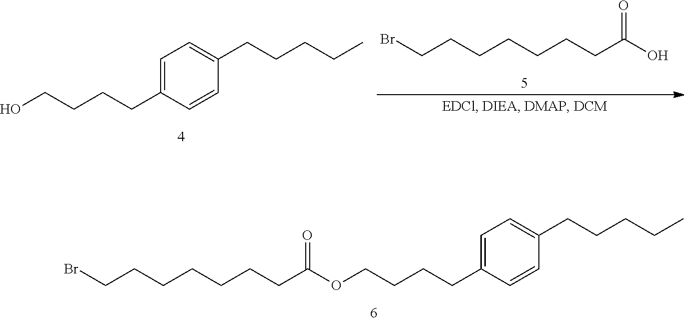

Compound 4 (180 mg, 0.82 mmol, 1.0 eq.) was dissolved in DCM (3.0 mL). Compound 5 (245 mg, 1.10 mmol, 1.2 eq.), EDCI (347 mg, 1.82 mmol, 2.0 eq.), DIEA (470 mg, 3.63 mmol, 4.0 eq.), and DMAP (45 mg, 0.36 mmol, 0.4 eq.) were added. Then, the reaction was carried out overnight at room temperature under nitrogen protection. TLC (PE/EA=30/1) showed that the raw materials were consumed completely and the desired product was formed. The reaction mixture was quenched with a HCl (1 M) solution, regulated to PH=5-6, and extracted with DCM (80 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-30/1, v/v) to obtain colorless oily compound 6 (220 mg, 63.6%).

### 4. Synthesis of SW-II-138-2

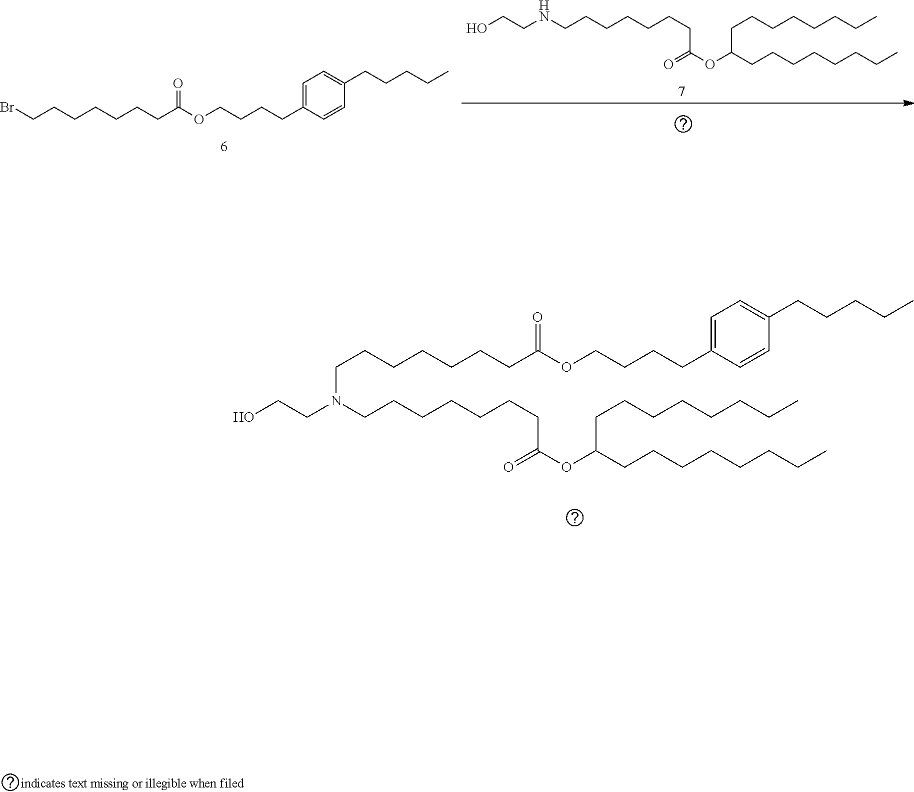 [SMILES: CCCCCCCCC(CCCCCCCC)OC(=O)CCCCCCCNCCO ; CCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCC)cc1]

Compound 6 (158 mg, 0.37 mmol, 1.5 eq.), compound 7 (110 mg, 0.25 mmol, 1.0 eq.), KI (83 mg, 0.50 mmol, 2.0 eq.), and CPME (2 mL) were dissolved in MeCN (2 mL). K₂CO₃ (172 mg, 1.25 mmol, 5.0 eq.) was added. Then, the reaction was carried out overnight at 90° C. under nitrogen protection. TLC (DCM/MeOH=10/1) showed that the raw materials were consumed completely and the desired product was formed. The reaction mixture was directly spin-dried under reduced pressure. The residue was purified by silica gel column, and eluted with DCM/MeOH (1/0-10/1, v/v) to obtain a colorless oily target product (100 mg, 51%, SW-II-138-2).

LCMS: Rt: 1.834 min; MS m/z (ELSD): 786.4[M+H]⁺;

HPLC: 99.20% purity, ELSD; RT=7.990 min.

¹H NMR (400 MHz, CDCl₃) δ7.00 (s, 4H), 4.88-4.73 (m, 2H), 4.00 (t, J=5.6 Hz, 2H), 3.81-3.54 (m, 2H), 3.00-2.81 (m, 2H), 2.81-2.65 (m, 4H), 2.50 (dd, J=16.4, 8.4 Hz, 4H), 2.20 (td, J=7.6, 3.2 Hz, 4H), 1.56 (ddd, J=18.4, 10.4, 5.2 Hz, 13H), 1.43 (d, J=5.6 Hz, 4H), 1.34-1.07 (m, 40H), 0.81 (dt, J=11.2, 5.6 Hz, 9H).

¹³C NMR (101 MHz, CDCl₃) δ 173.78 (s), 173.52 (s), 140.32 (s), 139.11 (s), 128.25 (d, J=11.6 Hz), 77.49 (s), 77.17 (s), 76.85 (s), 74.14 (s), 64.17 (s), 57.25 (s), 55.82 (s), 53.85 (s), 35.50 (s), 35.01 (s), 34.56 (s), 34.14 (d, J=7.2 Hz), 31.84 (s), 31.53 (s), 31.23 (s), 29.49 (d, J=2.8 Hz), 29.21 (s), 28.94 (dd, J=6.4, 4.4 Hz), 28.25 (s), 27.77 (s), 26.84 (d, J=4.4 Hz), 25.30 (s), 25.25-24.59 (m), 22.59 (d, J=11.2 Hz), 14.05 (d, J=7.6 Hz).

### R. SW-II-138-3

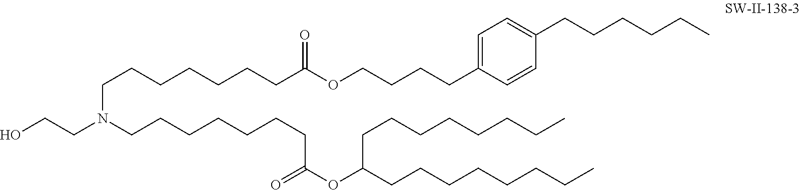 [SMILES: CCCCCCCCC(CCCCCCCC)OC(=O)CCCCCCCN(CCO)CCCCCCCC(=O)OCCCCc1ccc(CCCCCC)cc1]

### 1. Synthesis of Compound 3

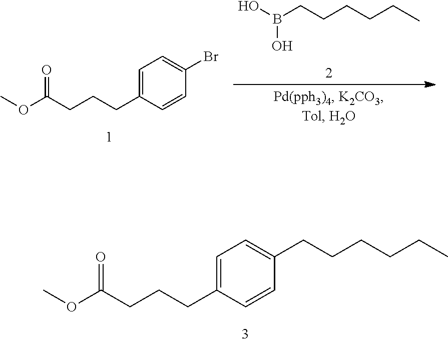

Compound 1 (500 mg, 1.95 mmol, 1.0 eq.), compound 2 (305 mg, 2.34 mmol, 1.2 eq.), Pd(PPh₃)₄ (225 mg, 0.20 mmol, 0.1 eq.), and K₂CO₃ (809 g, 5.86 mmol, 3.0 eq.) were dissolved in toluene (5.0 mL). Water (1 mL) was added. Then, the reaction was carried out at 110° C. for 3 hours under nitrogen protection. TLC (PE/EA=5/1) showed that the raw materials were consumed completely and the desired compound was formed. The reaction mixture was quenched with water (80 mL), and extracted with EA (80 mL×3). The organic phases were combined, washed with saturated salt solution (2×40 mL), dried with anhydrous Na₂SO₄, filtered, and spin-dried under reduced pressure. The residue was purified by silica gel column, and eluted with PE/EA (1/0-5/1, v/v) to obtain colorless oily compound 3 (260 mg, 51.3%).

### 2. Synthesis of Compound 4

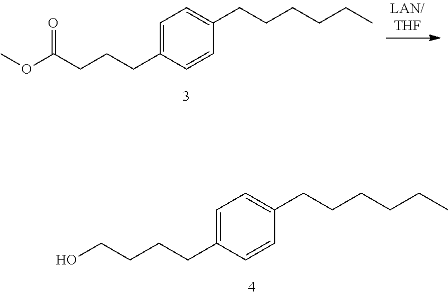 [SMILES: CCCCCCc1ccc(CCCC(=O)OC)cc1 ; CCCCCCc1ccc(CCCCO)cc1]

Compound 3 (260 mg, 0.99 mmol, 1.0 eq.) was dissolved in THF (4.0 mL). LAH (75 mg, 1.98 mmol, 2.0 eq.) was added at 0° C. Then, the reaction was carried out at room temperature for 2 hours under nitrogen protection. TLC (PE/EA=5/1) showed that the raw materials reacted completely and the desired compound was formed. The reaction mixture was quenched with a HCl (1 M, 4 mL) solution and H₂O (20 mL), and extracted with EA (50 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-5/1, v/v) to obtain colorless oily compound 4 (230 mg, 98%).

### 3. Synthesis of Compound 6

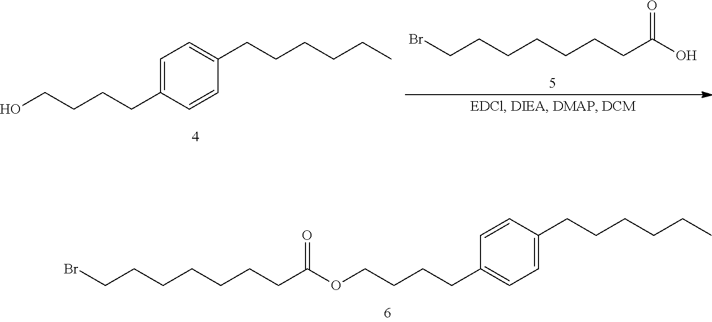

Compound 4 (240 mg, 1.03 mmol, 1.0 eq.) was dissolved in DCM (4.0 mL). Compound 5 (275 mg, 1.23 mmol, 1.2 eq.), EDCI (392 mg, 2.07 mmol, 2.0 eq.), DIEA (530 mg, 4.10 mmol, 4.0 eq.), and DMAP (50 mg, 0.41 mmol, 0.4 eq.) were added in sequence. Then the reaction was carried out overnight at room temperature under nitrogen protection. TLC (PE/EA=20/1) showed that the raw materials were consumed completely and the desired compound was formed. The reaction mixture was quenched with HCl (1 M), regulated to PH=5-6, and extracted with DCM (80 mL×3). The combined organic phases were washed with saturated salt solution (2×30 mL), dried with anhydrous Na₂SO₄, filtered, and spin-dried under reduced pressure. The residue was purified by silica gel column, and eluted with PE/EA (1/0-20/1, v/v) to obtain colorless oily compound 6 (180 mg, 40.9%).

### 4. Synthesis of SW-II-138-3

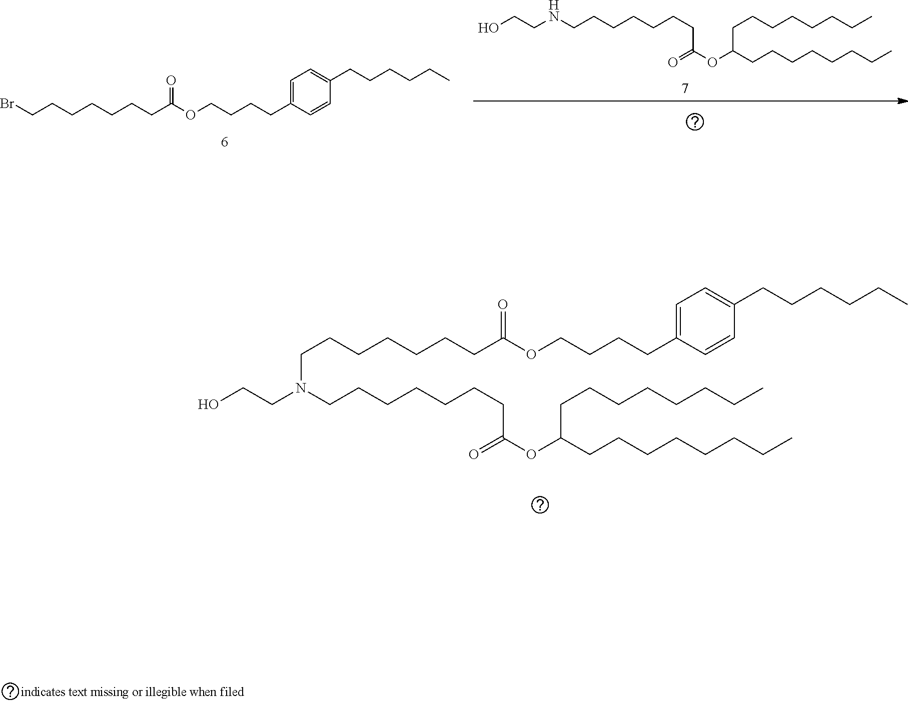 [SMILES: CCCCCCCCC(CCCCCCCC)OC(=O)CCCCCCCNCCO ; CCCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCCC)cc1]

Compound 6 (164 mg, 0.37 mmol, 1 eq), compound 7 (110 mg, 0.24 mmol, 1.0 eq.), KI (83 mg, 0.49 mmol, 2.0 eq.), and CPME (2 mL) were dissolved in MeCN (2 mL). K₂CO₃ (172 mg, 1.24 mmol, 5.0 eq.) was added. Then, the reaction was carried out overnight at 90° C. under nitrogen protection. TLC (DCM/MeOH=10/1) showed that the raw materials were consumed completely and the desired product was formed. The reaction mixture was directly spin-dried under reduced pressure. The residue was purified by silica gel column, and eluted with DCM/MeOH (1/0-10/1, v/v) to obtain a colorless oily target product (108 mg, 52.76%, SW-II-138-3).

LCMS: Rt: 2.007 min; MS m/z (ELSD): 800.4[M+H]⁺;

HPLC: 97.95% purity, ELSD; RT=9.455 min.

¹H NMR (400 MHz, CDCl₃) δ7.08 (s, 4H), 4.86 (p, J=6.4 Hz, 1H), 4.08 (s, 2H), 3.60 (t, J=5.2 Hz, 3H), 2.76-2.42 (m, 10H), 2.28 (td, J=7.6, 2.8 Hz, 4H), 1.70-1.42 (m, 18H), 1.28 (d, J=20.0 Hz, 41H), 0.88 (t, J=6.8 Hz, 9H).

¹³C NMR (101 MHz, CDCl₃) δ 173.85 (s), 173.59 (s), 140.39 (s), 139.15 (s), 128.27 (d, J=12.0 Hz), 77.38 (s), 77.07 (s), 76.75 (s), 74.13 (s), 64.17 (s), 58.06 (s), 55.75 (s), 53.92 (s), 35.57 (s), 35.03 (s), 34.66 (s), 34.22 (d, J=13.2 Hz), 31.81 (d, J=12.4 Hz), 31.54 (s), 29.52 (d, J=2.9 Hz), 29.34-28.95 (m), 28.29 (s), 27.79 (s), 27.16 (d, J=3.6 Hz), 26.50 (s), 25.32 (s), 24.99 (d, J=17.6 Hz), 22.64 (d, J=5.6 Hz), 14.10 (s).

### S. Compound SW-II-139-1

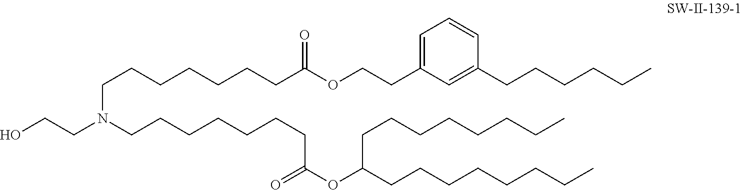 [SMILES: CCCCCCCCC(CCCCCCCC)OC(=O)CCCCCCCN(CCO)CCCCCCCC(=O)OCCc1cccc(CCCCCC)c1]

### 1. Synthesis of Compound 3

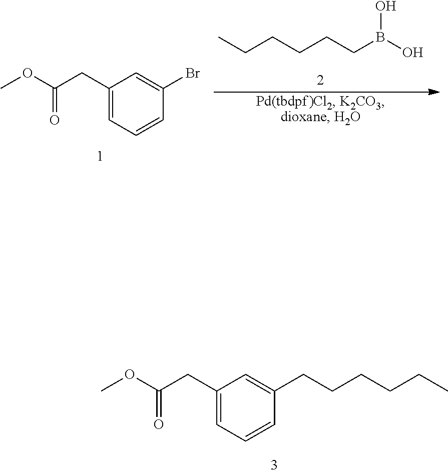

Pd(dtbpf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (852 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (691 mg, 68%).

### 2. Synthesis of Compound 4

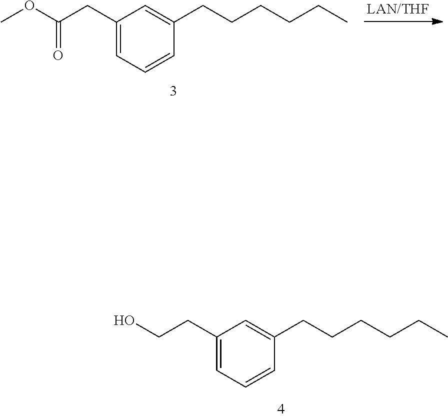 [SMILES: CCCCCCc1cccc(CC(=O)OC)c1 ; CCCCCCc1cccc(CCO)c1]

At 0° C. and under nitrogen environment, lithium aluminium hydride (3 mL, 2.95 mmol, 1 M, in THF, 1 eq.) was added into a mixture of compound 3 (691 mg, 2.95 mmol, 1 eq.) in THF (7 mL). The mixture was stirred at room temperature for 3 hours. TLC (PE/EA=5/1) showed that the reaction was complete and a new major spot was observed. The mixture was quenched with water (3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (547 mg, 90%), which did not need to be further purified.

### 3. Synthesis of Compound 6

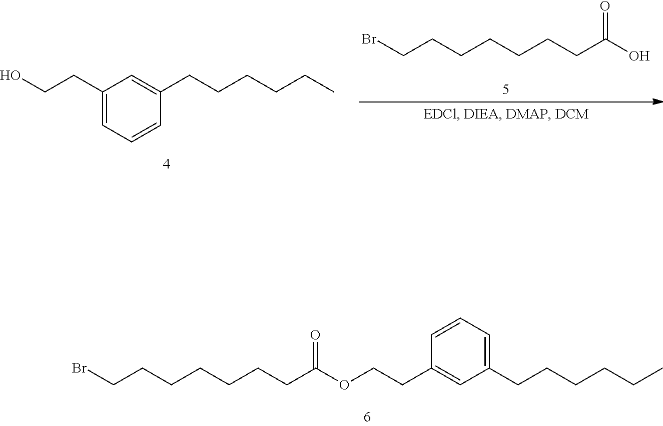 [SMILES: O=C(O)CCCCCCCBr ; CCCCCCc1cccc(CCO)c1 ; CCCCCCc1cccc(CCOC(=O)CCCCCCCBr)c1]

EDCI (833 mg, 4.34 mmol, 2 eq.) and DMAP (106 mg, 0.87 mmol, 0.4 eq.) were added into a mixture of compound 4 (447 mg, 2.17 mmol, 1 eq.) and compound 5 (581 mg, 2.6 mmol, 1.2 eq.) in DCM (5 mL), and then DIEA (1.12 g, 8.68 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (455 mg, 51%).

### 4. Synthesis of SW-II-139-1

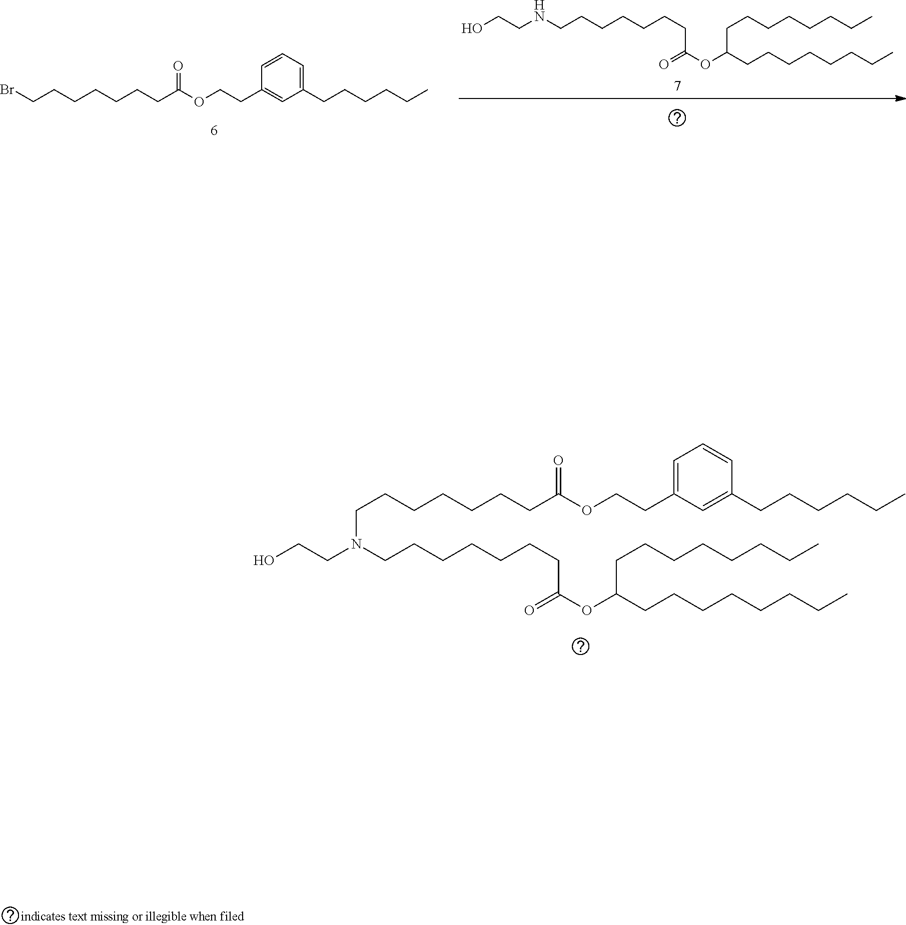 [SMILES: CCCCCCc1cccc(CCOC(=O)CCCCCCCBr)c1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1cccc(CCCCCC)c1]

Potassium carbonate (252 mg, 1.825 mmol, 6 eq.) and potassium iodide (121 mg, 0.73 mmol, 2 eq.) were added into a mixture of compound 6 (150 mg, 0.365 mmol, 1 eq.) and compound 7 (161 mg, 0.365 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-139-1 (54.53 mg, 19%).

LCMS: Rt: 1.521 min; MS m/z (ELSD): 772.4[M+H]⁺;

HPLC: 99.637% purity, ELSD; RT=12.347 min.

¹H NMR (400 MHz, CDCl₃) δ7.20 (t, J=7.7 Hz, 1H), 7.03 (t, J=6.8 Hz, 3H), 4.94-4.78 (m, 1H), 4.27 (t, J=7.2 Hz, 2H), 3.65 (t, J=5.1 Hz, 2H), 2.90 (t, J=7.2 Hz, 2H), 2.73 (t, J=4.9 Hz, 2H), 2.67-2.41 (m, 6H), 2.28 (td, J=7.5, 2.7 Hz, 4H), 1.67-1.45 (m, 14H), 1.41-1.19 (m, 42H), 0.88 (dd, J=7.9, 5.7 Hz, 9H).

¹³C NMR (101 MHz, CDCl₃) δ 173.65 (d, J=11.3 Hz), 143.17 (s), 137.67 (s), 129.04 (s), 128.34 (s), 126.61 (s), 126.11 (s), 77.30 (d, J=11.6 Hz), 77.04 (s), 76.72 (s), 74.16 (s), 64.85 (s), 57.88 (s), 55.93 (s), 53.97 (s), 35.94 (s), 35.13 (s), 34.64 (s), 34.20 (d, J=10.5 Hz), 31.80 (d, J=13.7 Hz), 31.50 (s), 29.52 (d, J=2.9 Hz), 29.34-28.92 (m), 27.08 (d, J=3.9 Hz), 26.10 (s), 25.33 (s), 25.05 (s), 24.82 (s), 22.64 (d, J=6.5 Hz), 14.11 (s).

### T. Compound SW-II-139-2

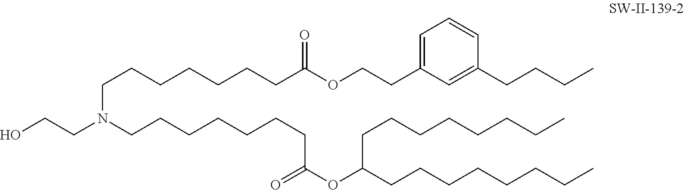 [SMILES: CCCCCCCCC(CCCCCCCC)OC(=O)CCCCCCCN(CCO)CCCCCCCC(=O)OCCc1cccc(CCCC)c1]

### 1. Synthesis of Compound 3

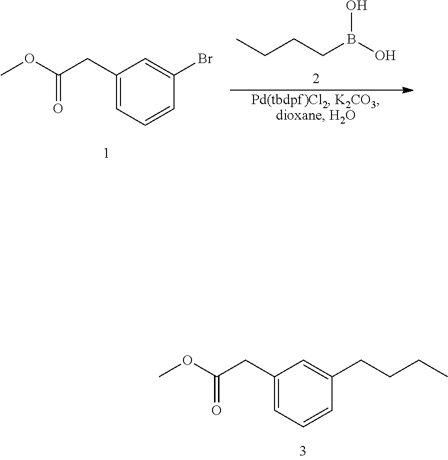

Pd(dtbpf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (668 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (605 mg, 67%).

### 2. Synthesis of Compound 4

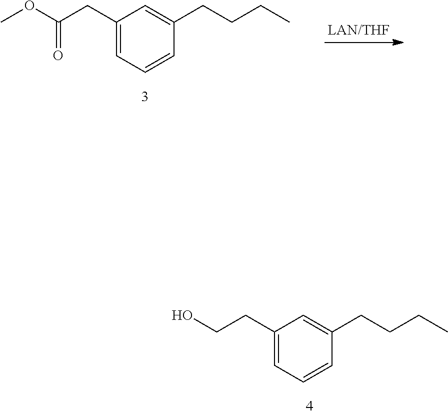

At 0° C. and under nitrogen environment, lithium aluminium hydride (3 mL, 2.94 mmol, 1 M, in THF, 1 eq.) was added into a mixture of compound 3 (605 mg, 2.94 mmol, 1 eq.) in THF (7 mL). The mixture was stirred at room temperature for 3 hours. TLC (PE/EA=5/1) showed that the reaction was complete and a new major spot was observed. The mixture was quenched with water (3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (534 mg, >100%), which did not need to be further purified.

### 3. Synthesis of Compound 6

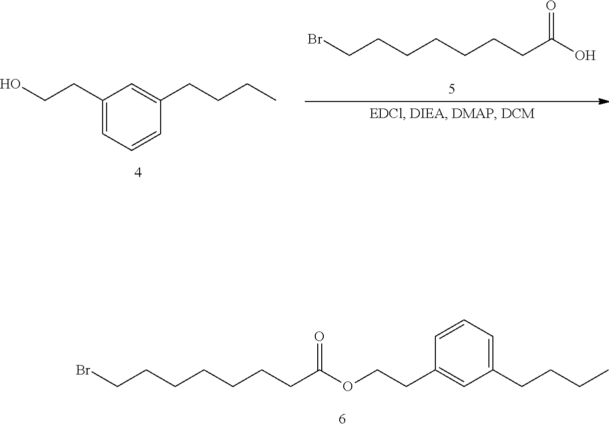 [SMILES: O=C(O)CCCCCCCBr ; CCCCc1cccc(CCO)c1 ; CCCCc1cccc(CCOC(=O)CCCCCCCBr)c1]

EDCI (937 mg, 4.88 mmol, 2 eq.) and DMAP (119 mg, 0.976 mmol, 0.4 eq.) were added into a mixture of compound 4 (434 mg, 2.44 mmol, 1 eq.) and compound 5 (652 mg, 2.93 mmol, 1.2 eq.) in DCM (5 mL), and then DIEA (1.259 g, 9.76 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (355 mg, 38%).

### 4. Synthesis of SW-II-139-2

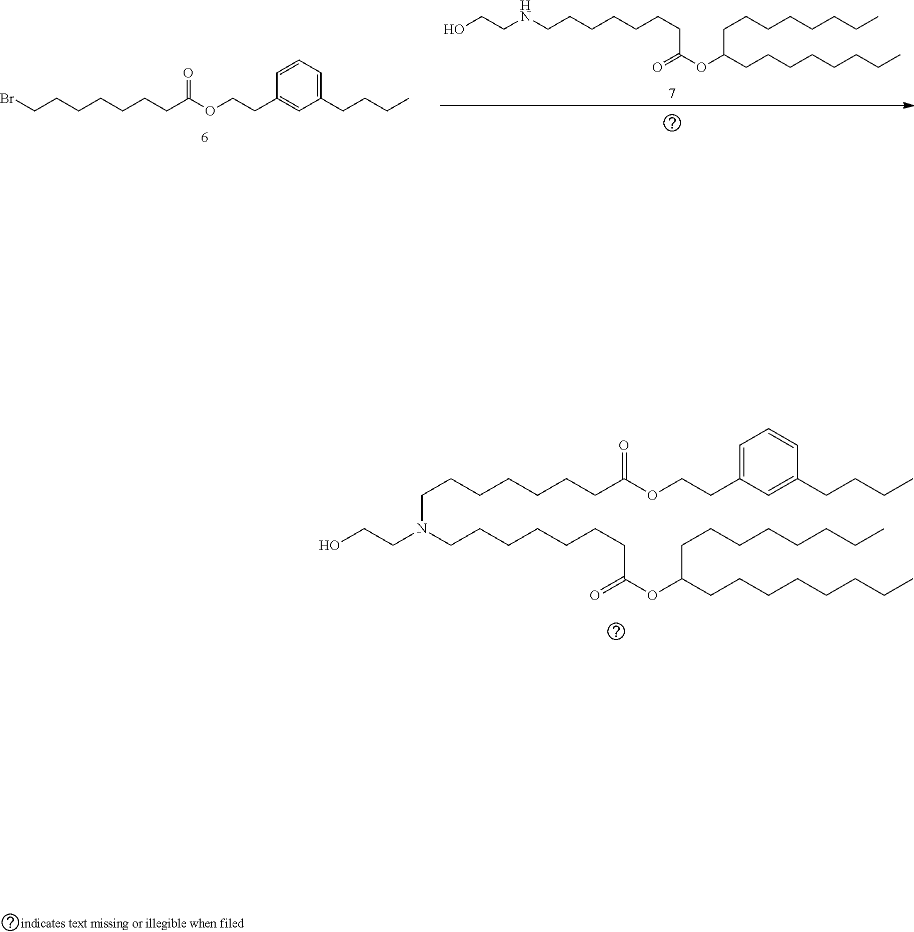 [SMILES: CCCCc1cccc(CCOC(=O)CCCCCCCBr)c1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1cccc(CCCC)c1]

Potassium carbonate (220 mg, 1.595 mmol, 5 eq.) and potassium iodide (106 mg, 0.638 mmol, 2 eq.) were added into a mixture of compound 6 (122 mg, 0.319 mmol, 1 eq.) and compound 7 (140 mg, 0.319 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-139-2 (45.48 mg, 19%).

LCMS: Rt: 1.346 min; MS m/z (ELSD): 744.3[M+H]⁺;

HPLC: 97.994% purity, ELSD; RT=11.235 min.

¹H NMR (400 MHz, CDCl₃) δ7.20 (t, J=7.8 Hz, 1H), 7.03 (t, J=7.6 Hz, 3H), 4.91-4.81 (m, 1H), 4.27 (t, J=7.2 Hz, 2H), 3.89-3.75 (m, 2H), 2.99-2.79 (m, 7H), 2.64-2.48 (m, 2H), 2.28 (td, J=7.5, 3.1 Hz, 4H), 1.74-1.08 (m, 53H), 0.90 (dt, J=13.6, 7.2 Hz, 9H).

¹³C NMR (101 MHz, CDCl₃) δ 173.60 (d, J=11.7 Hz), 143.13 (s), 137.65 (s), 129.06 (s), 128.34 (s), 126.64 (s), 126.11 (s), 77.30 (d, J=11.4 Hz), 77.04 (s), 76.72 (s), 74.22 (s), 64.88 (s), 57.28 (s), 56.55 (s), 54.11 (s), 35.60 (s), 35.12 (s), 34.56 (s), 34.15 (d, J=4.0 Hz), 33.68 (s), 31.86 (s), 29.52 (d, J=2.8 Hz), 29.24 (s), 28.91 (dd, J=7.0, 4.2 Hz), 26.81 (d, J=3.9 Hz), 25.33 (s), 25.12-24.98 (m), 24.83 (d, J=22.2 Hz), 22.67 (s), 22.40 (s), 14.04 (d, J=14.4 Hz).

### U. Compound SW-II-140-1

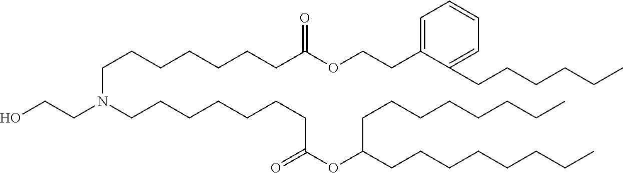 [SMILES: CCCCCCCCC(CCCCCCCC)OC(=O)CCCCCCCN(CCO)CCCCCCCC(=O)OCCc1ccccc1CCCCCC]

### 1. Synthesis of Compound 3

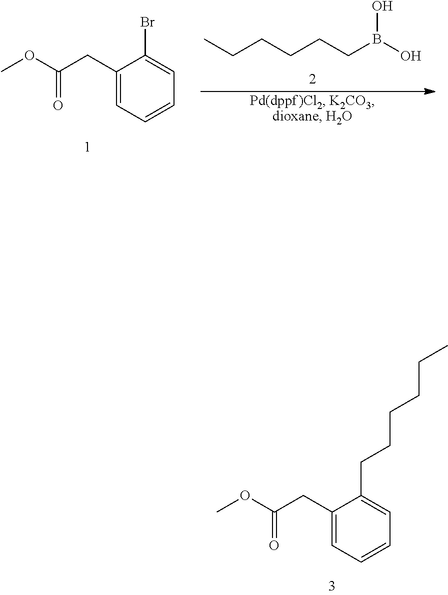

Pd(dppf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (852 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (748 mg, 73%).

### 2. Synthesis of Compound 4

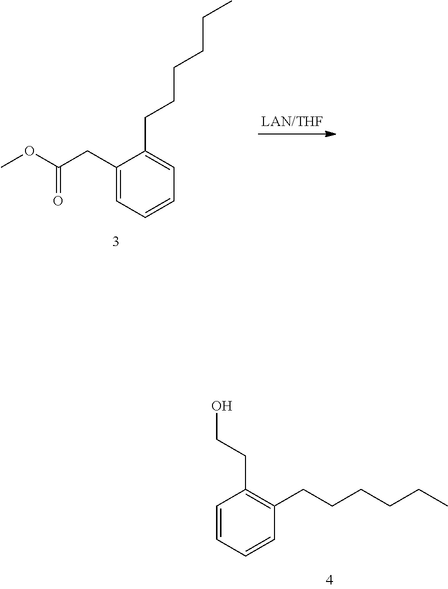 [SMILES: CCCCCCc1ccccc1CC(=O)OC ; CCCCCCc1ccccc1CCO]

At 0° C. and under nitrogen environment, lithium aluminium hydride (3.2 mL, 3.2 mmol, 1 M, in THF, 1 eq.) was added into a mixture of compound 3 (748 mg, 3.2 mmol, 1 eq.) in THF (8 mL). The mixture was stirred at room temperature for 3 hours. TLC (PE/EA=5/1) showed that the reaction was complete and a new major spot was observed. The mixture was quenched with water (3 mL) and treated with 2 N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (493 mg, 75%), which did not need to be further purified.

### 3. Synthesis of Compound 6

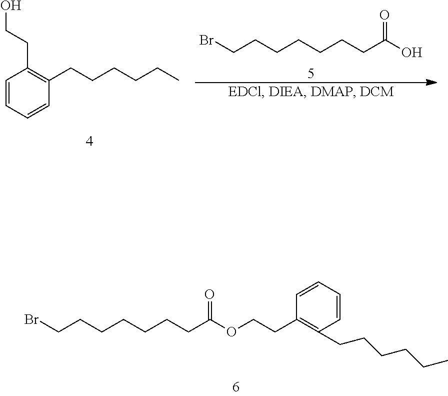

EDCI (733 mg, 3.82 mmol, 2 eq.) and DMAP (93 mg, 0.76 mmol, 0.4 eq.) were added into a mixture of compound 4 (393 mg, 1.91 mmol, 1 eq.) and compound 5 (511 mg, 2.29 mmol, 1.2 eq.) in DCM (5 mL), and then DIEA (986 mg, 7.64 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain compound 6 (327 mg, 42%), which was colorless oil.

### 4. Synthesis of SW-II-140-1

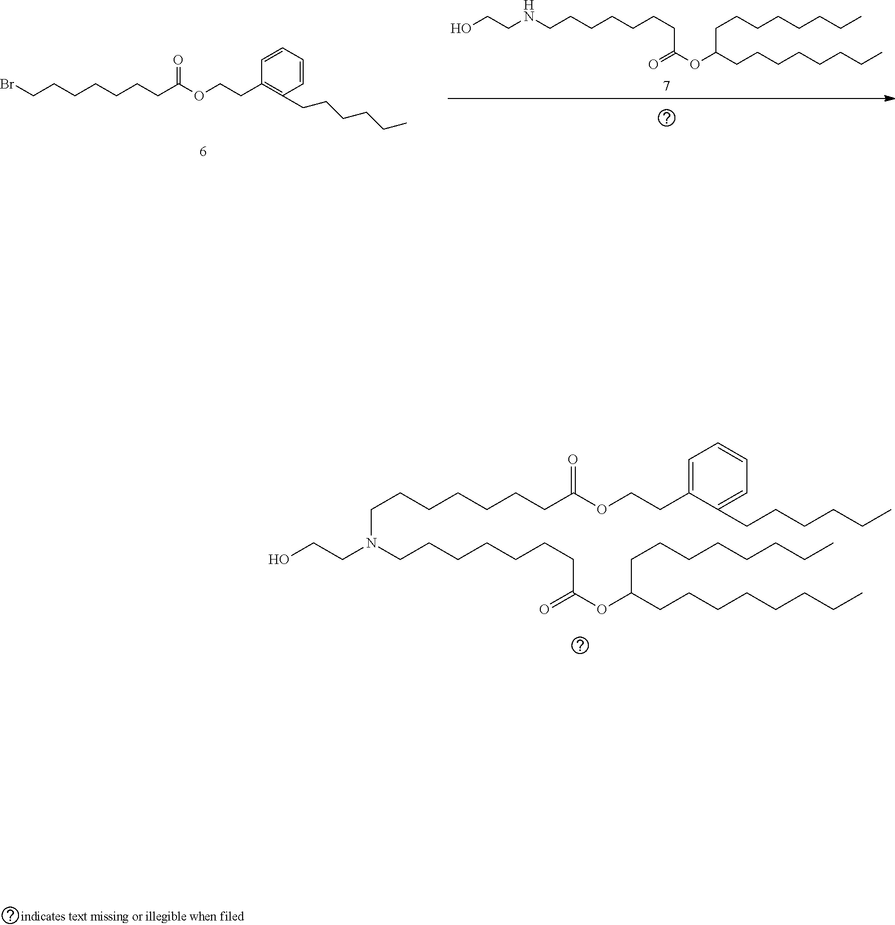 [SMILES: CCCCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccccc1CCCCCC]

Potassium carbonate (302 mg, 2.19 mmol, 6 eq.) and potassium iodide (121 mg, 0.73 mmol, 2 eq.) were added into a mixture of compound 6 (150 mg, 0.365 mmol, 1 eq.) and compound 7 (161 mg, 0.365 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-140-1 (180 mg, 64%).

LCMS: Rt: 1.568 min; MS m/z (ELSD): 772.4[M+H]⁺;

HPLC: 98.053% purity, ELSD; RT=8.702 min.

¹H NMR (400 MHz, CDCl₃) δ7.23-7.05 (m, 4H), 4.95-4.79 (m, 1H), 4.25 (t, J=7.4 Hz, 2H), 3.62 (t, J=4.8 Hz, 2H), 2.96 (dd, J=15.4, 8.0 Hz, 2H), 2.74-2.49 (m, 8H), 2.28 (dd, J=14.2, 7.2 Hz, 4H), 1.67-1.44 (m, 14H), 1.41-1.20 (m, 42H), 0.90 (dt, J=13.2, 7.1 Hz, 9H).

¹³C NMR (101 MHz, CDCl₃) δ 173.68 (d, J=10.2 Hz), 141.26 (s), 135.23 (s), 129.73 (s), 129.37 (s), 126.72 (s), 125.92 (s), 77.35 (s), 77.03 (s), 76.71 (s), 74.17 (s), 64.52 (s), 57.99 (s), 55.87 (s), 53.94 (s), 34.66 (s), 34.21 (d, J=11.5 Hz), 32.75 (s), 31.83 (d, J=9.8 Hz), 31.32 (s), 29.65-28.88 (m), 27.15 (d, J=3.7 Hz), 26.35 (s), 25.33 (s), 25.07 (s), 24.83 (s), 22.66 (d, J=3.4 Hz), 14.12 (s).

### V. SW-II-140-2

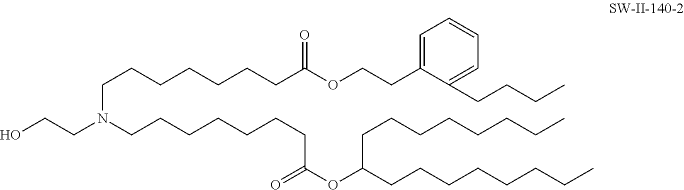 [SMILES: CCCCCCCCC(CCCCCCCC)OC(=O)CCCCCCCN(CCO)CCCCCCCC(=O)OCCc1ccccc1CCCC]

### 1. Synthesis of Compound 3

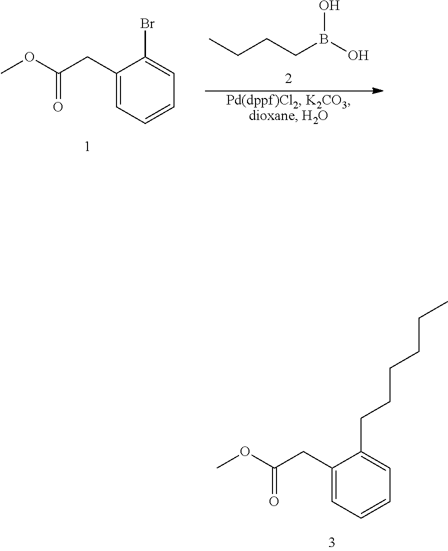

Pd(dppf)Cl₂ (286 mg, 0.437 mmol, 0.1 eq.) and potassium carbonate (1.8 g, 13.11 mmol, 3 eq.) were added into a mixture of compound 1 (1 g, 4.37 mmol, 1 eq.) and compound 2 (668 g, 6.55 mmol, 1.5 eq.) in 1,4-dioxane/water (10 mL/1 mL). The mixture was stirred overnight at 100° C. under nitrogen. TLC (PE/EA=20/1) showed that the reaction was complete and a new major spot was observed. The mixture was extracted with ethyl acetate and washed with water. The organic layer was dried with anhydrous sodium sulfate, filtered, and concentrated under vacuum. The residue was purified by silica gel column chromatography, and eluted with PE/EA (1/0-20/1) to obtain colorless oily compound 3 (406 mg, 45%).

### 2. Synthesis of Compound 4

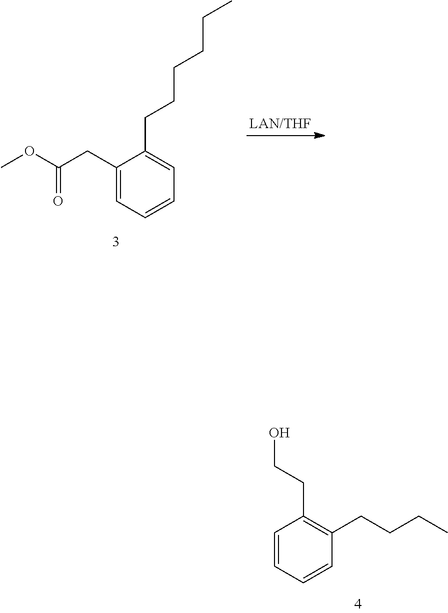 [SMILES: CCCCCCc1ccccc1CC(=O)OC ; CCCCc1ccccc1CCO]

At 0° C. and under nitrogen environment, lithium aluminium hydride (2 mL, 1.97 mmol, 1 M, in THF, 1 eq.) was added into a mixture of compound 3 (406 mg, 1.97 mmol, 1 eq.) in THF (5 mL). The mixture was stirred at room temperature for 3 hours. TLC (PE/EA=5/1) showed that the reaction was complete and a new major spot was observed. The mixture was quenched with water (2 mL) and treated with 2N hydrochloric acid to regulate the pH between 6 and 7, extracted with ethyl acetate and washed with brine. The organic layer was dried with anhydrous sodium sulfate, filtered, and concentrated under vacuum to obtain colorless oily compound 4 (341 mg, 97%), which did not need to be further purified.

### 3. Synthesis of Compound 6

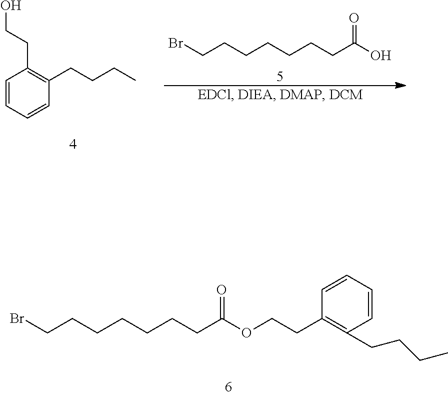

EDCI (518 mg, 2.7 mmol, 2 eq.) and DMAP (66 mg, 0.54 mmol, 0.4 eq.) were added into a mixture of compound 4 (241 mg, 1.35 mmol, 1 eq.) and compound 5 (361 mg, 1.62 mmol, 1.2 eq.) in DCM (3 mL), and then DIEA (697 mg, 5.4 mmol, 4 eq.) was added. The reaction mixture was stirred at room temperature for 16 hours under nitrogen. TLC (petroleum ether/ethyl acetate=15/1) showed that the desired product was formed. The reaction mixture was extracted with ethyl acetate and washed with water. The organic layer was dried with anhydrous sodium sulfate, filtered, and concentrated under vacuum. The residue was purified by silica gel column chromatography, and eluted with petroleum ether/ethyl acetate (1/0-20/1) to obtain colorless oily compound 6 (185 mg, 32%).

### 4. Synthesis of SW-II-140-2

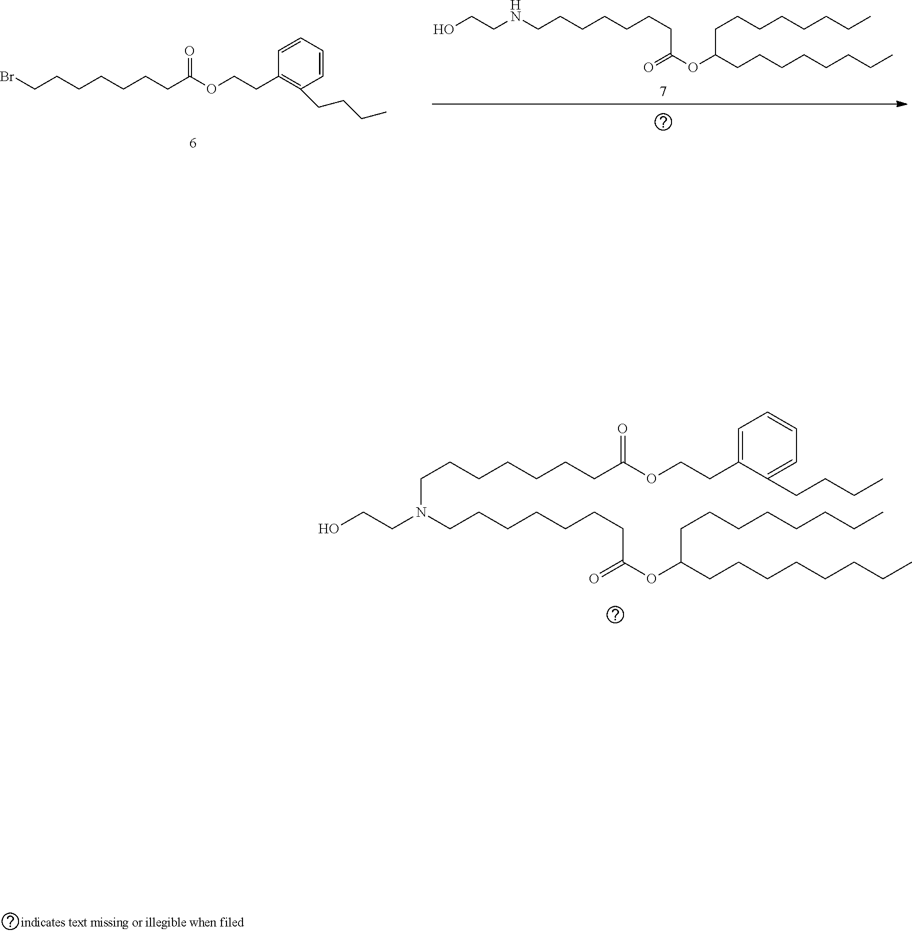 [SMILES: CCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccccc1CCCC]

Potassium carbonate (400 mg, 2.898 mmol, 6 eq.) and potassium iodide (160 mg, 0.966 mmol, 2 eq.) were added into a mixture of compound 6 (185 mg, 0.483 mmol, 1 eq.) and compound 7 (213 mg, 0.483 mmol, 1 eq.) in CPME/CH₃CN (2 mL/2 mL). After the addition, the mixture was stirred overnight at 90° C. under nitrogen. TLC (DCM/MeOH=10/1) showed that the reaction was complete and a new major spot was observed. The mixture was extracted with ethyl acetate and washed with water. The organic layer was dried with anhydrous sodium sulfate, filtered, and concentrated under reduced pressure. The residue was purified by silica gel column chromatography, and eluted with DCM/MeOH (1/0-10/1, v/v) to obtain yellow oily compound SW-II-140-2 (161 mg, 45%).

LCMS: Rt: 1.696 min; MS m/z (ELSD): 744.3[M+H]⁺;

HPLC: 94.658% purity, ELSD; RT=5.938 min.

¹H NMR (400 MHz, CDCl₃) δ7.22-7.03 (m, 4H), 4.94-4.78 (m, 1H), 4.25 (t, J=7.3 Hz, 2H), 3.70-3.54 (m, 2H), 2.96 (t, J=7.4 Hz, 2H), 2.77-2.41 (m, 8H), 2.28 (dd, J=14.3, 7.1 Hz, 4H), 1.65-1.18 (m, 52H), 0.91 (dt, J=13.3, 7.1 Hz, 9H).

¹³C NMR (101 MHz, CDCl₃) δ 173.67 (d, J=10.8 Hz), 141.22 (s), 135.23 (s), 129.73 (s), 129.39 (s), 126.72 (s), 125.92 (s), 77.36 (s), 77.04 (s), 76.72 (s), 74.17 (s), 64.52 (s), 57.92 (s), 55.92 (s), 53.96 (s), 34.66 (s), 34.21 (d, J=11.2 Hz), 33.51 (s), 32.44 (s), 31.83 (d, J=9.3 Hz), 29.53 (d, J=2.9 Hz), 29.14 (dd, J=11.3, 8.5 Hz), 27.12 (d, J=4.1 Hz), 26.23 (s), 25.33 (s), 25.06 (s), 24.82 (s), 22.73 (d, J=9.9 Hz), 14.08 (d, J=8.8 Hz).

### Example 2 Comparison of Preparations Prepared by Different Prescriptions with a Cationic Lipid being M5

### 2.1. Preparation of LNP-mRNA Preparations and LPP-mRNA Preparations

### 2.1.1 Preparation of Lipid Nanoparticle (LNP-mRNA) Preparations:

Preparation of an aqueous mRNA solution: a luciferase mRNA (SEQ ID NO: 1) was diluted into 0.083 mg/mL aqueous mRNA solution with 50 mM sodium citrate buffer (pH=4.0).

Preparation of a lipid solution: MC3:DSPC:cholesterol:PEG-DMG was dissolved in an ethanol solution in a molar ratio of 50:10:38.5:1.5 according to the lipid types and lipid ratios listed in Table 1, to prepare a 6 mg/mL lipid solution.

Preparation of LNP: by using a microfluidic technology (Micro & Nano (Shanghai) Biologics Co., Ltd., Model: Inano D), the lipid solution and the aqueous mRNA solution were mixed under the following conditions: volume=4.0 mL; flow rate ratio=3 (lipid solution):1 (aqueous mRNA solution), and total flow rate=12 mL/min, in a mass ratio of mRNA:lipid of 1:20 to obtain an LNP-mRNA solution.

Centrifugal ultrafiltration: the LNP-mRNA solution was added into an ultrafiltration tube for centrifugal ultrafiltration and concentration (centrifuge force of 3,400 g, centrifuge time of 60 minutes, and temperature of 4° C.), and the volume was adjusted to an mRNA concentration of 0.1 mg/mL, to obtain LNP-mRNA preparations numbered MC3.

### 2.1.2 Preparation of Lipopolyplex (LPP-mRNA) Preparations:

Preparation of an aqueous mRNA solution: a luciferase mRNA (SEQ ID NO: 1) was diluted into 0.1 mg/mL aqueous mRNA solution with 8 mM sodium citrate buffer (pH of 4.0).

Preparation of a lipid solution: lipids were dissolved in an ethanol solution according to the lipid ratios listed in Table 1, to prepare a 6 mg/mL lipid solution.

Preparation of a protamine sulfate solution: protamine sulfate was dissolved in nuclease-free water to prepare a protamine sulfate solution with a working concentration of 0.125 mg/mL.

Preparation of a core nanoparticle solution: by using a microfluidic technology, the protamine sulfate solution was mixed with the mRNA solution under the following conditions: volume=4.0 mL; flow rate ratio=5 (mRNA):1 (protamine solution), total flow rate=12 mL/min, start waste=0.35 mL, end waste=0.1 mL, and room temperature, in a mass ratio of 1:4 to obtain a core nanoparticle solution formed by protamine and mRNA.

Preparation of LPP: the core nanoparticle solution and the lipid solution were secondarily mixed under the following conditions: volume=4.0 mL, flow rate ratio=3 (lipid solution):1 (core nanoparticle solution), total flow rate=12 mL/min, start waste=0.35 mL, end waste=0.1 mL, and room temperature, in a mass ratio of mRNA:lipid of 1:20 to obtain an LPP-mRNA

### Solution

Centrifugal ultrafiltration: the LPP-mRNA solution was subjected to ultrafiltration and centrifugation to remove ethanol (centrifuge force of 2,000-3,000 rpm, centrifuge time of 20-40 minutes, repeating three times of centrifugal ultrafiltration, and temperature of 4° C.), and the volume was adjusted to an mRNA concentration of 0.1 mg/mL, to obtain LPP-mRNA preparations numbered A14, B11, B12, B17, B18, B19, and B23.

TABLE 1
Prescriptions with a cationic lipid being M5

| | M5 (%) | | DOPE (%) | | Cholesterol (%) | | PEG-DMG (%) | |
|---|---|---|---|---|---|---|---|---|
| | Mole | Mass | Mole | Mass | Mole | Mass | Mole | Mass |
| | (%) | (%) | (%) | (%) | (%) | (%) | (%) | (%) |
| A14 | 46.1 | 51.3 | 23 | 26.9 | 30 | 18.2 | 0.9 | 3.6 |
| B11 | 40 | 47.2 | 15 | 18.6 | 43.5 | 28.0 | 1.5 | 6.3 |
| B12 | 35 | 41.9 | 20 | 25.1 | 44 | 28.7 | 1 | 4.2 |
| B17 | 50 | 57.0 | 15 | 17.9 | 34 | 21.1 | 1 | 4.0 |
| B18 | 50 | 54.9 | 20 | 23.0 | 28.75 | 17.2 | 1.25 | 4.9 |
| B19 | 45 | 52.2 | 15 | 18.2 | 38.75 | 24.5 | 1.25 | 5.1 |
| B23 | 35 | 43.2 | 15 | 19.4 | 49 | 33.0 | 1 | 4.4 |

| | MC3 (%) | | DSPC (%) | | Cholesterol (%) | | PEG-DMG (%) | |
|---|---|---|---|---|---|---|---|---|
| | Mole | Mass | Mole | Mass | Mole | Mass | Mole | Mass |
| | (%) | (%) | (%) | (%) | (%) | (%) | (%) | (%) |
| MC3-LNP | 50 | 54.7 | 10 | 13.5 | 38.5 | 25.4 | 1.5 | 6.4 |

### 2.2. In Vivo Luciferase Expression of Preparations Prepared by Different Prescriptions

This example used the LPP solutions of MC3-LNP, A14, B11, B12, B17, B18, B19, and B23 as prepared in Examples 2.1.1 and 2.1.2 to test the in vivo luciferase expression of the preparations prepared by different prescriptions. The specific test methods were as follows:

Female Balb/C mice (Beijing Vital River Laboratory Animal Technology Co., Ltd.) of 6 weeks of age with a weight of around 20 g were used, and each mouse was injected subcutaneously unilaterally with 1×10⁶ murine-derived B-cell lymphoma cells, A20 cells. When tumors grew to a size of about 300-500 mm³, A20 subcutaneous tumor-bearing mice were divided into 9 groups (MC3-LNP, A14, B11, B12, B17, B18, B19, and B23 groups, with 4 mice per group), and LPP solutions (50 μL) containing 5 μg of luciferase mRNA were respectively taken and administered to the mice by intratumoral injection. 6 hours after the administration, mice were injected intraperitoneally with a 150 mg/kg D-fluorescein substrate. The bioluminescence was measured in a Xenogen IVIS-200 imaging system 7 minutes after substrate injection to evaluate the expression and distribution of the luciferase mRNA in the mice.

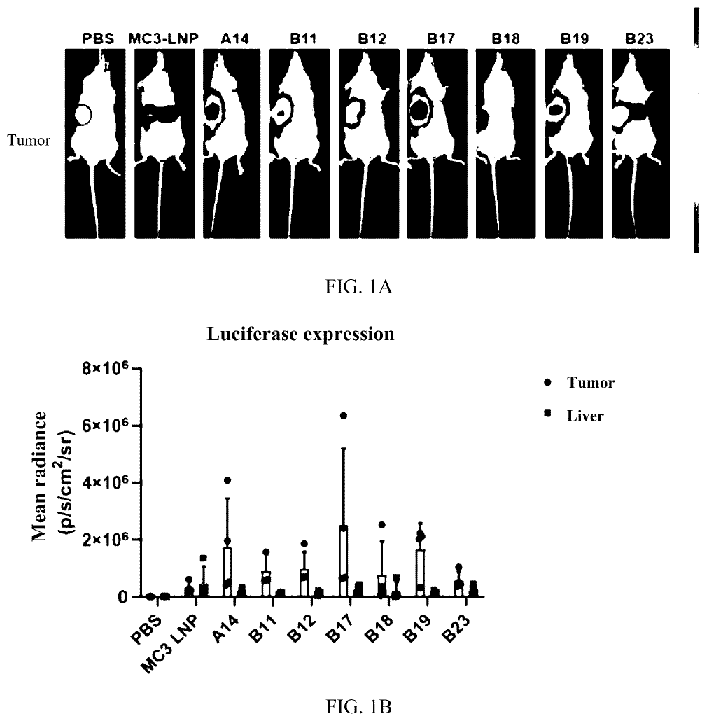

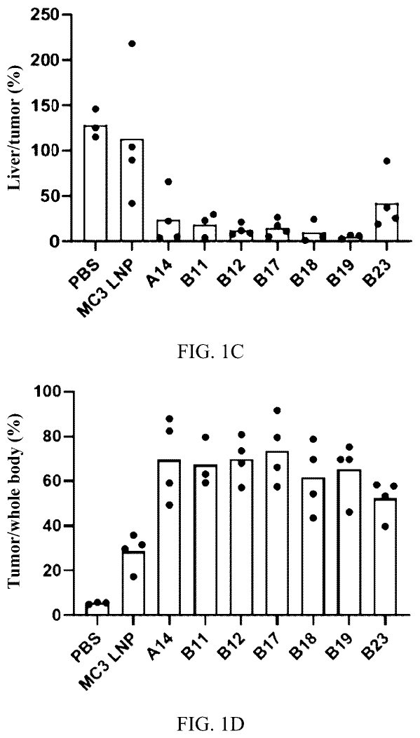

The results of luciferase signal expression of the preparations prepared by different prescriptions 6 hours after the administration are shown in FIG. 1A and FIG. 1B, wherein FIG. 1B shows the luciferase expression in the tumor and liver in each group from left to right. It is observed that the LPP solutions prepared by the prescriptions have higher luciferase expression in the tumor than MC3-LNP, indicating that the LPP solutions prepared by the prescriptions have higher expression efficiency in the tumor than the MC3-LNP solution.

The ratios of luciferase expression of liver/tumor are shown in FIG. 1C, where the LPP solutions prepared by the prescriptions have a lower ratio of luciferase expression of liver/tumor, which is significantly lower than MC3-LNP, indicating that the LPP solutions prepared by the prescriptions have a better tumor targeting effect than the MC3-LNP solution, less expression in liver, and low hepatotoxicity.

The ratios of luciferase expression of tumor/whole body are shown in FIG. 1D, where the LPP solutions prepared by the prescriptions have a higher ratio of luciferase expression of tumor/whole body, which is significantly higher than MC3-LNP, indicating that the LPP solutions prepared by the prescriptions have higher expression efficiency in the tumor and a better tumor targeting effect than the MC3-LNP solution, and have less expression in other tissues or organs except the tumor, and low systemic toxicity.

### 2.3 Effects of Tumor Suppression of Preparations Prepared by Different Prescriptions

This example used the LPP solutions of MC3-LNP, A14, B11, B12, B17, B18, B19, and B23 containing IL-12 mRNA (SEQ ID NO: 2) as prepared in Examples 2.1.1 and 2.1.2 to test the effects of tumor suppression of preparations prepared by different prescriptions. The specific test methods were as follows:

Female C57BL/6 mice (Beijing Vital River Laboratory Animal Technology Co., Ltd.) of 6 weeks of age with a weight of around 20 g were used, and each mouse was injected subcutaneously unilaterally with 1×10⁶ murine-derived cutaneous melanoma cells, B16F10 cells. When tumors grew to a size of about 100 mm³, B16F10 tumor-bearing mice were divided into 8 groups (PBS, A14, B11, B12, B17, B18, B19, and B23 groups, with 8 mice per group), and LPP solutions (50 μL) containing 5 μg of IL-12 mRNA were respectively administered to the mice by intratumoral injection at days 1, 4, 8, and 11. Each group of B16F10 subcutaneous tumor-bearing mice had a tumor volume of about 100 mm³ at day 0 before injection. All the mice were observed for 17 days from day 0, and the body weights and tumor volumes of the mice were recorded and plotted at days 0, 2, 4, 7, 9, 11, 14, and 17. The tumor volume reaching 2000-2500 mm³ was used as an observation endpoint.

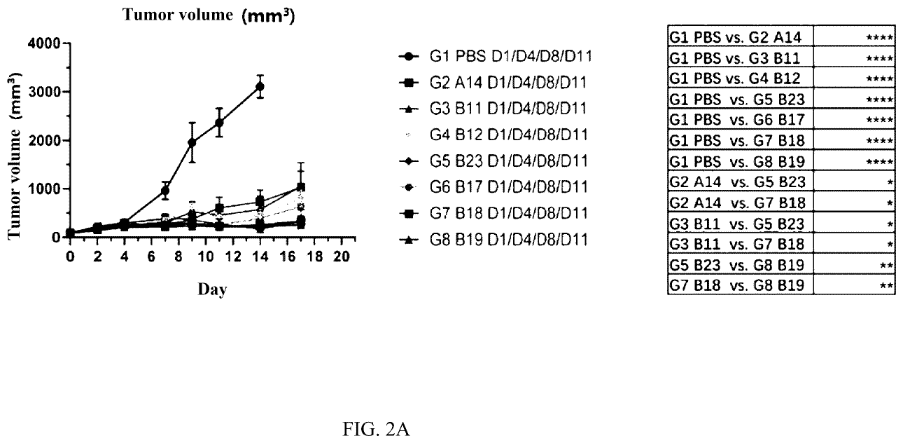

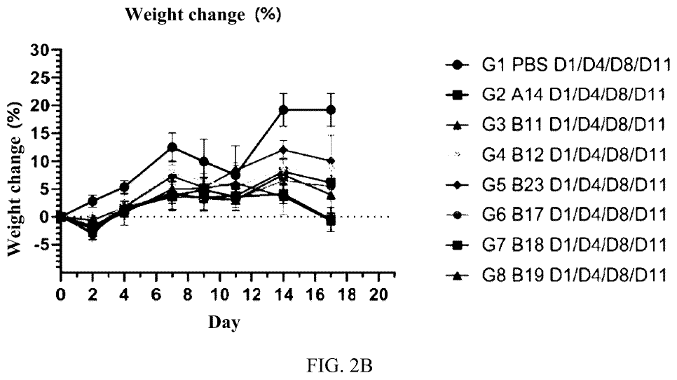

The changes in tumor volume of the mice are shown in FIG. 2A. The mice in the PBS group were euthanized at day 14 due to too large tumor volume. It was observed that at day 14, the preparation groups had a significant decrease in tumor volume compared with the PBS group. The tumor volumes of the mice in the remaining groups did not reach the observation endpoint at day 14, and were observed until day 17. At day 17, A14, B11, and B19 groups had also a significant decrease in tumor volume compared with B18 and B23 groups. The above results show that the preparations prepared by the prescriptions all have a significant effect of tumor suppression. However, the A14, B11 and B19 preparations have a better therapeutic effect than the B18 and B23 preparations, indicating that the change in the proportion of lipids in the lipid compositions can affect the therapeutic effect on tumors.

The changes in body weight of the mice are shown in FIG. 2B, where the mice in each group have no significant change in body weight, indicating that the preparations have no obvious toxicity, and have a small side effect on the mice and high safety.

It is considered that, the above experimental results show that the preparations prepared by other prescriptions have an effect of tumor suppression superior to B18 and B23 preparations. Thus, this example also used another mouse tumor model, A20 tumor-bearing mouse model, to further test the effect of tumor suppression of the preparations except B18 and B23, to investigate the influence of lipid ratio on the effect of tumor suppression. The specific test methods were as follows:

Female Balb/C mice (Beijing Vital River Laboratory Animal Technology Co., Ltd.) of 6 weeks of age with a weight of around 20 g were used, and each mouse was injected subcutaneously unilaterally with 1×10⁶ murine-derived B-cell lymphoma cells, A20 cells. When tumors grew to a size of about 200 mm³, A20 tumor-bearing mice were divided into 6 groups (PBS, A14, B11, B12, B17, and B19 groups, with 9 mice per group), and LPP solutions (50 μL) containing 5 μg of IL-12 mRNA were respectively administered to the mice by intratumoral injection at days 1, 4, 8, and 11. Each group of A20 subcutaneous tumor-bearing mice had a tumor volume of about 200 mm³ at day 0 before injection. All the mice were observed for 25 days from day 0 before injection, and the body weights and tumor volumes of the mice were recorded and plotted at days 0, 3, 5, 7, 9, 11, 15, 18, 22, and 25.

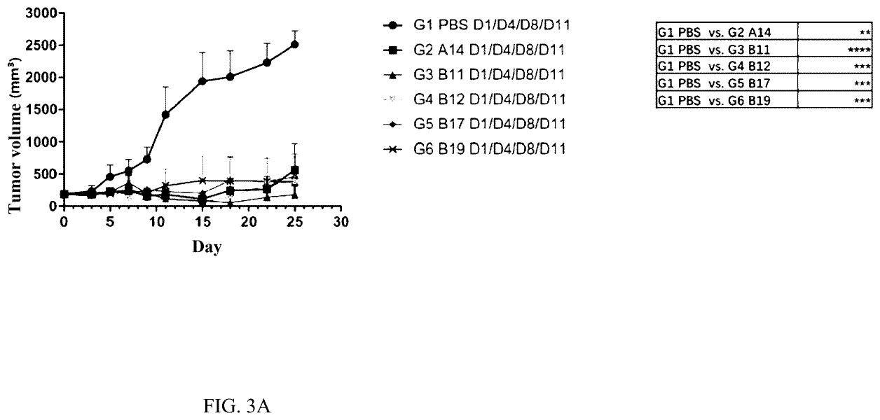

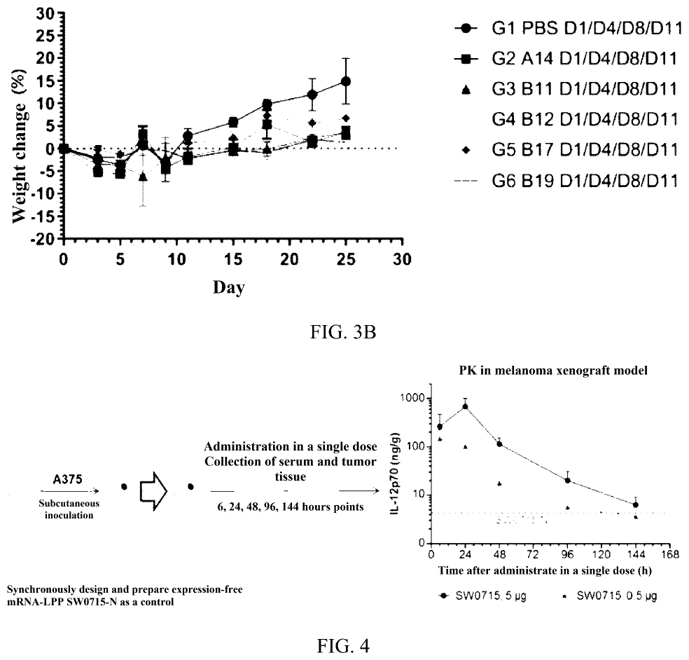

The changes in tumor volume of the mice are shown in FIG. 3A, where the preparation groups have a significant decrease in tumor volume compared with the PBS group, in which the decrease in tumor volume of the mice in the B11 group is most significant, and the preparation groups have no significant difference. The above results show that the preparations prepared by the prescriptions all also have a significant effect of tumor suppression in the A20 tumor-bearing mice. However, the B11 preparation has a better therapeutic effect than the preparations prepared by other preparations, indicating that the change in the proportion of lipids in the lipid compositions can affect the therapeutic effect on tumors. Preferably, the lipid composition includes 40 mol % of M5, 15 mol % of DOPE, 43.5 mol % of the cholesterol, and 1.5 mol % of DMG-PEG.

The changes in body weight of the mice are shown in FIG. 3B, where the A20 tumor-bearing mice in each preparation group have no significant change in body weight, indicating that the preparations have no obvious toxicity, and have a small side effect on the mice and high safety.

### Example 3 Comparison of Preparations Prepared by Different Prescriptions with Cationic Lipids being ALC-0315, SW-II-127, and SW-II-135-1

This example used the preparation methods as described in Examples 2.1.1 and 2.1.2 to prepare LPP and LNP preparations with cationic lipids being ALC-0315, SW-II-127, and SW-II-135-1 respectively as shown in Table 2, so as to test the luciferase signal expression of the LPP and LNP preparations prepared by different prescriptions in the mice. The specific test methods were as follows:

Female C57BL/6 mice (Beijing Vital River Laboratory Animal Technology Co., Ltd.) of 6 weeks of age with a weight of around 20 g were used, and each mouse was injected subcutaneously unilaterally with 1×10⁶ murine-derived cutaneous melanoma cells, B16F10 cells. When tumors grew to a size of about 450 mm³, B16F10 tumor-bearing mice were divided into 6 groups (1-1, 1-2, 1-3, 1-4, 1-5, and 1-6 groups, with 3 mice per group), and LPP and LNP solutions (50 μL) containing 5 μg of luciferase mRNA prepared by the prescriptions were respectively taken and administered to the mice by intratumoral injection. 6 hours after the administration, mice were injected intraperitoneally with a 150 mg/kg D-fluorescein substrate. The bioluminescence was measured in a Xenogen IVIS-200 imaging system 7 minutes after substrate injection to evaluate the expression and distribution of the luciferase mRNA in the mice.

The ratios of luciferase expression of tumor/whole body of the preparations prepared by different prescriptions are shown in Table 3, where the results of all the preparations show that under the same prescription, the ratios of luciferase expression of tumor/whole body of the mice in the LPP groups are higher than the LNP groups, indicating that under the same prescription, although the cationic lipids are different, the LPP preparations can have higher expression efficiency, a better targeting effect, and lower systemic toxicity than the LNP preparations. SW-11-135-1 has the highest ratio of luciferase expression of tumor/whole body, indicating that it has high expression and excellent targeting effect in the tumor and is a preferred cationic lipid.

TABLE 2
Different prescriptions with cationic lipids
being ALC-0315, SW-II-127, and SW-II-135-1

| | Cationic | | | | |
|---|---|---|---|---|---|
| | lipid | Phospholipid | PEG | Cholesterol | |
| 1 - 1 | ALC-0315 | DSPC | PEG-DMG | Cholesterol | LNP |
| 1 - 2 | SW-II-127 | | | | |
| 1 - 3 | SW-II-135-1 | | | | |
| 1 - 4 | ALC-0315 | DSPC | PEG-DMG | Cholesterol | LPP |
| 1 - 5 | SW-II-127 | | | | |
| 1 - 6 | SW-II-135-1 | | | | |
| Mole | 50 | 10 | 1.5 | 38.5 | |
| ratio | | | | | |

TABLE 3
Ratios of luciferase expression of tumor/whole body
of preparations prepared by different prescriptions

| | LNP (tumor/whole body)% | LPP (tumor/whole body)% |
|---|---|---|
| ALC-0315 | 4.9 | 11.2 ± 7.9 |
| SW-II-127 | 15.0 ± 18.5 | 25.7 ± 15.1 |
| SW-II-135-1 | 43.6 ± 34.7 | 58.6 ± 36.4 |

### Example 4 Comparison of Preparations Prepared by Different Prescriptions with a Cationic Lipid being SW-II-127

To further investigate the lipid types of LPP lipid compositions suitable for intratumoral injection, this example used the preparation methods as described in Examples 2.1.1 and 2.1.2 to prepare LPP and LNP preparations with cationic lipids being SW-II-127 and with different phospholipid types and PEG types as shown in Table 4, so as to test the luciferase signal expression of different preparations in the mice. The specific test methods were as follows:

Female C57BL/6 mice (Beijing Vital River Laboratory Animal Technology Co., Ltd.) of 6 weeks of age with a weight of around 20 g were used, and each mouse was injected subcutaneously unilaterally with 1×10⁶ murine-derived cutaneous melanoma cells, B16F10 cells. When tumors grew to a size of about 450 mm³, B16F10 tumor-bearing mice were divided into 8 groups (2-1, 2-2, 2-3, 2-4, 2-5, 2-6, 2-7, and 2-8 groups, with 3 mice per group), and solutions (50 μL) containing 5 μg of luciferase mRNA prepared by the prescriptions were respectively taken and administered to the mice by intratumoral injection. 6 hours after the administration, mice were injected intraperitoneally with a 150 mg/kg D-fluorescein substrate. The bioluminescence was measured in a Xenogen IVIS-200 imaging system 7 minutes after substrate injection to evaluate the expression and distribution of the luciferase mRNA in the mice.

The ratios of luciferase expression of tumor/whole body of preparations prepared by different prescriptions are shown in Table 5, where the mice administrated with the LPP preparations in the 2-5 and 2-7 groups have a higher ratio of luciferase expression of tumor/whole body than the mice administrated with the LNP preparations in the 2-1 and 2-3 groups with the same prescription. The above results show the influence of the PEG types on the expression and targeting effect of the LPP preparations in the tumor. When PEG is PEG-DMG, the LPP preparations have higher expression and superior targeting effect in the tumor than the LNP preparations, in particular, when combined with DOPE, they are superior in expression and targeting effect.

TABLE 4
Different prescriptions with a cationic lipid being SW-II-127

| | Cationic | Cationic | | | | | | |
|---|---|---|---|---|---|---|---|---|
| | lipid | lipid % | Phospholipid | Phospholipid % | PEG | PEG % | Cholesterol % | |
| 2 - 1 | SW-II-127 | 50 | DOPE | 10 | PEG-DMG | 1.5 | 38.5 | LNP |
| 2 - 2 | | | | | PEG-DSPE | 1.5 | 38.5 | |
| 2 - 3 | | | DSPC | 10 | PEG-DMG | 1.5 | 38.5 | |
| 2 - 4 | | | | | PEG-DSPE | 1.5 | 38.5 | |
| 2 - 5 | SW-II-127 | 50 | DOPE | 10 | PEG-DMG | 1.5 | 38.5 | LPP |
| 2 - 6 | | | | | PEG-DSPE | 1.5 | 38.5 | |
| 2 - 7 | | | DSPC | 10 | PEG-DMG | 1.5 | 38.5 | |
| 2 - 8 | | | | | PEG-DSPE | 1.5 | 38.5 | |

TABLE 5
Ratios of luciferase expression of tumor/whole
body of preparations prepared by different prescriptions
with a cationic lipid being SW-II-127

| | LNP | | LPP |
|---|---|---|---|
| | (tumor/whole body)% | | (tumor/whole body)% |
| 2 - 1 | 19.2 ± 14.5 | 2 - 5 | 51.3 ± 36.8 |
| 2 - 2 | 39.6 ± 24.1 | 2 - 6 | 26.5 ± 17.4 |
| 2 - 3 | 15.0 ± 18.5 | 2 - 7 | 25.7 ± 15.1 |
| 2 - 4 | 49.6 ± 15.6 | 2 - 8 | 30.2 ± 10.0 |

### Example 5 Comparison of Preparations Prepared by Different Prescriptions with a Cationic Lipid being SW-II-138-1

To further investigate the lipid ratios of LPP lipid compositions suitable for intratumoral injection, this example used the preparation methods as described in Examples 2.1.1 and 2.1.2 to prepare LPP and LNP preparations with the same lipid type and with different lipid ratios as shown in Table 6, so as to test the luciferase signal expression of different preparations in the mice. The specific test methods were as follows:

Female C57BL/6 mice (Beijing Vital River Laboratory Animal Technology Co., Ltd.) of 6 weeks of age with a weight of around 20 g were used, and each mouse was injected subcutaneously unilaterally with 1×10⁶ murine-derived cutaneous melanoma cells, B16F10 cells. When tumors grew to a size of about 450 mm³, the mice were divided into 8 groups (3-1, 3-2, 3-3, 3-4, 3-5, 3-6, 3-7, 3-8 groups, with 3 mice per group), and solutions (50 μL) containing 5 μg of luciferase mRNA prepared by the prescriptions were respectively taken and administered to the mice by intratumoral injection. 6 hours after the administration, mice were injected intraperitoneally with a 150 mg/kg D-fluorescein substrate. The bioluminescence was measured in a Xenogen IVIS-200 imaging system 7 minutes after substrate injection to evaluate the expression and distribution of the luciferase mRNA in the mice.

The ratios of luciferase expression of tumor/whole body of preparations prepared by different prescriptions are shown in Table 7, where the mice administrated with the LPP preparations in the 3-5 and 3-6 groups have a higher ratio of luciferase expression of tumor/whole body than the mice administrated with the LNP preparations in the 3-1 and 3-2 groups with the same prescription. The above results show the influence of the lipid ratios on the expression and targeting effect of the LPP preparations in the tumor, and under the same conditions, when the mole % of the cationic lipid is less than 50%, the LPP preparations have higher expression and a superior targeting effect in the tumor.

TABLE 6
Different prescriptions with a cationic lipid being SW-II-138-1

| | Cationic | Cationic | | | | | | |
|---|---|---|---|---|---|---|---|---|
| | lipid | lipid % | Phospholipid | Phospholipid % | PEG | PEG % | Cholesterol % | |
| 3 - 1 | SW-II-138-1 | 30 | DOPE | 20 | PEG-DMG | 1.5 | 48.5 | LNP |
| 3 - 2 | | 40 | | 15 | | 1.25 | 43.75 | |
| 3 - 3 | | 50 | | 10 | | 1.5 | 38.5 | |
| 3 - 4 | | 60 | | 10 | | 1 | 29 | |
| 3 - 5 | SW-II-138-1 | 30 | DOPE | 20 | PEG-DMG | 1.5 | 48.5 | LPP |
| 3 - 6 | | 40 | | 15 | | 1.25 | 43.75 | |
| 3 - 7 | | 50 | | 10 | | 1.5 | 38.5 | |
| 3 - 8 | | 60 | | 10 | | 1 | 29 | |

TABLE 7
Ratios of luciferase expression of tumor/whole body
of preparations prepared by different prescriptions
with a cationic lipid being SW-II-138-1

| | LNP | | LPP |
|---|---|---|---|
| | (tumor/whole body)% | | (tumor/whole body)% |
| 3 - 1 | 29.1 ± 27.4 | 3 - 5 | 58.3 ± 33.8 |
| 3 - 2 | 22.4 ± 18.5 | 3 - 6 | 61.1 ± 7.9 |
| 3 - 3 | 47.0 ± 41.9 | 3 - 7 | 42.5 ± 54.0 |
| 3 - 4 | 69.3 ± 20.6 | 3 - 8 | 44.5 ± 27.5 |

### Example 6 Preparation of Human IL-12 mRNA

### 6.1 Design and Synthesis of DNA Template

The applicant designed and synthesized an optimized DNA open reading frame (ORF) sequence encoding IL-12 (p70) (an amino acid sequence of SEQ ID NO: 3). Nucleic acid IL-12 JC encoding IL-12 (p70) is shown in Table 8.

T7 promoter sequence (SEQ ID NO: 14), 5′-UTR sequence (SEQ ID NO: 10), and 3′-UTR sequence (SEQ ID NO: 11) were further designed. Kozak sequence was included in the 5′-UTR sequence (SEQ ID NO: 10).

Then, connection was conducted according to the order of the T7 promoter sequence, the 5′-UTR sequence, the DNA ORF, and the 3′-UTR sequence, and whole gene synthesis was conducted using pUC57 as a vector (Suzhou Genewiz Biotechnology Co., Ltd.), to obtain a plasmid DNA template.

PCR amplification was conducted using a pair of tailing PCR primers (upstream primer (SEQ ID NO: 15): 5′TTGGACCCTCGTACAGAAGCTAATACG3′; and downstream poly(T) long primer (SEQ ID NO: 16): 5′TTTTTTTTTTTTTTTTTTTTTTTTTTTTTTTTTTTTTTTTTTTTTTTTTTTTTTTTTTTTTT TTTTTTTTTTTTTACTTCCTACTCAGGCTTTATTCAAAGACCA 3′) and a high-fidelity DNA polymerase-based PCR amplification kit to obtain a DNA template.

6.2. In Vitro Transcription of mRNA from DNA Template

The PCR product after purification (Takara purification kit) as prepared in Example 6.1 was used as a template to conduct a transcription capping reaction via a T7 RNA polymerase for in vitro transcription of mRNA, thereby producing Cap1mRNA. In in vitro transcription, 1-methyl-pseudouridine-triphosphate was used to replace uridine triphosphate (UTP). Thus, the modification proportion of 1-methyl-pseudouracil in Cap1mRNA for in vitro transcription was 100%. After the transcription was completed, DNaseI (Thermo Fisher Scientific Inc.) was used to digest the DNA template to reduce the risk posed by the residual DNA template.

DynabeadsMyone (Thermo Fisher Scientific Inc.) was used to purify the mRNA. The purified mRNA was dissolved in 1 mM sodium citrate buffer (pH 6.5+/−0.1), sterilely filtered, and cryopreserved at −80° C. until use. The sequence of the obtained mRNA is shown in Table 8.

TABLE 8
Sequences of Nucleic acid IL-12 JC

SEQ ID NO:

| | RNA ORF | DNA ORF | mRNA | DNA |
|---|---|---|---|---|
| Name | sequence | sequence | sequence | sequence |
| IL-12 JC | 4 | 5 | 6 | 7 |

Based on the above method, the applicant also designed and synthesized an expression-free control nucleic acid sequence for base deletion and mutation of an optimized DNA ORF sequence encoding IL-12 (p70), which does not express IL-12 (for a corresponding nucleic acid sequence, see SEQ ID NOs: 18, 19, 20, and 21).

### Examples 7 Preparation of mRNA Vaccine Preparations

Preparation of an aqueous mRNA solution: IL-12JC mRNA as prepared in Example 6.2 was diluted into 0.2 mg/mL mRNA solution with 10 mM citric acid-sodium citrate buffer (pH of 4.0).

Preparation of a lipid solution: cationic lipid (M5):DOPE:cholesterol:mPEG2000-DMG was dissolved in absolute ethyl alcohol in a molar ratio of 40:15:43.5:1.5, to prepare a 10 mg/mL lipid solution.

Preparation of a protamine sulfate solution: protamine sulfate was dissolved in nuclease-free water to prepare a protamine sulfate solution with a working concentration of 0.25 mg/mL.

Preparation of a core nanoparticle solution: by using a microfluidic technology (Micro & Nano (Shanghai) Biologics Co., Ltd., Model: Inano D), the protamine sulfate solution was mixed with the mRNA solution under the following conditions: volume=4.0 mL; flow rate ratio=5 (mRNA): 1 (protamine solution), total flow rate=12 mL/min, start waste=0.35 mL, end waste=0.1 mL, and room temperature, to obtain a core nanoparticle solution formed by protamine and mRNA.

Preparation of LPP: the core nanoparticle solution and the lipid solution were secondarily mixed under the following conditions: volume=4.0 mL, flow rate ratio=1 (lipid solution):3 (core nanoparticle solution), total flow rate=12 mL/min, pre-waste=0.35 mL, post-waste=0.1 mL, and room temperature, and diluted with PBS, to obtain an LPP solution.

Centrifugal ultrafiltration: the LPP solution was subjected to ultrafiltration and centrifugation 2-3 times to remove ethanol (rotation speed of 3,000 rpm, centrifuge time of 30 minutes, and temperature of 4° C.), with an ultrafiltration solution being a 9% sucrose solution, to obtain LPP preparation SW0715 containing IL-12 JC mRNA.

Moreover, by using the same preparation method, LPP preparation SW0715-N containing an expression-free control mRNA sequence was prepared as a control.

### Example 8 Expression of SW0715 in A375 Tumor-Bearing Mice

This example used LPP preparation SW0715 containing IL-12 JC mRNA as prepared in Example 7 to test its expression in vivo. The specific test methods were as follows:

Female BALB/c nude mice (Shanghai Lingchang Biotechnology Co., Ltd.) of 6-8 weeks of age with a weight of around 18-22 μg were used, and each mouse was injected subcutaneously unilaterally with human malignant melanoma cells, A375 cells (the Cell Bank of the Chinese Academy of Sciences). When tumors grew to a size of about 100-150 mm³, A375 tumor-bearing mice were divided into 3 groups (SW0715 5 μg group, SW0715 0.5 μg group, and SW0715-N group, with 15 mice in each of the SW0715 5 μg group and the SW0715 0.5 μg group and 3 mice in the SW0715-N group), and 50 μL of SW0715 containing 5 μg of IL-12 JC mRNA, 50 μL of SW0715 containing 0.5 g of IL-12 JC mRNA, and 50 μL of SW0715-N containing 5 μg of expression-free control mRNA were administrated to the mice in a single dose by intratumoral injection, respectively. The serum and tumor tissue of the mice were collected 6, 24, 48, 96 and 144 hours after the administration, respectively. The content of serum IL-12 (p70) was tested by a human IL-12 (p70) ELISA kit (Human IL12 p70 DuoSet ELISA KIT, Cat. No.: DY1270-05, Supplier: R&D Systems), so as to test the in vivo expression of SW0715 (the immunization procedure is shown in FIG. 4).

The operation steps for ELISA (enzyme-linked immunosorbent assay) of IL-12 (p70) were as follows: briefly, a 96-well plate (100 mL/well) was coated overnight with human IL-12 p70 capture antibody diluted with PBS according to the manufacturer's instructions. The next day, the liquids in the wells were sucked and discarded, and the plate was washed 3 times. The well plate was blocked with a diluent (1% BSA in PBS) for 1 hour (300 mL/well). After blocking, the liquids in the wells were sucked and discarded, and the plate was washed 3 times. A diluent solution (100 mL/well) was added to a blank hole. The samples or the standard substances of different concentrations were added to the remaining corresponding holes (100 mL/well). After uniform mixing, a plate sealing film was put on. Incubation was carried out at room temperature for 2 hours. After 2 hours, the liquids in the wells were sucked and discarded, and the plate was washed 3 times. Subsequently, a detection antibody was added to each well (100 mL/well). After uniform mixing, a plate sealing film was put on. Incubation was carried out at room temperature for 2 hours. After 2 hours, the liquids in the wells were sucked and discarded, and the plate was washed 3 times. Then, a Streptavidin-HRP working solution was added to each well (100 mL/well). After uniform mixing, the plate was incubated at room temperature for 20 minutes. After 20 minutes, the liquids in the wells were sucked and discarded, and the plate was washed 3 times. Finally, a chromogenic substrate (TMB) was added with 100 mL/well, the plate was incubated for 20 minutes at room temperature in dark, then a reaction stop solution was added with 50 mL/well, and the OD450 value was measured immediately after uniform mixing. The content of IL-12 (p70) in each sample was calculated according to the standard curve.

The experimental results are shown in FIG. 4, where IL-12 JC mRNA contained in SW0715 is highly expressed in vivo and the expressed IL-12 (p70) has a longer half-life; the content of IL-12 (p70) expressed in the serum of the mice in the SW0715 5 g group is still much higher than the negative control group (SW0715-N) 144 hours after the administration.

### Example 9 In Vitro Expression of SW0715

To evaluate the in vitro expression of SW0715, the applicant transfected different amounts of SW0715 into cells and tested the level of human IL-12 (p70) in the cells. The specific test methods were as follows.

A375 cells and human breast carcinoma cells MDA-MB-231 (the Cell Bank of the Chinese Academy of Sciences) were inoculated in a 96-well plate with 6×10′ cells/well. 18 hours after cell inoculation, LPP preparation SW0715 containing 2.5 g of IL-12 JC mRNA and SW0715-N containing 2.5 g of expression-free control mRNA were added into A375 cells and MDA-MB-231, respectively. The transfected cells were placed in a cell incubator to continue to be incubated with 5% CO₂ at 37° C. for 24 hours. Then, the cell supernatant was collected to test the level of human IL-12 (p70) in the cells using a human IL-12 (p70) ELISA kit (ELISA Human IL12 p70 DuoSet ELISA KIT, Cat. No.: DY1270-05, Supplier: R&D Systems) according to the manufacturer's instructions. For the specific operation methods, see Example 8.

The test results are shown in FIG. 5, where whether in A375 cells or MDA-MB-231 cells, the level of human IL-12 (p70) expressed by SW0715 is progressively increased with increasing amounts, indicating that the in vitro expression of SW0715 shows dose-dependent. The ELISA results for SW0715-N are not shown in the figure due to being below the lower limit of quantification.

### Example 10 Cellular Immune Response Induced by SW0715

This example used LPP preparation SW0715 containing IL-12 JC mRNA as prepared in Example 7 to test the cellular immune response induced thereby, in particular, to test whether the expression product of SW0715 could activate CD8⁺T cells. The specific test methods were as follows:

A375 cells were inoculated in a 96-well plate with 6×10′ cells/well. 18 hours after cell inoculation, LPP preparation SW0715 containing 2.5 g of IL-12 JC mRNA and SW0715-N containing 2.5 g of expression-free control mRNA were added into A375 cells, respectively. The transfected cells were placed in a cell incubator to continue to be incubated with 5% CO₂ at 37° C. for 48 hours. The cell supernatant was collected for test.

Human peripheral blood mononuclear cells (PBMCs) were sorted and isolated from the CD8⁺T cells by magnetic beads (CD8 MicroBeads, human, Miltenyi Biotec, Cat. No.: 130-045-201). Based on baseline stimulation (1 g/mL phytohemagglutinin-L (PHA-L) stimulation, Phytohemagglutinin-L Solution. Invitrogen, Cat. No.: 00-4977-03), the CD8⁺T cells were stimulated (stimulation concentration of 1 g/mL) with the supernatant of the cells treated with SW0715 or the supernatant of the cells treated with SW0715-N described above (as a control). After treatment, incubation was carried out for 48 hours. The cell supernatant was collected to test the level of IFN-γ in the cells using a human IFN-γ ELISA kit (Human IFN-γ Precoated ELISA kit, DAKEWE, Cat. No.: 1110002) according to the manufacturer's instructions. In short, plate strips required for the test were removed from a sealed bag equilibrated to room temperature, diluted buffer R (100 mL/well) was added to a blank well, and the samples or the standard substances of different concentrations were added to the remaining corresponding wells (100 mL/well). Subsequently, a biotinylated antibody working solution was added to each well (50 mL/well). After uniform mixing, a plate sealing film was put on. Incubation was carried out at room temperature for 2 hours. After 2 hours, the plate was washed 3 times. Then, a Streptavidin-HRP working solution was added to each well (100 mL/well). After uniform mixing, a plate sealing film was put on. The plate was incubated at room temperature for 20 minutes. The plate was washed 3 times. Finally, a chromogenic substrate (TMB) was added with 100 mL/well, the plate was incubated for 15 minutes at room temperature in dark, then a reaction stop solution was added with 100 mL/well, and the OD450 value was measured immediately after uniform mixing. The content of IFN-γ in each sample was calculated according to the standard curve. The negative control was the cell supernatant treated with SW0715-N, and the positive control was recombinant human IL-12 protein (rhIL12, synthesized by Sino Biological, with an amino acid sequence shown in SEQ ID NO: 17).

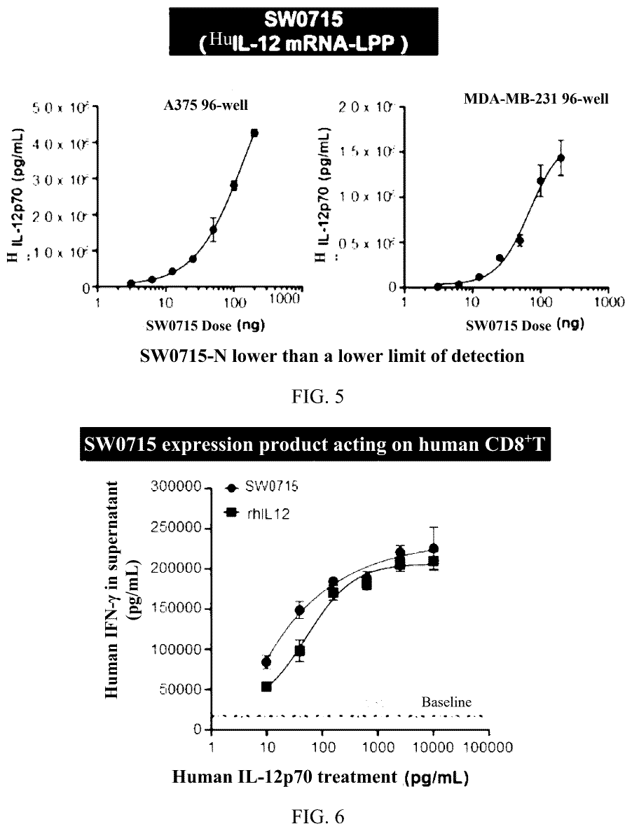

The experimental results are shown in FIG. 6, where SW0715 can induce a cellular immune response, and the expression product thereof can effectively activate primary CD8⁺T cells in vitro.

### Example 11 Effect of Tumor Suppression of SW0715

This example used LPP preparation SW0715 containing IL-12 JC mRNA as prepared in Example 7 to test the effect of tumor suppression thereof. The specific test methods were as follows:

Female NCG immunodeficient mice (Jiangsu GemPharmatech Co., Ltd) of 6-8 weeks of age with a weight of around 18-22 g were used, and each mouse was injected subcutaneously unilaterally with 5×10⁶ human breast carcinoma cells, MDA-MB-231 cells; and 5 days after inoculation of MDA-MB-231 tumor cells, the mice were injected with human PBMCs at the tail vein (5×10⁶ cells/mouse), so as to obtain an NCG mouse model for reconstitution of the human immune system. When tumors grew to a size of about 80-120 mm³, MDA-MB-231 tumor-bearing mice were divided into 4 groups (SW0715 0.4 g group, SW0715 2.0 g group, SW0715 10.0 g group, and SW0715-N 10.0 g group, with 8 mice per group), and 50 L of SW0715 containing 0.4, 2.0, and 10.0 g of IL-12 mRNA and 50 L of SW0715-N containing 10.0 g of expression-free control mRNA were respectively administered to the mice by intratumoral injection at days 0, 7, 14, and 21. All the mice were observed for 27 days from day 0. The length and width diameters of tumors were measured twice a week using a vernier caliper. The formula for calculating the volume of tumors was V=0.5×a×b², where a and b represented the length and width diameters of tumors, respectively. All data were analyzed by Graphpad, and represented as mean±standard error of mean (Mean±SEM). The differences between the test groups and the control group were compared using One-way ANOVA LSD(L)test in Graphpad, and if p<0.05, it was considered to have a significant difference.

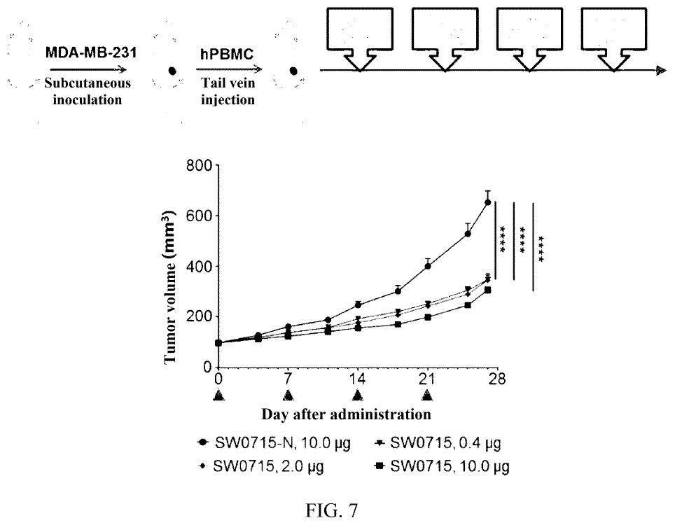

The changes in tumor volume of the mice are shown in FIG. 7, where 27 days after the administration, the mean tumor volume of the SW0715-N control group is 652.63±45.31 mm³; and the mean tumor volumes of the mice in the SW0715 0.4 g group, the SW0715 2.0 g group and the SW0715 10.0 g group were 347.61±19.12 mm³, 345.74±28.81 mm³, and 307.42±12.70 mm³, respectively. It can be seen from this that the SW0715 0.4 g group, the SW0715 2.0 g group and the SW0715 10.0 g group have a significant decrease in the tumor volume of the mice compared with the SW0715-N 10.0 g group; and the SW0715 0.4 g group, the SW0715 2.0 g group and the SW0715 10.0 g group have a significant slowdown in the growth trend of the tumor volume of the mice compared with the SW0715-N 10.0 g group. SW0715 with the lowest dose of 0.4 g can achieve a significant effect of tumor suppression, and SW0715 with the highest dose of 10.0 g has the best effect of tumor suppression.

Although the present disclosure has been disclosed above by preferred examples, it is not intended to limit the present disclosure. Anyone familiar with the art can make various changes and modifications without departing from the spirit and scope of the present disclosure. Therefore, the scope of protection of the present disclosure shall be subject to the scope of protection claimed by the claims.

### SEQUENCE LISTING

Sequence Listing

| SEQ | | |
|---|---|---|
| ID | | |
| NO: | Description | Sequence |
| 1 | Fluc | AUGGAGGACGCCAAGAACAUCAAGAAGGGCCCCGCCCCCUUCUACCCCCUGGAGGAC |
| | encoding | GGCACCGCCGGCGAGCAGCUGCACAAGGCCAUGAAGCGGUACGCCCUGGUGCCCGG |
| | sequence | CACCAUCGCCUUCACCGACGCCCACAUCGAGGUGGACAUCACCUACGCCGAGUACUU |
| | | CGAGAUGAGCGUGCGGCUGGCCGAGGCCAUGAAGCGGUACGGCCUGAACACCAACC |
| | | ACCGGAUCGUGGUGUGCAGCGAGAACAGCCUGCAGUUCUUCAUGCCCGUGCUGGG |
| | | CGCCCUGUUCAUCGGCGUGGCCGUGGCCCCCGCCAACGACAUCUACAACGAGCGGG |
| | | AGCUGCUGAACAGCAUGGGCAUCAGCCAGCCCACCGUGGUGUUCGUGAGCAAGAAG |
| | | GGCCUGCAGAAGAUCCUGAACGUGCAGAAGAAGCUGCCCAUCAUCCAGAAGAUCAU |
| | | CAUCAUGGACAGCAAGACCGACUACCAGGGCUUCCAGAGCAUGUACACCUUCGUGA |
| | | CCAGCCACCUGCCCCCCGGCUUCAACGAGUACGACUUCGUGCCCGAGAGCUUCGACC |
| | | GGGACAAGACCAUCGCCCUGAUCAUGAACAGCAGCGGCAGCACCGGCCUGCCCAAGG |
| | | GCGUGGCCCUGCCCCACCGGACCGCCUGCGUGCGGUUCAGCCACGCCCGGGACCCCA |
| | | UCUUCGGCAACCAGAUCAUCCCCGACACCGCCAUCCUGAGCGUGGUGCCCUUCCACC |
| | | ACGGCUUCGGCAUGUUCACCACCCUGGGCUACCUGAUCUGCGGCUUCCGGGUGGU |
| | | GCUGAUGUACCGGUUCGAGGAGGAGCUGUUCCUGCGGAGCCUGCAGGACUACAAG |
| | | AUCCAGAGCGCCCUGCUGGUGCCCACCCUGUUCAGCUUCUUCGCCAAGAGCACCCU |
| | | GAUCGACAAGUACGACCUGAGCAACCUGCACGAGAUCGCCAGCGGCGGCGCCCCCCU |
| | | GAGCAAGGAGGUGGGCGAGGCCGUGGCCAAGCGGUUCCACCUGCCCGGCAUCCGGC |
| | | AGGGCUACGGCCIUGACCGAGACCACCAGCGCCAUCCUGAUCACCCCCGAGGGCGACG |
| | | ACAAGCCCGGCGCCGUGGGCAAGGUGGUGCCCUUCUUCGAGGCCAAGGUGGUGGA |
| | | CCUGGACACCGGCAAGACCCUGGGCGUGAACCAGCGGGGCGAGCUGUGCGUGGGG |
| | | GCCCCAUGAUCAUGAGCGGCUACGUGAACAACCCCGAGGCCACCAACGCCCUGAUCG |
| | | ACAAGGACGGCUGGCUGCACAGCGGCGACAUCGCCUACUGGGACGAGGACGAGCAC |
| | | UUCUUCAUCGUGGACCGGCUGAAGAGCCUGAUCAAGUACAAGGGCUJACCAGGUGG |
| | | CCCCCGCCGAGCUGGAGAGCAUCCUGCUGCAGCACCCCAACAUCUUCGACGCCGGCG |
| | | UGGCCGGCCUGCCCGACGACGACGCCGGCGAGCUGCCCGCCGCCGUGGUGGUGCUG |
| | | GAGCACGGCAAGACCAUGACCGAGAAGGAGAUCGUGGACUACGUGGCCAGCCAGGU |
| | | GACCACCGCCAAGAAGCUGGGGGGGGCGUGGUGUUCGUGGACGAGGUGCCCAAG |
| | | GGCCUGACCGGCAAGCUGGACGCCCGGAAGAUCCGGGAGAUCCUGAUCAAGGCCAA |
| | | GAAGGGCGGCAAGAUCGCCGUGUGA |
| 2 | IL-12 | AUGUGUCCUCAGAAGCUAACCAUCUCCUGGUUUGCCAUCGUUUUGCUGGUGUCUC |
| | encoding | CACUCAUGGCCAUGUGGGAGCUGGAGAAAGACGUUUAUGUUGUAGAGGUGGACU |
| | sequence | GGACUCCCGAUGCCCCUGGAGAAACAGUGAACCUCACCUGUGACACGCCUGAAGAA |
| | | GAUGACAUCACCUGGACCUCAGACCAGAGACAUGGAGUCAUAGGCUCUGGAAAGAC |
| | | CCUGACCAUCACUGUCAAAGAGUUUCUAGAUGCUGGCCAGUACACCUGCCACAAAG |
| | | GAGGCGAGACUCUGAGCCACUCACAUCUGCUGCUCCACAAGAAGGAAAAUGGAAUU |
| | | UGGUCCACUGAAAUUUUAAAAAAUUUCAAAAACAAGACUUUCCUGAAGUGUGAAG |
| | | CACCAAAUUACUCCGGACGGUUCACGUGCUCAUGGCUGGUGCAAAGAAACAUGGAC |
| | | UUGAAGUUCAACAUCAAGAGCAGUAGCAGUUCCCCUGACUCUCGGGCAGUGACAU |
| | | GUGGAAUGGCGUCUCUGUCUGCAGAGAAGGUCACACUGGACCAAAGGGACUAUGA |
| | | GAAGUAUUCAGUGUCCUGCCAGGAGGAUGUCACCUGCCCAACUGCCGAGGAGACCC |
| | | UGCCCAUUGAACUGGCGUUGGAAGCACGGCAGCAGAAUAAAUAUGAGAACUACAGC |
| | | ACCAGCUUCUUCAUCAGGGACAUCAUCAAACCAGACCCGCCCAAGAACUUGCAGAUG |
| | | AAGCCUUUGAAGAACUCACAGGUGGAGGUCAGCUGGGAGUACCCUGACUCCUGGA |
| | | GCACUCCCCAUUCCUACUUCUCCCUCAAGUUCUUUGUUCGAAUCCAGCGCAAGAAA |
| | | GAAAAGAUGAAGGAGACAGAGGAGGGGUGUAACCAGAAAGGUGCGUUCCUCGUAG |
| | | AGAAGACAUCUACCGAAGUCCAAUGCAAAGGCGGGAAUGUCUGCGUGCAAGCUCAG |
| | | GAUCGCUAUUACAAUUCCUCAUGCAGCAAGUGGGCAUGUGUUCCCUGCAGGGUCC |
| | | GAUCCGGUGGCGGUGGCUCGGGCGGUGGUGGGUCGGGUGGCGGCGGAUCUAGGG |
| | | UCAUUCCAGUCUCUGGACCUGCCAGGUGUCUUAGCCAGUCCCGAAACCUGCUGAAG |
| | | ACCACAGAUGACAUGGUGAAGACGGCCAGAGAAAAACUGAAACAUUAUUCCUGCAC |
| | | UGCUGAAGACAUCGAUCAUGAAGACAUCACACGGGACCAAACCAGCACAUUGAAGA |
| | | CCUGUUUACCACUGGAACUACACAAGAACGAGAGUUGCCUGGCUACUAGAGAGACU |
| | | UCUUCCACAACAAGAGGGAGCUGCCUGCCCCCACAGAAGACGUCUUUGAUGAUGAC |
| | | CCUGUGCCUUGGUAGCAUCUAUGAGGACUUGAAGAUGUACCAGACAGAGUUCCAG |
| | | GCCAUCAACGCAGCACUUCAGAAUCACAACCAUCAGCAGAUCAUUCUAGACAAGGGC |
| | | AUGCUGGUGGCCAUCGAUGAGCUGAUGCAGUCUCUGAAUCAUAAUGGCGAGACUC |
| | | UGCGCCAGAAACCUCCUGUGGGAGAAGCAGACCCUIJACAGAGUGAAAAUGAAGCUC |
| | | UGCAUCCUGCUUCACGCCUUCAGCACCCGCGUCGUGACCAUCAACAGGGUGAUGGG |
| | | CUAUCUGAGCUCCGCCUAA |
| 3 | Amino acid | MCHQQLVISWFSLVFLASPLVAIWELKKDVYVVELDWYPDAPGEMVVLTCDTPEEDGITW |
| | sequence of | TLDQSSEVLGSGKTLTIQVKEFGDAGQYTCHKGGEVLSHSLLLLHKKEDGIWSTDILKDQKEP |
| | IL-12 | KNKTFLRCEAKNYSGRFTCWWLTTISTDLTFSVKSSRGSSDPQGVTCGAATLSAERVRGDNK |
| | | EYEYSVECQEDSACPAAEESLPIEVMVDAVHKLKYENYTSSFFIRDIIKPDPPKNLQLKPLKNSR |
| | | QVEVSWEYPDTWSTPHSYFSLTFCVQVQGKSKREKKDRVFTDKTSATVICRKNASISVRAQD |
| | | RYYSSSWSEWASVPCSGGGGGGSRNLPVATPDPGMFPCLHHSQNLLRAVSNMLQKARQT |
| | | LEFYPCTSEEIDHEDITKDKTSTVEACLPLELTKNESCLNSRETSFITNGSCLASRKTSFMMALC |
| | | LSSIYEDLKMYQVEFKTMNAKLLMDPKRQIFLDQNMLAVIDELMQALNENSETVPQKSSLE |
| | | EPDFYKTKIKLCILLHAFRIRAVTIDRVMSYLNAS |
| 4 | IL-12 RNA | AUGUGCCACCAGCAGCUGGUGAUCAGCUGGUUCAGCCUGGUGUUCCUGGCCAGCCC |
| | ORF | CCUGGUGGCCAUCUGGGAGCUGAAGAAGGACGUGUACGUGGUGGAGCUGGACUG |
| | | GUACCCCGACGCCCCCGGCGAGAUGGUGGUGCUGACCUGCGACACCCCCGAGGAGG |
| | | ACGGCAUCACCUGGACCCUGGACCAGAGCAGCGAGGUGCUGGGCAGCGGCAAGACC |
| | | CUGACCAUCCAGGUGAAGGAGUUCGGCGACGCCGGCCAGUACACCUGCCACAAGGG |
| | | CGGCGAGGUGCUGAGCCACAGCCUGCUGCUGCUGCACAAGAAGGAGGACGGCAUCU |
| | | GGAGCACCGACAUCCUGAAGGACCAGAAGGAGCCCAAGAACAAGACCUUCCUGCGC |
| | | UGCGAGGCCAAGAACUACAGCGGCCGCUUCACCUGCUGGUGGCUGACCACCAUCAG |
| | | CACCGACCUGACCUUCAGCGUCAAGAGCAGCCGCGGCAGCAGCGACCCCCAGGGCGU |
| | | GACCUGCGGCGCCGCCACCCUGAGCGCCGAGCGCGUGCGCGGCGACAACAAGGAGU |
| | | ACGAGUACAGCGUGGAGUGCCAGGAGGACAGCGCCUGCCCCGCCGCCGAGGAGAGC |
| | | CUGCCCAUCGAGGUGAUGGUGGACGCCGUGCACAAGCUGAAGUACGAGAACUACAC |
| | | CAGCAGCUUCUUCAUCCGCGACAUCAUCAAGCCCGACCCCCCCAAGAACCUGCAGCU |
| | | GAAGCCCCUGAAGAACAGCCGCCAGGUGGAGGUGAGCUGGGAGUACCCCGACACCU |
| | | GGAGCACCCCCCACAGCUACUUCAGCCUGACCUUCUGCGUGCAGGUGCAGGGCAAG |
| | | AGCAAGCGCGAGAAGAAGGACCGCGUGUUCACCGACAAGACCAGCGCCACCGUGAU |
| | | CUGCCGCAAGAACGCCAGCAUCAGCGUGCGCGCCCAGGACCGCUACUACAGCAGCAG |
| | | CUGGAGCGAGUGGGCCAGCGUGCCCUGCAGCGGCGGCGGCGGCGGCGGCAGCCGCA |
| | | ACCUGCCCGUGGCCACCCCCGACCCCGGCAUGUUCCCCUGCCUGCACCACAGCCAGA |
| | | ACCUGCUGCGCGCCGUGAGCAACAUGCUGCAGAAGGCCCGCCAGACCCUGGAGUUC |
| | | UACCCCUGCACCAGCGAGGAGAUCGACCACGAGGACAUCACCAAGGACAAGACCAGC |
| | | ACCGUGGAGGCCUGCCUGCCCCUGGAGCUGACCAAGAACGAGAGCUGCCUGAACAG |
| | | CCGCGAGACCAGCUUCAUCACCAACGGCAGCUGCCUGGCCAGCCGCAAGACCAGCUU |
| | | CAUGAUGGCCCUGUGCCUGAGCAGCAUCUACGAGGACCUGAAGAUGUACCAGGUG |
| | | GAGUUCAAGACCAUGAACGCCAAGCUGCUGAUGGACCCCAAGCGCCAGAUCUUCCU |
| | | GGACCAGAACAUGCUGGCCGUGAUCGACGAGCUGAUGCAGGCCCUGAACUUCAACA |
| | | GCGAGACCGUGCCCCAaAAGAGCAGCCUGGAGGAGCCCGACUUCUACAAGACCAAGA |
| | | UCAAGCUGUGCAUCCUGCUGCACGCCUUCCGCAUCCGCGCCGUGACCAUCGACCGC |
| | | GUGAUGAGCUACCUGAACGCCAGCUAAUAG |
| 5 | IL-12 DNA | ATGTGCCACCAGCAGCTGGTGATCAGCTGGTTCAGCCTGGTGTTCCTGGCCAGCCCCCT |
| | ORF | GGTGGCCATCTGGGAGCTGAAGAAGGACGTGTACGTGGTGGAGCTGGACTGGTACCCC |
| | | GACGCCCCCGGCGAGATGGTGGTGCTGACCTGCGACACCCCCGAGGAGGACGGCATCA |
| | | CCTGGACCCTGGACCAGAGCAGCGAGGTGCTGGGCAGCGGCAAGACCCTGACCATCCA |
| | | GGTGAAGGAGTTCGGCGACGCCGGCCAGTACACCTGCCACAAGGGCGGCGAGGTGCT |
| | | GAGCCACAGCCTGCTGCTGCTGCACAAGAAGGAGGACGGCATCTGGAGCACCGACATC |
| | | CTGAAGGACCAGAAGGAGCCCAAGAACAAGACCTTCCTGCGCTGCGAGGCCAAGAACT |
| | | ACAGCGGCCGCTTCACCTGCTGGTGGCTGACCACCATCAGCACCGACCTGACCTTCAGC |
| | | GTcAAGAGCAGCCGCGGCAGCAGCGACCCCCAGGGCGTGACCTGCGGCGCCGCCACCC |
| | | TGAGCGCCGAGCGCGTGCGCGGCGACAACAAGGAGTACGAGTACAGCGTGGAGTGCC |
| | | AGGAGGACAGCGCCTGCCCCGCCGCCGAGGAGAGCCTGCCCATCGAGGTGATGGTGG |
| | | ACGCCGTGCACAAGCTGAAGTACGAGAACTACACCAGCAGCTTCTTCATCCGCGACATC |
| | | ATCAAGCCCGACCCCCCCAAGAACCTGCAGCTGAAGCCCCTGAAGAACAGCCGCCAGGT |
| | | GGAGGTGAGCTGGGAGTACCCCGACACCTGGAGCACCCCCCACAGCTACTTCAGCCTG |
| | | ACCTTCTGCGTGCAGGTGCAGGGCAAGAGCAAGCGCGAGAAGAAGGACCGCGTGTTCA |
| | | CCGACAAGACCAGCGCCACCGTGATCTGCCGCAAGAACGCCAGCATCAGCGTGCGCGC |
| | | CCAGGACCGCTACTACAGCAGCAGCTGGAGCGAGTGGGCCAGCGTGCCCTGCAGCGGC |
| | | GGCGGCGGCGGCGGCAGCCGCAACCTGCCCGTGGCCACCCCCGACCCCGGCATGTTCC |
| | | CCTGCCTGCACCACAGCCAGAACCTGCTGCGCGCCGTGAGCAACATGCTGCAGAAGGCC |
| | | CGCCAGACCCTGGAGTTCTACCCCTGCACCAGCGAGGAGATCGACCACGAGGACATCAC |
| | | CAAGGACAAGACCAGCACCGTGGAGGCCTGCCTGCCCCTGGAGCTGACCAAGAACGAG |
| | | AGCTGCCTGAACAGCCGCGAGACCAGCTTCATCACCAACGGCAGCTGCCTGGCCAGCCG |
| | | CAAGACCAGCTTCATGATGGCCCTGTGCCTGAGCAGCATCTACGAGGACCTGAAGATGT |
| | | ACCAGGTGGAGTTCAAGACCATGAACGCCAAGCTGCTGATGGACCCCAAGCGCCAGAT |
| | | CTTCCTGGACCAGAACATGCTGGCCGTGATCGACGAGCTGATGCAGGCCCTGAACTTCA |
| | | ACAGCGAGACCGTGCCCCAaAAGAGCAGCCTGGAGGAGCCCGACTTCTACAAGACCAA |
| | | GATCAAGCTGTGCATCCTGCTGCACGCCTTCCGCATCCGCGCCGTGACCATCGACCGCG |
| | | TGATGAGCTACCTGAACGCCAGCTAATAG |
| 6 | IL-12 | AGGAAAUUCCAUUUGGCUGCAGCUUCUGGAGGGAGCCGACAGGAGACGUGGGGAG |
| | mRNA | ACGGCCACCAUGUGCCACCAGCAGCUGGUGAUCAGCUGGUUCAGCCUGGUGUUCCU |
| | | GGCCAGCCCCCUGGUGGCCAUCUGGGAGCUGAAGAAGGACGUGUACGUGGUGGAG |
| | | CUGGACUGGUACCCCGACGCCCCCGGCGAGAUGGUGGUGCUGACCUGCGACACCCC |
| | | CGAGGAGGACGGCAUCACCUGGACCCUGGACCAGAGCAGCGAGGUGCUGGGCAGCG |
| | | GCAAGACCCUGACCAUCCAGGUGAAGGAGUUCGGCGACGCCGGCCAGUACACCUGC |
| | | CACAAGGGCGGCGAGGUGCUGAGCCACAGCCUGCUGCUGCUGCACAAGAAGGAGGA |
| | | CGGCAUCUGGAGCACCGACAUCCUGAAGGACCAGAAGGAGCCCAAGAACAAGACCU |
| | | UCCUGCGCUGCGAGGCCAAGAACUACAGCGGCCGCUUCACCUGCUGGUGGCUGACC |
| | | ACCAUCAGCACCGACCUGACCUUCAGCGUCAAGAGCAGCCGCGGCAGCAGCGACCCC |
| | | CAGGGCGUGACCUGCGGCGCCGCCACCCUGAGCGCCGAGCGCGUGCGCGGCGACAA |
| | | CAAGGAGUACGAGUACAGCGUGGAGUGCCAGGAGGACAGCGCCUGCCCCGCCGCCG |
| | | AGGAGAGCCUGCCCAUCGAGGUGAUGGUGGACGCCGUGCACAAGCUGAAGUACGA |
| | | GAACUACACCAGCAGCUUCUUCAUCCGCGACAUCAUCAAGCCCGACCCCCCCAAGAA |
| | | CCUGCAGCUGAAGCCCCUGAAGAACAGCCGCCAGGUGGAGGUGAGCUGGGAGUACC |
| | | CCGACACCUGGAGCACCCCCCACAGCUACUUCAGCCUGACCUUCUGCGUGCAGGUGC |
| | | AGGGCAAGAGCAAGCGCGAGAAGAAGGACCGCGUGUUCACCGACAAGACCAGCGCC |
| | | ACCGUGAUCUGCCGCAAGAACGCCAGCAUCAGCGUGCGCGCCCAGGACCGCUACUAC |
| | | AGCAGCAGCUGGAGCGAGUGGGCCAGCGUGCCCUGCAGCGGCGGCGGCGGCGGCG |
| | | GCAGCCGCAACCUGCCCGUGGCCACCCCCGACCCCGGCAUGUUCCCCUGCCUGCACC |
| | | ACAGCCAGAACCUGCUGCGCGCCGUGAGCAACAUGCUGCAGAAGGCCCGCCAGACCC |
| | | UGGAGUUCUACCCCUGCACCAGCGAGGAGAUCGACCACGAGGACAUCACCAAGGAC |
| | | AAGACCAGCACCGUGGAGGCCUGCCUGCCCCUGGAGCUGACCAAGAACGAGAGCUG |
| | | CCUGAACAGCCGCGAGACCAGCUUCAUCACCAACGGCAGCUGCCUGGCCAGCCGCAA |
| | | GACCAGCUUCAUGAUGGCCCUGUGCCUGAGCAGCAUCUACGAGGACCUGAAGAUG |
| | | UACCAGGUGGAGUUCAAGACCAUGAACGCCAAGCUGCUGAUGGACCCCAAGCGCCA |
| | | GAUCUUCCUGGACCAGAACAUGCUGGCCGUGAUCGACGAGCUGAUGCAGGCCCUGA |
| | | ACUUCAACAGCGAGACCGUGCCCCAaAAGAGCAGCCUGGAGGAGCCCGACUUCUACA |
| | | AGACCAAGAUCAAGCUGUGCAUCCUGCUGCACGCCUUCCGCAUCCGCGCCGUGACCA |
| | | UCGACCGCGUGAUGAGCUACCUGAACGCCAGCUAAUAGGCUGCCUUCUGCGGGGCU |
| | | UGCCUUCUGGCCAUGCCCUUCUUCUCUCCCUUGCACCUGUACCUCUUGGUCUUUGA |
| | | AUAAAGCCUGAGUAGGAAGUAAAAAAAAAAAAAAAAAAAAAAAAAAAAAAAAAAAA |
| | | AAAAAAAAAAAAAAAAAAAAAAAAAAAAAAAAAAAAAAA |
| 7 | IL-12 DNA | AGGAAATTCCATTTGGCTGCAGCTTCTGGAGGGAGCCGACAGGAGACGTGGGGAGAC |
| | | GGCCACCATGTGCCACCAGCAGCTGGTGATCAGCTGGTTCAGCCTGGTGTTCCTGGCCA |
| | | GCCCCCTGGTGGCCATCTGGGAGCTGAAGAAGGACGTGTACGTGGTGGAGCTGGACTG |
| | | GTACCCCGACGCCCCCGGCGAGATGGTGGTGCTGACCTGCGACACCCCCGAGGAGGAC |
| | | GGCATCACCTGGACCCTGGACCAGAGCAGCGAGGTGCTGGGCAGCGGCAAGACCCTGA |
| | | CCATCCAGGTGAAGGAGTTCGGCGACGCCGGCCAGTACACCTGCCACAAGGGCGGCGA |
| | | GGTGCTGAGCCACAGCCTGCTGCTGCTGCACAAGAAGGAGGACGGCATCTGGAGCACC |
| | | GACATCCTGAAGGACCAGAAGGAGCCCAAGAACAAGACCTTCCTGCGCTGCGAGGCCA |
| | | AGAACTACAGCGGCCGCTTCACCTGCTGGTGGCTGACCACCATCAGCACCGACCTGACC |
| | | TTCAGCGTCAAGAGCAGCCGCGGCAGCAGCGACCCCCAGGGCGTGACCTGCGGCGCCG |
| | | CCACCCTGAGCGCCGAGCGCGTGCGCGGCGACAACAAGGAGTACGAGTACAGCGTGG |
| | | AGTGCCAGGAGGACAGCGCCTGCCCCGCCGCCGAGGAGAGCCTGCCCATCGAGGTGAT |
| | | GGTGGACGCCGTGCACAAGCTGAAGTACGAGAACTACACCAGCAGCTTCTTCATCCGCG |
| | | ACATCATCAAGCCCGACCCCCCCAAGAACCTGCAGCTGAAGCCCCTGAAGAACAGCCGC |
| | | CAGGTGGAGGTGAGCTGGGAGTACCCCGACACCTGGAGCACCCCCCACAGCTACTTCA |
| | | GCCTGACCTTCTGCGTGCAGGTGCAGGGCAAGAGCAAGCGCGAGAAGAAGGACCGCG |
| | | TGTTCACCGACAAGACCAGCGCCACCGTGATCTGCCGCAAGAACGCCAGCATCAGCGTG |
| | | CGCGCCCAGGACCGCTACTACAGCAGCAGCTGGAGCGAGTGGGCCAGCGTGCCCTGCA |
| | | GCGGCGGCGGCGGCGGCGGCAGCCGCAACCTGCCCGTGGCCACCCCCGACCCCGGCAT |
| | | GTTCCCCTGCCTGCACCACAGCCAGAACCTGCTGCGCGCCGTGAGCAACATGCTGCAGA |
| | | AGGCCCGCCAGACCCTGGAGTTCTACCCCTGCACCAGCGAGGAGATCGACCACGAGGA |
| | | CATCACCAAGGACAAGACCAGCACCGTGGAGGCCTGCCTGCCCCTGGAGCTGACCAAG |
| | | AACGAGAGCTGCCTGAACAGCCGCGAGACCAGCTTCATCACCAACGGCAGCTGCCTGG |
| | | CCAGCCGCAAGACCAGCTTCATGATGGCCCTGTGCCTGAGCAGCATCTACGAGGACCTG |
| | | AAGATGTACCAGGTGGAGTTCAAGACCATGAACGCCAAGCTGCTGATGGACCCCAAGC |
| | | GCCAGATCTTCCTGGACCAGAACATGCTGGCCGTGATCGACGAGCTGATGCAGGCCCTG |
| | | AACTTCAACAGCGAGACCGTGCCCCAaAAGAGCAGCCTGGAGGAGCCCGACTTCTACAA |
| | | GACCAAGATCAAGCTGTGCATCCTGCTGCACGCCTTCCGCATCCGCGCCGTGACCATCG |
| | | ACCGCGTGATGAGCTACCTGAACGCCAGCTAATAGGCTGCCTTCTGGGGGGCTTGCCTT |
| | | CTGGCCATGCCCTTCTTCTCTCCCTTGCACCTGTACCTCTTGGTCTTTGAATAAAGCCTGA |
| | | GTAGGAAGTAAAAAAAAAAAAAAAAAAAAAAAAAAAAAAAAAAAAAAAAAAAAAAAA |
| | | AAAAAAAAAAAAAAAAAAAAAAAAAAA |
| 8 | 5′ UTR | AGGAAAUUCCAUUUGGCUGCAGCUUCUGGAGGGAGCCGACAGGAGACGUGGGGAG |
| | RNA | ACGGCCACC |
| 9 | 3′ UTR | GCUGCCUUCUGCGGGGCUUGCCUUCUGGCCAUGCCCUUCUUCUCUCCCUUGCACCU |
| | RNA | GUACCUCUUGGUCUUUGAAUAAAGCCUGAGUAGGAAGU |
| 10 | 5′ UTR | AGGAAATTCCATTTGGCTGCAGCTTCTGGAGGGAGCCGACAGGAGACGTGGGGAGAC |
| | DNA | GGCCACC |
| 11 | 3′ UTR | GCTGCCTTCTGCGGGGCTTGCCTTCTGGCCATGCCCTTCTTCTCTCCCTTGCACCTGTACC |
| | DNA | TCTTGGTCTTTGAATAAAGCCTGAGTAGGAAGT |
| 12 | PolyA RNA | AAAAAAAAAAAAAAAAAAAAAAAAAAAAAAAAAAAAAAAAAAAAAAAAAAAAAAAAAA |
| | | AAAAAAAAAAAAAAAAAA |
| 13 | PolyA DNA | AAAAAAAAAAAAAAAAAAAAAAAAAAAAAAAAAAAAAAAAAAAAAAAAAAAAAAAAAA |
| | | AAAAAAAAAAAAAAAAAA |
| 14 | T7 promoter | TAATACGACTCACTATA |
| 15 | Upstream | TTGGACCCTCGTACAGAAGCTAATACG |
| | primer | |
| 16 | Downstrea | TTTTTTTTTTTTTTTTTTTTTTTTTTTTTTTTTTTTTTTTTTTTTTTTTTTTTTTTTTTTTTTTTTTT |
| | m primer | TTTTTTTACTTCCTACTCAGGCTTTATTCAAAGACCA |
| 17 | rhIL12 | MCHQQLVISWFSLVFLASPLVAIWELKKDVYVVELDWYPDAPGEMVVLTCDTPEEDGITW |
| | | TLDQSSEVLGSGKTLTIQVKEFGDAGQYTCHKGGEVLSHSLLLLHKKEDGIWSTDILKDQKEP |
| | | KNKTFLRCEAKNYSGRFTCWWLTTISTDLTFSVKSSRGSSDPQGVTCGAATLSAERVRGDNK |
| | | EYEYSVECQEDSACPAAEESLPIEVMVDAVHKLKYENYTSSFFIRDIIKPDPPKNLQLKPLKNSR |
| | | QVEVSWEYPDTWSTPHSYFSLTFCVQVQGKSKREKKDRVFTDKTSATVICRKNASISVRAQD |
| | | RYYSSSWSEWASVPCSGGGGGGSRNLPVATPDPGMFPCLHHSQNLLRAVSNMLQKARQT |
| | | LEFYPCTSEEIDHEDITKDKTSTVEACLPLELTKNESCLNSRETSFITNGSCLASRKTSFMMALC |
| | | LSSIYEDLKMYQVEFKTMNAKLLMDPKRQIFLDQNMLAVIDELMQALNFNSETVPQKSSLE |
| | | EPDFYKTKIKLCILLHAFRIRAVTIDRVMSYLNAS |
| 18 | Expression- | UGUGACCACCAGCAGCUGGUGAUCAGCUGGUUCAGCCUGGUGUUCCUGGCCAGCCC |
| | free control | CCUGGUGGCCAUCUGGGAGCUGAAGAAGGACGUGUACGUGGUGGAGCUGGACUG |
| | RNA ORF | GUACCCCGACGCCCCCGGCGAGUGAGUGGUGCUGACCUGCGACACCCCCGAGGAGG |
| | sequence | ACGGCAUCACCUGGACCCUGGACCAGAGCAGCGAGGUGCUGGGCAGCGGCAAGACC |
| | | CUGACCAUCCAGGUGAAGGAGUUCGGCGACGCCGGCCAGUACACCUGCCACAAGGG |
| | | CGGCGAGGUGCUGAGCCACAGCCUGCUGCUGCUGCACAAGAAGGAGGACGGCAUCU |
| | | GGAGCACCGACAUCCUGAAGGACCAGAAGGAGCCCAAGAACAAGACCUUCCUGCGC |
| | | UGCGÅGGCCAAGAACUACAGCGGCCGCUUCACCUGCUGGUGGCUGACCACCAUCAG |
| | | CACCGACCUGACCUUCAGCGUCAAGAGCAGCCGCGGCAGCAGCGACCCCCAGGGCGU |
| | | GACCUGCGGCGCCGCCACCCUGAGCGCCGAGCGCGUGCGCGGCGACAACAAGGAGU |
| | | ACGAGUACAGCGUGGAGUGCCAGGAGGACAGCGCCUGCCCCGCCGCCGAGGAGAGC |
| | | CUGCCCAUCGAGGUGUGAGUGGACGCCGUGCACAAGCUGAAGUACGAGAACUACAC |
| | | CAGCAGCUUCUUCAUCCGCGACAUCAUCAAGCCCGACCCCCCCAAGAACCUGCAGCU |
| | | GAAGCCCCUGAAGAACAGCCGCCAGGUGGAGGUGAGCUGGGAGUACCCCGACACCU |
| | | GGAGCACCCCCCACAGCUACUUCAGCCUGACCUUCUGCGUGCAGGUGCAGGGCAAG |
| | | AGCAAGCGCGAGAAGAAGGACCGCGUGUUCACCGACAAGACCAGCGCCACCGUGAU |
| | | CUGCCGCAAGAACGCCAGCAUCAGCGUGCGCGCCCAGGACCGCUACUACAGCAGCAG |
| | | CUGGAGCGAGUGGGCCAGCGUGCCCUGCAGCGGCGGCGGCGGCGGCGGCAGCCGCA |
| | | ACCUGCCCGUGGCCACCCCCGACCCCGGCUGAUUCCCCUGCCUGCACCACAGCCAGA |
| | | ACCUGCUGCGCGCCGUGAGCAACUGACUGCAGAAGGCCCGCCAGACCCUGGAGUUC |
| | | UACCCCUGCACCAGCGAGGAGAUCGACCACGAGGACAUCACCAAGGACAAGACCAGC |
| | | ACCGUGGAGGCCUGCCUGCCCCUGGAGCUGACCAAGAACGAGAGCUGCCUGAACAG |
| | | CCGCGAGACCAGCUUCAUCACCAACGGCAGCUGCCUGGCCAGCCGCAAGACCAGCUU |
| | | CUGUGAAGCCCUGUGCCUGAGCAGCAUCUACGAGGACCUGAAGUGAUACCAGGUG |
| | | GAGUUCAAGACCUGAAACGCCAAGCUGCUGUGAGACCCCAAGCGCCAGAUCUUCCU |
| | | GGACCAGAACUGACUGGCCGUGAUCGACGAGCUGUGACAGGCCCUGAACUUCAACA |
| | | GCGAGACCGUGCCCCAAAAGAGCAGCCUGGAGGAGCCCGACUUCUACAAGACCAAG |
| | | AUCAAGCUGUGCAUCCUGCUGCACGCCUUCCGCAUCCGCGCCGUGACCAUCGACCGC |
| | | GUGUGAAGCUACCUGAACGCCAGCUAAUAG |
| 19 | Expression- | TGTGACCACCAGCAGCTGGTGATCAGCTGGTTCAGCCTGGTGTTCCTGGCCAGCCCCCT |
| | free control | GGTGGCCATCTGGGAGCTGAAGAAGGACGTGTACGTGGTGGAGCTGGACTGGTACCCC |
| | DNA ORF | GACGCCCCCGGCGAGTGAGTGGTGCTGACCTGCGACACCCCCGAGGAGGACGGCATCA |
| | sequence | CCTGGACCCTGGACCAGAGCAGCGAGGTGCTGGGCAGCGGCAAGACCCTGACCATCCA |
| | | GGTGAAGGAGTTCGGCGACGCCGGCCAGTACACCTGCCACAAGGGCGGCGAGGTGCT |
| | | GAGCCACAGCCTGCTGCTGCTGCACAAGAAGGAGGACGGCATCTGGAGCACCGACATC |
| | | CTGAAGGACCAGAAGGAGCCCAAGAACAAGACCTTCCTGCGCTGCGAGGCCAAGAACT |
| | | ACAGCGGCCGCTTCACCTGCTGGTGGCTGACCACCATCAGCACCGACCTGACCTTCAGC |
| | | GTCAAGAGCAGCCGCGGCAGCAGCGACCCCCAGGGCGTGACCTGCGGCGCCGCCACCC |
| | | TGAGCGCCGAGCGCGTGCGCGGCGACAACAAGGAGTACGAGTACAGCGTGGAGTGCC |
| | | AGGAGGACAGCGCCTGCCCCGCCGCCGAGGAGAGCCTGCCCATCGAGGTGTGAGTGG |
| | | ACGCCGTGCACAAGCTGAAGTACGAGAACTACACCAGCAGCTTCTTCATCCGCGACATC |
| | | ATCAAGCCCGACCCCCCCAAGAACCTGCAGCTGAAGCCCCTGAAGAACAGCCGCCAGGT |
| | | GGAGGTGAGCTGGGAGTACCCCGACACCTGGAGCACCCCCCACAGCTACTTCAGCCTG |
| | | ACCTTCTGCGTGCAGGTGCAGGGCAAGAGCAAGCGCGAGAAGAAGGACCGCGTGTTCA |
| | | CCGACAAGACCAGCGCCACCGTGATCTGCCGCAAGAACGCCAGCATCAGCGTGCGCGC |
| | | CCAGGACCGCTACTACAGCAGCAGCTGGAGCGAGTGGGCCAGCGTGCCCTGCAGCGGC |
| | | GGGGGGGGCGGCGGCAGCCGCAACCTGCCCGTGGCCACCCCCGACCCCGGCTGATTCC |
| | | CCTGCCTGCACCACAGCCAGAACCTGCTGCGCGCCGTGAGCAACTGACTGCAGAAGGCC |
| | | CGCCAGACCCTGGAGTTCTACCCCTGCACCAGCGAGGAGATCGACCACGAGGACATCAC |
| | | CAAGGACAAGACCAGCACCGTGGAGGCCTGCCTGCCCCTGGAGCTGACCAAGAACGAG |
| | | AGCTGCCTGAACAGCCGCGAGACCAGCTTCATCACCAACGGCAGCTGCCTGGCCAGCCG |
| | | CAAGACCAGCTTCTGTGAAGCCCTGTGCCTGAGCAGCATCTACGAGGACCTGAAGTGAT |
| | | ACCAGGTGGAGTTCAAGACCTGAAACGCCAAGCTGCTGTGAGACCCCAAGCGCCAGAT |
| | | CTTCCTGGACCAGAACTGACTGGCCGTGATCGACGAGCTGTGACAGGCCCTGAACTTCA |
| | | ACAGCGAGACCGTGCCCCAAAAGAGCAGCCTGGAGGAGCCCGACTTCTACAAGACCAA |
| | | GATCAAGCTGTGCATCCTGCTGCACGCCTTCCGCATCCGCGCCGTGACCATCGACCGCG |
| | | TGTGAAGCTACCTGAACGCCAGCTAATAG |
| 20 | Expression- | AGGAAAUUCCAUUUGGCUGCAGCUUCUGGAGGGAGCCGACAGGAGACGUGGGGAG |
| | free control | ACGGCCACCUGUGACCACCAGCAGCUGGUGAUCAGCUGGUUCAGCCUGGUGUUCCU |
| | RNA | GGCCAGCCCCCUGGUGGCCAUCUGGGAGCUGAAGAAGGACGUGUACGUGGUGGAG |
| | sequence | CUGGACUGGUACCCCGACGCCCCCGGCGAGUGAGUGGUGCUGACCUGCGACACCCC |
| | | CGAGGAGGACGGCAUCACCUGGACCCUGGACCAGAGCAGCGAGGUGCUGGGCAGCG |
| | | GCAAGACCCUGACCAUCCAGGUGAAGGAGUUCGGCGACGCCGGCCAGUACACCUGC |
| | | CACAAGGGCGGCGAGGUGCUGAGCCACAGCCUGCUGCUGCUGCACAAGAAGGAGGA |
| | | CGGCAUCUGGAGCACCGACAUCCUGAAGGACCAGAAGGAGCCCAAGAACAAGACCU |
| | | UCCUGCGCUGCGAGGCCAAGAACUACAGCGGCCGCUUCACCUGCUGGUGGCUGACC |
| | | ACCAUCAGCACCGACCUGACCUUCAGCGUCAAGAGCAGCCGCGGCAGCAGCGACCCC |
| | | CAGGGCGUGACCUGCGGCGCCGCCACCCUGAGCGCCGAGCGCGUGCGCGGCGACAA |
| | | CAAGGAGUACGAGUACAGCGUGGAGUGCCAGGAGGACAGCGCCUGCCCCGCCGCCG |
| | | AGGAGAGCCUGCCCAUCGAGGUGUGAGUGGACGCCGUGCACAAGCUGAAGUACGA |
| | | GAACUACACCAGCAGCUUCUUCAUCCGCGACAUCAUCAAGCCCGACCCCCCCAAGAA |
| | | CCUGCAGCUGAAGCCCCUGAAGAACAGCCGCCAGGUGGAGGUGAGCUGGGAGUACC |
| | | CCGACACCUGGAGCACCCCCCACAGCUACUUCAGCCUGACCUUCUGCGUGCAGGUGC |
| | | AGGGCAAGAGCAAGCGCGAGAAGAAGGACCGCGUGUUCACCGACAAGACCAGCGCC |
| | | ACCGUGAUCUGCCGCAAGAACGCCAGCAUCAGCGUGCGCGCCCAGGACCGCUACUAC |
| | | AGCAGCAGCUGGAGCGAGUGGGCCAGCGUGCCCUGCAGCGGCGGCGGCGGCGGCG |
| | | GCAGCCGCAACCUGCCCGUGGCCACCCCCGACCCCGGCUGAUUCCCCUGCCUGCACC |
| | | ACAGCCAGAACCUGCUGCGCGCCGUGAGCAACUGACUGCAGAAGGCCCGCCAGACCC |
| | | UGGAGUUCUACCCCUGCACCAGCGAGGAGAUCGACCACGAGGACAUCACCAAGGAC |
| | | AAGACCAGCACCGUGGAGGCCUGCCUGCCCCUGGAGCUGACCAAGAACGAGAGCUG |
| | | CCUGAACAGCCGCGAGACCAGCUUCAUCACCAACGGCAGCUGCCUGGCCAGCCGCAA |
| | | GACCAGCUUCUGUGAAGCCCUGUGCCUGAGCAGCAUCUACGAGGACCUGAAGUGA |
| | | UACCAGGUGGAGUUCAAGACCUGAAACGCCAAGCUGCUGUGAGACCCCAAGCGCCA |
| | | GAUCUUCCUGGACCAGAACUGACUGGCCGUGAUCGACGAGCUGUGACAGGCCCUGA |
| | | ACUUCAACAGCGAGACCGUGCCCCAAAAGAGCAGCCUGGAGGAGCCCGACUUCUACA |
| | | AGACCAAGAUCAAGCUGUGCAUCCUGCUGCACGCCUUCCGCAUCCGCGCCGUGACCA |
| | | UCGACCGCGUGUGAAGCUACCUGAACGCCAGCUAAUAGGCUGCCUUCUGCGGGGCU |
| | | UGCCUUCUGGCCAUGCCCUUCUUCUCUCCCUUGCACCUGUACCUCUUGGUCUUUGA |
| | | AUAAAGCCUGAGUAGGAAGUAAAAAAAAAAAAAAAAAAAAAAAAAAAAAAAAAAAA |
| | | AAAAAAAAAAAAAAAAAAAAAAAAAAAAAAAAAAAAAAA |
| 21 | Expression- | AGGAAATTCCATTTGGCTGCAGCTTCTGGAGGGAGCCGACAGGAGACGTGGGGAGAC |
| | free control | GGCCACCTGTGACCACCAGCAGCTGGTGATCAGCTGGTTCAGCCTGGTGTTCCTGGCCA |
| | DNA | GCCCCCTGGTGGCCATCTGGGAGCTGAAGAAGGACGTGTACGTGGTGGAGCTGGACTG |
| | sequence | GTACCCCGACGCCCCCGGCGAGTGAGTGGTGCTGACCTGCGACACCCCCGAGGAGGAC |
| | | GGCATCACCTGGACCCTGGACCAGAGCAGCGAGGTGCTGGGCAGCGGCAAGACCCTGA |
| | | CCATCCAGGTGAAGGAGTTCGGCGACGCCGGCCAGTACACCTGCCACAAGGGCGGCGA |
| | | GGTGCTGAGCCACAGCCTGCTGCTGCTGCACAAGAAGGAGGACGGCATCTGGAGCACC |
| | | GACATCCTGAAGGACCAGAAGGAGCCCAAGAACAAGACCTTCCTGCGCTGCGAGGCCA |
| | | AGAACTACAGCGGCCGCTTCACCTGCTGGTGGCTGACCACCATCAGCACCGACCTGACC |
| | | TTCAGCGTCAAGAGCAGCCGCGGCAGCAGCGACCCCCAGGGCGTGACCTGCGGCGCCG |
| | | CCACCCTGAGCGCCGAGCGCGTGCGCGGCGACAACAAGGAGTACGAGTACAGCGTGG |
| | | AGTGCCAGGAGGACAGCGCCTGCCCCGCCGCCGAGGAGAGCCTGCCCATCGAGGTGTG |
| | | AGTGGACGCCGTGCACAAGCTGAAGTACGAGAACTACACCAGCAGCTTCTTCATCCGCG |
| | | ACATCATCAAGCCCGACCCCCCCAAGAACCTGCAGCTGAAGCCCCTGAAGAACAGCCGC |
| | | CAGGTGGAGGTGAGCTGGGAGTACCCCGACACCTGGAGCACCCCCCACAGCTACTICA |
| | | GCCTGACCTTCTGCGTGCAGGTGCAGGGCAAGAGCAAGCGCGAGAAGAAGGACCGCG |
| | | TGTTCACCGACAAGACCAGCGCCACCGTGATCTGCCGCAAGAACGCCAGCATCAGCGTG |
| | | CGCGCCCAGGACCGCTACTACAGCAGCAGCTGGAGCGAGTGGGCCAGCGTGCCCTGCA |
| | | GCGGCGGCGGCGGCGGCGGCAGCCGCAACCTGCCCGTGGCCACCCCCGACCCCGGCTG |
| | | ATTCCCCTGCCTGCACCACAGCCAGAACCTGCTGCGCGCCGTGAGCAACTGACTGCAGA |
| | | AGGCCCGCCAGACCCTGGAGTTCTACCCCTGCACCAGCGAGGAGATCGACCACGAGGA |
| | | CATCACCAAGGACAAGACCAGCACCGTGGAGGCCTGCCTGCCCCTGGAGCTGACCAAG |
| | | AACGAGAGCTGCCTGAACAGCCGCGAGACCAGCTTCATCACCAACGGCAGCTGCCTGG |
| | | CCAGCCGCAAGACCAGCTTCTGTGAAGCCCTGTGCCTGAGCAGCATCTACGAGGACCTG |
| | | AAGTGATACCAGGTGGAGTTCAAGACCTGAAACGCCAAGCTGCTGTGAGACCCCAAGC |
| | | GCCAGATCTTCCTGGACCAGAACTGACTGGCCGTGATCGACGAGCTGTGACAGGCCCTG |
| | | AACTTCAACAGCGAGACCGTGCCCCAAAAGAGCAGCCTGGAGGAGCCCGACTTCTACA |
| | | AGACCAAGATCAAGCTGTGCATCCTGCTGCACGCCTTCCGCATCCGCGCCGTGACCATC |
| | | GACCGCGTGTGAAGCTACCTGAACGCCAGCTAATAGGCTGCCTTCTGCGGGGCTTGCCT |
| | | TCTGGCCATGCCCTTCTTCTCTCCCTTGCACCTGTACCTCTTGGTCTTTGAATAAAGCCTG |
| | | AGTAGGAAGTAAAAAAAAAAAAAAAAAAAAAAAAAAAAAAAAAAAAAAAAAAAAAAA |
| | | AAAAAAAAAAAAAAAAAAAAAAAAAAAA |

1. A lipid composition, comprising a therapeutic agent or a prophylactic agent and a lipid encapsulating the therapeutic agent or the prophylactic agent, wherein the lipid encapsulating the therapeutic agent or the prophylactic agent comprises a cationic lipid, a phospholipid, a steroid, and a polyethylene glycol modified lipid; and the composition further comprises a cationic polymer, wherein the cationic polymer and the therapeutic agent or the prophylactic agent are associated as a complex and co-encapsulated in the lipid to form a lipopolyplex; and wherein the cationic lipid comprises a lipid compound of formula (I), or a pharmaceutically acceptable salt thereof, 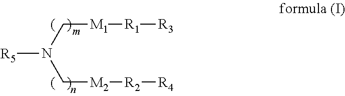
where
R₁ and R₂ are each independently selected from C₁-C₁₂ alkyl and C₂-C₁₂ alkenyl;
R₃ and R₄ are each independently selected from C₁-C₁₂ alkyl, C₂-C₁₂ alkenyl, C₆-C₁₀ aryl, and 5-10 membered heteroaryl;
R₃ and R₄ are each independently optionally substituted with t R₆, t being an integer selected from 1-5;
R₆ is independently selected from C₁-C₁₂ alkyl and C₂-C₁₂ alkenyl; and
M₁ and M₂ are each independently selected from —OC(O)—, —C(O)O—, —SC(S)—, and —C(S)S—;
R₅ is selected from —C₁₋₁₂ alkylene-Q, Q is selected from —OR₇ and —SR₇, and R₇ is independently selected from H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamido, C₆-C₁₀ aryl, and 5-10 membered heteroaryl; and
m and n are each independently an integer selected from 1-12.

2. The lipid composition of claim 1, wherein the therapeutic agent or the prophylactic agent is a nucleic acid, such as an RNA, in particular an mRNA.

3. The lipid composition of claim 1, wherein the cationic lipid comprises a lipid compound having a structure shown below, or a pharmaceutically acceptable salt thereof:

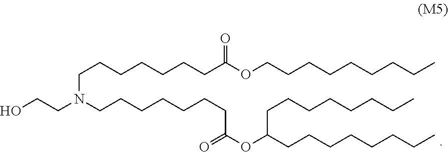 [SMILES: CCCCCCCCCOC(=O)CCCCCCCN(CCO)CCCCCCCC(=O)OC(CCCCCCCC)CCCCCCCC]

4. The lipid composition of claim 1, wherein
at least one of R₃ and R₄ is C₆-C₁₀ aryl or 5-10 membered heteroaryl.

5. The lipid composition of claim 4, wherein
R₁ and R₂ are each independently selected from C₁-C₁₂ alkyl.

6. The lipid composition of claim 4, wherein
R₃ and R₄ are each independently selected from C₁-C₁₂ alkyl and C₆-C₁₀ aryl,
with a proviso that one of R₃ and R₄ is C₆-C₁₀ aryl, and the other is C₁-C₁₂ alkyl;
R₃ and R₄ are each independently substituted with t R₆, t being an integer selected from 1-3; and
R₆ is independently selected from C₁-C₁₂ alkyl.

7. The lipid composition of claim 6, wherein
M₁ and M₂ are each independently selected from: —OC(O)— and —C(O)O—.

8. The lipid composition of claim 7, wherein
R₅ is selected from —C₁₋₅ alkylene-Q, and Q is —OH.

9. The lipid composition of claim 8, wherein
m and n are each independently an integer selected from 2-7.

10. The lipid composition of claim 9, wherein
R₄ is substituted at a 1-position or a last position of R₂; and/or
R₃ is substituted at a 1-position or a last position of R₁.

11. The lipid composition of claim 10, wherein
t is 1 or 2, and R₆ is substituted at a meta-position and/or a para-position on a benzene ring relative to R₁ or R₂.

12. The lipid composition of claim 11, wherein
t is 1 or 2, and R₆ is independently selected from C₁-C₁₀ alkyl.

13. The lipid composition of claim 12, wherein the cationic lipid comprises a compound of formula (II), or a pharmaceutically acceptable salt thereof: 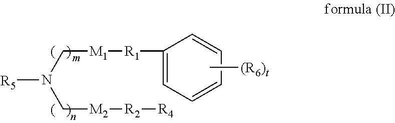
where R₁, R₂, R₄, R₅, R₆, M₁, M₂, t, m, and n are as defined in claim 12; and
preferably, in formula (II),
R₁ is selected from C₁-C₆ alkyl;
R₂ is selected from C₁-C₁₀ alkyl;
R₄ is selected from C₁-C₁₀ alkyl;
M₁ and M₂ are each independently selected from: —OC(O)— and —C(O)O—;
R₅ is selected from —C₁₋₅ alkylene-Q, Q is selected from —OR₇ and —SR₇, and R₇ is independently selected from H, C₁-C₁₂ alkyl, and C₂-C₁₂ alkenyl;
m and n are each independently an integer selected from 2-9;
t is an integer selected from 1-3; and
R₆ is independently selected from C₁-C₁₂ alkyl and C₂-C₁₂ alkenyl.

14. The lipid composition of claim 12, wherein the cationic lipid comprises a compound of formula (III), or a pharmaceutically acceptable salt thereof: 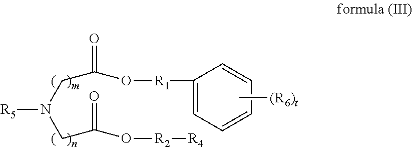
where R₁, R₂, R₄, R₅, R₆, t, m, and n are as defined in claim 12; and
preferably, in formula (III),
R₁ is selected from C₁-C₆ alkyl;
R₂ is selected from C₁-C₁₀ alkyl;
R₄ is selected from C₁-C₁₀ alkyl;
R₅ is selected from —C₁₋₃ alkylene-Q, and Q is selected from —OH and —SH;
t is 1 or 2;
R₆ is selected from C₁-C₁₂ alkyl and C₂-C₁₂ alkenyl; and
m and n are each independently an integer selected from 2-7.

15. The lipid composition of claim 12, wherein the cationic lipid comprises a compound of formula (IV), or a pharmaceutically acceptable salt thereof: 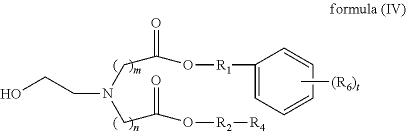
where R₁, R₂, R₄, R₆, t, m, and n are as defined in claim 12; and
preferably, in formula (IV),
R₁ is selected from C₁-C₆ alkyl;
R₂ is selected from C₁-C₁₀ alkyl;
R₄ is selected from C₁-C₁₀ alkyl;
t is 1 or 2;
R₆ is independently selected from C₁-C₁₂ alkyl and C₂-C₁₂ alkenyl; and
m and n are each independently an integer selected from 2-7.

16. The lipid composition of claim 4, wherein the cationic lipid comprises a lipid compound having a structure shown below, or a pharmaceutically acceptable salt thereof. 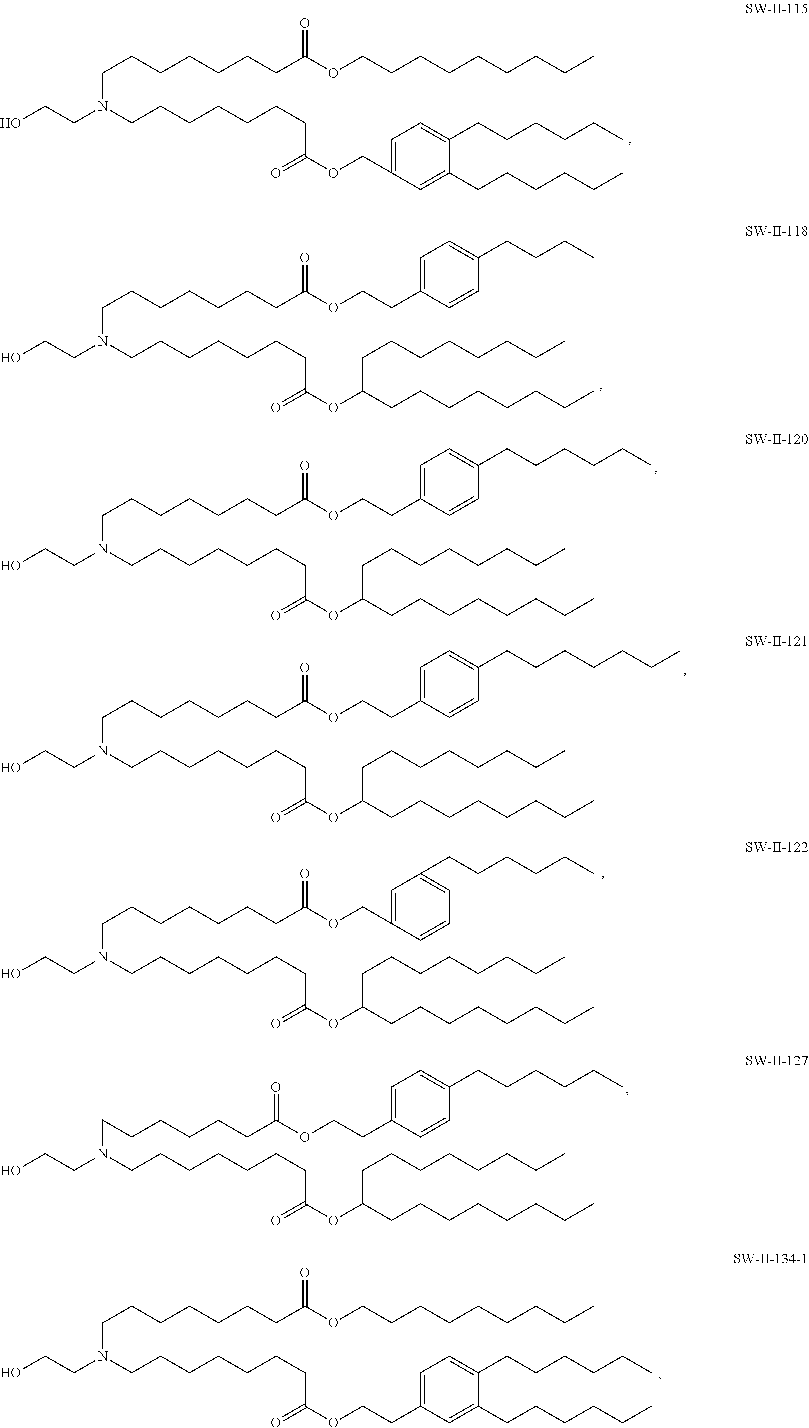 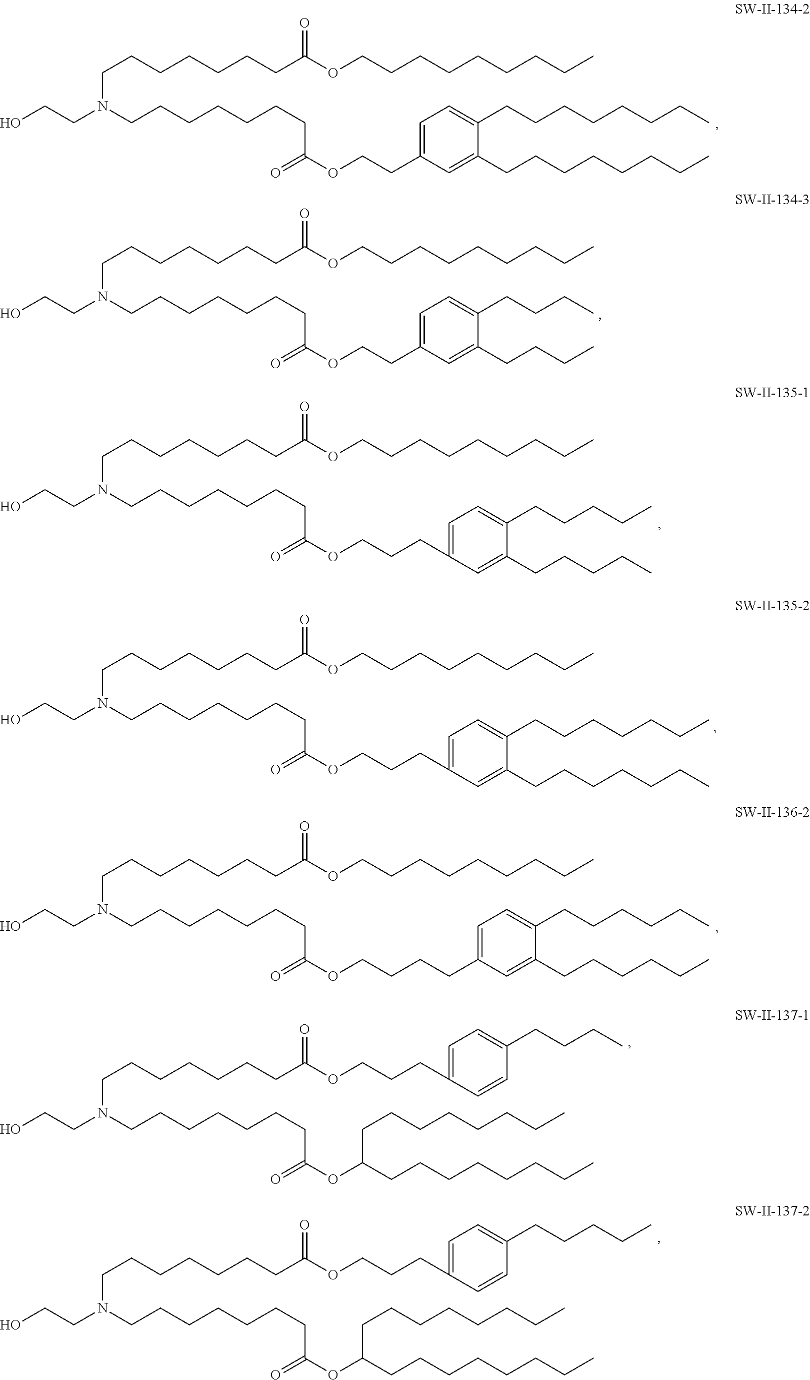 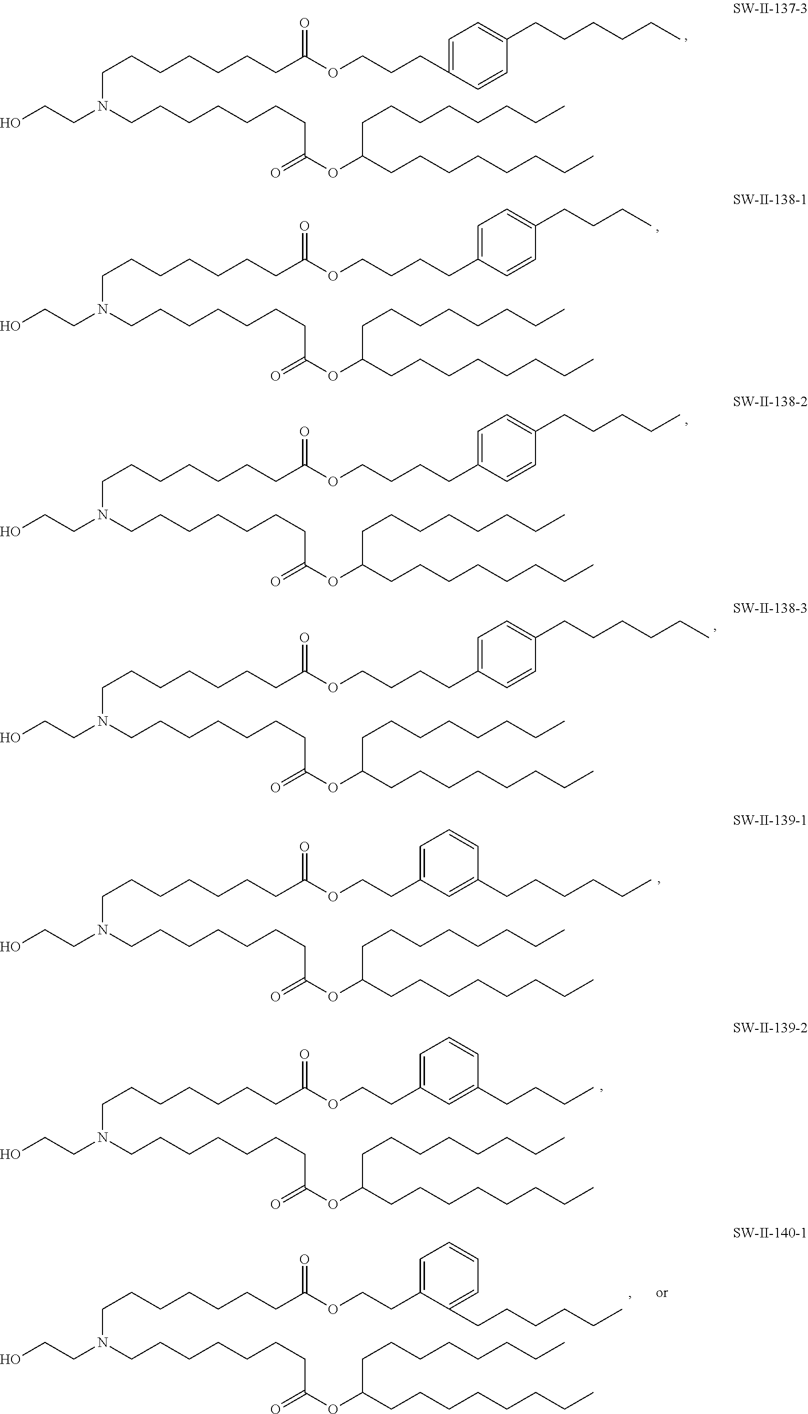 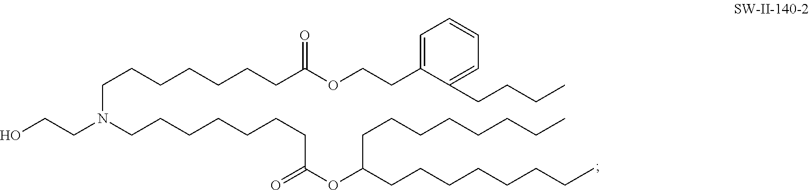 [SMILES: CCCCCCCCC(CCCCCCCC)OC(=O)CCCCCCCN(CCO)CCCCCCCC(=O)OCCc1ccccc1CCCC]
and preferably, the cationic lipid is SW-II-127, SW-II-135-1, or SW-II-138-1.

17. The lipid composition of claim 16, wherein the phospholipid comprises 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₆ Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleylphosphatidylethanolamine (POPE), distearoyl-phosphatidyl-ethanolamine (DSPE), dipalmitoylphosphatidylethanolamine (DPPE), dimyristoylphosphoethanolamine (DMPE), 1-stearoyl-2-oleyl-stearylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE), or a combination thereof, and
preferably, DSPC, DOPE, or a combination thereof.

18. The lipid composition of claim 17, wherein the steroid comprises cholesterol, coprosterol, sitosterol, ergosterol, campesterol, stigmasterol, brassicasterol, tomatidine, ursolic acid, α-tocopherol, and derivatives thereof, and preferably, the steroid is the cholesterol.

19. The lipid composition of claim 18, wherein the polyethylene glycol modified lipid comprises 1,2-dimyristoyl-rac-glycero-3-methoxypolyethylene glycol (DMG-PEG), 1,2-dioleoyl-rac-glycerol, methoxy-polyethylene glycol (DOGPEG), and 1,2-distearoyl-sn-glycero-3-phosphoethanolamine-poly(ethylene glycol) (DSPE-PEG), and preferably, DSPE-PEG, DMG-PEG, or a combination thereof.

20. The lipid composition of claim 19, comprising
10-70 mol % of the cationic lipid, 10-70 mol % of the phospholipid, 10-70 mol % of the steroid, and 0.05-20 mol % of the polyethylene glycol modified lipid;
preferably comprising 30-45 mol % of the cationic lipid, 10-20 mol % of the phospholipid, 30-48.5 mol % of the steroid, and 1-1.5 mol % of the polyethylene glycol modified lipid; and/or
the cationic lipid, DOPE, the cholesterol, and DMG-PEG; and
preferably comprising 40% of the cationic lipid, 15% of DOPE, 43.5% of the cholesterol, and 1.5% of DMG-PEG.

21-33. (canceled)
